(19) United States
(12) Patent Application Publication
(10) Pub. No.: US 2023/0263434 A1
(43) Pub. Date: Aug. 24, 2023

(54) SENSING SYSTEMS AND METHODS FOR PROVIDING DECISION SUPPORT AROUND KIDNEY HEALTH AND/OR DIABETES

(71) Applicant: Dexcom, Inc., San Diego, CA (US)

(72) Inventors: Matthew L. JOHNSON, San Diego, CA (US); Partha Pratim RAY, San Diego, CA (US); Qi AN, San Diego, CA (US); Rush BARTLETT, Austin, TX (US); John PADERI, San Francisco, CA (US)

(21) Appl. No.: 18/173,753

(22) Filed: Feb. 23, 2023

Related U.S. Application Data
(60) Provisional application No. 63/268,417, filed on Feb. 23, 2022.
(60) Provisional application No. 63/377,332, filed on Sep. 27, 2022.
(60) Provisional application No. 63/403,568, filed on Sep. 2, 2022.
(60) Provisional application No. 63/403,582, filed on Sep. 2, 2022.

(51) Int. Cl. A61B 5/145; A61B 5/1486; A61B 5/00; A61B 5/0205

(52) U.S. Cl. CPC A61B 5/14546; A61B 5/14865; A61B 5/7275; A61B 5/0205; A61B 5/7267; A61B 5/0004

(57) ABSTRACT

Certain aspects of the present disclosure relate to methods and systems for providing decision support around kidney disease. In certain aspects, a method includes monitoring one or more analytes of the patient during a plurality of time periods to obtain analyte data, the one or more analytes including at least potassium and the analyte data containing potassium data, processing the analyte data from the plurality of time periods to determine at least one rate of change of potassium for the patient based on the potassium data, and generating a disease prediction using the analyte data for the one or more analytes, including the potassium data and the at least one rate of change of potassium for the patient.

### CROSS-REFERENCE TO RELATED APPLICATIONS

This application claims priority to and benefit of U.S. Provisional Patent Application No. 63/268,417, filed Feb. 23, 2022, and U.S. Provisional Application No. 63/377,332, filed Sep. 27, 2022, which are hereby assigned to the assignee hereof and hereby expressly incorporated by reference in their entirety as if fully set forth below and for all applicable purposes.

### BACKGROUND

The kidney is responsible for many critical functions within the human body including, filtering waste and excess fluids, which are excreted in the urine, and removing acid that is produced by the cells of the body to maintain a healthy balance of water, salts, and minerals (e.g., such as sodium, calcium, phosphorus, and potassium) in the blood. In other words, the kidney plays a major role in homeostasis by renal mechanisms that transport and regulate water, salt, and mineral secretion, reabsorption, and excretion. Further, kidneys secrete renin (e.g., angiotensinogenase), which forms part of the renin-angiotensin-aldosterone system (RAAS) that mediates extracellular fluid and arterial vasoconstriction (e.g., blood pressure). More specifically, high blood pressure (e.g., hypertension) can be regulated through RAAS inhibitors such as angiotensin-converting enzyme (ACE) inhibitors and angiotensin receptor blockers (ARBs). Should the kidney become diseased or injured, the impairment or loss of these functions can cause significant damage to the human body.

Kidney disease occurs when the kidney becomes diseased or injured. Kidney disease is generally classified as either acute or chronic based upon the duration of the disease. Acute kidney injury (AKI) (also referred to as “acute renal failure”) is usually caused by an event that leads to kidney malfunction, such as dehydration, blood loss from major surgery or injury, and/or the use of medicines. On the other hand, chronic kidney disease (CKD) is usually caused by a long-term disease, such as high blood pressure or diabetes, which slowly damages the kidneys and reduces their function over time.

Symptoms of decreased kidney function, such as fluid buildup or electrolyte imbalance, are more likely to develop with AKI, regardless of how long the kidney has been malfunctioning. Such symptoms may reflect the actual cause of the kidney problem. For example, dehydration may cause extreme thirst, lightheadedness or faintness, and/or a weak, rapid pulse. On the other hand, symptoms of CKD develop slowly and may not be apparent until very little kidney function remains. Depending upon how severe the kidney disease is in a person, loss of kidney function can cause, to name a few, numbness, tingling, shortness of breath, muscle weakness, and/or arrhythmia.

In some cases, elevated potassium levels of a patient with CKD may lead to hyperkalemia, while lower potassium levels of a patient with CKD may lead to hypokalemia. In particular, hyperkalemia is the medical term that describes a potassium level in the blood that is higher than normal (e.g., higher than normal blood potassium levels between 3.6 and 5.2 millimoles per liter (mmol/L)). Hyperkalemia increases the risk of cardiac arrhythmia episodes and sudden death. Symptoms of consistent or mild hyperkalemia are muscle weakness, numbness, tingling, nausea, or other unusual feelings or no symptoms noticeable by the patient at all. Noticed symptoms of highly elevated potassium levels include heart palpitations, shortness of breath, chest pain, nausea, or vomiting. On the other hand, hypokalemia is the medical term that describes a potassium level in the blood that is lower than normal. In particular, CKD patients may develop hypokalemia due to gastrointestinal potassium loss from diarrhea or vomiting or renal potassium loss from non-potassium-sparing diuretics (e.g., diuretics used to increase the amount of fluid passed from the body in urine, without regard for the amount of potassium being lost from the body in the urine). The lack of readily noticeable symptoms in many patients is why hyperkalemia and hypokalemia are often referred to as silent killers, especially when patients have become sensitized to irregular potassium levels. Severe hypokalemia and hyperkalemia may lead to severe symptoms of respiratory failure, sudden cardiac death, or other mortality-driven event, where a diagnosis, e.g., being hypokalemia or hyperkalemia, as the mediating mechanism, may not be readily apparent by the time a patient is evaluated by medical personnel.

Further, CKD may alter glucose homeostasis of a patient, thereby making CKD an independent risk factor for hypoglycemia and hyperglycemia. In particular, the kidneys also play an important role in the regulation of blood glucose (e.g., blood sugar). With respect to renal involvement in glucose homeostasis, the primary mechanisms include release of glucose into circulation via gluconeogenesis, uptake of glucose from the circulation to satisfy the kidneys' energy needs, and reabsorption of glucose at the level of the proximal tubule of the kidney. For example, gluconeogenesis is the formation of glucose from precursors lactate, glycerol and/or amino acids). Glucose is formed by the kidneys and released into circulation. Gluconeogenesis occurs to maintain glucose homeostasis by preventing low blood glucose.

As kidney function declines however, the formation of glucose also declines, and thus limits the kidney's ability to react to falling blood glucose. In some cases, a reduction in the kidney's ability to react to falling blood glucose levels may lead to hypoglycemia. Hypoglycemia is the medical term that describes a blood sugar (e.g., glucose) level that is lower than normal (e.g., a blood sugar level below 70 milligrams per deciliter (mg/dL), or 3.9 millimoles per liter (mmol/L)). 1.7% of hospitalizations annually are due to hypoglycemia for early-stage CKD (CKD<3); for CKD stage 3 and 4, the annual hospitalization rate is 2.3%. Further, for end stage renal disease (ESRD)-related hospitalizations, 4.3% are due to hypoglycemia with a 30% mortality rate. In particular, severe hypoglycemia can lead to damage of the heart muscle, neurocognitive dysfunction, and in certain cases, seizures or even death.

As such, kidney dysfunction may result in lower and/or longer low glucose levels. Impaired kidneys may be slower, and, in some cases, less effective, at combating falling glucose levels resulting in poor glucose control (e.g., given kidneys filter insulin and generate glucose through gluconeogenesis). Hypoglycemic events in kidney disease result from decreased insulin clearance and impaired gluconeogenesis by the kidneys. For ESRD-related hospitalizations, dialysis is also a compounding factor.

Further, decreased insulin metabolism and clearance may occur as a result of declining kidney health. Insulin is a hormone that allows the body to use glucose for energy, or store glucose as fat. In other words, insulin stimulates potassium and glucose uptake by a patient's cells, reducing serum (e.g., extracellular) potassium and glucose levels. Insulin is cleared by the kidneys; thus, as kidney function declines, insulin is cleared more slowly. Accordingly, a typical dose of insulin may have a prolonged and/or pronounced effect on glucose in a patient with kidney dysfunction. As kidney dysfunction progresses, insulin may have an even more prolonged and/or pronounced effect on glucose. Thus, a patient with kidney disease may be at risk for hypoglycemia, and the risk increases with disease progression. For example, a patient with CKD may find that an insulin dose that is predictive of glucose clearance at one point in time later results in hypoglycemia.

Kidney dysfunction may also result in higher and/or longer high glucose levels. For example, impaired kidneys may be slower and, in some cases, less effective at reducing glucose levels (i.e., clearing glucose) given kidneys are response for filtering, reabsorbing, and consuming glucose from the blood. Patients suffering from higher and/or longer high glucose levels may be diagnosed as hyperglycemic. Hyperglycemia is the medical term that describes a blood sugar (e.g., glucose) level that is higher than normal (e.g., significantly elevated blood sugar levels, usually elevated above 180 to 200 mg/dL, or 10 to 11.1 mmol/L). Patients with hyperglycemia may suffer from an array of negative physiological effects (for example, nerve damage (neuropathy), kidney failure, skin ulcers, diabetic ketoacidosis, or bleeding into the vitreous of the eye) associated with the deterioration of small blood vessels.

Other problems may also develop with CKD, such as increased levels of phosphates in the blood (e.g., hyperphosphatemia). Hyperphosphatemia (e.g., abnormally high serum phosphate levels) can result from increased phosphate intake, decreased phosphate excretion, or a disorder that shifts intracellular phosphate to extracellular space. This increase in serum phosphate levels is associated with decreased renal ion excretion, as well as, the use of medications to reduce the progression of CKD or to control associated diseases such as diabetes mellitus and heart failure.

### BRIEF DESCRIPTION OF THE DRAWINGS

So that the manner in which the above-recited features of the present disclosure can be understood in detail, a more particular description, briefly summarized above, may be had by reference to aspects, some of which are illustrated in the drawings. It is to be noted, however, that the appended drawings illustrate only certain typical aspects of this disclosure and are therefore not to be considered limiting of its scope, for the description may admit to other equally effective aspects.

FIG. 1 illustrates aspects of an example decision support system that may be used in connection with implementing embodiments of the present disclosure.

FIG. 2 is a diagram conceptually illustrating an example continuous analyte monitoring system including example continuous analyte sensor(s) with sensor electronics, in accordance with certain aspects of the present disclosure.

FIG. 3 illustrates example inputs and example metrics that are calculated based on the inputs for use by the decision support system of FIG. 1, according to some embodiments disclosed herein.

FIG. 4A is an example workflow for determining, using one or more models or rules, the likelihood of one or more symptoms commonly associated with potassium imbalance occurring in real-time or within a specified period of time and retraining or updating the one or more models or rules, according to certain embodiments of the present disclosure.

FIG. 4B is an example workflow for determining the likelihood of one or more symptoms commonly associated with potassium imbalance occurring in real-time or within a specified period of time and providing one or more recommendations for treatment for the user based the determined likelihood, according to certain embodiments of the present disclosure.

FIG. 5 is a flow diagram illustrating an example method for providing decision support using a continuous analyte sensor including, at least, a continuous potassium sensor, in accordance with some example aspects of the present disclosure.

FIG. 6 is a flow diagram depicting a method for training machine learning models to provide predictions associated with kidney disease, according to certain embodiments of the present disclosure.

FIG. 7 is an example workflow for stratifying a user's risk of disease(s) commonly associated with kidney disease, including hyperkalemia and hypokalemia, according to certain embodiments of the present disclosure.

FIGS. 8A and 8B illustrate an example method used for providing decision support around kidney disease for a user determined to be at risk of hyperkalemia, according to certain embodiments of the present disclosure.

FIG. 9 illustrates an example method used for providing recommendations of treatment for kidney disease and/or glucose homeostasis for a user determined to be at risk of hyperkalemia, according to certain embodiments of the present disclosure.

FIG. 10 illustrates an example method used for providing diet recommendations for a user determined to be at risk of hyperkalemia, according to certain embodiments of the present disclosure.

FIG. 11 illustrates an example method used for providing decision support around kidney disease for a user determined to be at risk of hypokalemia, according to certain embodiments of the present disclosure.

FIG. 12 illustrates an example method used for providing recommendations of treatment for kidney disease and/or glucose homeostasis for a user determined to be at risk of hypokalemia, according to certain embodiments of the present disclosure.

FIG. 13 illustrates an example method used for providing diet recommendations for a user determined to be at risk of hypokalemia, according to certain embodiments of the present disclosure.

FIG. 14 is a block diagram depicting a computing device configured to perform the operations of FIGS. 4A-13, according to certain embodiments of the present disclosure.

FIGS. 15A-15B depict exemplary enzyme domain configurations for a continuous multi-analyte sensor, according to certain embodiments of the present disclosure.

FIGS. 15C-15D depict exemplary enzyme domain configurations for a continuous multi-analyte sensor, according to certain embodiments of the present disclosure.

FIG. 15E depicts an exemplary enzyme domain configuration for a continuous multi-analyte sensor, according to certain embodiments of the present disclosure.

FIGS. 16A-16B depict alternative views of an exemplary dual electrode enzyme domain configuration for a continuous multi-analyte sensor, according to certain embodiments of the present disclosure.

FIGS. 16C-16D depict alternative views of an exemplary dual electrode enzyme domain configuration for a continuous multi-analyte sensor, according to certain embodiments of the present disclosure.

FIG. 16E depicts an exemplary dual electrode configuration for a continuous multi-analyte sensor, according to certain embodiments of the present disclosure.

FIG. 17A depicts an exemplary enzyme domain configuration for a continuous multi-analyte sensor, according to certain embodiments of the present disclosure.

FIGS. 17B-17C depict alternative exemplary enzyme domain configurations for a continuous multi-analyte sensor, according to certain embodiments of the present disclosure.

FIG. 18 depicts an exemplary enzyme domain configuration for a continuous multi-analyte sensor, according to certain embodiments of the present disclosure.

FIGS. 19A-19D depict alternative views of exemplary dual electrode enzyme domain configurations G1-G4 for a continuous multi-analyte sensor, according to certain embodiments of the present disclosure.

FIGS. 20A-20B schematically illustrate an example configuration and component of a device for measuring an electrophysiological signal and/or concentration of a target ion in a biological fluid in vivo, according to certain embodiments of the present disclosure.

FIG. 21 schematically illustrates additional example configurations and component of a device for measuring an electrophysiological signal and/or a concentration of a target ion in a biological fluid in vivo, according to certain embodiments of the present disclosure.

FIGS. 22A-22C schematically illustrate example configurations and components of a device for measuring an electrophysiological signal and/or concentration of a target analyte in a biological fluid in vivo, according to certain embodiments of the present disclosure.

FIG. 23 is a diagram depicting an example continuous analyte monitoring system configured to measure target ions and/or other analytes as discussed herein, according to certain embodiments of the present disclosure.

To facilitate understanding, identical reference numerals have been used, where possible, to designate identical elements that are common to the figures. It is contemplated that elements disclosed in one aspect may be beneficially utilized on other aspects without specific recitation.

### DETAILED DESCRIPTION

Accurate assessment of kidney function (as well as heart function, in some cases) is important as a screening tool and for monitoring disease progression and guiding prognosis at least with respect to kidney disease, and further hyperkalemia, hypokalemia, hyperglycemia, and hypoglycemia. However, conventional disease diagnostic methods and systems for such diseases, including, but not limited to, electrocardiogram (ECG) monitoring, albumin-to-creatinine ratio (ACR) tests, glomerular filtration rate (GFR) tests, and blood tests for monitoring potassium levels of a patient, face many challenges with respect to accuracy and reliability. Further, conventional disease diagnostic methods and systems generally fail to provide an efficient and complete analysis of factors which may likely contribute to each of these diseases.

For example, ECG monitoring has been touted as a method used to recognize the arrhythmogenic effects of severe hyperkalemia, such as peaked T wave, QRS widening, PR shortening (e.g., the PR interval is the time from the beginning of the P wave (atrial depolarization) to the beginning of the QRS complex (ventricular depolarization), and a shortened PR interval may indicate a certain disease), and bradycardia (e.g., slower-than-expected heart rate). However, by the time an ECG device is able to recognize arrhythmogenic effects of severe hyperkalemia or hypokalemia, a patient may be in a severe hyperkalemic or hypokalemic situation. Similarly, by the time an ECG device recognizes arrhythmogenic effects of severe hypokalemia (e.g., ST segment depression, U waves, T-wave morphology changes), the patient is already in a dangerous situation. Accordingly, the only treatments that may be available may be treatments provided for the patient in an emergency room setting. Such treatment may not always be feasible or performed in sufficient time, thereby causing undesirable outcomes, and in some cases, death of the patient. A further treatment paradox exists in that ECG devices, or the ability to interpret such ECG devices, are not readily accessible to most patients, for example in their own homes. As such, a patient's only reasonable option in a severe disease state may be to present to emergency personnel.

Further, evidence is conflicting as to whether in-clinic ECG findings are reliable, especially in patients with chronic hyperkalemia. For example, high false-positives and high-false negatives are often seen with the use of ECG monitors. As used herein, a false positive is a result that indicates a given condition exists when the condition does not, and a false negative is a result that indicates a given condition does not exist when, in fact, the condition does exist. Further, such monitoring lacks the ability for continuous monitoring which is needed to provide a complete picture as to a patient's health.

Additionally, in some cases, T wave abnormalities detected by ECG monitoring may be attributed to factors unrelated to potassium. For example, T wave abnormalities may be present in subarachnoid hemorrhage, ischemic stroke, subdural hematoma, heart failure, myocardial edema, viral infection (e.g., Covid-19), traumatic brain injury, rare diseases, and specific oncologic pathways such as, but not limited to, pheochromocytoma. The value of using ECG alone to diagnose clinically significant hyperkalemia is further complicated by situations where T wave abnormalities are co-morbidities to kidney disease. In these cases, it is even more difficult to determine if the result of the T wave abnormality is a result of the kidney disease, or another clinical situation. Accordingly, T wave abnormalities recognized by ECG monitoring may not always be caused by decreased kidney function (e.g., abnormal potassium levels may, in some cases, be attributed to decreased kidney function). Thus, ECG alone may not provide sufficient information for assessing kidney health, nor provide sufficient information about the extent of kidney disease in a patient's body.

As another example, ACR and GFR tests are commonly used for screening and diagnosing kidney disease. In particular, an ACR test measures both albumin and creatinine in a one-time sample, also known as a spot urine sample. Urine albumin test is the first method of preference to detect elevated protein, specifically albumin, in the urine. Persistent increased albumin levels in the urine, i.e., increased albumin excretion, measured using the ACR test provides a marker of kidney damage; however, the test is not without flaw. For example, a failure to consider the influence of creatinine excretion in the current use of ACR tests may lead to inaccurate results, and consequently be misleading. Further, spot urine samples for ACR are more vulnerable to transient changes in excretion of creatinine and albumin than timed collections that average such changes over a longer time period; thus, random spot urine ACR results are less consistent than timed urine samples.

On the other hand, GFR is the flow rate of filtered fluid through the kidney. Creatinine clearance rate is the volume of blood plasma that is cleared of creatinine per unit time and is used to approximate the GFR. GFR can be measured (e.g., measured GFR (mGFR)) with gold standard methods or estimated (e.g., eGFR) with formulas. eGFR provides a more convenient and rapid analysis for evaluating kidney function; however, the equations for estimating GFR have been found to have limitations and have not been generalizable across all populations. Thus, more frequent use of measured GFR tests is recommended. However, determining the mGFR requires measurement of an exogenous marker such as inulin which may prove to be time-consuming and tedious. Further, both tests fail to consider serum potassium levels, as well as insulin levels, of the patient.

Another deficiency of the current methods and techniques for diagnosing, staging, and predicting symptoms of kidney disease is that the current clinical standard for potassium measurement (for kidney health assessment purposes), is a blood test. In some cases, whole blood samples are obtained by pricking the finger with a lancet. In some cases, blood samples are obtained using a venous blood draw. In venous blood sampling, a needle is inserted into a vein to collect a sample of blood for testing. Venous blood samples are often ordered for a patient on a weekly basis. Measuring whole blood, however, comes with a risk of hemolysis (e.g., the rupturing of red blood cells from external forces), particularly common in the finger prick method of blood collection, which can result in false positive measurements due to the high intracellular concentration of potassium that is released upon cell rupture. Of all routine blood tests, plasma/serum potassium measurement is one of the most sensitive measurements to the effect of hemolysis because red-cell potassium concentration is much higher than that of plasma (approximately 20 times higher). Accordingly, even slight hemolysis may cause a spuriously high plasma potassium concentration, which may make diagnosing, staging, and predicting symptoms of kidney disease based on potassium measurements unreliable.

Further, ensuring a patient continues to partake in such potassium monitoring activities, such as daily or weekly blood tests, may present a problem in itself. A patient who forgoes engaging in such potassium monitoring activities, which help to stabilize the user's chronic condition, may fail to manage the condition outside of such tests. Where the condition is left unmanaged for too long, the patient's condition may significantly deteriorate, additional health issues may arise, and, in some cases, lead to an increased risk or likelihood of mortality.

Beyond the issues mentioned above for conventional disease diagnostic methods and systems used for assessing and/or monitoring kidney disease, hyperkalemia, hypokalemia, hyperglycemia, and/or hypoglycemia in a patient, such diagnostic methods and systems also fail to consider one or more symptoms of a patient when providing a diagnosis. In particular, while the above conventional tests may provide diagnostics signs used to aid in the recognition of declining kidney health for purposes of kidney disease diagnosis (and/or hyperkalemia, hypokalemia, hyperglycemia, hypoglycemia diagnosis), the identification and prediction of one or more symptoms in a personalized manner, in addition to such diagnostic signs may provide additional insight into the diagnosis, treatment, and management of the disease. In particular, a sign is an objective, observable phenomenon that can be identified by another person or device. On the other hand, a symptom is a subjective experience of a patient. Typically, a medical condition, such as kidney disease (and/or hyperkalemia, hypokalemia, hyperglycemia, hypoglycemia), may be more easily diagnosed with knowledge of both signs and symptoms of a patient.

Accordingly, understanding and/or being able to predict symptoms of a patient may play an important role in the evaluation of kidney health, including diagnosing kidney disease and other kidney health issues. For example, knowledge that a patient is experiencing numbness, tingling, shortness of breath, and/or muscle weakness may suggest that the patient's potassium levels are too high or too low, and in some cases, such high and low potassium levels may be attributed to declining kidney function of the patient. However, in cases where potassium levels are too high or too low, many patients are often asymptomatic, or mildly symptomatic, until they are in immediate danger or at risk of severe medical complications or death.

Further, understanding and/or being able to predict one or more symptoms of a patient in real-time or near-real-time before the one or more symptoms occur may allow a patient to avoid the one or more symptoms before they occur. For example, where a patient is predicted to experience an arrhythmic event within the next hour, the patient may be able to take one or more actions to prevent the arrhythmia from occurring, or at a minimum seek medical help before the event occurs. However, with conventional methods of measuring potassium, predicting one or more symptoms of a patient in real-time before they occur may not be feasible, unless a patient is constantly (e.g., every few minutes) getting their blood drawn to allow for the continuous assessment of potassium levels of the patient over time. Such continuous measurement using conventional methods is not generally feasible, nor desired.

Even assuming conventional methods are capable of continuously measuring potassium levels of a patient, potassium levels used to predict different symptoms of a patient may vary from patient to patient. For example, at a potassium level of 5.8 mmol/L of blood, a first patient may experience severe symptoms, while at the same potassium level, a second patient may have no symptoms. Accordingly, to accurately predict one or more symptoms of a patient using conventional methods for continuously measuring potassium levels of a patient, additional techniques may be needed to determine specific, personalized potassium thresholds, which may be constantly changing for a patient. Determining such dynamic thresholds for predicting different symptoms of the patient may not be possible using conventional methods, such as blood draws.

Overall, existing diagnostic methods suffer from a first technical problem of failing to continuously monitor the concentration of a changing analyte, such as potassium, to give a continuous readout. As used herein, the term “continuous” may mean fully continuous, semi-continuous, periodic, etc. Such continuous monitoring of a particular analyte is advantageous in diagnosing and staging a disease of a patient given the continuous measurements provide continuously up to date measurements as well as information on the trend (e.g., rate of change, direction of trend, variability, mean, average, etc.) of analyte change over a continuous period. Such information may be used to make more informed decisions in the assessment of kidney health and treatment of kidney disease.

Second, existing diagnostics methods suffer from another technical problem of failing to continuously monitor the concentration of a plurality of changing analytes simultaneously. In particular, the continuous, semi-continuous, or periodic monitoring of multiple analytes such as potassium, glucose, creatinine, lactate, blood urea nitrogen (BUN), ammonia, C-peptide, and/or cystatin C may provide additional insight when assessing the presence and/or severity of kidney disease, hyperkalemia, hypokalemia, hyperglycemia, and/or hypoglycemia in a patient. The additional insight gained from using a combination of analytes, and not just a single analyte such as potassium, may help to increase the accuracy of the prediction, as well as make more informed patient-specific treatment decisions and/or recommendations for preventing progression of the disease(s) (and in some cases, for disease regression).

Third, existing diagnostics methods suffer from another technical problem of failing to predict symptoms of one or more diseases of a patient, and more specifically, predict symptoms of one or more diseases of a patient as they vary from patient to patient. Knowing in advance if a patient will present certain symptoms may have significant implications in terms of health strategy and intervention. For instance, specific intervention strategies may be recommended to that patient to reduce the likelihood of such symptoms. Additionally, a predictive model of disease symptoms may represent a valuable input for a model designed to assess the presence and/or severity of one or more diseases in a patient.

As a result of these technical problems, diagnosing kidney disease, hyperkalemia, hypokalemia, hyperglycemia, and/or hypoglycemia, or a risk thereof, as well as identification and prediction of a patient's symptoms of such disease(s) using conventional techniques may not only be inaccurate but also impossible, which, in some cases, might prove to be life threatening for a patient with one or more of these diseases. Specifically, predicting the progression of kidney health and its corresponding symptoms in a personalized manner with reasonable accuracy may be necessary given the dynamic and covert nature of kidney disease in the early stages, as well as patient heterogeneity. Further, predicting hyperkalemia, hypokalemia, hyperglycemia, and/or hypoglycemia and their corresponding symptoms in a personalized manner with reasonable accuracy may be necessary given such diseases may contribute to the development of serious or life-threatening conditions (e.g., such as cardiac arrhythmias) and, in some cases, lead to death. Thus, improved methods for detecting and predicting symptoms of a patient, diagnosing kidney disease, hyperkalemia, hypokalemia, hyperglycemia, and/or hypoglycemia, and understanding the interplay between such conditions in a patient is desired.

Accordingly, certain embodiments described herein provide a technical solution to the technical problems described above by providing decision support around kidney health, such as kidney disease, using continuous analyte measurements, including at least, continuous potassium measurements using a continuous analyte monitoring system. In some examples, the continuous potassium measurements are obtained using a continuous potassium monitor (CPM). Further, certain embodiments described herein provide a technical solution to the technical problems described above by providing decision support around kidney disease and/or other comorbid conditions, such as diabetes, using continuous potassium and glucose measurements. For example, a continuous analyte monitoring system, including, at least, a CPM and a continuous glucose monitor (CGM) may be provided to enable providing decision support around kidney disease and/or other conditions such as diabetes.

The decision support may be provided in the form of risk assessment, diagnosis, staging, recommendations for the treatment or prevention of kidney disease (or associated diseases such as hyperkalemia and hypokalemia), and/or (2) achieving or maintaining glucose homeostasis, as described in more detail herein. As used herein, risk assessment may refer to the evaluation or estimation of kidney disease, hyperkalemia, hypokalemia, hyperglycemia, and/or hypoglycemia of a patient reaching a more advanced stage, mortality risk, a risk of being diagnosed with one or more other diseases, a risk of experiencing one or more symptoms, and the like.

In certain embodiments, the continuous analyte monitoring system may provide decision support to a patient based on a variety of collected data, including analyte data, patient information, secondary sensor data (e.g., non-analyte data), etc. For example, the analyte data may include continuously monitored potassium data in addition to other continuously monitored analyte data, such as glucose, creatinine, blood urea nitrogen (BUN), ammonia, C-peptide, and cystatin C.

Potassium helps to regulate fluid balance, muscle contractions, and nerve signals in the body. A high-potassium diet may also help to reduce blood pressure and water retention, protect against stroke, and prevent osteoporosis and kidney stones. As mentioned herein, the kidney plays a major role in potassium homeostasis by renal mechanisms that transport and regulate potassium secretion, reabsorption and excretion. Thus, the continuously monitored potassium data may indicate, or be used for determining, the patient's potassium levels and/or rates of change of the patient's potassium levels over time to assess kidney health and function for a patient.

Further, potassium imbalance may be improved with the continuous measurement and assessment of glucose. As mentioned, kidney disease may result in glucose imbalance; thus, continuously monitoring glucose levels of a patient with, or at risk of, poor and/or declining kidney health may be appropriate to reduce the risk of kidney disease (and/or associated diseases) and for earlier detection. Additionally, insulin reduces both serum glucose and serum potassium levels by stimulating glucose and potassium uptake by cells. Exogenous insulin (e.g., exogenous insulin refers to the insulin people inject or infuse, for example, via an insulin pump) is often used to manage glucose in diabetic patients. However, insulin may also impact potassium levels and, in a patient with CKD, longer insulin half-life increases the patient's risk of hypokalemia. As such, the continuously monitored glucose data, in addition to the continuously monitored potassium data, may be used to better inform the management of CKD of a patient, and more specifically, provide more informed recommendations for treatment and/or prevention of CKD, hyperkalemia, hypokalemia, hyperglycemia, and/or hypoglycemia.

Certain embodiments of the present disclosure provide techniques and systems for correcting a patient's potassium levels by using measurements associated with other analyte sensor data, the secondary sensor data, and/or other patient information, as further described below. As described above, the other analyte sensor data may include glucose, creatinine, lactate, blood urea nitrogen (BUN), ammonia, C-peptide, and/or cystatin C data. Other patient information includes patient information, which may include information related to age, gender, family history of kidney disease, other health conditions, etc. Secondary sensor data may include accelerometer data, heart rate data (ECG, HRV, HR, etc.), temperature, blood pressure, time of sensor initiation or remaining sensor life relative to the initiation time, or any other sensor data other than analyte data.

According to certain embodiments of the present disclosure, the decision support system presented herein is designed to predict the risk or likelihood of one or more symptoms of kidney disease occurring in-real time or within a specified period of time for a patient. In particular, the decision support system presented herein may be designed to predict the risk or likelihood of a patient experiencing (in real-time or at a later time) (1) numbness, (2) tingling, (3) shortness of breath (or chest pain), (4) muscle weakness, and/or (4) arrhythmia. Especially in the early stages of kidney disease, a patient might have few symptoms related to kidney disease. Further, the patient may not realize they have kidney disease until the condition is advanced. Thus, being able to identify that a patient is experiencing a symptom of kidney disease, although they may not realize they are experiencing such symptoms, may be critical to the early detection of kidney disease in a patient.

According to embodiments of the present disclosure, the decision support system presented herein is designed to provide a diagnosis for patients with, or at risk of, kidney disease as well as disease decision support to assist the patient in managing their kidney disease, or risk thereof. Providing kidney disease decision support may involve using collected data, including for example, the analyte data, patient information, and secondary sensor data mentioned above, to (1) automatically detect and classify abnormal kidney function, (2) assess the presence and severity of kidney disease, (3) risk stratify patients to identify those patients with a high risk of kidney disease, (4) identify risks (e.g., mortality risk, hyperkalemia risk, hypokalemia risk, etc.) associated with a current kidney disease diagnosis, (5) make patient-specific treatment decisions or recommendations for kidney disease (and/or associated diseases), and (6) provide information on the effect of an intervention (e.g., an effect of a lifestyle change of the patient, an effect of a surgical procedure, an effect of a dialysis session, an effect of the patient taking new medication, etc.). In other words, the decision support system presented herein may offer information to direct and help improve care for patients with, or at risk, of kidney disease, hyperkalemia, hypokalemia, hyperglycemia, and/or hypoglycemia.

In certain embodiments, the decision support system described herein may use various algorithms or artificial intelligence (AI) models, such as machine-learning models, trained based on patient-specific data and/or population data to provide real-time decision support to a patient based on the collected information about the patient. For example, certain aspects are directed to algorithms and/or machine-learning models designed to assess the presence and severity of kidney disease in a patient. The algorithms and/or machine-learning models may be used in combination with one or more continuous analyte sensors, including at least a CPM, to provide real-time kidney disease assessment and staging. In particular, the algorithms and/or machine-learning models may take into account parameters, such as potassium levels, trends of potassium levels over time such as rates of change of potassium levels of the patient over time, and physiological parameters of a patient when experiencing different symptoms commonly associated with kidney disease, when diagnosing and staging kidney disease.

Based on these parameters, the algorithms and/or machine-learning models may provide a risk assessment of different kidney disease stages (and their corresponding severity), as well the progression a patient has made towards one or more of those kidney disease stages. The algorithms and/or machine-learning models may take into consideration population data, personalized patient-specific data, or a combination of both when diagnosing and staging kidney disease for a patient.

As described in more detail herein, the population data may be provided in a form of a dataset including data records of historical patients with varying stages of kidney disease and/or experiencing different symptoms associated with potassium imbalance, and in some cases, kidney disease. Each data record may be used as input into the machine learning models to optimize such models to generate accurate predictions associated with kidney disease (e.g., predictions of kidney disease presence and severity in a patient, predictions of one or more symptom of kidney disease occurring in real-time or within a specified period of time, etc.). According to certain embodiments, prior to deployment, the machine learning models are trained with training data, e.g., including user-specific data and/or population data.

According to certain embodiments, the decision support system described herein is designed to provide decision support in the form of risk assessment and treatment for kidney disease and/or glucose homeostasis. For example, in certain embodiments, the decision support system is designed to continuously measure serum potassium levels of a patient and provide recommendations for treatment, and specifically, potassium imbalance associated with kidney disease. In certain other embodiments, the decision support system is designed to continuously measure both serum potassium levels and glucose levels of a patient (e.g., via multiple sensors or a sensor capable of measuring both analytes) and provide recommendations for treatment of potassium imbalance and glucose imbalance.

In certain embodiments, the decision support system described herein provides risk stratification and treatment recommendations through an algorithm and/or a method for the treatment and/or prevention of kidney disease, hyperkalemia, hypokalemia, hyperglycemia, and/or hypoglycemia. In certain embodiments, method is a tree-like rules-based model for providing decision support to a patient. Other types of algorithms and rules-based models are also within the scope of the disclosure. In certain embodiments, the method described herein may be used for effective decision making with respect to treatment and/or preventative medical recommendations. In particular, the method described herein includes different nodes (or steps) to be used in determining treatment recommendations, and in some cases, risk stratification in order to inform treatment recommendations. For example, in medical decision making, the method described herein may be used to aid in making diet-related decisions, and for example, whether and/or when to consume potassium and/or glucose and/or water for purposes of maintaining potassium and/or glucose homeostasis. As another example, in medical decision making, the method described herein may be used to aid insulin dosing, dialysis, potassium binder ingestion decisions, as well as identify when a patient may need to consult with a medical profession and/or go to the emergency room for diagnosis and/or treatment.

In certain embodiments, decision support risk stratification and treatment recommendations may be based on a patient's potassium levels, trends, and/or thresholds. Different thresholds may be set based on risk(s) of potassium related states such as hyperkalemia and/or hypokalemia, available treatments, effectiveness of treatments, characteristics of the patient, patient activity, and/or the like. In addition, thresholds may be set by the user or a health professional, may be fixed and therefore not able to be set by the user, may default to a specific value, or dynamically set and automatically or manually adjusted based on inputs 128. Thresholds may be absolute values or correspond to or be based on trends of the user.

Further, in certain embodiments, decision support risk stratification and treatment recommendations may be based on a patient's glucose levels, rates of change, trends and/or thresholds. Different glucose thresholds may be set based on risk(s) of glucose related states such as hyperglycemia and/or hypoglycemia, as well as risk(s) of hyperkalemia and/or hypokalemia, available treatments, effectiveness of treatments, characteristics of the patient, patient activity, and/or the like.

The combination of a continuous analyte monitoring system with machine learning models and/or algorithms for (1) predicting one or more symptoms associated with potassium imbalance (e.g., abnormal or out of range potassium levels) and (2) predicting (e.g., assessing) kidney health of the user, including diagnosing, staging, and assessing risks of kidney disease, provided by the decision support system described herein enables real-time diagnosis to allow early intervention. In particular, the decision support system may be used to provide an early alert of declining kidney function and/or deliver information about other complications related to the kidney. Early detection of such decompensation and/or other complications may allow for intervention at the earliest possible stage to improve kidney health and/or kidney disease outcomes. For example, early intervention may reduce hospitalization, complications, and death, in some cases. In addition, potassium levels and changes in potassium levels provided by the continuous analyte monitoring system may be used as input into the machine learning models and/or algorithms to triage patients for more urgent care. In patients at risk for cardiac arrhythmia episodes and/or death, sudden increases in potassium may be used to inform urgent medical intervention, even before patients are able to report noticeable physiologic symptoms.

In addition, through the combination of a continuous analyte monitoring system with machine learning models and/or algorithms (e.g., rules-based models) for (1) predicting one or more symptoms associated with potassium imbalance and (2) predicting kidney health of the user, including diagnosing, staging, and assessing risk of kidney disease, the decision support system described herein may provide the necessary accuracy and reliability patients expect. For example, biases, human errors, and emotional influence may be minimized when assessing the presence and severity of kidney health in patients. Further, machine learning models and algorithms (e.g. rules-based models) in combination with analyte monitoring systems may provide insight into patterns and/or trends of decreasing health of a patient, at least with respect to the kidney and/or heart, which may have been previously missed. Accordingly, the decision support system described herein may assist in the identification of kidney health for diagnosis, preventive, and treatment purposes.

### Example Decision Support System Including an Example Analyte Sensor

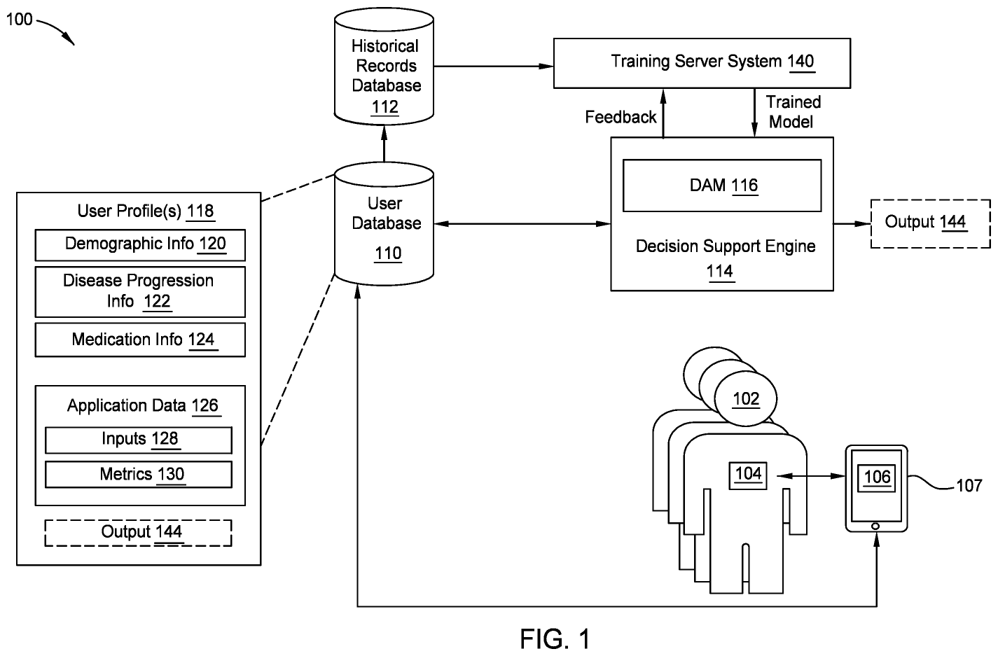

FIG. 1 illustrates an example decision support system 100 (also referred to herein as the “monitoring system”) for (1) predicting one or more symptoms associated with potassium imbalance and/or (2) predicting kidney health of the user, including diagnosing, staging, treating, and assessing risks of kidney disease in relation to users 102 (individually referred to herein as a user and collectively referred to herein as users), using a continuous analyte monitoring system 104, including, at least, a continuous potassium monitor (CPM). A user, in certain embodiments, may be the patient or, in some cases, the patient's caregiver. In certain embodiments, decision support system 100 includes continuous analyte monitoring system 104, a display device 107 that executes application 106, a decision support engine 114, a user database 110, a historical records database 112, a training server system 140, and a decision support engine 114, each of which is described in more detail below.

The term “analyte” as used herein is a broad term used in its ordinary sense, including, without limitation, to refer to a substance or chemical constituent in a biological fluid (for example, blood, interstitial fluid, cerebral spinal fluid, lymph fluid or urine) that can be analyzed. Analytes can include naturally occurring substances, artificial substances, metabolites, and/or reaction products. Analytes for measurement by the devices and methods may include, but may not be limited to, potassium, glucose, acarboxyprothrombin; acylcarnitine; adenine phosphoribosyl transferase; adenosine deaminase; albumin; alpha-fetoprotein; amino acid profiles (arginine (Krebs cycle), histidine/urocanic acid, homocysteine, phenylalanine/tyrosine, tryptophan); androstenedione; antipyrine; arabinitol enantiomers; arginase; benzoylecgonine (cocaine); biotinidase; biopterin; c-reactive protein; carnitine; carnosinase; CD4; ceruloplasmin; chenodeoxycholic acid; chloroquine; cholesterol; cholinesterase; conjugated 1-β hydroxy-cholic acid; cortisol; creatine kinase; creatine kinase MM isoenzyme; cyclosporin A; d-penicillamine; de-ethylchloroquine; dehydroepiandrosterone sulfate; DNA (acetylator polymorphism, alcohol dehydrogenase, alpha 1-antitrypsin, glucose-6-phosphate dehydrogenase, hemoglobin A, hemoglobin S, hemoglobin C, hemoglobin D, hemoglobin E, hemoglobin F, D-Punjab, hepatitis B virus, HCMV, HIV-1, HTLV-1, MCAD, RNA, PKU, Plasmodium vivax, 21-deoxycortisol); desbutylhalofantrine; dihydropteridine reductase; diptheria/tetanus antitoxin; endogenous insulin; erythrocyte arginase; erythrocyte protoporphyrin; esterase D; fatty acids/acylglycines; free β-human chorionic gonadotropin; free erythrocyte porphyrin; free thyroxine (FT4); free tri-iodothyronine (FT3); fumarylacetoacetase; galactose/gal-1-phosphate; galactose-1-phosphate uridyltransferase; gentamicin; glucose-6-phosphate dehydrogenase; glutathione; glutathione perioxidase; glycocholic acid; glycosylated hemoglobin; halofantrine; hemoglobin variants; hexosaminidase A; human erythrocyte carbonic anhydrase I; 17-alpha-hydroxyprogesterone; hypoxanthine phosphoribosyl transferase; immunoreactive trypsin; lactate; lead; lipoproteins ((a), B/A-1, β); lysozyme; mefloquine; netilmicin; phenobarbitone; phenytoin; phytanic/pristanic acid; progesterone; prolactin; prolidase; purine nucleoside phosphorylase; quinine; reverse tri-iodothyronine (rT3); selenium; serum pancreatic lipase; sisomicin; somatomedin C; specific antibodies recognizing any one or more of the following that may include (adenovirus, anti-nuclear antibody, anti-zeta antibody, arbovirus, Aujeszky's disease virus, dengue virus, Dracunculus medinensis, Echinococcus granulosus, Entamoeba histolytica, enterovirus, Giardia duodenalisa, Helicobacter pylori, hepatitis B virus, herpes virus, HIV-1, IgE (atopic disease), influenza virus, Leishmania donovani, leptospira, measles/mumps/rubella, Mycobacterium leprae, Mycoplasma pneumoniae, Myoglobin, Onchocerca volvulus, parainfluenza virus, Plasmodium falciparum, poliovirus, Pseudomonas aeruginosa, respiratory syncytial virus, rickettsia (scrub typhus), Schistosoma mansoni, Toxoplasma gondii, Trepenoma pallidium, Trypanosoma cruzi/rangeli, vesicular stomatis virus, Wuchereria bancrofti, yellow fever virus); specific antigens (hepatitis B virus, HIV-1); succinylacetone; sulfadoxine; theophylline; thyrotropin (TSH); thyroxine (T4); thyroxine-binding globulin; trace elements; transferrin; UDP-galactose-4-epimerase; urea; uroporphyrinogen I synthase; vitamin A; white blood cells; and zinc protoporphyrin. Salts, sugar, protein, fat, vitamins, and hormones naturally occurring in blood or interstitial fluids can also constitute analytes in certain implementations. Ions are a charged atom or compounds that may include the following (sodium, potassium, calcium, chloride, nitrogen, or bicarbonate, for example). The analyte can be naturally present in the biological fluid, for example, a metabolic product, a hormone, an antigen, an antibody, an ion and the like. Alternatively, the analyte can be introduced into the body or exogenous, for example, a contrast agent for imaging, a radioisotope, a chemical agent, a fluorocarbon-based synthetic blood, a challenge agent analyte (e.g., introduced for the purpose of measuring the increase and or decrease in rate of change in concentration of the challenge agent analyte or other analytes in response to the introduced challenge agent analyte), or a drug or pharmaceutical composition, including but not limited to exogenous insulin; glucagon, ethanol; cannabis (marijuana, tetrahydrocannabinol, hashish); inhalants (nitrous oxide, amyl nitrite, butyl nitrite, chlorohydrocarbons, hydrocarbons); cocaine (crack cocaine); stimulants (amphetamines, methamphetamines, Ritalin, Cylert, Preludin, Didrex, PreState, Voranil, Sandrex, Plegine); depressants (barbiturates, methaqualone, tranquilizers such as Valium, Librium, Miltown, Serax, Equanil, Tranxene); hallucinogens (phencyclidine, lysergic acid, mescaline, peyote, psilocybin); narcotics (heroin, codeine, morphine, opium, meperidine, Percocet, Percodan, Tussionex, Fentanyl, Darvon, Talwin, Lomotil); designer drugs (analogs of fentanyl, meperidine, amphetamines, methamphetamines, and phencyclidine, for example, Ecstasy); anabolic steroids; and nicotine The metabolic products of drugs and pharmaceutical compositions are also contemplated analytes. Analytes such as neurochemicals and other chemicals generated within the body can also be analyzed, such as, for example, ascorbic acid, uric acid, dopamine, noradrenaline, 3-methoxytyramine (3MT), 3,4-Dihydroxyphenylacetic acid (DOPAC), Homovanillic acid (HVA), 5-Hydroxytryptamine (5HT), and 5-Hydroxyindoleacetic acid (FHIAA), and intermediaries in the Citric Acid Cycle.

While the analytes that are measured and analyzed by the devices and methods described herein include potassium, and in some cases glucose, creatinine, blood urea nitrogen (BUN), ammonia, C-peptide, and cystatin C, other analytes listed, but not limited to, above may also be considered and measured by, for example, analyte monitoring system 104.

In certain embodiments, continuous analyte monitoring system 104 is configured to continuously measure one or more analytes and transmit the analyte measurements to an electric medical records (EMR) system (not shown in FIG. 1). An EMR system is a software platform which allows for the electronic entry, storage, and maintenance of digital medical data. An EMR system is generally used throughout hospitals and/or other caregiver facilities to document clinical information on patients over long periods. EMR systems organize and present data in ways that assist clinicians with, for example, interpreting health conditions and providing ongoing care, scheduling, billing, and follow up. Data contained in an EMR system may also be used to create reports for clinical care and/or disease management for a patient. In certain embodiments, the EMR may be in communication with decision support engine 114 (e.g., via a network) for performing the techniques described herein. In particular, as described herein, decision support engine 114 may obtain data associated with a user, use the obtained data as input into one or more trained model(s), and output a prediction. In some cases, the EMR may provide the data to decision support engine 114 to be used as input into the one or more models. Further, in some cases, decision support engine 114, after making a prediction, may provide the output prediction to the EMR.

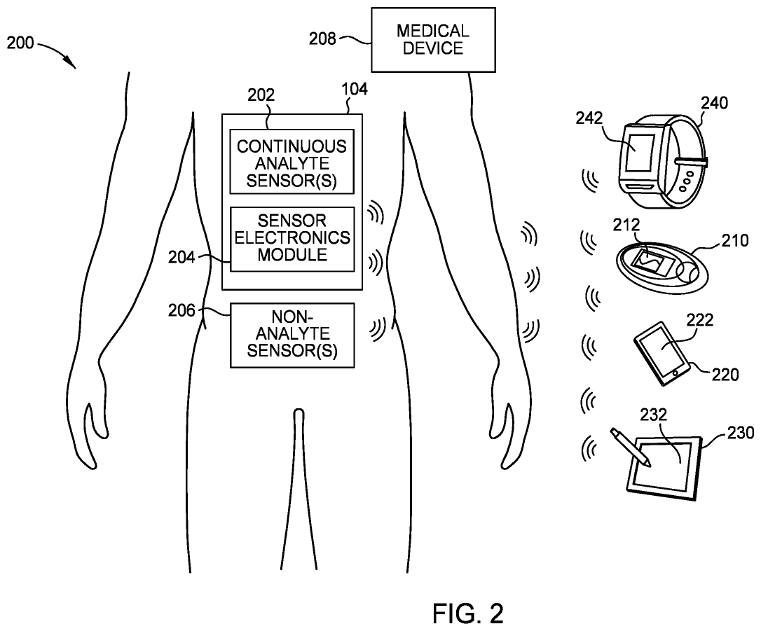

In certain embodiments, continuous analyte monitoring system 104 is configured to continuously measure one or more analytes and transmit the analyte measurements to display device 107 for use by application 106. In some embodiments, continuous analyte monitoring system 104 transmits the analyte measurements to display device 107 through a wireless connection (e.g., Bluetooth connection). In certain embodiments, display device 107 is a smart phone. However, in certain other embodiments, display device 107 may instead be any other type of computing device such as a laptop computer, a smart watch, a tablet, or any other computing device capable of executing application 106. In some embodiments, continuous analyte monitoring system 104 and/or analyte sensor application 106 transmit the analyte measurements to one or more other individuals having an interest in the health of the patient (e.g., a family member or physician for real-time treatment and care of the patient). Continuous analyte monitoring system 104 may be described in more detail with respect to FIG. 2.

Application 106 is a mobile health application that is configured to receive and analyze analyte measurements from analyte monitoring system 104. In particular, application 106 stores information about a user, including the user's analyte measurements, in a user profile 118 associated with the user for processing and analysis, as well as for use by decision support engine 114 to provide decision support recommendations or guidance to the user.

Decision support engine 114 refers to a set of software instructions with one or more software modules, including data analysis module (DAM) 116. In certain embodiments, decision support engine 114 executes entirely on one or more computing devices in a private or a public cloud. In such embodiments, application 106 communicates with decision support engine 114 over a network (e.g., Internet). In some other embodiments, decision support engine 114 executes partially on one or more local devices, such as display device 107, and partially on one or more computing devices in a private or a public cloud. In some other embodiments, decision support engine 114 executes entirely on one or more local devices, such as display device 107. As discussed in more detail herein, decision support engine 114 may provide decision support recommendations to the user via application 106. Decision support engine 114 provides decision support recommendations based on information included in user profile 118.

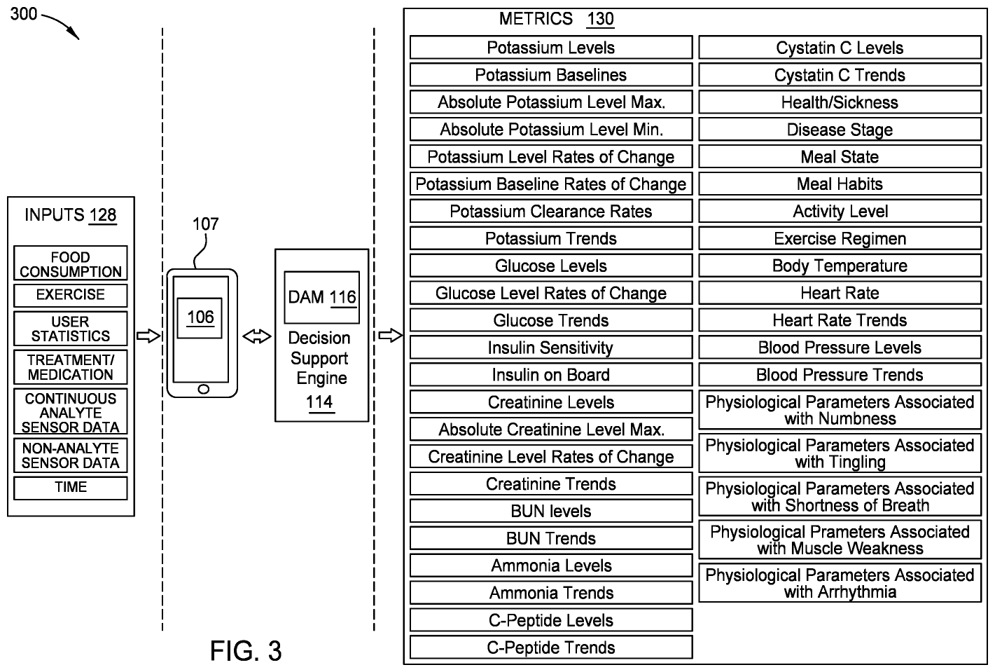

User profile 118 may include information collected about the user from application 106. For example, application 106 provides a set of inputs 128, including the analyte measurements received from continuous analyte monitoring system 104, that are stored in user profile 118. In certain embodiments, inputs 128 provided by application 106 include other data in addition to analyte measurements received from continuous analyte monitoring system 104. For example, application 106 may obtain additional inputs 128 through manual user input, one or more other non-analyte sensors or devices, non-continuous analyte lab test results (e.g., urine albumin test, GFR, or ACR), other applications executing on display device 107, etc. Non-analyte sensors and devices include one or more of, but are not limited to, an insulin pump, an electrocardiogram (ECG) sensor or heart rate monitor, a blood pressure sensor, a respiratory sensor, a thermometer, a peritoneal dialysis machine, a hemodialysis machine, sensors or devices provided by display device 107 (e.g., accelerometer, camera, global positioning system (GPS), heart rate monitor, etc.) or other user accessories (e.g., a smart watch), or any other sensors or devices that provide relevant information about the user. Inputs 128 of user profile 118 provided by application 106 are described in further detail below with respect to FIG. 3.

DAM 116 of decision support engine 114 is configured to process the set of inputs 128 to determine one or more metrics 130. Metrics 130, discussed in more detail below with respect to FIG. 3, may, at least in some cases, be generally indicative of the health or state of a user, such as one or more of the user's physiological state, trends associated with the health or state of a user, etc. In certain embodiments, metrics 130 may then be used by decision support engine 114 as input for providing guidance to a user. As shown, metrics 130 are also stored in user profile 118.

User profile 118 also includes demographic info 120, disease progression info 122, and/or medication info 124. In certain embodiments, such information may be provided through user input or obtained from certain data stores (e.g., electronic medical records (EMRs), etc.). In certain embodiments, demographic info 120 may include one or more of the user's age, body mass index (BMI), ethnicity, gender, etc. In certain embodiments, disease progression info 122 may include information about a disease of a user, such as whether the user has been previously diagnosed with acute kidney injury (AKI) or chronic kidney disease (CKD), or have had a history of hyperkalemia, hypokalemia, etc. In certain embodiments, information about a user's disease may also include the length of time since diagnosis, the level of disease control, level of compliance with disease management therapy, predicted kidney function, other types of diagnosis (e.g., diabetes, heart disease, obesity) or measures of health (e.g., heart rate, exercise, stress, sleep, etc.), and/or the like.

In certain embodiments, medication info 124 may include information about the amount, frequency, and type of a medication taken by a user. In certain embodiments, the amount, frequency, and type of a medication taken by a user is time-stamped and correlated with the user's analyte levels, thereby, indicating the impact the amount, frequency, and type of the medication had on the user's analyte levels. In certain embodiments, medication information may include information about the consumption of one or more diuretics. Diuretics may be prescribed to a patient for the purpose of treating excessive fluid accumulation caused by, for example, congestive heart failure (CHF), liver failure, and/or nephritic syndrome. For example, CHF is a condition in which the heart is unable to efficiently pump blood to meet the body's oxygen and nutrient needs. The inability of the heart to efficiently pump blood impairs normal blood circulation and leads to excess fluid in the blood. The excess fluid leaks out of the blood vessels and accumulates in the lungs and other tissues. Accordingly, a patient may be prescribed diuretics to help the kidneys flush out the excess fluid and maintain normal blood volume.

Different types of diuretics prescribed to a patient may include loop diuretics, thiazide and thiazide-like diuretics, and potassium-sparing diuretics. Loop diuretics inhibit a protein found in a part of the nephron known as the loop of Henle. Loop diuretics may include, for example, Furosemide (Lasix), Bumetanide (Bumex), Torsemide (Demadex), Ethacrynic acid (Edecrin). Thiazide diuretics are commonly used to treat high blood pressure (hypertension), but also to manage heart failure. Thiazide diuretics inhibit a different protein than the loop diuretics do, which also helps in mineral reabsorption. Thiazide diuretics may include, for example, Chlorothiazide (Diuril), Hydrochlorothizaide (Hydrodiuril), Metolazone (Zytonix). Lastly, potassium sparing diuretics (e.g., such as Spironolactone) are weak diuretics used to increase the amount of fluid passed from the body in urine, while also preventing too much potassium from being lost from the body in the urine. As described in more detail below, decision support system 100 may be configured to use medication info 124 to determine optimal diuretics to be prescribed to different users. In particular, decision support system 100 may be configured to identify one or more optimal diuretics for prescription based on the health of the patient when one or more diuretics are prescribed, as well as the condition(s) of the patient to be treated.

In certain embodiments, medication information may include information about consumption of one or more drugs known to damage the kidney. One or more drugs known to damage the kidney may include nonsteroidal anti-inflammatory drugs (NSAIDS) such as ibuprofen (e.g., Advil, Motrin) and naproxen (e.g., Aleve), vancomycin, iodinated radiocontrast (e.g., refers to any contrast dyes used in diagnostic testing), angiotensin-converting enzyme (ACE) such as lisinopril, enalapril, and ramipril, aminoglycoside antibiotics such as neomycdin, gentamicin, tobramycin, and amikacin, antiviral human immunodeficiency virus (HIV) medications, zoledronic acid (e.g., Zometa, Reclast), foscarnet, and the like.

In certain embodiments, medication information may include information about consumption of one or more drugs known to control the complications of kidney disease. One or more drugs known to control the complications of kidney disease may include medications to lower blood pressure and preserve kidney function such as ACE inhibitors or angiotensin II receptor blockers, medications to treat anemia such as supplements of the hormone erythropoietin, medications used to lower cholesterol levels such as statins, medications used to prevent weak bones such as calcium and vitamin D supplements, phosphate binders, and the like.

In certain embodiments, user profile 118 is dynamic because at least part of the information that is stored in user profile 118 may be revised over time and/or new information may be added to user profile 118 by decision support engine 114 and/or application 106. Accordingly, information in user profile 118 stored in user database 110 provides an up-to-date repository of information related to a user.

User database 110, in some embodiments, refers to a storage server that operates in a public or private cloud. User database 110 may be implemented as any type of datastore, such as relational databases, non-relational databases, key-value datastores, file systems including hierarchical file systems, and the like. In some exemplary implementations, user database 110 is distributed. For example, user database 110 may comprise a plurality of persistent storage devices, which are distributed. Furthermore, user database 110 may be replicated so that the storage devices are geographically dispersed.

User database 110 includes user profiles 118 associated with a plurality of users who similarly interact with application 106 executing on the display devices 107 of the other users. User profiles stored in user database 110 are accessible to not only application 106, but decision support engine 114, as well. User profiles in user database 110 may be accessible to application 106 and decision support engine 114 over one or more networks (not shown). As described above, decision support engine 114, and more specifically DAM 116 of decision support engine 114, can fetch inputs 128 from user database 110 and compute a plurality of metrics 130 which can then be stored as application data 126 in user profile 118.

In certain embodiments, user profiles 118 stored in user database 110 may also be stored in historical records database 112. User profiles 118 stored in historical records database 112 may provide a repository of up-to-date information and historical information for each user of application 106. Thus, historical records database 112 essentially provides all data related to each user of application 106, where data is stored according to an associated timestamp. The timestamp associated with information stored in historical records database 112 may identify, for example, when information related to a user has been obtained and/or updated.

Further, historical records database 112 may maintain time series data collected for users over a period of time, including for users who use continuous analyte monitoring system 104 and application 106. For example, analyte data for a user who has used continuous analyte monitoring system 104 and application 106 for a period of five years to manage the user's kidney condition may have time series analyte data associated with the user maintained over the five-year period.

Further, in certain embodiments, historical records database 112 may include data for one or more patients who are not users of continuous analyte monitoring system 104 and/or application 106. For example, historical records database 112 may include information (e.g., user profile(s)) related to one or more patients analyzed by, for example, a healthcare physician (or other known method), and not previously diagnosed with kidney disease, as well as information (e.g., user profile(s)) related to one or more patients who were analyzed by, for example, a healthcare physician (or other known method) and were previously diagnosed with (varying types and stages of) kidney disease. Data stored in historical records database 112 may be referred to herein as population data.

Data related to each patient stored in historical records database 112 may provide time series data collected over the disease lifetime of the patient. For example, the data may include information about the patient prior to being diagnosed with kidney disease and information associated with the patient during the lifetime of the disease, including information related to each stage of the kidney disease as it progressed and/or regressed in the patient, as well as information related to other diseases, such as hyperkalemia, hypokalemia, diabetes, heart conditions and diseases, or similar diseases that are co-morbid in relation to kidney disease. Such information may indicate symptoms of the patient, physiological states of the patient, potassium levels of the patient, glucose levels of the patient, creatinine levels of patient, BUN levels of the patient, ammonia levels of the patient, C-peptide levels of the patient, cystatin C levels of the patient, states/conditions of one or more organs of the patient, habits of the patient (e.g., activity levels, food consumption, etc.), medication prescribed, etc. throughout the lifetime of the disease.

Although depicted as separate databases for conceptual clarity, in some embodiments, user database 110 and historical records database 112 may operate as a single database. That is, historical and current data related to users of continuous analyte monitoring system 104 and application 106, as well as historical data related to patients that were not previously users of continuous analyte monitoring system 104 and application 106, may be stored in a single database. The single database may be a storage server that operates in a public or private cloud.

As mentioned previously, decision support system 100 is configured to predict the kidney health of the user, including diagnosing, staging, treating, and assessing risks of kidney disease, as well as predict the likelihood of the user experiencing one or more symptoms commonly associated with potassium imbalance, for a user using continuous analyte monitoring system 104, including, at least, a CPM. In certain embodiments, decision support engine 114 is configured to provide real-time and or non-real-time decision support around kidney health to the user and or others, including but not limited, to healthcare providers, family members of the user, caregivers of the user, researchers, artificial intelligence (AI) engines, and/or other individuals, systems, and/or groups supporting care or learning from the data. In particular, decision support engine 114 may be used to collect information associated with a user in user profile 118 stored in user database 110, to perform analytics thereon for determining the likelihood of the user experiencing or more kidney disease symptoms and, in some cases, providing recommendations or warnings to the user based on the determined likelihood. Decision support engine 114 may also be used to collect information associated with a user in user profile 118 to perform analytics thereon for determining the probability of the presence and/or severity of kidney disease for the user and providing one or more recommendations for treatment based, at least in part, on the determination. User profile 118 may be accessible to decision support engine 114 over one or more networks (not shown) for performing such analytics.

In certain embodiments, decision support system 100 is designed to predict the risk or likelihood of one or more symptoms of kidney disease occurring in real-time (including near real-time), or within a specified period of time for a patient. In certain embodiments, to enable such prediction, decision support engine 114 is configured to collect information associated with a user in user profile 118 stored in user database 110, to perform analytics thereon for predicting the risk or likelihood of a patient experiencing (in real-time or at a later time) (1) potassium fluctuations (indicative of hyper or hypokalemia) (2) numbness, (3) tingling, (4) shortness of breath (or chest pain), (5) muscle weakness, (6) arrhythmia, or other symptoms. In certain embodiments, decision support engine 114 may attempt to confirm whether a user is experiencing the predicted one or more symptoms and store physiological parameters (e.g., potassium levels, glucose levels, C-peptide levels, heart rate, blood pressure, etc.) associated with the user where the user, or one or more other diagnostic tests, confirms the user is, in fact, experiencing the one or more predicted symptoms. Recording such physiological parameters may provide insight into what potassium thresholds (in addition to other measured values) correlate to what symptoms (e.g., common symptoms of kidney disease) for each specific user. Further, in certain embodiments, each user's potassium thresholds recorded for the different symptoms over time may be analyzed to provide an indication of the improvement or the deterioration of the patient's kidney disease.

In certain embodiments, decision support engine 114 may utilize one or more trained machine learning models and/or algorithms (e.g., rules-based models) capable of predicting symptoms for a user based on information provided by user profile 118. In the illustrated embodiment of FIG. 1, decision support engine 114 may utilize trained machine learning model(s) provided by a training server system 140. Although depicted as a separate server for conceptual clarity, in some embodiments, training server system 140 and decision support engine 114 may operate as a single server. That is, the model may be trained and used by a single server, or may be trained by one or more servers and deployed for use on one or more other servers. In certain embodiments, the model may be trained on one or many virtual machines (VMs) running, at least partially, on one or many physical servers in relational and or non-relational database formats.

Training server system 140 is configured to train the machine learning model(s) using training data, which may include data (e.g., from user profiles) associated with one or more patients (e.g., users or non-users of continuous analyte monitoring system 104 and/or application 106) previously diagnosed with varying stages of kidney disease, as well as patients not previously diagnosed with kidney disease (e.g., healthy patients). The training data may be stored in historical records database 112 and may be accessible to training server system 140 over one or more networks (not shown) for training the machine learning model(s). The training data may also, in some cases, include user-specific data for a user over time.

The training data refers to a dataset that has been featurized and labeled. For example, the dataset may include a plurality of data records, each including information corresponding to a different user profile stored in user database 110, where each data record is featurized and labeled. In machine learning and pattern recognition, a feature is an individual measurable property or characteristic. Generally, the features that best characterize the patterns in the data are selected to create predictive machine learning models. Data labeling is the process of adding one or more meaningful and informative labels to provide context to the data for learning by the machine learning model.

As an illustrative example, each relevant characteristic of a user, which is reflected in a corresponding data record, may be a feature used in training the machine learning model. Such features may include age, gender, change (e.g., delta) in potassium levels from a first timestamp to a second timestamp, change (e.g., delta) in kidney disease stage or severity from a first timestamp to a second timestamp, change (e.g., delta) in potassium thresholds of a user experiencing different symptoms from a first timestamp to a subsequent timestamp, the derivative of the measured linear system of potassium measurement at a point at a specific timestamp and or the difference in derivatives to determine rates of change in the slope of the increase or decrease in potassium values, etc. In addition, the data record is labeled with an indication as to the symptoms experienced, a kidney disease diagnosis, an assigned disease severity, and/or an identified risk of kidney disease, etc. associated with a patient of the user profile.

The model(s) are then trained by training server system 140 using the featurized and labeled training data. In particular, the features of each data record may be used as input into the machine learning model(s), and the generated output may be compared to label(s) associated with the corresponding data record. The model(s) may compute a loss based on the difference between the generated output and the provided label(s). This loss is then used to modify the internal parameters or weights of the model. By iteratively processing each data record corresponding to each historical patient, in certain embodiments, the model(s) may be iteratively refined to generate accurate predictions associated with kidney health of the user, including kidney disease risk, presence, progression, improvement, and severity in a patient. Further, in certain other embodiments, by iteratively processing each data record corresponding to each historical patient, in certain embodiments, the model(s) may be iteratively refined to generate accurate predictions the risk and/or presence of one or more symptoms associated with potassium imbalance.

As illustrated in FIG. 1, training server system 140 deploys these trained model(s) to decision support engine 114 for use during runtime. For example, decision support engine 114 may obtain user profile 118 associated with a user, use information in user profile 118 as input into the trained model(s), and output a prediction. The prediction may be indicative of the kidney health of the user, including the presence and/or severity of kidney disease for the user or indicative of the presence or a risk of the user experiencing one or more symptoms of kidney disease in real-time or within a certain time (e.g., shown as output 144 in FIG. 1). Output 144 generated by decision support engine 114 may also provide one or more recommendations for treatment based on the predictions. Output 144 may be provided to the user (e.g., through application 106), to a user's caretaker (e.g., a parent, a relative, a guardian, a teacher, a nurse, etc.), to a user's physician, or any other individual that has an interest in the wellbeing of the user for purposes of improving the user's health, such as, in some cases by effectuating the recommended treatment.

In certain embodiments, the user's own data is used to personalize the one or more models that are initially trained based on population data. For example, a model (e.g., trained using population data) may be deployed for use by decision support engine 114 to predict the presence or a risk of a specific user experiencing a symptom of kidney disease in real-time. After making a prediction using the model, decision support engine 114 may be configured to ask the user whether they are experiencing the predicted symptom and/or use one or more diagnostic tests to confirm the user is experiencing the predicted symptom. In some cases, the user's answer and/or results from the diagnostic test(s) performed may deny the user is experiencing the predicted symptom. Accordingly, the model may continue to be retrained and personalized using the user's answer, diagnostic test results, and/or physiological parameters of the user used as input into the model to personalize the model for the user.

In certain embodiments, output 144 generated by decision support engine 114 may be stored in user profile 118. In certain embodiments, output 144 may be a predicted likelihood of a user currently experiencing one or more kidney disease symptoms or a predicted likelihood of a user experiencing one or more kidney disease symptom within a specified period of time. In certain embodiments, output 144 may be patient-specific treatment decisions or recommendations for preventing one or more predicted kidney disease predictions from occurring in the patient. In certain embodiments, output 144 may be a prediction as to the kidney health of the user, including the presence and/or severity of kidney disease in a user. In certain embodiments, output 144 may be a prediction as to the risk of a user having kidney disease, hyperkalemia, and/or hypokalemia. In certain embodiments, output 144 may be a prediction as to a morality risk of the patient. In certain embodiments, output 144 may be patient-specific treatment decisions or recommendations for kidney disease for the patient. Output 144 stored in user profile 118 may be continuously updated by decision support engine 114. Accordingly, previous diagnoses and/or physiological parameters of the user corresponding to different symptoms commonly associated with kidney disease, originally stored as outputs 144 in user profile 118 in user database 110 and then passed to historical records database 112, may provide an indication of the progression of kidney disease in a user over time, as well as provide an indication as to the effectiveness of different treatments (e.g., medications) recommended to a user to help stop progression of the disease.

In certain embodiments, a user's own historical data may be used to provide decision support and insight around the user's kidney health such as kidney function and/or disease. For example, a user's historical data may be used by an algorithm as a baseline to indicate improvements or deterioration in the user's kidney function. As an illustrative example, a user's data from a specific duration of time, e.g., one month prior, two weeks prior, etc., may be used as a baseline that can be compared with the user's current data to identify whether the user's kidney function has improved or deteriorated. In certain embodiments, the user's own historical data may be used by training server system 140 to train a personalized model that may further be able to predict or project out the user's kidney function or the kidney's future improvement/deterioration based on the user's recent pattern of data (e.g., exercise data, food consumption data, etc.).

In certain embodiments, the machine learning model and or algorithm (e.g., rules-based model) may be trained or be configured to provide lifestyle recommendations, exercise recommendations, food intake recommendations, hydration recommendations, medication recommendations, and other types of decision support recommendations to help the user prevent symptoms, treat symptoms, and improve their kidney health and function based on the user's historical data, including how different types of medication, food, and treatment (e.g., such as dialysis) have impacted the user's kidney function in the past. In certain embodiments, the model may be trained to predict the underlying cause of certain improvements or deteriorations in the patient's kidney function. For example, application 106 may display a user interface with a graph that shows the patient's kidney functionality or a score thereof with trend lines and indicate, e.g., retrospectively, what caused the functionality of the kidney to suffer at certain points in time (e.g., excess potassium intake, ingestion of non-potassium sparing diuretics, etc.), or what caused a certain symptom to occur.

FIG. 2 is a diagram 200 conceptually illustrating an example continuous analyte monitoring system 104 including example continuous analyte sensor(s) with sensor electronics, in accordance with certain aspects of the present disclosure. For example, system 104 may be configured to continuously monitor one or more analytes of a user, in accordance with certain aspects of the present disclosure.

Continuous analyte monitoring system 104 in the illustrated embodiment includes sensor electronics module 204 and one or more continuous analyte sensor(s) 202 (individually referred to herein as continuous analyte sensor 202 and collectively referred to herein as continuous analyte sensors 202) associated with sensor electronics module 204. Sensor electronics module 204 may be in wireless communication (e.g., directly or indirectly) with one or more of display devices 210, 220, 230, and 240. In certain embodiments, sensor electronics module 204 may also be in wireless communication (e.g., directly or indirectly) with one or more medical devices, such as medical devices 208 (individually referred to herein as medical device 208 and collectively referred to herein as medical devices 208), and/or one or more other non-analyte sensors 206 (individually referred to herein as non-analyte sensor 206 and collectively referred to herein as non-analyte sensor 206).

In certain embodiments, a continuous analyte sensor 202 may comprise a sensor for detecting and/or measuring analyte(s). The continuous analyte sensor 202 may be a multi-analyte sensor configured to continuously measure two or more analytes or a single analyte sensor (e.g., a potassium sensor) configured to continuously measure a single analyte as a non-invasive device, a subcutaneous device, a transcutaneous device, a transdermal device, and/or an intravascular device. In certain embodiments, the continuous analyte sensor 202 may be configured to continuously measure analyte levels of a user using one or more measurement techniques, such as enzymatic, chemical, physical, electrochemical, spectrophotometric, polarimetric, calorimetric, iontophoretic, radiometric, immunochemical, and the like. In certain aspects the continuous analyte sensor 202 provides a data stream indicative of the concentration of one or more analytes in the user. The data stream may include raw data signals, which are then converted into a calibrated and/or filtered data stream used to provide estimated analyte value(s) to the user.

In certain embodiments, continuous analyte sensor 202 may be a multi-analyte sensor, configured to continuously measure multiple analytes in a user's body. For example, in certain embodiments, the continuous multi-analyte sensor 202 may be a single sensor configured to measure potassium, glucose, lactate, ketones, creatinine, blood urea nitrogen (BUN), ammonia, C-peptide, and cystatin C in the user's body.

In certain embodiments, one or more multi-analyte sensors may be used in combination with one or more single analyte sensors. As an illustrative example, a multi-analyte sensor may be configured to continuously measure potassium and glucose and may, in some cases, be used in combination with an analyte sensor configured to measure only BUN levels. Information from each of the multi-analyte sensor(s) and single analyte sensor(s) may be combined to provide kidney disease decision support using methods described herein.

In certain embodiments, sensor electronics module 204 includes electronic circuitry associated with measuring and processing the continuous analyte sensor data, including prospective algorithms associated with processing and calibration of the sensor data. Sensor electronics module 204 can be physically connected to continuous analyte sensor(s) 202 and can be integral with (non-releasably attached to) or releasably attachable to continuous analyte sensor(s) 202. Sensor electronics module 204 may include hardware, firmware, and/or software that enables measurement of levels of analyte(s) via a continuous analyte sensor(s) 202. For example, sensor electronics module 204 can include a potentiostat, a power source for providing power to the sensor, other components useful for signal processing and data storage, and a telemetry module for transmitting data from the sensor electronics module to one or more display devices. Electronics can be affixed to a printed circuit board (PCB), or the like, and can take a variety of forms. For example, the electronics can take the form of an integrated circuit (IC), such as an Application-Specific Integrated Circuit (ASIC), a microcontroller, and/or a processor.

Display devices 210, 220, 230, and/or 240 are configured for displaying displayable sensor data, including analyte data, which may be transmitted by sensor electronics module 204. Each of display devices 210, 220, 230, or 240 can include a display such as a touchscreen display 212, 222, 232, /or 242 for displaying sensor data to a user and/or receiving inputs from the user. For example, a graphical user interface (GUI) may be presented to the user for such purposes. In some embodiments, the display devices may include other types of user interfaces such as a voice user interface instead of, or in addition to, a touchscreen display for communicating sensor data to the user of the display device and/or receiving user inputs. Display devices 210, 220, 230, and 240 may be examples of display device 107 illustrated in FIG. 1 used to display sensor data to a user of FIG. 1 and/or receive input from the user.

In some embodiments, one, some, or all of the display devices are configured to display or otherwise communicate the sensor data as it is communicated from the sensor electronics module (e.g., in a data package that is transmitted to respective display devices), without any additional prospective processing required for calibration and real-time display of the sensor data.

The plurality of display devices may include a custom display device specially designed for displaying certain types of displayable sensor data associated with analyte data received from sensor electronics module. In certain embodiments, the plurality of display devices may be configured for providing alerts/alarms based on the displayable sensor data. Display device 210 is an example of such a custom device. In some embodiments, one of the plurality of display devices is a smartphone, such as display device 220 which represents a mobile phone, using a commercially available operating system (OS), and configured to display a graphical representation of the continuous sensor data (e.g., including current and historic data). Other display devices can include other hand-held devices, such as display device 230 which represents a tablet, display device 240 which represents a smart watch, medical device 208 (e.g., an insulin delivery device or a blood glucose meter), and/or a desktop or laptop computer (not shown).

Because different display devices provide different user interfaces, content of the data packages (e.g., amount, format, and/or type of data to be displayed, alarms, and the like) can be customized (e.g., programmed differently by the manufacture and/or by an end user) for each particular display device. Accordingly, in certain embodiments, a plurality of different display devices can be in direct wireless communication with a sensor electronics module (e.g., such as an on-skin sensor electronics module 204 that is physically connected to continuous analyte sensor(s) 202) during a sensor session to enable a plurality of different types and/or levels of display and/or functionality associated with the displayable sensor data. In certain embodiments, the type of alarms customized for each particular display device, the number of alarms customized for each particular display device, the timing of alarms customized for each particular display device, and/or the threshold levels configured for each of the alarms (e.g., for triggering) are based on output 144 (e.g., as mentioned, output 144 may be indicative of the current health of a user, the state of a user's kidney, current treatment recommended to a user, and/or physiological parameters of a user when experiencing different symptoms) stored in user profile 118 for each user.

As mentioned, sensor electronics module 204 may be in communication with a medical device 208. Medical device 208 may be a passive device in some example embodiments of the disclosure. For example, medical device 208 may be an insulin pump for administering insulin to a user. For a variety of reasons, it may be desirable for such an insulin pump to receive and track potassium and/or glucose values transmitted from continuous analyte monitoring system 104, where continuous analyte sensor 202 is configured to measure potassium and/or glucose.

Further, as mentioned, sensor electronics module 204 may also be in communication with other non-analyte sensors 206. In some cases, any one or more non-analyte sensors mentioned below may be integrated into continuous analyte monitoring system 104, or may be distinct and separate from the continuous analyte monitoring system 104. Non-analyte sensors 206 may include, but are not limited to, an altimeter sensor, an accelerometer sensor, a temperature sensor, a respiration rate sensor, a sweat sensor, etc. Non-analyte sensors 206 may also include monitors such as heart rate monitors, ECG monitors, blood pressure monitors, pulse oximeters, caloric intake, and medicament delivery devices. Non-analyte sensors 206 may also include data systems for measuring non-patient specific phenomena such as time, ambient pressure, or ambient temperature which could include an atmospheric pressure sensor, an external air temperature sensor or a clock, timer, or other time measure of when in time the sensor was first insertion or the time of sensor life remaining compared to insertion time could be used as calibration or other data inputs for an algorithmic model. One or more of these non-analyte sensors 206 may provide data to decision support engine 114 described further below. In some aspects, a user may manually provide some of the data for processing by training server system 140 and/or decision support engine 114 of FIG. 1.

In certain embodiments, the non-analyte sensors 206 may be combined in any other configuration, such as, for example, combined with one or more continuous analyte sensors 202. As an illustrative example, a non-analyte sensor, e.g., a temperature sensor, may be combined with a continuous analyte sensor 202 configured to measure potassium to form a potassium/temperature sensor used to transmit sensor data to sensor electronics module 204 using common communication circuitry. As another illustrative example, a non-analyte sensor, e.g., a temperature sensor, may be combined with a multi-analyte sensor 202 configured to measure potassium and glucose to form a potassium/glucose/temperature sensor used to transmit sensor data to the sensor electronics module 204 using common communication circuitry.

In certain embodiments, a wireless access point (WAP) may be used to couple one or more of continuous analyte monitoring system 104, the plurality of display devices, medical device(s) 208, and/or non-analyte sensor(s) 206 to one another. For example, WAP 138 may provide Wi-Fi and/or cellular connectivity among these devices. Near Field Communication (NFC) and or Bluetooth may also be used among devices depicted in diagram 200 of FIG. 2.

FIG. 3 illustrates example inputs and example metrics that are calculated based on the inputs for use by the decision support system of FIG. 1, according to some embodiments disclosed herein. In particular, FIG. 3 provides a more detailed illustration of example inputs and example metrics introduced in FIG. 1.

FIG. 3 illustrates example inputs 128 on the left, application 106 and decision support engine 114 including DAM 116 in the middle, and metrics 130 on the right. In certain embodiments, each one of metrics 130 may correspond to one or more values, e.g., discrete numerical values, ranges, or qualitative values (high/medium/low, stable/unstable, etc.). Application 106 obtains inputs 128 through one or more channels (e.g., manual user input, sensors/monitors, other applications executing on display device 107, EMRs, etc.). As mentioned previously, in certain embodiments, inputs 128 may be processed by DAM 116 and/or decision support engine 114 to output metrics 130. Inputs and metrics 130 may be used by decision support engine 114 to provide decision support to the user. For example, inputs 128 and metrics 130 may be used by training server system 140 to train and deploy one or more machine learning models for use by decision support engine 114 for providing decision support around kidney health, including the presence and/or severity of kidney disease.

In certain embodiments, starting with inputs 128, food consumption information may include information about one or more of meals, snacks, and/or beverages, such as one or more of the size, content (milligrams (mg) of potassium, carbohydrate, fat, protein, etc.), sequence of consumption, and time of consumption. In certain embodiments, food consumption may be provided by a user through manual entry, by providing a photograph through an application that is configured to recognize food types and quantities, and/or by scanning a bar code or menu. In various examples, meal size may be manually entered as one or more of calories, quantity (e.g., “three cookies”), menu items (e.g., “Royale with Cheese”), and/or food exchanges (e.g., 1 fruit, 1 dairy). In some examples, meal information may be received via a convenient user interface provided by application 106.

In certain embodiments, food consumption information (the type of food (e.g., liquid or solid, snack or meal, etc.) and/or the composition of the food (e.g., carbohydrate, fat, protein, etc.)) may be determined automatically based on information provided by one or more sensors. Some example sensors may include body sound sensors (e.g., abdominal sounds may be used to detect the types of meal, e.g., liquid/solid food, snack/meal, etc.), radio-frequency sensors, cameras, hyperspectral cameras, and/or analyte (e.g., insulin, glucose, lactate, etc.) sensors to determine the type and/or composition of the food.

In certain embodiments, food consumption entered by a user may relate to potassium consumed by the user. Potassium for consumption may include any natural or designed food or beverage that contains potassium, such as apricot juice, a banana, or potatoes, for example.

In certain embodiments, exercise information is also provided as an input. Exercise information may be any information surrounding activities, such as activities requiring physical exertion by the user. For example, exercise information may range from information related to low intensity (e.g., walking a few steps) and high intensity (e.g., five mile run) physical exertion. In certain embodiments, exercise information may be provided, for example, by an accelerometer sensor on a wearable device such as a watch, fitness tracker, and/or patch. In certain embodiments, exercise information may also be provided through manual user input and/or through a surrogate sensor and prediction algorithm measuring changes to heart rate (or other cardiac metrics). When predicting that a user is exercising based on his/her sensor data, the user may be asked to confirm if exercise is occurring, what type of exercise, and or the level of strenuous exertion being used during the exercise over a specific period. This data may be used to train the system to learn about the user's exercise patterns to reduce the need for confirmation questions as time progresses. Other analytes and sensor data may also be included in this training set, including analytes and other measured elements described herein including temporal elements such as time and day.

In certain embodiments, user statistics, such as one or more of age, height, weight, BMI, body composition (e.g., % body fat), stature, build, or other information may also be provided as an input. In certain embodiments, user statistics are provided through a user interface, by interfacing with an electronic source such as an electronic medical record, and/or from measurement devices. In certain embodiments, the measurement devices include one or more of a wireless, e.g., Bluetooth-enabled, weight scale and/or camera, which may, for example, communicate with the display device 107 to provide user data.

In certain embodiments, treatment/medication information is also provided as an input. Medication information may include information about the type, dosage, and/or timing of when one or more medications are to be taken by the user. As mentioned herein, the medication information may include information about one or more diuretics, one or more drugs known to reduce potassium, one or more drugs known to damage the kidney, one or more drugs known to control the complications of kidney disease that are prescribed to the user, and/or one or more medications for treating one or more symptoms of kidney disease, hyperkalemia, hypokalemia, diabetes, and/or other conditions and diseases the user may have. Treatment information may include information regarding different lifestyle habits, surgical procedures, and/or other non-invasive procedures recommended by the user's physician. For example, the user's physician may recommend a user increase/decrease their potassium intake, exercise for a minimum of thirty minutes a day, or increase an insulin dosage or other medication to maintain, and/or improve, kidney health, reduce hyper- and/or hypokalemic episodes, etc.

As another example, a healthcare professional may recommend that a user engage in at-home dialysis treatment and/or dialysis treatment at a clinic. Dialysis is a treatment for kidney failure that rids the body of unwanted toxins, waste products, and excess fluids by filtering a user's blood. Users with end stage renal disease (ESRD) (e.g., CKD stage 5) may be prescribed dialysis treatment to supplement and/or replace filtering generally performed by the kidney, given dialysis helps to keep the potassium, phosphorus, and sodium levels in a patient's body balanced. Dialysis treatment may be hemodialysis or peritoneal dialysis. In hemodialysis, blood is pumped out of a user's body to an artificial kidney machine, and returned to the body by tubes that connect the user to the machine. In peritoneal dialysis, the inside lining of the user's stomach acts as a natural filter. As such, information about dialysis treatment for the user may be included in the treatment/medication information. In certain embodiments, treatment/medication information may be provided through manual user input.

In certain embodiments, analyte sensor data may also be provided as input, for example, through continuous analyte monitoring system 104. In certain embodiments, analyte sensor data may include potassium data (e.g., a user's potassium values) measured by at least a CPM (or multi-analyte sensor configured to measure at least potassium) that is a part of continuous analyte monitoring system 104. In certain embodiments, analyte sensor data may include glucose data measured by at least a glucose sensor (or multi-analyte sensor configured to measure at least glucose) that is a part of or separate from the continuous analyte monitoring system 104. In certain embodiments, analyte sensor data may include creatinine data measured by at least a creatinine sensor (or multi-analyte sensor configured to measure at least creatinine) that is separate from or a part of continuous analyte monitoring system 104. In certain embodiments, analyte sensor data may include BUN data measured by at least a BUN sensor (or multi-analyte sensor configured to measure at least BUN) that is separate from or a part of continuous analyte monitoring system 104.

In certain embodiments, analyte sensor data may include ammonia data measured by at least an ammonia sensor (or multi-analyte sensor configured to measure at least ammonia) that is a part of continuous analyte monitoring system 104. In certain embodiments, analyte sensor data may include C-peptide data measured by at least a C-peptide sensor (or multi-analyte sensor configured to measure at least C-Peptide) that is a part of continuous analyte monitoring system 104. In certain embodiments, analyte sensor data may include cystatin C data measured by at least a cystatin C sensor (or multi-analyte sensor configured to measure at least cystatin C) in continuous analyte monitoring system 104. In certain embodiments, analyte sensor data may include lactate data measured by at least a lactate sensor (or multi-analyte sensor configured to measure at least lactate) that is a part of continuous analyte monitoring system 104.

In certain embodiments, input may also be received from one or more non-analyte sensors, such as non-analyte sensors 206 described with respect to FIG. 2. Input from such non-analyte sensors 206 may include information related to a heart rate, heart rate variability (e.g., the variance in time between the beats of the heart), ECG data, a respiration rate, oxygen saturation, a blood pressure, or a body temperature (e.g. to detect illness, physical activity, etc.) of a user. In certain embodiments, electromagnetic sensors may also detect low-power radio frequency (RF) fields emitted from objects or tools touching or near the object, which may provide information about user activity or location.

In certain embodiments, input received from non-analyte sensors may include input relating to a user's insulin delivery. In particular, input related to the user's insulin delivery may be received, via a wireless connection on a smart pen, via user input, and/or from an insulin pump. Insulin delivery information may include one or more of insulin volume, time of delivery, etc. Other parameters, such as insulin action time or duration of insulin action, may also be received as inputs.

In certain embodiments, time may also be provided as an input, such as time of day or time from a real-time clock. For example, in certain embodiments, input analyte data may be timestamped to indicate a date and time when the analyte measurement was taken for the user.

User input of any of the above-mentioned inputs 128 may be through a user interface, such a user interface of display device 107 of FIG. 1.

As described above, in certain embodiments, DAM 116 and/or decision support engine (e.g., using one or more trained models) determines or computes the user's metrics 130 based on inputs 128. An example list of metrics 130 is shown in FIG. 3.

In certain embodiments, potassium levels may be determined from sensor data (e.g., potassium measurements obtained from a CPM of continuous analyte monitoring system 104, sweat sensor configured to measure potassium in sweat, where the sweat sensor may be one of non-analyte sensor(s) 206). For example, potassium levels refer to time-stamped potassium measurements or values that are continuously generated and stored over time.

In certain embodiments, a potassium baseline may be determined from sensor data (e.g., potassium measurements obtained from a continuous lactate sensor of continuous analyte monitoring system 104). For example, potassium baseline may be determined based on historical levels of potassium at rest (e.g., 2 weeks prior), an average of potassium levels at rest over time, or time segmented data of the user's potassium levels at rest. A potassium baseline represents a user's normal potassium levels during periods where significant fluctuations in potassium production is typically not expected. A user's baseline potassium is generally expected to remain constant over time, unless challenged through an action such as the consumption of potassium or potassium rich foods or changed as a result of declining kidney health or kidney dysfunction.

Further, each user may have a different potassium baseline. In certain embodiments, a user's potassium baseline may be determined by calculating an average of potassium levels of the user over a specified amount of time where significant fluctuations are not expected. For example, the baseline potassium for a user may be determined over a period of time when the user is sleeping, sitting in a chair, or other periods of time where the user is sedentary and not consuming food or medication which would reduce or increase potassium levels (e.g., where no external conditions exist that would affect the potassium baseline exist). In certain embodiments, DAM 116 may continuously calculate a potassium baseline, time-stamp the calculated potassium baseline, and store the corresponding information in the user's profile 118.

In certain other embodiments, DAM 116 may use potassium levels measured over a period of time where the user is, at least for a subset of the period of time, engaging in exercise and/or consuming potassium and/or a an external condition exists that would affect the potassium baseline. In such embodiments, DAM 116 may, in some examples, first identify which measured potassium values are not to be used for calculating the potassium baseline by identifying which potassium values have been affected by an external event, such as the consumption of food, exercise, medication, or other perturbation that would disrupt the capture of a potassium baseline measurement. DAM 116 may then exclude such measurements when calculating the potassium baseline of the user. In some other examples, DAM 116 may calculate the potassium baseline by first determining a percentage of the number of potassium values measured during this time period that represent the lowest lactate values measured. DAM 116 may then take an average of such potassium values to determine the potassium baseline level.

In certain embodiments, an absolute maximum potassium level may be determined from sensor data (e.g., potassium measurements obtained from a continuous CPM of continuous analyte monitoring system 104), health/sickness metrics (e.g., described in more detail below), and/or disease stage metrics (e.g., described in more detail below). The absolute maximum potassium level represents a user's maximum potassium level determined to be unsafe over a period of time (e.g., hourly, weekly, daily, etc.). In certain embodiments, the absolute maximum potassium level may be consistent across all users (e.g., set to 5.5 mmol/L based on current medical guidelines). In certain other embodiments, each patient may have a different absolute maximum potassium level. For example, an absolute maximum potassium level may be lower for a user diagnosed with stage 1 CKD (e.g., normal or high GFR (GFR>90 mL/min)) than a user diagnosed with stage 5 end stage CKD (e.g., GFR<15 mL/min) who also has hyperkalemia. In certain embodiments, the absolute maximum potassium level per patient may change over time. For example, a user may be initially assigned an absolute maximum potassium level based on clinical input. This assigned absolute maximum potassium level may be adjusted over time based on other sensor data, disease stages, comorbidities, etc. for the patient.

For example, a user's absolute maximum potassium level may vary over time as a user's kidney health, kidney disease, and/or one or more other diseases progress and/or improve. In certain embodiments, a first absolute maximum potassium level may be determined for periods of time where no external conditions exist that would affect the potassium level, and a second absolute maximum potassium level may be determined for periods of time where external conditions do exist that would affect the potassium level (e.g., during periods of time when the user is consuming potassium, exercising, taking medication that affects potassium levels, etc.).

In certain embodiments, an absolute minimum potassium level may be determined from sensor data (e.g., potassium measurements obtained from a continuous potassium sensor of continuous analyte monitoring system 104), health/sickness metrics (e.g., described in more detail below), and/or disease stage metrics (e.g., described in more detail below). The absolute minimum potassium level represents a user's minimum potassium level determined to be unsafe over a period of time (e.g., hourly, weekly, daily, etc.). In certain embodiments, the absolute minimum potassium level may be consistent across all users (e.g., set based on current medical guidelines). In certain other embodiments, each user may have a different absolute minimum potassium level. For example, an absolute minimum potassium level may be lower for a user diagnosed with stage 1 CKD (e.g., normal or high GFR (GFR>90 mL/min)) than a user diagnosed with stage 5 end stage CKD (e.g., GFR<15 mL/min) who also has hypokalemia. In certain embodiments, the absolute minimum potassium level per patient may change over time. For example, a user may be initially assigned an absolute minimum potassium level based on clinical input. This assigned absolute minimum potassium level may be adjusted over time based on other sensor data, disease stages, comorbidities, etc. for the patient.

For example, a user's absolute minimum potassium level may vary over time as a user's kidney health, kidney disease, and/or one or more other diseases progress and/or improve. In certain embodiments, a first absolute minimum potassium level may be determined for periods of time where no external conditions exist that would affect the potassium level, and a second absolute minimum potassium level may be determined for periods of time where external conditions do exist that would affect the potassium level (e.g., during periods of time when the user is consuming potassium, exercising, taking medication that affects potassium levels, etc.).

In certain embodiments, potassium level rates of change may be determined from sensor data (e.g., potassium measurements obtained from a CPM of continuous analyte monitoring system 104 over time). For example, a potassium level rate of change refers to a rate that indicates how one or more time-stamped potassium measurements or values change in relation to one or more other time-stamped potassium measurements or values. Potassium level rates of change may be determined over one or more seconds, minutes, hours, days, etc.

In certain embodiments, potassium level rates of change may be determined for a number of potassium measurements using a line of best fit representing the average rate of change of the number of potassium measurements. In this example, as current potassium measurements are obtained from a sensor (e.g., continuous potassium sensor of continuous analyte monitoring system 104), the line of best fit may change to better represent the rate of change based on current potassium measurements. Further, based on the rate of change progression over time, different statistics (e.g., mean, standard deviation, etc.) may be calculated to determine when large changes in potassium level rates of change occur.

In certain embodiments, determined potassium level rates of change may be marked as “increasing rapidly” or “decreasing rapidly”. As used herein, “rapidly” may describe potassium level rates of change that are clinically significant and pointing towards a trend of the potassium level of the patient likely breaching the absolute maximum potassium level or the absolute minimum potassium level (e.g., potassium threshold) within a next period of defined time. In other words, a predictive trend (e.g., produced by decision support engine 114 using one or more trained models) may, in some cases, indicate that a patient is likely to hit, for example, the absolute maximum potassium level within a specified time period (e.g., one or two hours) based on the determined potassium level rate of change. Accordingly, such a potassium level rate of change may be marked as “increasing rapidly”. Similarly, a predictive trend (e.g., produced by decision support engine 114 using one or more trained models) may, in some cases, indicate that a patient is likely to hit the absolute minimum potassium level within a specified time period (e.g., one or two hours) based on the potassium level rate of change determined. Accordingly, such a potassium level rate of change may be marked as “decreasing rapidly”.

In certain embodiments, determined potassium level rates of change may be considered in light of non-analyte sensor data to determine whether there are one or more external factors influencing the user's potassium levels. For example, where the potassium level rate of change is marked as “increasing rapidly,” and it is known that the user is consuming a meal or completing an exercise session, the potassium level rates of change may be compared with other potassium level rates of change measurements when the user is consuming a meal or completing an exercise session. In other words, potassium level rates of change when the user is consuming a meal or completing an exercise session may be analyzed separately from potassium level rates of change at times when the user is at rest.

In certain embodiments, potassium baseline rates of change may be determined from potassium baselines determined for a user over time. For example, a potassium baseline rate of change refers to a rate that indicates how one or more time-stamped potassium baselines for a user change in relation to one or more other time-stamped potassium baselines for the same user. Potassium baseline rates of change may be determined over one or more seconds, minutes, hours, days, etc.

In certain embodiments, a potassium clearance rate may be determined from sensor data (e.g., potassium measurements obtained from a CPM of continuous analyte monitoring system 104) following the consumption of a known, or estimated, amount of potassium. Potassium clearance rates analyzed over time may be indicative of kidney function. In particular, the slope of a curve of potassium clearance during a first time period (e.g., after consuming a known amount of potassium) compared to the slope of a curve of potassium clearance during a second time period (e.g., after consuming the same amount of potassium) may be indicative of a kidney's ability to function, and more particularly, to maintain potassium homeostasis (e.g., a potassium clearance rate may be slower when a user's kidney is impaired than when a user's kidney is healthy).

In certain embodiments, the potassium clearance rate may be determined by calculating a slope between an initial potassium value (e.g., during a period of increased potassium levels) and a potassium baseline associated with the user. In certain embodiments, a potassium clearance rate may be calculated over time until the increased potassium levels of the user reach some value relative to the user's potassium baseline (e.g., % of a user's potassium baseline). Potassium clearance rates calculated over time may be time-stamped and stored in the user's profile 118.

In certain embodiments, potassium trends may be determined based on potassium levels over certain periods of time. In certain embodiments, potassium trends may be determined based on potassium baselines over certain periods of time. In certain embodiments, potassium trends may be determined based on absolute minimum potassium levels over certain periods of time. In certain embodiments, potassium trends may be determined based on absolute maximum potassium levels over certain periods of time. In certain embodiments, potassium trends may be determined based on potassium level rates of change over certain periods of time. In certain embodiments, potassium trends may be determined based on potassium baseline rates of change over certain periods of time. In certain embodiments, potassium trends may be determined based on calculated potassium clearance rates, potassium level variability, potassium threshold based trends, and/or potassium level averages over certain periods of time.

In certain embodiments, glucose levels may be determined from sensor data (e.g., blood glucose measurements obtained from a continuous lactate sensor of continuous analyte monitoring system 104).

In certain embodiments, glucose level rates of change may be determined from sensor data (e.g., glucose measurements obtained from a continuous glucose monitor (CGM) of continuous analyte monitoring system 104 over time). For example, a glucose level rate of change refers to a rate that indicates how one or more time-stamped glucose measurements or values change in relation to one or more other time-stamped glucose measurements or values. Glucose level rates of change may be determined over one or more seconds, minutes, hours, days, etc.

In certain embodiments, a blood glucose trend may be determined based on glucose levels over a certain period of time. In certain embodiments, glucose trends may be determined based on glucose level rates of change over certain periods of time.

In certain embodiments, insulin sensitivity may be determined using historical data, real-time data, or a combination thereof, and may, for example, be based upon one or more inputs 128, such as one or more of food consumption information, continuous analyte sensor data, non-analyte sensor data (e.g., insulin delivery information from an insulin device), etc. Insulin sensitivity refers to how responsive a user's cells are to insulin. Improving insulin sensitivity for a user may help to reduce insulin resistance in the user.

In certain embodiments, insulin on board may be determined using non-analyte sensor data input (e.g., insulin delivery information) and/or known or learned (e.g. from user data) insulin time action profiles, which may account for both basal metabolic rate (e.g., update of insulin to maintain operation of the body) and insulin usage driven by activity or food consumption.

In certain embodiments, creatinine levels may be determined from sensor data (e.g., creatinine measurements obtained from continuous analyte monitoring system 104).

In certain embodiments, an absolute maximum creatinine level may be determined from sensor data (e.g., creatinine measurements obtained from a continuous creatinine sensor of continuous analyte monitoring system 104), health/sickness metrics (e.g., described in more detail below), and/or disease stage metrics (e.g., described in more detail below). The absolute maximum creatinine level represents a user's maximum creatinine level determined to be unsafe over a period of time (e.g., hourly, weekly, daily, etc.). Each user may have a different absolute maximum creatinine level. A user's absolute maximum creatinine level may vary over time as a user's kidney health, kidney disease, and/or one or more other diseases progress and/or improve.

In certain embodiments, creatinine level rates of change may be determined from sensor data (e.g., creatinine measurements obtained from a creatinine sensor of continuous analyte monitoring system 104 over time). For example, a creatinine level rate of change refers to a rate that describes how one or more time-stamped creatinine measurements or values change in relation to one or more other time-stamped creatinine measurements or values. Creatinine level rates of change may be determined over one or more seconds, minutes, hours, days, etc. In certain embodiments, average creatinine levels may be calculated for determining rates of change of the calculated average creatinine levels of the user.

In certain embodiments, creatinine trends may be determined based on creatinine levels over certain periods of time.

In certain embodiments, BUN levels may be determined from sensor data (e.g., BUN measurements obtained from continuous analyte monitoring system 104). In certain embodiments, BUN trends may be determined based on BUN levels over certain periods of time.

In certain embodiments, ammonia levels may be determined from sensor data (e.g., ammonia measurements obtained from continuous analyte monitoring system 104). In certain embodiments, ammonia trends may be determined based on ammonia levels over certain periods of time.

In certain embodiments, C-peptide levels may be determined from sensor data (e.g., C-peptide measurements obtained from continuous analyte monitoring system 104). In certain embodiments, C-peptide trends may be determined based on C-peptide levels over certain periods of time.

In certain embodiments, cystatin C levels may be determined from sensor data (e.g., cystatin C measurements obtained from continuous analyte monitoring system 104). In certain embodiments, cystatin C trends may be determined based on cystatin C levels over certain periods of time.

In certain embodiments, health and sickness metrics may be determined, for example, based on one or more of user input (e.g., pregnancy information or known sickness or disease information), from physiologic sensors (e.g., temperature), activity sensors, or a combination thereof. In certain embodiments, based on the values of the health and sickness metrics, for example, a user's state may be defined as being one or more of healthy, ill, rested, or exhausted.

In certain embodiments, disease stage metrics, such as for kidney disease, may be determined, for example, based on one or more of user input or output provided by decision support engine 114 illustrated in FIG. 1. In certain embodiments, example disease stages for kidney disease, can include AKI, stage 1 CKD with normal or high GFR (e.g., GFR>90 mL/min), stage 2 mild CKD (e.g., GFR=60-89 mL/min), stage 3A moderate CKD (e.g., GFR=45-59 mL/min), stage 3B moderate CKD (e.g., GFR=30-44 mL/min), stage 4 severe CKD (e.g., GFR=15-29 mL/min), and stage 5 end stage CKD (e.g., GFR<15 mL/min). In certain embodiments, example disease stages may be represented as a GFR value/range, severity score, and the like.

In certain embodiments, the meal state metric may indicate the state the user is in with respect to food consumption. For example, the meal state may indicate whether the user is in one of a fasting state, pre-meal state, eating state, post-meal response state, or stable state. In certain embodiments, the meal state may also indicate nourishment on board, e.g., meals, snacks, or beverages consumed, and may be determined, for example from food consumption information, time of meal information, and/or digestive rate information, which may be correlated to food type, quantity, and/or sequence (e.g., which food/beverage was eaten first).

In certain embodiments, meal habits metrics are based on the content and the timing of a user's meals. For example, if a meal habit metric is on a scale of 0 to 1, the better/healthier meals the user eats the higher the meal habit metric of the user will be to 1, in an example. Also, the more the user's food consumption adheres to a certain time schedule or a recommended diet, the closer their meal habit metric will be to 1, in the example.

In certain embodiments, medication adherence is measured by one or more metrics that are indicative of how committed the user is towards their medication regimen. In certain embodiments, medication adherence metrics are calculated based on one or more of the timing of when the user takes medication (e.g., whether the user is on time or on schedule), the type of medication (e.g., is the user taking the right type of medication), and the dosage of the medication (e.g., is the user taking the right dosage). In certain embodiments, medication adherence of a user may be determined in a clinical trial where medication consumption and timing of such medication consumption is monitored, through user input, and/or based on analyte data received from analyte monitoring system 104.

In certain embodiments, the activity level metric may indicate the user's level of activity. In certain embodiments, the activity level metric be determined, for example based on input from an activity sensor or other physiologic sensors, such as non-analyte sensors 206. In certain embodiments, the activity level metric may be calculated by DAM 116 based on one or more of inputs 128, such as one or more of exercise information, non-analyte sensor data (e.g., accelerometer data), time, user input, etc. In certain embodiments, the activity level may be expressed as a step rate of the user. Activity level metrics may be time-stamped so that they can be correlated with the user's lactate levels at the same time.

In certain embodiments, exercise regimen metrics may indicate one or more of what type of activities the user engages in, the corresponding intensity of such activities, frequency the user engages in such activities, etc. In certain embodiments, exercise regimen metrics may be calculated based on one or more of analyte sensor data input (e.g., from a lactate monitor, a glucose monitor, etc.), non-analyte sensor data input (e.g., non-analyte sensor data input from an accelerometer, a heart rate monitor, a respiration rate sensor, etc.), calendar input, user input, etc.

In certain embodiments, body temperature metrics may be calculated by DAM 116 based on inputs 128, and more specifically, non-analyte sensor data from a temperature sensor. In certain embodiments, heart rate metrics (e.g., including heart rate and heart rate variability) may be calculated by DAM 116 based on inputs 128, and more specifically, non-analyte sensor data from a heart rate sensor. In certain embodiments, respiratory metrics may be calculated by DAM 113 based on inputs 128, and more specifically, non-analyte sensor data from a respiratory rate sensor.

In certain embodiments, as described in more detail below, physiological parameters (e.g., potassium levels, potassium level rates of change, glucose levels, C-peptide levels, heart rate, blood pressure, etc.) associated with the user may be stored as metrics 130 when the user is confirmed to be experiencing one or more symptoms associated with potassium imbalance. In particular, physiological parameters associated with the user when the user is experiencing (1) potassium fluctuations, (2) numbness, (3) tingling, (4) shortness of breath (or chest pain), (5) muscle weakness, and/or (6) arrhythmia may be stored as metrics 130. In certain embodiments, such physiological parameters may be analyzed over time to provide an indication of the improvement or the deterioration of a user's kidney disease. In certain embodiments, the user specific values of the physiological parameters for different symptoms experienced by the user may be a valuable input for training one or models designed to assess the presence and/or severity of kidney disease in a user. In certain embodiments, the user specific values of the physiological parameters for different symptoms experienced by the user may be used to create one or more personalized models specific to the user for more accurately predicting (1) the kidney health of the user, including the presence and/or severity of kidney diseaseand (2) one or more symptoms of the user associated with potassium imbalance.

### Example Methods and Systems for Providing Decision Support Around Kidney Health

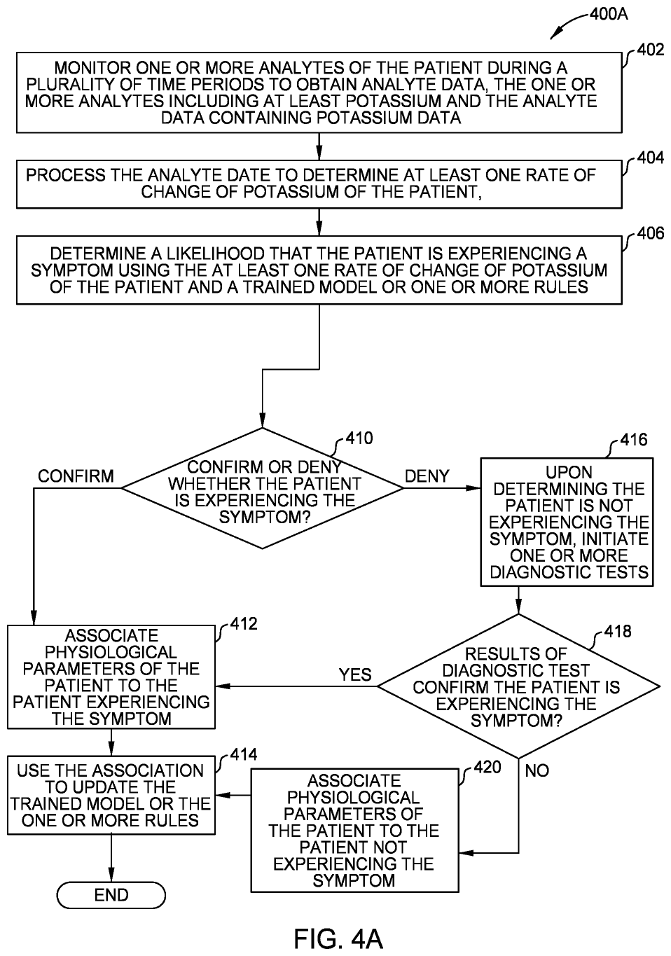

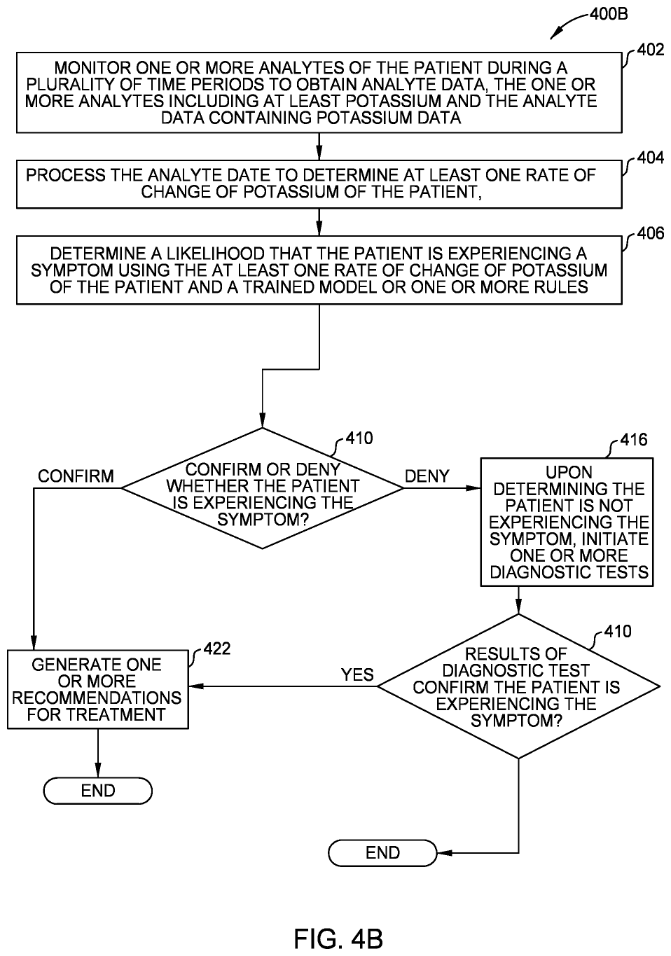

FIG. 4A is an example workflow 400A for determining (e.g., using a machine learning model and/or an algorithm (e.g., rules-based model)) the likelihood of one or more symptoms commonly associated with potassium imbalance (e.g., in some cases, due to kidney disease) occurring in real-time or within a specified period of time and retraining or updating the model based on patient input and/or diagnostics tests, according to certain embodiments of the present disclosure. FIG. 4B is an example workflow 400B for determining the likelihood of one or more symptoms commonly associated with potassium imbalance occurring in real-time or within a specified period of time using the model and providing one or more recommendations for treatment for the user based the determined likelihood, according to certain embodiments of the present disclosure. For example, workflow 400B may be performed to provide a prediction of one or more symptoms to a user, using a continuous analyte monitoring system 104 including, at least, a continuous potassium monitor (CPM) 202, as illustrated in FIGS. 1 and 2, and further provide one or more recommendations for treatment for the user based the prediction. In certain embodiments, operations 400A and 400B may be combined (e.g., such that in addition to providing symptom predictions using a model and retraining the model, treatment recommendations are also provided to the patient based on the predictions) and, in some other embodiments, operations 400A and 400B may be performed separately. Workflow 400A and workflow 400B may be performed by decision support system 100 to collect data, such a inputs 128 and metrics 130, including for example, analyte data, patient information, and non-analyte sensor data mentioned above, to provide a prediction of the risk or likelihood of one or more symptoms of potassium imbalance occurring in-real time (e.g., the user is currently experiencing these one or more symptoms) or within a specified period of time for a patient (e.g., the user is expected to experience these one or more symptoms within five minutes, thirty minutes, one hour, etc.). In certain embodiments, the prediction may be presented as a percentage likelihood of one or more symptoms of potassium imbalance occurring in-real time or within a specified period of time for a patient.

In certain embodiments, decision support system 100 presented herein may be configured to identify or predict that a patient is going to experience one or more symptoms commonly associated with potassium imbalance, even in cases where the patient does not realize they are experiencing such symptoms. In particular, in the early stages of kidney disease (e.g., where potassium imbalance is common), a patient might have few symptoms related to kidney disease; however, the patient may not realize they have kidney disease until the condition is advanced. Thus, by being able to identify the occurrence of such symptoms of potassium imbalance and confirm the occurrence of such symptoms either based on user input or through one or more sensor data (e.g., generated by non-analyte sensor(s) 206), decision support system 100 presented herein offers information which may be critical to the early detection of kidney disease in a patient.

In certain embodiments, decision support engine 114 of decision support system 100 may use various algorithms or artificial intelligence (AI) models, such as machine-learning models, trained based on patient-specific data and/or population data to provide kidney disease symptom predictions. The algorithms and/or machine-learning models may take into account one or more inputs 128 and/or metrics 130 described with respect to FIG. 3 for a patient when predicting whether the patient is experiencing, or is likely to experience, one or more different symptoms commonly associated with potassium imbalance.

In certain embodiments, the one or more machine learning models may include multiple input, single output (MISO) models that are trained to predict the risk or likelihood of a patient experiencing one of the symptoms of potassium imbalance (e.g., (1) potassium fluctuations (hypokalemia, hyperkalemia, rapid rates of change, variability, etc.), (2) numbness, (3) tingling, (4) shortness of breath, (5) muscle weakness, (6) arrhythmia, or (7) death). For example, one model may be trained to predict the likelihood of the present or future occurrence of numbness, while another model may be trained to predict the present or future occurrence of tingling.

In certain embodiments, the one or more machine learning models may include multiple input, multiple output (MIMO) models that are trained to predict the risk or likelihood of a patient experiencing two or more of the symptoms of potassium imbalance. For example, a single model may be trained to predict the likelihood of the present or future occurrence of numbness and tingling. In certain embodiments, the model may be trained to output a vector having multiple values, where each value corresponds a likelihood of one of the symptoms for which the model is trained to predict the likelihood of occurrence. As an illustrative example, a vector output by a MIMO model may include five values, each value corresponding to one of the six symptoms of potassium imbalance (e.g., listed above). A first value that indicates 90% may indicate that there is a 90% chance that a patient is experiencing numbness or that the patient has a 90% risk or likelihood of experiencing numbness within a predefined time limit. Meanwhile, a second value that indicates 0% may indicate that there is a 0% change that the patient is experiencing tingling or that the patient has a 0% risk or likelihood of experiencing tingling within a predefined time limit.

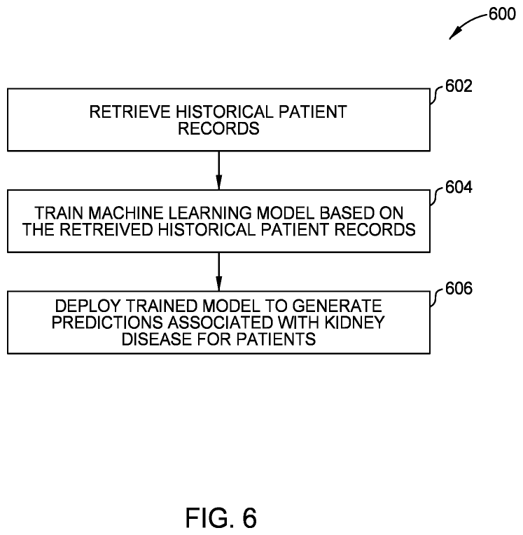

The one or more machine-learning models described herein for making such predictions may be initially trained using population data. A method for training the one or more machine learning models may be described in more detail below with respect to FIG. 6.

In certain embodiments, as an alternative to using machine learning models, decision support engine 114 may use rules-based models to predict the risk or likelihood of a patient experiencing one of the symptoms. Rules-based models involve using a set of rules for analyzing data. These rules are sometimes referred to as ‘If statements’ as they tend to follow the line of ‘If X happens then do or conclude Y’. In particular, decision support engine 114 may apply rule-statements (e.g., if, then statements) to predict one or more kidney disease symptoms for a patient.

Such rules may be defined and maintained by decision support engine 114 in a reference library. For example, the reference library may maintain ranges of potassium levels and ranges of potassium level rates of change which may be mapped to different symptoms experienced by patients with kidney disease. In certain embodiments, such rules may be determined based on empirical research or an analysis of historical patient records, such as the records stored in historical records database 112. In some cases, the reference library may become very granular. For example, other factors may be used in the reference library to create such “rules”. Other factors may include gender, age, diet, disease history, family disease history, body mass index (BMI), etc. Increased granularity may provide more accurate outputs.

As mentioned, FIG. 4A illustrates a workflow 400A for updating a model, used to determine the likelihood of one or more symptoms commonly associated with kidney disease occurring in real-time or within a specified period of time. The model may be a (1) machine-learning model initially trained based on the population data or (2) rules-based model. Workflow 400A is described below with reference to FIGS. 1 and 2 and their components.

At block 402, workflow 400 begins by continuously monitoring one or more analytes of a patient, such as user 102 illustrated in FIG. 1, during a plurality of time periods to obtain analyte data. The one or more analytes monitored may include at least potassium; thus, the analyte data may contain potassium data. Block 402 may be performed by continuous analyte monitoring system 104 illustrated in FIGS. 1 and 2, and more specifically, continuous analyte sensor(s) 202 illustrated in FIG. 2, in certain embodiments. For example, continuous analyte monitoring system 104 may comprise a CPM 202 configured to measure the patient's potassium levels.

As mentioned, potassium is one of the most important minerals in the body. Potassium helps to regulate fluid balance, muscle contractions, and nerve signals. A high-potassium diet may also help to reduce blood pressure and water retention, protect against stroke, and prevent osteoporosis and kidney stones. Approximately 98% of the body's potassium is stored intracellularly while the remaining 2% is stored extracellularly. The kidney is primarily responsible for maintaining total body potassium content by matching potassium intake with potassium excretion. Adjustments in renal potassium excretion occur over several hours; therefore, changes in extracellular potassium concentration are initially buffered by movement of potassium into or out of skeletal muscle.

The regulation of potassium distribution between the intracellular and extracellular space is referred to as internal potassium balance. The most important factors regulating this movement under normal conditions are insulin and catecholamines (e.g., dopamine, epinephrine (adrenaline), and norepinephrine). In other words, the kidney plays a major role in potassium homeostasis by renal mechanisms that transport and regulate potassium secretion, reabsorption and excretion. However, when the kidney becomes damaged, or loses its ability to function, the kidney may no longer be capable of removing excess potassium; thus, potassium levels may build up in the body. Accordingly, potassium monitoring may prove to be useful for assessing kidney health, diagnosing or monitoring kidney disease, and/or predicting and preventing symptoms associated with hyperkalemia, which may occur as a result of kidney disease.

In certain embodiments, techniques may be introduced to account for inaccurate potassium levels. For example, the same device, or another device, may measure free hemoglobin and/or uses colorimetric measure to observe fluid abnormalities. Based on this information, the device, in some cases, may not report potassium levels and/or may alert the user that the potassium levels are likely inaccurate. In some cases, where an exact amount of free hemoglobin may be measured, then a correction factor may be applied to provide more accurate potassium levels for the patient.

While the main analyte for measurement described herein is potassium, in certain embodiments, other analytes may be considered. In particular, combining potassium measurements with additional analyte data may help to further inform the analysis around kidney disease symptom prediction. For example, monitoring additional types of analytes such as glucose, creatinine, lactate, blood urea nitrogen (BUN), ammonia, C-peptide, and/or cystatin C measured by continuous analyte monitoring system 104, may provide additional insight into the prediction of one or more symptoms of a patient that are commonly associated with kidney disease.

The additional insight gained from using a combination of analytes, and not just potassium, may increase the accuracy of the prediction. For example, the probability of accurately predicting symptoms associated with kidney disease may be a function of a number of analytes measured for a patient. For example, in some examples, a probability of accurately predicting that a patient is experiencing numbness using only potassium data (in addition to other non-analyte data) may be less than a probability of accurately predicting the patient is experiencing numbness using potassium and glucose data (in addition to other non-analyte data), which may also be less than a probability of accurately predicting the patient is experiencing numbness using potassium, glucose, and creatinine data (in addition to other non-analyte data) for analysis.

Additionally, a plurality of sensors, or a single CPM, measuring data over time to confirm or reject the presence of symptoms, may be used to understand whether the patient reported data is accurate. For example, where a patient is entering a clinically dangerous threshold of hypokalemia or hyperkalemia, yet the patient is not yet confirming they are experiencing symptoms of potassium imbalance, the patient may be unaware of the pending danger. The use of additional analytes for analysis may help to increase the confidence that the lack of patient confirmed symptoms is a false negative response by the patient, and patient is, in fact, in a state of danger. Accordingly, based on this confidence level, the system may be able to take one or more actions to help protect the patient. The one or more actions may include, but are not limited to, notifying family members, healthcare providers, emergency personnel, patient entered key contacts, and/or a monitoring service company trained to identify false negatives or to confirm that the patient is stable. In certain aspects, the same personnel mediated triage method may be employed to alert of false positives for patient reported symptoms where sensors are malfunctioning, or where such sensors are functioning as expected and reading normal levels for the patient, yet the patient is reporting severe symptoms (e.g., either intentionally or accidentally). The triage protocol or a secondary confirmatory source, such as a key contact, family member, trained service company employee, or medical professional previously designated by the patient, their care team, or their insurance provider, may be used to confirm or deny whether the patient is, in fact, experiencing the symptoms they allege to be experiencing.

In certain embodiments described herein, analyte combinations, e.g., measured and collected by one (e.g., multianalyte) or more sensors, for kidney disease symptom prediction, include potassium and at least one of creatinine, BUN, ammonia, C-peptide, lactate, and cystatin C; however, other analyte combinations may be considered.

For example, in certain embodiments, at block 402, continuous analyte monitoring system 104 may continuously monitor potassium and glucose levels of a patient during a plurality of time periods. In certain embodiments, the measured glucose concentrations may be used in conjunction with potassium levels for predicting one or more symptoms of the patient.

In particular, glucose is a simple sugar (e.g., a monosaccharide). Glucose can be both ingested, as well as, produced in the body from protein, fat, and carbohydrates. Increasing glucose stimulates insulin release. Insulin causes the cells to take in glucose and potassium for fuel. Thus, insulin stimulates potassium and glucose uptake by cells, thereby reducing serum (e.g., extracellular) potassium and glucose levels. In some cases, where glucose levels of a patient are increased and rate(s) of change of glucose levels in the patient's body are high, excess insulin may be produced thereby causing movement of potassium intracellularly. On the other hand, where glucose levels of a patient are decreased and rate(s) of change of glucose levels in the patient's body are low, there may be less insulin secretion. Low insulin may lead to limited access of glucose and potassium by the cells; thus, extracellular glucose and potassium levels may increase.

In some cases, a patient may suffer from insulin resistance. Insulin resistance occurs when cells in the patient's muscles, fat, and liver don't respond well to insulin. Accordingly, glucose metabolism, as well as potassium movement intracellularly may be impaired. As a result, the patient's pancreas makes more insulin to help glucose and insulin enter the patient's cells.

Insulin resistance may have a different effect on glucose metabolism as compared to potassium metabolism. Further, the effect of insulin resistance on glucose metabolism and potassium metabolism may be different for different patients. In particular, a patient with a first insulin resistance may require an X dose of insulin to reduce extracellular potassium levels by Y, while another patient with a second insulin resistance may require a Z dose of insulin to reduce extracellular potassium levels by Y. For example, a diabetic patient with insulin resistance, may be at higher risk for hyperkalemia and may require higher insulin concentrations when using insulin for hyperkalemia management.

In other words, while glucose levels of a patient may affect the amount of insulin produced by the body, which in turn is expected to decrease the amount of extracellular potassium that is available to be measured by CPM 202 (e.g., which may measure potassium in the interstitial fluid), the effect of insulin resistance on potassium metabolization may cause less than an expected amount of potassium to be moved intracellularly, even in cases where glucose and/or insulin for the patient are elevated. Accordingly, understanding the effect of insulin resistance on glucose metabolism, as well as the effect of insulin resistance on potassium metabolism, for the patient may be necessary to make accurate predictions about potassium levels of the patient and better understand the true health of a patient's kidney(s). For example, in some cases, glucose levels, in addition to the patient's insulin resistance, may aid in understanding whether abnormal potassium levels for the patient are, in fact, attributed to declining health of the patient's kidneys, or some other reason, such as increased insulin resistance, where insulin resistance may be caused by heart disease, liver disease, obesity, etc.

Further, in certain embodiments, the measured glucose concentrations, in addition to insulin resistance of the patient, may be used as an additional input when predicting one or more symptoms of the patient. For example, increased glucose in the bloodstream over time may cause the kidneys to filter too much blood. Over time, this extra work puts more pressure on the nephrons, which often results in the nephrons losing their vital filtering ability, thereby damaging the function of the kidney. Accordingly, the assessment of glucose levels over time may provide insight into the overall health of a patient's kidney which may aid in determining whether the patient is experiencing or will experience one or more symptoms of kidney disease and/or determining the likelihood of the patient developing kidney disease in the future.

In another example, at block 402, continuous analyte monitoring system 104 may continuously monitor potassium and creatinine levels of a patient during a plurality of time periods. In certain embodiments, the measured levels of creatinine may be used to assign a confidence level to measured potassium levels of the patient, where the confidence level indicates the level of certainty that the potassium levels measured for the patient reflect the patient's actual potassium levels. Further, in certain embodiments, the measured levels of creatinine may be used to inform the analysis as to whether the patient is experiencing, or is likely to experience one or more symptoms associated with potassium imbalance.

In particular, creatinine is a waste product produced by muscles from the breakdown of a compound called creatine. Creatinine is removed from the body by the kidneys, which filter almost all of the creatinine from the blood and release the creatinine into the urine. Accordingly, creatinine levels may provide insight into kidney health and function. Thus, a patient experiencing high levels of measured extracellular potassium and is assumed to have damaged kidney function (e.g., given excess potassium is not being filtered from the body), may also be expected to be experiencing high levels of measured creatinine (e.g., given a damaged kidney would not likely be capable of removing the creatinine from the blood). Accordingly, where measured creatinine levels are also high, a higher confidence level may be assigned to potassium levels measured for the patient. Further, the assumption that the patient's kidney(s) are damaged may be strengthened, thereby increasing the likelihood that the patient's kidney(s) are not working properly and increasing the likelihood that the patient is, in fact, experiencing (or about to experience) one or more symptoms commonly associated with kidney disease.

In yet another example, at block 402, continuous analyte monitoring system 104 may continuously monitor potassium and BUN levels of a patient during a plurality of time periods. In certain embodiments, the measured levels of BUN may also be used to assign a confidence level to measured potassium levels of the patient. Further, in certain embodiments, the measured levels of BUN may be used to inform the analysis as to whether the patient is experiencing, or is likely to, experience one or more symptoms associated with potassium imbalance.

In particular, the liver produces ammonia, which contains nitrogen, after the liver breaks down proteins used by cells in the body. The nitrogen combines with other elements, such as carbon, hydrogen and oxygen, to form urea, which is a chemical waste product. The urea travels from the liver to the kidneys through the bloodstream. Healthy kidneys filter urea and remove other waste products from the blood, and the filtered waste products leave the body through urine. Accordingly, BUN levels (e.g., the levels of nitrogen content in urea) may provide insight into kidney health and function. Thus, a patient experiencing high levels of measured extracellular potassium and is assumed to have damaged kidney function, may also be expected to be experiencing high levels of measured BUN (e.g., given a damaged kidney would not likely be capable of filtering urea and removing other waste products from the blood). Accordingly, where measured BUN levels are also high, a higher confidence level may be assigned to potassium levels measured for the patient. Further, the assumption that the patient's kidney(s) are damaged may be strengthened, thereby increasing the likelihood that kidney(s) of the patient are not working properly and increasing the likelihood that the patient is, in fact, experiencing (or about to experience) one or more symptoms commonly associated with kidney disease.

In yet another example, at block 402, continuous analyte monitoring system 104 may continuously monitor potassium and ammonia levels of a patient during a plurality of time periods. In certain embodiments, the measured levels of ammonia may be used as an additional input when predicting one or more symptoms of the patient.

In particular, ammonia is a waste product made by the body during the digestion of protein. Normally, ammonia is processed in the liver, where it is changed into another waste product called urea. Accordingly, ammonia levels of a patient may be assessed for monitoring decompensation of the liver of the patient. Endogenous insulin is processed by the liver, and up to 80% is cleared and not released by the liver back into circulation. However, with decreased liver function, more endogenous insulin may be in circulation, which as mentioned previously, may cause extracellular potassium to be moved intracellularly due to the surplus in insulin in a patient that is not experiencing insulin resistance. In other words, the amount of extracellular potassium that is available to be measured by CPM 202 may be decreased.

Thus, a patient without kidney disease and not experiencing insulin resistance, who may not be expected to be experiencing one or more symptoms, may not show “normal” extracellular measured potassium levels because, although the kidney is functioning properly, the patient may have a surplus in endogenous insulin. Accordingly, in these cases, insulin levels of the patient may be masking the actual health of a patient's kidney(s) (e.g., that the kidney(s) are in good health), thus providing abnormal potassium levels for the patient that are caused by factors other than declining kidney health. Accordingly, the assessment of ammonia levels over time may provide insight into the health of a patient's liver which may provide additional insight on insulin levels and their impact on measured potassium levels. With this information, more accurate predictions may be made when determining whether the patient is experiencing one or more symptoms of kidney disease. For example, by continuously measuring ammonia, one or more models presented herein may be able to determine low measured potassium levels of the patient is not attributed to the patient having a damaged kidney, but instead attributed to the fact that the patient's liver is deteriorating, which may results from a number of mechanisms including an excess of insulin pushing potassium intracellularly.

In yet another example, at block 402, continuous analyte monitoring system 104 may continuously monitor potassium and C-Peptide levels of a patient during a plurality of time periods. In certain embodiments, the measured levels of C-peptide may be used to determine endogenous insulin levels of a patient to provide additional context around potassium measurements gathered by the continuous analyte sensor 202.

In particular, C-peptide is a substance that is created when the hormone insulin is produced and released into the body. Because no method currently exits for measuring insulin in the body, the level of C-peptide in the blood can be measured to show how much insulin is being made by the pancreas. Often, C-peptide is measured to tell the difference between insulin the body produces and insulin that is injected into the body. Understanding insulin levels of a patient may provide insight into why measured potassium levels of a patient are displaying lower than normal or greater than normal values for the patient to better predict one or more symptoms of the patient.

In yet another example, at block 402, continuous analyte monitoring system 104 may continuously monitor potassium and cystatin C levels of a patient during a plurality of time periods. In certain embodiments, the measured levels of cystatin C may be used as an additional input when predicting one or more symptoms of the patient. For example, cystatin C is a low molecular weight protein member of the cystatin superfamily of cysteine protease inhibitors. Cystatin C is produced by nucleated cells and exhibits a stable production rate. Cystatin C is freely filtered by the glomerulus and metabolized after tubular reabsorption. Cystatin C has been proposed as a valuable alternative marker tested in urine and in blood, particularly in situations in which creatinine-based estimates of GFR fail to provide an accurate estimate. If kidney function and GFR decline, the blood levels of Cystatin C are expected to rise. In some cases, serum levels of cystatin C provide are a more precise test of kidney function (as represented by the GFR) than serum creatinine levels. Accordingly, the assessment of cystatin C levels over time may provide insight into the overall health of a patient's kidney which may aid in determining whether the patient is experiencing one or more symptoms of kidney disease.

In certain embodiments, the one or more algorithms and/or models described herein, e.g., for predicting the risk or likelihood of a patient experiencing one or more different symptoms, may be configured to use input from one or more sensors measuring one or more of the multiple analytes described above. Parameters and/or thresholds of such algorithms and/or models may be altered based, at least in part, on a number of analytes being measured for input to reflect the knowledge attained from each of the other analytes being measured.

In addition to continuously monitoring one or more analytes of a user during a first time period to obtain analyte data at block 402, optionally, in certain embodiments, workflow 400 may also include monitoring other sensor data (e.g., non-analyte data) during the plurality of time periods using one or more other non-analyte sensors or devices (e.g., such as non-analyte sensors 206 and/or medical device 208 of FIG. 2).

As mentioned previously, non-analyte sensors and devices may include one or more of, but are not limited to, an insulin pump, a haptic sensor, an electrocardiogram (ECG) sensor or heart rate monitor, a blood pressure sensor, a sweat sensor, a respiratory sensor, a thermometer, an impedance sensor, a peritoneal dialysis machine, a hemodialysis machine, a continuous positive airway pressure (CPAP) machine, sensors or devices provided by display device 107 (e.g., accelerometer, camera, global positioning system (GPS), heart rate monitor, etc.) or other user accessories (e.g., a smart watch), or any other sensors or devices that provide relevant information about the user. Metrics, such as metrics 130 illustrated in FIG. 3, may be calculated using measured data from each of these additional sensors. Further as illustrated in FIG. 3, metrics 130 calculated from non-analyte sensor or device data may include body temperature, heart rate (including heart rate variability), respiratory rate, etc. In certain embodiments, described in more detail below, metrics 130 calculated from non-analyte sensor or device data may be used to further inform the analysis around kidney disease symptom prediction.

In certain embodiments, one or more of these non-analyte sensors and/or devices may be worn by a user to aid in the detection of periods of increased physical exertion by the user. Such non-analyte sensors and/or devices may include an accelerometer, an ECG sensor, a blood pressure sensor, a heart rate monitor, an impedance sensor, a dialysis machine, and the like. In certain embodiments, measured and collected data from periods of increased physical exertion and periods of sedentary activity by the user may be used to analyze potassium levels during each of these identified periods to predict whether a patient is experiencing one or more symptoms of kidney disease.

As an example, in certain embodiments, sudden patterns of sedentary activity, detected by such non-analyte sensors and/or devices, in a patient who is generally active may be a trigger to ask a patient whether they are feeling well or experiencing one or more symptoms. As another example, in certain embodiments, sudden patterns of sedentary activity, detected by such non-analyte sensors and/or devices, in a patient who is generally active may be a trigger for the patient to engage in one or more diagnostic tests to determine whether the patient is not feeling well and/or experiencing one or more symptoms (e.g., a resting respiratory rate test to determine whether the patient is experiencing shortness of breath, which is a common symptom of kidney disease). Embodiments relating to the use of non-analyte sensor and/or device data to detect abnormal patterns in patients for symptom prediction is described in more detail below with respect to block 418 in FIG. 4.

In certain embodiments, one or more non-analyte sensors and/or devices that may be worn by a patient may include a temperature sensor. A temperature sensor may be worn to aid in correcting measured potassium levels for predicting one or more symptoms of the patient. In particular, a correlation exists between body temperature and potassium release in the human body.

For example, at higher body temperatures, the body sweats, and potassium is excreted through sweat. Accordingly, higher body temperatures may induce lower potassium levels. In some cases where measured potassium levels of a patient may appear to be lower than normal for that patient, one may conclude the lower than normal potassium levels of the patient are due to an inability to concentrate urine due to impaired renal tubule response to vasopressin (ADH), which leads to excretion of large amounts of dilute urine, including, in some cases, potassium. However, such decreased potassium levels may actually be due to excessive sweat, e.g., excessive potassium leaving the patient's body. In other words, high body temperature of a patient may affect the amount of extracellular potassium that is measured by CPM 202. Accordingly, in certain aspects, secondary sensors, such as a temperature sensor, may be used to show that dynamic and sudden changes to potassium may be a result of sweating (e.g., associated with exercise, hot and/or humid weather, etc.) as compared to a negative health event.

A correlation may also exist between the temperature of the environment and potassium levels of the user. For example, during exercise, a user may experience high potassium levels when exercising outdoors in high temperatures. Therefore, when the user is outdoors in an environment with high temperatures, intensity may need to be reduced in order to avoid high potassium levels, despite potassium being excreted through sweat.

As another example, in some cases, measured potassium levels for a patient initially experiencing hypothermia (e.g., occurs when the body loses heat faster than the body can produce heat, causing a dangerously low body temperature) may appear to be lower than normal potassium levels associated with the patient. Such decreased potassium levels may be attributed to the patient experiencing an onset of hypothermia. In particular, hypothermia may cause an initial decrease of extracellular potassium levels. Hypothermic hypokalemia is linked to an intracellular shift rather than an actual net loss. The intracellular shift is caused by a variety of factors such as enhanced functioning of Na+K+ATPase, beta-adrenergic stimulation, pH and membrane stabilization in deep hypothermia.

As hypothermia progresses in the patient, however, irreversible cell damage may occur. In particular, the body may experience a lack of enzyme functioning at cold temperatures and blocked active transport. Thus, as hypothermia progresses, measured potassium levels of the patient may increase from levels that are lower than normal to levels that are higher than normal. In other words, low body temperature of a patient may affect the amount of extracellular potassium that is measured by CPM 202 over time. Accordingly, monitoring the body temperature of a patient may help to inform measured potassium levels of the patient such that the measured potassium levels may be corrected prior to predicting one or more symptoms of the patient. Additionally, in certain aspects, an external temperature probe may be used to measure the temperature immediately around (e.g., the area very close to) the patient to predict whether the patient is, in fact, experiencing hypothermia or decreased body temperatures. Further, in certain aspects, an accelerometer or a piezoelectric sensor may be used to identify whether a patient is shivering to determine whether a patient is experiencing decreased body temperatures that may be affecting the measured potassium levels of the patient.

In certain embodiments, one or more non-analyte sensors and/or devices that may worn by a patient may include a blood pressure sensor. Blood pressure measurements collected from a blood pressure sensor be used to provide additional insight into kidney health of the patient.

In particular, CKD and high blood pressure are closely related. Typically as blood pressure rises, kidney function declines. Accordingly, the assessment of blood pressure levels of a patient over time may provide additional insight into kidney health of the patient. Thus, a patient experiencing high levels of measured extracellular potassium and is assumed to have damaged kidney function (e.g., given excess potassium is not being filtered from the body), may also be expected to be experiencing high blood pressure levels. Accordingly, where blood pressure levels for the patient are also high, the assumption that the patient's kidney(s) are damaged may be strengthened, thereby increasing the likelihood that kidney(s) of the patient are not working properly and increasing the likelihood that the patient is, in fact, experiencing (or about to experience) one or more symptoms commonly associated with kidney disease.

In certain embodiments, one or more non-analyte sensors and/or devices that may worn by a patient may include an ECG sensor and/or a heart rate monitor. As is known in the art, an ECG device is a device that measures the electric activity of the heartbeat. In certain embodiments, heart rate measurements, as well as heart rate variability information, collected from an ECG sensor and/or a heart rate monitor may be used in combination with a CPM to more accurately predict the risk or likelihood of a patient being diagnosed with hyperkalemia and/or experiencing one or more cardiac event(s) (e.g., arrhythmia and/or sudden cardiac death).

In particular, potassium levels of a patient measured using a CPM may be used to detect hyperkalemia or hypokalemia, including a level at which a cardiac event is likely to occur. Hyperkalemia increases the risk of cardiac arrhythmia episodes and sudden death. However, in some cases, potassium measurements alone may not be an adequate index of the severity of hyperkalemia; thus, certain embodiments described herein propose using, in combination, a CPM and an ECG/heart rate monitor to make more accurate predictions or determinations about the risk or likelihood of a patient experiencing one or more cardiac event(s).

For example, occasionally, higher than normal levels of potassium measured for a patient may lead to changes in heart behavior that may be reflected on an ECG. Specifically, classic teachings of the chronological ECG changes of hyperkalemia may include peaked T waves, prolonged PR intervals, widening QRS complex, loss of the P wave, a “sine wave”, and an asystole (e.g., colloquially referred to as a flat line, represents the cessation of electrical and mechanical activity of the heart). Thus, ECG measurements, in combination with CPM measurements, for a patient may be vital for providing a whole-picture assessment of the physiologic significance of hyperkalemia. Further, ECG findings generally correlate with measured potassium levels; however, potentially life-threatening arrhythmias can occur without warning at almost any level of hyperkalemia. In other words, an ECG/heart rate monitor may be used, in combination with a CPM, to better predict the risk or likelihood of a patient experiencing an arrhythmia to (1) in some cases, avoid the arrhythmia where the arrhythmia is catastrophic, and (2) in some cases, intervene before the arrhythmia becomes deadly.

Further, in certain embodiments, heart rate measurements, as well as heart rate variability information, collected from an ECG sensor and/or a heart rate monitor may be used in combination with measured potassium levels for predicting one or more symptoms of the patient. More specifically, heart rate measurements, as well as heart rate variability information, may be analyzed to determine a time delay constant between measured local potassium levels (e.g., potassium levels in the local area where the potassium of the patient is being measured) and their impact on the heart.

In particular, a CPM 202 that measures extracellular potassium may show a different potassium level in the local area where potassium is being measured than potassium levels in the full system or in the cardiac tissue of a patient. This is because potassium levels rise first systemically, then rise interstitially. In other words, interstitial potassium levels may lag systemic potassium levels. For example, potassium levels at the cardiac tissue may precede interstitial levels depending on where a potassium sample for determining such potassium levels of the patient is taken from.

Accordingly, in certain embodiments, a standardized or a personalized delay constant may be calculated, where the delay constant corresponds to the amount of time it takes for interstitial potassium levels of the patient to reach systemic potassium levels of the patient. The calculated delay constant may be applied to measured potassium levels to better understand systemic potassium levels (e.g., higher potassium levels) for more accurate prediction of hyperkalemia and/or one or more cardiac events. In particular, in certain embodiments, one or more algorithms may use the calculated delay constant, in combination with the patient's interstitial potassium trend, interstitial potassium rates of change, and/or measured interstitial potassium levels to predict the risk or the likelihood of the patient currently experiencing, or about to experience in a specified amount of time, an arrhythmia (or become diagnosed with hyperkalemia).

Further, in certain embodiments, instances of vomiting and/or instances of diarrhea recorded for a patient may be collected and used in combination with measured potassium levels for predicting one or more symptoms of the patient. In particular, in some cases, prolonged periods of vomiting, diarrhea, or both can result in excessive potassium loss from the digestive system.

At block 404, workflow 400 continues by processing the analyte data from the plurality of time periods to determine at least one rate of change of potassium of the patient. Block 404, in certain embodiments, may be performed by decision support engine 114.

As mentioned, a potassium level rate of change refers to a rate that indicates the change of one or more time-stamped potassium measurements or values in relation to one or more other time-stamped potassium measurements or values. In certain embodiments, machine-learning models, described herein, used to provide symptom predictions may include one or more features not only related to potassium levels of the patient, but also potassium level rates of change of the patient. For example, an example machine-learning model may include weights applied to features associated with the one or more rates of change of potassium levels. Thus, in certain embodiments, prior to use of the machine-learning model, at least one potassium level rate of change for the patient may need to be calculated for input into the model.

Further, in certain embodiments, rules-based models, described herein, used to provide abnormal potassium level symptom predictions, may include one or more rules not only related to potassium levels of the patient, but also potassium level rates of change of the patient. For example, a reference library, used to define one or more rules for the rules-based models, may maintain ranges of potassium levels and ranges of potassium level rates of change which may be mapped to different symptoms experienced by patients with kidney disease. Thus, prior to use of the rules-based model, at least one potassium level rate of change for the patient may need to be calculated for input into the model. Additional features, such as potassium level rates of change, added to the model may, in some cases, allow for a more accurate prediction of one or more symptoms of kidney disease for the patient.

At block 406, workflow 400 continues by determining a likelihood that the patient is experiencing (or will experience) a symptom using (1) the at least one rate of change of potassium of the patient (e.g., determined at block 404), and (2) a trained model or one or more rules (e.g., rules-based models).

As mentioned, different methods for determining the likelihood that the patient is experiencing (or will experience) a symptom may be used by decision support engine 114. One method may include using machine-learning model(s) while another method may include using rules-based model(s) defining one or more rules. In certain embodiments, such machine-learning model(s) are trained and such rules-based model(s) include rules determined based on empirical research or an analysis of historical patient records from historical records database 112. Either the machine learning model(s) or the rules-based model(s) may be deployed for a particular patient that is using, at least, a CPM 202.

After determining the likelihood that the patient is experiencing (or will experience) a symptom, at block 410, workflow 400 continues by decision support engine 114 confirming or denying whether the patient is experiencing the symptom which decision support engine 114 predicted for the patient at block 406. For example, the presence of symptoms may be confirmed or denied through manual input by the patient, contextual data (e.g., cameras), other input data (e.g., sensor data), or observations and/or inputs by a person other than the patient (e.g., a healthcare professional).

In certain embodiments, decision support engine 114 may confirm or deny whether the patient is experiencing the symptom by transmitting an indication to the patient prompting the patient to confirm or deny whether the patient is, in fact, experiencing the predicted symptom. In certain embodiments, the indication may be transmitted to the patient as a message via application 106.

In certain embodiments, operations at block 410 may occur only where the likelihood determined at block 406 is above a threshold. For example, where decision support engine 114 determines there is 20% chance a patient is experiencing numbness, and a predefined threshold is set to 70%, decision support engine 114 may not determine whether the patient is experiencing the symptom at block 410. In certain embodiments, operations at block 410 may occur when the model is less confident as to whether or not the patient is experiencing the symptom. For example, in certain aspects, a prediction may be considered to be uncertain (e.g., less confident) where decision support engine 114 determines there is between a 40%-60% chance the patient is experiencing a symptom. In this case, decision support engine 114 may determine whether the patient is experiencing the symptom at block 410. In certain other embodiments, operations at block 410 may occur periodically (e.g., not necessarily in response to determining a likelihood at block 406) such that decision support engine 114 determines periodically whether the patient is experiencing a symptom.

Although the embodiment of FIG. 4A illustrates the use of a MISO model used to predict whether a patient is experiencing (or is likely to experience) only one symptom commonly associated with kidney disease, in embodiments where a MIMO model is used (e.g. model used to predict whether a patient is experiencing (or is likely to experience) two or more symptoms commonly associated with kidney disease), different thresholds may be predefined for different symptoms. Further, the determination at block 410 for whether the patient is experiencing a symptom may be based on the predefined threshold for that symptom and the determined percentage of likelihood that the patient is experiencing that symptom. Accordingly, in some cases, decision support engine 114 may make one or more determinations at block 410, where a MIMO model is used and a vector output for the MIMO model indicates percentages for two or more symptoms that are greater than their respective predefined thresholds.

Where at block 410, the decision support engine 114 confirms that the patient is experiencing the symptom predicted by decision support engine 114 (e.g., decision support engine predicted the patient is experiencing numbness and decision support engine 114 confirms the patient is experiencing numbness), at block 412, workflow 400 continues by associating physiological parameters of the patient to the patient experiencing the symptom. Specifically, in certain embodiments, after confirming the patient is experiencing a symptom commonly associated with kidney disease, decision support engine 114 may be configured to determine what physiological parameters of the patient are associated with the patient experiencing the identified symptom. For example, decision support engine 114 may determine a potassium level range, a potassium level rate of change range, a heart rate, a respiration rate, values for other metrics 130, etc. for the patient at the time the patient is confirmed to be experiencing numbness. In certain embodiments, decision support engine 114 may determine the physiological parameters of the patient based on which the prediction was made (e.g., the inputs into the model) as the physiological parameters that correspond to the symptom for the specific patient. In certain embodiments, decision support engine 114 may be configured to measure one or more physiological parameters of the patient after confirming the patient is experiencing the symptom, and associated these physiological parameters with the patient experiencing the identified symptom.

In certain embodiments where the patient is prompted to confirm or deny they are experiencing the symptom, decision support engine 114 may be configured to record the answer supplied by the patient at block 410. In certain embodiments, the answer may be timestamped to indicate a date and time when the answer for the patient was recorded. By recording such answers of the patient, decision support engine 114 may learn about different physiological parameter thresholds and their association with different subjective feelings (e.g., feelings of numbness, feelings of muscle weakness, etc.) being experienced by the patient.

In certain embodiments, decision support engine 114 may be configured to record such physiological parameters in metrics 130 stored for the patient in user profile 118. In certain embodiments, the physiological parameters may be timestamped to indicate a date and time when the physiological parameters for the patient were recorded.

In certain embodiments, the patient specific values of the physiological parameters for different symptoms experienced by the user may be used to train the machine learning models (e.g., originally trained using only population data) and update one or more rules for the rules-based model (e.g., where the rules were originally created based on population data) to create one or more personalized models specific to the patient for more accurately predicting one or more symptoms of the patient associated with potassium imbalance. Accordingly, at block 414, workflow 400 continues by using the association created at block 412 to update the trained machine-learning model or the one or more rules used for the rules-based model.

In other words, each model may use the information provided by decision support engine 114 and/or the patient as feedback to personalize the model for the patient. In particular, each model may learn through this feedback loop about the patient's personalized symptom thresholds (e.g., a potassium level threshold when the patient will experience tingling, etc.). This approach may also allow each model to be personalized for each patient and continually learn over time, even as the patient's baseline and symptom thresholds (e.g., potassium levels, potassium rates of change, etc.) change (e.g., as kidney health of the patient gets better or worsens).

In certain embodiments, updating the one or more rules for the rules-based model may include updating existing rules and/or creating new rules based on an analysis of the parameters collected for the patient when the patient was confirmed to be experiencing an identified symptom. In certain embodiments, updating the trained machine-learning model may include re-training the machine learning model using physiological parameters collected for the patient when the patient was confirmed to be experiencing an identified symptom. In some cases, re-training the machine learning model may include adjusting one or more weights for one or more features of the machine-learning model.

For example, a machine-learning model initially trained based on population data to predict whether a patient is experiencing tingling may include, at least, one or more features related to different potassium ranges, potassium rates of change, and/or other parameters discussed herein. Based on feedback from the patient indicating that the patient is, or is not, experiencing tingling, as predicted by the model, the weights applied features of the model may be unchanged or altered (e.g., increased or decreased).

Similar methods for adjusting weights of the machine-learning models based on different feedback provided by the patient may be used to optimize and personalize the model based on the patient's specific data.

Referring back to block 410, upon determining the patient is not experiencing the symptom predicted by decision support engine 114 (e.g., decision support engine predicted the patient is experiencing numbness and the patient indicates to decision support engine 114 that they are not experiencing numbness), at block 416, workflow 400 continues by decision support engine 114 initiating one or more diagnostic tests. As used herein, diagnostic tests are a variety of assessments performed to screen for and detect certain symptoms of the patient.

In certain embodiments, the one or more diagnostic tests initiated by decision support engine 114 may include using haptic feedback to communicate with the patient after the patient has denied experiencing numbness (e.g., the symptom predicted by decision support engine 114 using workflow 400). Decision support engine 114 may use the response of the patient, or lack thereof, to confirm/deny whether the patient is experiencing numbness.

As an illustrative example, decision support engine 114, using a deployed machine-learning model, may predict that there is an 80% chance the patient is experiencing numbness. Accordingly, decision support engine 114 may confirm or deny whether the patient is experiencing such numbness, and the patient, in some cases, may identify that they are not experiencing numbness. To confirm the determination, decision support engine 114 may be configured to request that the patient engage with haptic sensors (e.g., placed on the user's body as another sensor or as part of the CPM). The haptic sensor may be configured to create a combination of force, vibration, and/or motion sensations to the patient. The patient may confirm or deny whether they were able to feel this sensation. In situations where the patient was not able to feel this sensation, decision support engine 114 may determine that although the patient reported not experiencing numbness, the patient was, in fact, experiencing numbness; however, the patient was unaware.

In certain embodiments, the one or more diagnostic tests initiated by decision support engine 114 may include using a respiration sensor to determine a respiration rate of the patient after the patient has denied experiencing a shortness of breath (e.g., the symptom predicted by decision support engine 114 using workflow 400). Decision support engine 114 may use the determined respiration rate of the patient to confirm/deny whether the patient is experiencing a shortness of breath.

In certain embodiments, the one or more diagnostic tests initiated by decision support engine 114 may include using haptic feedback to communicate with the patient after the patient has denied experiencing tingling (e.g., the symptom predicted by decision support engine 114 using workflow 400). Decision support engine 114 may use the response of the patient, or lack thereof, to confirm/deny whether the patient is experiencing tingling.

In certain embodiments, the one or more diagnostic tests initiated by decision support engine 114 may include using an electromyography (EMG) sensor to determine a muscle response of the patient after the patient has denied experiencing muscle weakness (e.g., the symptom predicted by decision support engine 114 using workflow 400). As is known in the art, an EMG sensor is a sensor that measures small electrical signals generated by a patient's muscles when a patient moves their muscles. Decision support engine 114 may use the determined muscle response of the patient to confirm/deny whether the patient is experiencing muscle weakness.

In certain embodiments, the one or more diagnostic tests initiated by decision support engine 114 may include triggering the patient to squeeze a widget (e.g., a stress ball) after the patient has denied experiencing muscle weakness (e.g., the symptom predicted by decision support engine 114 using workflow 400). In certain embodiments, decision support engine 114 may determine a strength of the patient by asking the patient whether the patient was able to fully squeeze the widget, or not and confirming with the patient as to whether they were able to fully squeeze the widget. In certain embodiments, the widget may include pressure sensors; thus, decision support engine 114 may determine a strength of the patient based on the pressure measured by the pressure sensors in the widget. Decision support engine 114 may use the strength of the patient in squeezing the widget, or lack thereof, to confirm/deny whether the patient is experiencing muscle weakness.

In certain embodiments, the one or more diagnostic tests initiated by decision support engine 114 may include using a heart rate monitor (in combination with an one or more sensors used to detect the patient is sedentary) to determine a resting heart rate of the patient after the patient has denied experiencing an arrhythmia (e.g., the symptom predicted by decision support engine 114 using workflow 400). Decision support engine 114 may use the determined resting heart rate of the patient to confirm/deny whether the patient is experiencing an arrhythmia.

In certain embodiments, one or more other diagnostic tests similar to those mentioned herein may be used to confirm/deny whether the patient is experiencing the symptom predicted by decision support engine 114 using workflow 400.

In certain embodiments, decision support engine 114 may be triggered to initiate one or more diagnostics tests at block 416 after determining a likelihood that the patient is experiencing (or will experience) a symptom. In other words, in certain embodiments, operations at block 410 may be skipped. Instead, decision support engine 114 may be configured to initiate one or more diagnostic tests without decision support engine 114 determining whether the patient is experiencing the symptom, to determine whether the patient is experiencing the identified symptom. In some cases, a patient may not be engaged or fail to respond to a request asking the user to confirm/deny they are experiencing the identified symptom; thus, skipping operations at block 410 may prove to be more efficient for predicting one or more symptoms of the patient. In certain embodiments, where at block 406, decision support engine 114 determines there is a high likelihood that the patient is currently experiencing a symptom, decision support engine 114 may be triggered to automatically initiate one or more tests. On the other hand, where at block 406, decision support engine 114 determines there is a high likelihood that the patient is going to experience a symptom within a specified period of time (e.g., 5 minutes), decision support engine 114 may wait the specified period of time prior to initiating the one or more diagnostic tests.

At block 418, workflow 400 continues by decision support engine 114 determining whether results of the one or more diagnostic tests confirm the patient is experiencing the identified symptom (e.g., the symptom predicted by decision support engine 114 using workflow 400). Where, at block 418, decision support engine 114 determines the results of the one or more diagnostic tests confirm the patient is experiencing the identified symptom, at block 412, workflow 400 continues by associating physiological parameters of the user to the user experiencing the symptom. Specifically, in certain embodiments, after confirming the patient is experiencing a symptom commonly associated with kidney disease (in some cases, even after a patient has denied experiencing the symptom), decision support engine 114 may be configured to determine what physiological parameters of the patient are associated with the patient experiencing the identified symptom. At block 414, workflow 400 continues by using the association created at block 412 to update the trained machine-learning model or the one or more rules used for the rules-based model.

On the other hand, where, at block 418, decision support engine 114 determines the results of the one or more diagnostic tests do not confirm the patient is experiencing the identified symptom, at block 420, workflow 400 continues by associating physiological parameters of the user to the user not experiencing the symptom. Specifically, in certain embodiments, after confirming the patient is not experiencing a symptom commonly associated with kidney disease, decision support engine 114 may be configured to determine what physiological parameters of the patient are associated with the patient not experiencing the identified symptom. At block 414, workflow 400 continues by using the association created at block 412 to update the trained machine-learning model or the one or more rules used for the rules-based model.

In certain embodiments, updating the one or more rules for the rules-based model may include decision support engine 114 or some other server analyzing the parameters collected for the patient when the patient was confirmed not to be experiencing an identified symptom to update a rules-based model and defining and/or revising one or more rules based on the updated rules-based model. In certain embodiments, updating the trained machine-learning model may include training server system 140 re-training the machine learning model using the results of the diagnostics tests and/or the physiological parameters collected for the patient when the patient was confirmed not to be experiencing an identified symptom. In some cases, re-training the machine learning model may include adjusting one or more weights for one or more features of the machine-learning model.

Although the embodiment of FIG. 4A illustrates decision support engine 114 determining whether the patient is experiencing the symptom, at block 410, in certain other embodiments, decision support engine 114 may not provide a determination. Instead, in certain embodiments, diagnostic tests may be performed to confirm or deny the predicted symptom. In certain other embodiments, neither determination from decision support engine 114, nor results from diagnostic tests, may be obtained; instead, after block 406, decision support engine 114 may associate physiological parameters of the patient to the patient experiencing the symptom (e.g., as illustrated at block 412) or associate physiological parameters of the patient to the patient not experiencing the symptom (e.g., as illustrated at block 420).

Further, although the embodiment of FIG. 4A illustrates the initiation of one or more diagnostic tests only after decision support engine has determined the patient is not experiencing the symptom, in certain other embodiments, one or more diagnostic tests may similarly be initiated when decision support engine 114 confirms the patient is experiencing the symptom to further increase the level of confidence that the patient is, in fact, experiencing the predicted symptom that decision support engine 114 has also confirmed.

Further, although the embodiment of FIG. 4A illustrates the initiation of one or more diagnostic tests after decision support engine 114 has determined the patient is not experiencing the symptom, in certain other embodiments, one or more diagnostic tests may not be used. Instead, in certain embodiments, only decision support engine 114 may determine whether the patient is experiencing the predicted symptom, and based on the determination of decision support engine 114, decision support engine 114 may associate physiological parameters of the patient to the patient experiencing the symptom (e.g., as illustrated at block 412) or associate physiological parameters of the patient to the patient not experiencing the symptom (e.g., as illustrated at block 420).

As mentioned previously, a model (e.g., a machine learning model or rules-based model) utilized and personalized for the patient based on the operations illustrated in workflow 400A may be deployed to provide a prediction of the patient experiencing one or more symptoms and further provide one or more recommendations for treatment based the prediction. FIG. 4B is an example workflow 400B for determining the likelihood of one or more symptoms commonly associated with potassium imbalance occurring in real-time or within a specified period of time using the model and providing one or more recommendations for treatment based the determined likelihood, according to certain embodiments of the present disclosure.

Operations 402-406, 410, 416, and 418 of FIG. 4B may be similar to operations 402-406, 410, 416, and 418 illustrated in FIG. 4A; however, in FIG. 4B, further shows decision support (e.g., one or more recommendations) being provided to the patient based on the predicted one or more symptoms.

Although FIG. 4B illustrates providing decision support to a patient after (1) decision support engine 114 determines whether the patient is experiencing a symptom predicted at block 406 or (2) decision support engine 114 determines the patient is not experiencing the predicted symptom and a diagnostic test confirms the patient is actually experiencing the symptom, in certain other embodiments, decision support may be provided to the patient either after (3) determining a likelihood that the patient is experiencing, or will experience, a symptom at block 406, or (4) receiving feedback from the patient at block 410.

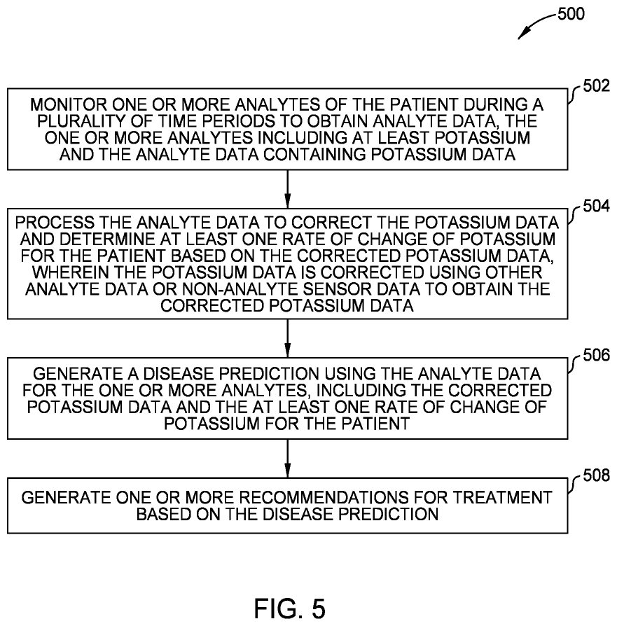

In certain embodiments, the decision support provided to the patient may tell the patient how to control potassium levels to avoid a symptom before it occurs where decision support engine 114 predicted the patient was likely to experience the symptom in the near future. In certain embodiments, the decision support provided to the patient may provide one or more recommendations for avoiding the symptom in the future (e.g., consume less potassium, adjust insulin dosage, etc.). In particular, decision support engine 114 may provide recommendations for increasing or decreasing potassium levels of the patient such as to correct the patient's potassium imbalance currently causing the predicted one or more symptoms. Such recommendations may include diet recommendations, hydration recommendations, lifestyle recommendations, treatment recommendations, service intervention recommendations, insulin dosage recommendations, medication recommendations, alarm/alert recommendations, or other recommendations for managing potassium levels in the patient. Decision support engine 114 may output such recommendations for treatment to the user (e.g., through application 106). Each of these recommendations may be described in more detail with respect to FIG. 5.

In certain embodiments, decision support engine 114 may use the predicted one or more symptoms of the patient to understand the patient's risk of hospital admission/readmission. In certain embodiments, the predicted one or more symptoms of the patient may be used to better understand post hospital discharge stability of the patient when determining whether to discharge the patient. In certain embodiments, decision support engine 114 may use the predicted one or more symptoms of the patient to stratify the level of care a patient should receive upon hospital admittance. This may be described in more detail with respect to FIG. 5.

In certain embodiments, decision support engine 114 may use one or more other machine-learning models, trained based on patient-specific data and/or population data, to provide recommendations for the treatment or prevention of symptoms associated with potassium imbalance. The algorithms and/or machine-learning models may take into account one or more inputs 128 and/or metrics 130 (e.g., including potassium levels and/or potassium levels rates of change) described with respect to FIG. 3 for a patient to determine optimal recommendations for prevention and/or management of such symptoms.

In certain embodiments, as an alternative to using machine-learning models, decision support engine 114 may use one or more methods to provide recommendations for the prevention of symptoms associated with potassium imbalance. The method may be rule-based and may include one or more rules used to provide a recommendation regarding the management of the patient's potassium levels.

The one or more models deployed and optimized in workflow 400 for a patient may provide insight into baseline potassium levels of a patient, as well as how these baselines change over time for the patient. The one or more models may also provide insight into what potassium level ranges and/or potassium level rates of change ranges (in addition to other physiological parameters) correlate to different symptoms experienced by a patient, as well as how these ranges change over time for the patient. In particular, the patient's personalized potassium thresholds (as well as other physiological parameters) corresponding to various symptoms may be analyzed over time to provide an indication as to the improvement or the deterioration of the patient's kidney, as well as other organs in the body and/or conditions/diseases of the patient.

In certain embodiments, the user-specific values of the physiological parameters for different symptoms experienced by the user may be a valuable input for one or models designed to assess the kidney health of a user, including the presence and/or severity of kidney disease.

FIG. 5 is a flow diagram illustrating an example method 500 for providing decision support using a continuous analyte sensor including, at least, a CPM, in accordance with certain example aspects of the present disclosure. For example, method 500 may be performed to provide decision support to a user, using a continuous analyte monitoring system 104 including, at least, a CPM 202, as illustrated in FIGS. 1 and 2.

Method 500 may be performed by decision support system 100 to collect/generate data such as inputs 128 and metrics 130, including for example, analyte data, patient information, and non-analyte sensor data mentioned above, to (1) automatically detect and classify abnormal kidney conditions, (2) assess the kidney health of the user, including presence and severity of kidney disease, hyperkalemia, and/or hypokalemia, (3) risk stratify patients to identify those patients with a high risk of kidney disease, hyperkalemia, and/or hypokalemia, (4) identify risks (e.g., mortality risk, significant cardiac event risk, etc.) associated with a current kidney disease diagnosis, (5) make patient-specific treatment decisions or recommendations for kidney disease, (6) provide information on the effect of an intervention (e.g., an effect of a lifestyle change of the patient, an effect of a surgical procedure, an effect of the patient taking new medication, etc.). In other words, the decision support system presented herein may offer information to direct and help improve care for patients with, or at risk, of kidney disease. Method 500 is described below with reference to FIGS. 1 and 2 and their components.

At block 502, method 500 begins by continuously monitoring one or more analytes of a patient, such as user 102 illustrated in FIG. 1, during a plurality of time periods to obtain analyte data. The one or more analytes monitored may include at least potassium; thus, the analyte data may contain potassium data. Block 502 may be performed by continuous analyte monitoring system 104 illustrated in FIGS. 1 and 2, and more specifically, continuous analyte sensor(s) 202 illustrated in FIG. 2, in certain embodiments. For example, continuous analyte monitoring system 104 may comprise a CPM 202 configured to measure the patient's potassium levels.

Similar to workflow 400 of FIG. 4, while the main analyte for measurement described herein is potassium, in certain embodiments, other analytes may be considered. In particular, combining potassium measurements with additional analyte data may help to further inform the analysis around diagnosing and staging kidney disease. For example, monitoring additional types of analytes, such as glucose, creatinine, lactate, BUN, ammonia, C-peptide, and/or cystatin C measured by continuous analyte monitoring system 104, may be used to correct measured potassium levels for a patient, provide additional insight into the kidney disease diagnostics, and/or supplement information used to determine optimal treatment for preventing progression of the disease (and in some cases, for disease regression).

The additional insight gained from using a combination of analytes, and not just potassium, may increase the accuracy of kidney disease diagnosis. For example, the probability of accurately diagnosing and/or staging kidney disease may be a function of a number of analytes measured for a user. More specifically, in some examples, a probability of accurately staging kidney disease using only potassium data (in addition to other non-analyte data) may be less than a probability of accurately staging kidney disease using potassium and other analyte data for one or more other analytes for analysis.

In certain embodiments, how other analytes are used to provide a kidney disease diagnosis may vary per patient. For example, different models may be created for different groups of patients having a common defining characteristic (e.g., a first group having patients who regularly experience high potassium levels, a second group having patients who regularly experience low potassium levels, etc.). The role and weight each of these other analytes play in making a kidney disease diagnosis may be different for each determined group of patients.

In certain embodiments described herein, analyte combinations, e.g., measured and collected by one (e.g., multianalyte sensor) or more sensors, for kidney disease staging, include potassium and at least one of glucose, creatinine, BUN, ammonia, C-peptide, and/or cystatin C; however, other analyte combinations may be considered for informing measured potassium levels (e.g., to gain a better understanding as to why measured potassium levels of a patient are lower or higher than expected potassium levels for the specific patient), correcting measured potassium levels, and diagnosing and staging kidney disease.

In certain embodiments, AI models, such as machine learning models and/or algorithms (e.g., rules-based models) may be used to provide real-time decision support for kidney disease diagnosis and staging. In certain embodiments, such models may be configured to use input from one or more sensors measuring multiple analyte data to diagnose, stage, treat, and assess risks of kidney disease. Accordingly, given the interaction of such comorbidities, parameters and/or thresholds of such algorithms and/or models may be altered based, at least in part, on a number of analytes being measured for input to reflect the knowledge attained from each of the other analytes being measured/morbidities associated with the additional analytes being measured.

In addition to continuously monitoring one or more analytes of a patient during a plurality of time periods to obtain analyte data at block 502, optionally, in certain embodiments, method 500 may also include monitoring other sensor data (e.g., non-analyte data) during the plurality of time periods using one or more other non-analyte sensors or devices (e.g., such as non-analyte sensors 206 and/or medical device 208 of FIG. 2).

As mentioned previously, non-analyte inputs may include outputs from one or more of, but not limited to, an insulin pump, a haptic sensor, an ECG sensor or heart rate monitor, a blood pressure sensor, a sweat sensor, a respiratory sensor, a thermometer, a peritoneal dialysis machine, a hemodialysis machine, sensors or devices provided by display device 107 (e.g., accelerometer, camera, global positioning system (GPS), heart rate monitor, etc.) or other user accessories (e.g., a smart watch), or any other sensors or devices that provide relevant information about the user.

At block 504, method 500 continues by processing the analyte data from the plurality of time periods to determine at least one rate of change of potassium of the patient. Block 504, in certain embodiments, may be performed by decision support engine 114. Block 504 in method 500 of FIG. 5 may be similar to block 404 in workflow 400 of FIG. 4.

At block 506, method 500 continues by generating a disease prediction using the analyte data associated with the one or more analytes and the at least one rate of change of potassium levels. Block 506 may be performed by decision support engine 114 illustrated in FIG. 1, in certain embodiments.

Different methods for generating a disease prediction may be used by decision support engine 114. In particular, in certain embodiments, decision support engine 114 may use a rules-based model to provide real-time decision support for kidney disease diagnosis and staging. As mentioned previously, rules-based models involve using a set of rules for analyzing data. In particular, decision support engine 114 may apply rule-statements (e.g., if, then statements) to assess the presence and severity of kidney disease in a patient, perform kidney disease risk stratification for a patient, and/or identify risks associated with a current kidney disease diagnosis of the patient.

For example, one rule may be related to an absolute maximum potassium level for the patient currently, or based on changes of the absolute maximum potassium level over time. Another rule may be related to an absolute minimum potassium level for the patient currently, or based on changes of the absolute minimum potassium level over time. Another rule may be based on changes of the potassium baselines of a patient over time. Another rule may be related to potassium level rates of change for the patient, whether such potassium level rates of change have been marked as “increasing rapidly” or “decreasing rapidly” (e.g., as described with respect to FIG. 3), or based on changes of the potassium level rates of change over time. Another rule may be related to, insulin metrics, creatinine metrics, BUN metrics, ammonia metrics, and/or C-peptide metrics as described with respect to FIG. 3 or based on changes of such metrics over time.

Another rule may be related to whether a patient experiences acute or abrupt increases (and what that increase is) in creatinine concentrations due to AKI (e.g., blood loss, vomiting, diarrhea, heart failure, etc.). Another rule may be related to a patient's potassium clearance rate following consumption of a known, or estimated, amount of potassium. For example, in a patient with impaired kidney function, the rate of clearance may be slower than in a healthy control subject.

Another rule may be related to the absolute maximum potassium level following some event such as consumption of a known or estimated amount of potassium. For example, the absolute maximum potassium level following the consumption of a known, or estimated, amount of potassium may be greater in a patient with impaired kidney function than that of a healthy control subject. Another rule may be related to a correlation between the absolute maximum potassium level of a patient and the patient's potassium clearance rate. For example, increased absolute maximum potassium levels with reduced potassium clearance may be observed as kidney disease progresses in a patient. One or more other rules may be based on data from one or more non-analyte sensors in combination with measured potassium levels of the patient. Any of the above identified rules may be used in combination with each other, or one or more other rules, when using the rules-based model.

Such rules may be defined and maintained by decision support engine 114 in a reference library. For example, the reference library may maintain ranges of potassium levels and/or potassium level rates of changes which may be mapped to different severities of kidney disease. In certain embodiments, such rules may be determined based on empirical research as well as analyzing historical patient records from historical records database 112.

In certain embodiments, as an alternative to using a rules-based model, AI models, such as machine-learning models may be used to provide real-time decision support for kidney disease diagnosis and staging. In certain embodiments, decision support engine 114 may deploy one or more of these machine learning models for performing diagnosis, staging, and risk stratification of kidney disease in a patient. Risk stratification may refer to the process of assigning a health risk status to a patient, and using the risk status assigned to the patient to direct and improve care.

In particular, decision support engine 114 may obtain information from a user profile 118 associated with a patient, stored in user database 110, featurize information for the patient stored in user profile 118 into one or more features, and use these features as input into such models. Alternatively, information provided by the user profile 118 may be featurized by another entity and the features may then be provided to decision support engine 114 to be used as input into the ML models. In certain embodiments, features associated with the patient may be used as input into one or more of the models to assess the kidney health of the patient, including the presence and severity of kidney disease.

In certain embodiments, features associated with the patient may be used as input into one or more of the models to risk stratify the patient to identify whether there is a high or low risk of the patient developing kidney disease. In certain embodiments, features associated with the patient may be used as input into one or more of the models to identify risks (e.g., mortality risk, risk of being diagnosed with one or more other diseases, etc.) associated with a current kidney disease diagnosis of the patient. In certain embodiments, features associated with the patient may be used as input into one or more of the models to perform any combination of the above-mentioned functions. Details associated with how one or more machine-learning models can be trained to provide real-time decision support for kidney disease diagnosis and staging are further discussed in relation to FIG. 6.

As mentioned, in certain embodiments, other analyte data, in addition to potassium, may be used by decision support engine 114 to generate a disease prediction for a patient, at block 506. Analyte data, including potassium data, lactate data, creatinine data, BUN data, ammonia data, C-peptide data, and/or cystatin C data (e.g., from measurements by continuous analyte monitoring system 104), may be used as input into such machine learning models and/or rules-based models to predict the kidney health of the user, including the presence and severity of kidney disease of a user.

Decision support engine 114 may use the machine learning models and/or the rules-based models to generate a disease prediction based on continuous analysis of data (e.g., analyte data and, in some cases, non-analyte data) for the patient collected over various time periods. Analysis of data collected for the patient over various time periods may provide insight into whether the health and/or a disease of the patient is improving or deteriorating. For example, a patient previously diagnosed with kidney disease using the models discussed herein may continue to be constantly monitored (e.g., continuously collect for the patient) to determine whether the disease is getting worse or better, etc. As an example, comparison of potassium levels, potassium baselines, absolute maximum potassium levels, absolute minimum potassium levels, potassium level rates of change (e.g., in response to consuming known amount of potassium), potassium baseline rates of change, and/or physiological parameters recorded for different symptoms for a patient over multiple months may be indicative of the patient's disease progression.

In certain embodiments, at block 506, decision support engine 114 outputs the generated disease prediction to the user. In some embodiments, method 500 ends at block 506 by outputting the disease prediction. In other words, in certain embodiments, the operation at block 508 may be skipped and decision support engine 114 may not provide further decision support on recommendations for treatment.

In other cases, method 500 continues at block 508 by decision support engine 114 generating one or more recommendations for treatment, based, at least in part, on the disease prediction generated at block 506. In particular, decision support engine 114 may provide recommendations for the treatment or prevention of kidney disease, such as lifestyle recommendations, medication recommendations, service intervention recommendations, or other recommendations for managing kidney health. Decision support engine 114 may output such recommendations for treatment to the user (e.g., through application 106).

In certain embodiments, decision support engine 114 may use one or more other machine-learning models, trained based on patient-specific data and/or population data, to provide recommendations for the treatment or prevention of kidney disease. The algorithms and/or machine-learning models may take into account one or more inputs 128 and/or metrics 130 (e.g., including potassium levels and/or potassium levels rates of change) described with respect to FIG. 3 for a patient to determine optimal recommendations for prevention and/or management of the patient's kidney disease. In certain embodiments, the model may look at different patterns of analyte measurements collected for the patient to guide the patient in the management of their disease.

In certain embodiments, as an alternative to using machine-learning models, decision support engine 114 may use one or more methods to provide recommendations for the treatment or prevention of kidney disease. The method may be rule-based, such that the method includes one or more rules used to provide a recommendation regarding the management of the patient's kidney disease.

Recommendations for the treatment or prevention of kidney disease may, in some cases, be based on a determined optimal balance of potassium (e.g., intracellular and extracellular) and insulin levels for a patient. In particular, in certain embodiments, one or more algorithms may be used to determine an optimal balance of potassium and insulin levels of a patient that is then used to form one or more recommendations for the patient regarding a diet, lifestyle change, treatment, insulin dosage, and/or medication.

As mentioned herein, insulin stimulates potassium uptake by cells, thereby reducing serum (e.g., extracellular) potassium levels, in cases where a patient is not experiencing insulin resistance or insulin sensitivity (e.g., where potassium levels are not as responsive to insulin, and is often caused by chronic hyperinsulinemia). In some cases, as a patient's kidney function decreases, a patient may need to rely on excess insulin to move some of the excess extracellular potassium intracellularly. While this provides an immediate solution in avoiding hyperkalemia and symptoms thereof, the solution may not be a long-term fix. In particular, at some point, potassium inside the cells may need to be pushed out of the cells so that the kidney may remove the excess potassium (e.g., through urine). Whether the patient's kidney is capable of clearing the amount of potassium pushed extracellularly (e.g., into the blood) may be a point of concern. In particular, the rate of potassium movement from inside a patient's cells to extracellular fluid may need to match a rate of potassium clearance by the kidney to ensure that excess levels of potassium are not present in the extracellular fluid. In some other cases, an excess of insulin may cause too much potassium to move intracellularly. Accordingly, determining an optimal balance of intracellular potassium, extracellular potassium, and insulin for a patient, based on kidney conditions for the patient, may help to better inform the recommendations made to patients for managing their kidney health and/or disease.

In certain embodiments, diet recommendations may include a recommendation for the patient to consume a fixed amount of potassium daily, weekly, etc. For example, a patient may be recommended to eat a fixed amount of potassium per day based on a level of potassium the patient's kidney is currently clearing. Monitoring potassium consumption may help to ensure that excess levels of potassium are not pushed intracellularly (e.g., due to excess insulin) while also ensuring that the potassium consumed is capable of being cleared by the patient's kidney.

In certain embodiments, diet recommendations may include a recommendation for the patient to increase their potassium consumption. For example, a patient may be recommended to increase potassium consumption where excess insulin and/or one or more diuretics are causing significant drops in measured extracellular potassium for the patient (e.g., which may lead to hypokalemia).

In certain embodiments, hydration recommendations may include a recommendation for the patient to increase their water intake. For example, a patient may be recommended to increase water intake to allow the kidneys to process potassium properly and therefore decrease potassium levels in the blood. Lack of proper hydration may cause hyperkalemia as the body cannot process potassium effectively.

In certain embodiments, lifestyle recommendations (e.g., including exercise recommendations) may include a recommendation for the patient to increase their physical activity daily, weekly, etc. given increased physical activity may be one method for removing excess potassium from the body, e.g., through sweating. For example, for patients with hyperkalemia, one or more models may be used to determine when such patients should engage in physical activity based on potassium levels, kidney function, and/or current insulin production/injection for the patient. A patient may be recommended to engage in a modified physical activity schedule or engage in additional rest breaks based on the determined schedule around when patients should engage in physical activity. In certain embodiments, an exercise recommendation may be made to optimize sweating while limiting the physical exertion of the patient, in order to reduce potassium, while preventing potassium from increasing due to exercise exertion by the patient. In certain embodiments, other analyte data or non-analyte data, such as heart rate or respiratory rate data, may be used in combination with potassium data to provide such exercise recommendations.

In certain embodiments, treatment recommendations may include a recommendation for dialysis for the patient. In certain embodiments, determining an optimal balance of potassium and insulin levels of a patient may help to inform whether dialysis is a recommended treatment for the patient. In particular, dialysis is a treatment for kidney failure that rids the body of unwanted toxins, waste products, and excess fluids by filtering a patient's blood. Dialysis helps to keep the potassium, phosphorus, and sodium levels in a patient's body balanced. Thus, understanding the optimal balance of potassium and insulin in the body may help to inform such treatment where dialysis is recommended.

In certain embodiments, service intervention recommendation may include a recommendation for the patient to seek medical aid. For example, in certain embodiments, the service intervention recommendation may indicate to a patient, or another individual with an interest in the patient's well-being, that the patient needs to immediately go to the emergency room and/or contact their physician. In certain other embodiments, the service intervention recommendation may automatically alert the physician of the patient as to the condition of the patient for intervention by the physician. In certain other embodiments, the service intervention recommendation may alert medical personnel to send aid to the patient, e.g., trigger ambulance services or paramedic services to provide urgent pre-hospital treatment and stabilization to the patient and/or transport of the patient to definitive care. In certain embodiments, decision support engine 114 may make a service intervention recommendation based on a patient's ability to seek medical help and/or the accessibility of the patient to medical help.

In certain embodiments, an insulin dosage recommendation may include a recommendation of a combination dosage, such as insulin/glucose (e.g., to prevent hypoglycemia but restore eukalemia). In certain embodiments, one or more algorithms may be used to determine the combination dosage to be recommended to the patient. In particular, insulin can be used to treat hyperkalemia such that increased doses of insulin can be used to reduce extracellular potassium. In a CKD patient who is also diabetic and on insulin, and has increasing potassium levels, an algorithm may be created and used for the administration of insulin to avoid or treat hyperkalemia while also preventing hypoglycemia. For example, the algorithm may calculate the amount of insulin needed to reduce potassium levels by a value, X (e.g., where X is a value greater than zero), and the amount of glucose and timing for glucose consumption to prevent hypoglycemia. Further, the insulin dosage recommendation may be individualized per patient to account for differences in insulin resistance across patients. For example, insulin resistance alters the ability of insulin to push potassium into cells; thus, a patient with a higher insulin resistance may require a different insulin dosage as compared to another patient with a different level of insulin resistance. In certain aspects, the individualized insulin dosage recommendation may be modified over time as insulin resistance increases, or decreases, in the patient.

In certain embodiments, medication recommendations may include a recommendation for the patient to take a new medication where the patient has not been previously taking similar medications. In certain embodiments, medication recommendations may include a recommendation for the patient to stop taking a previously prescribed medication, and in some cases, recommend an alternative medication for consumption by the patient. In certain embodiments, medication recommendations may include a recommendation for the patient to take a lower or higher dosage of a previously prescribed medication. In certain embodiments, medication recommendations may include a recommendation for the titration of a dosage of medication previously prescribed to the patient to determine an ideal dosage for the patient (e.g., while monitoring kidney and heart health of the user).

In certain embodiments, decision support engine 114 may determine kidney disease in a patient is progressing and correlate such progression to a drug previously prescribed for the patient. Decision support engine 114 may make this determination based on input medication consumption information for the patient (in combination with other factors). In certain embodiments, determining an optimal balance of potassium and insulin levels of a patient, as well as understanding the interplay between potassium levels, insulin, and one or more types of medicines for the patient, may help to inform which medicine (including dosage and frequency) is best suited for the patient.

In certain embodiments, medication recommendations may include a recommendation for the patient to take a potassium binder. For example, in certain embodiments, medication recommendations may include a recommendation for the patient to take a potassium binder as an enema rectally. In certain embodiments, medication recommendations may include a recommendation for the patient to take an oral potassium binder, such as Valtassa.

In certain embodiments, medication recommendations may include a recommendation for the patient to take a potassium binder in combination with insulin. For example, in certain embodiments, insulin may be used to reduce serum potassium levels since it stimulates potassium uptake by cells. However, while insulin facilitates movement of extracellular potassium into cells, it does not decrease total body potassium. Thus, to prevent a subsequent increase in total body potassium levels after an insulin dose, a potassium binder may be taken in combination therewith to clear potassium.

In certain embodiments, medication recommendations may include a recommendation for the patient to particular type of diuretic and/or dosage. As mentioned previously, medication, such as diuretics, may be prescribed to a patient for the purpose of treating excessive fluid accumulation caused by congestive heart failure (CHF), liver failure, and/or nephritic syndrome. Types of diuretics prescribed may include loop diuretics, thiazide and thiazide-like diuretics, and/or potassium-sparing diuretics. Each of the identified diuretic types may be prescribed to a patient for a different purpose. Thus, in certain embodiments, decision support engine 114 may determine the optimal diuretic for prescription based on the health of the patient and the condition(s) of the patient to be treated. For example, a patient with CHF (without kidney disease) may typically be prescribed a thiazide-like diuretic. In particular, thiazide-like diuretics may help to get rid of the excess fluid caused by CHF; however, such diuretics may, in some cases, dehydrate the patient. Dehydration of a patient with impaired kidneys may further damage the kidneys of the patient. In particular, dehydration may clog the kidneys with muscle proteins (myoglobin). Thus, where the patient is only experiencing CHF, and not kidney disease, prescribing the patient a thiazide-like diuretic may not cause significant harm to the patient's kidneys. However, where the patient is experiencing kidney failure, such diuretics may not be optimal for prescription. Accordingly, another diuretic type may be considered.

In certain embodiments, medication recommendations may include a recommendation for the patient to take a diuretic in combination with an insulin dose. Again, insulin facilitates movement of extracellular potassium into cells, but does not decrease total body potassium. Thus, to prevent an increase in total body potassium levels after an insulin dose, a diuretic may be taken in combination therewith.

Further, in certain embodiments, decision support engine 114 may determine the optimal diuretic for prescription by also considering possible side effects the diuretic prescribed may have on other organs of the patient. By considering the impact different medications have on other organs, decision support engine 114 may aid a patient in managing conditions of other organs within the patient's body. In certain embodiments, CPM 202 may aid in making this determination, at least with respect to the patient's kidneys. For example, CPM 202 may be used to monitor the effect of medication prescribed to a patient for the purpose of treating CHF to better determine its side effect(s) on kidney functions of the patient. Where potassium levels are observed to be decreasing over a period of time after the patient has been prescribed a diuretic for CHF, one may assume the patient is experiencing dehydration, and further conclude, such dehydration may be adversely impacting the kidneys of the patient. Accordingly, a new diuretic may be considered for prescription.

In certain embodiments, alarm/alert recommendations may include a recommendation for the addition of a new type of alarm/alert, the removal of an existing alarm/alert, an increase or decrease in the frequency of existing alarms/alerts, and/or a change in existing threshold levels for existing alarms/alerts configured for a device used by the patient. As mentioned, in certain embodiments, the type of alarms/alerts customized for each particular display device, the number of alarms/alerts customized for each particular display device, the timing of alarms/alerts customized for each particular display device, and/or the threshold levels configured for each of the alarms (e.g., for triggering) are based on the current health of a patient, the state of a patient's kidney, current treatment recommended to a patient, physiological parameters of a patient when experiencing different symptoms stored in user profile 118 for each patient, and/or, in some cases, the disease prediction generated at block 506.

In certain embodiments, the disease prediction generated at block 506 may be used to stratify the patient's risk of hospital admission/readmission. In certain embodiments, the disease prediction generated at block 506 may be used to better understand post hospital discharge stability of the patient when determining whether to discharge the patient. In certain embodiments, the disease prediction generated at block 506 may be used to monitor patient's post hospital discharge stability and to determine if and when a patient should be readmitted. In certain embodiments, the disease prediction generated at block 506 may be used to stratify the level of care a patient should receive upon hospital admittance. For example, a patient admitted to a hospital may be one of many patients in the hospital. Accordingly, in certain aspects, the disease prediction generated for the patient may be compared to similarly generated disease predictions of other patients in the hospital to better inform medical personnel in the hospital where the patient ranks among other patients, in terms of the level of care needed, as well as the urgency of attention needed by the patient among other patients. This may be especially important where a patient is at high risk of experiencing an acute deadly event within a small amount of time after being admitted into the hospital (e.g., as opposed to waiting four, or more, hours prior to receiving assistance or care by medical personnel).

In certain embodiments, machine learning models deployed by decision support engine 114 include one or more models trained by training server system 140, as illustrated in FIG. 1. FIG. 6 describes in further detail techniques for training the machine learning model(s) deployed by decision support engine 114 for generating predictions associated with kidney health, according to certain embodiments of the present disclosure.

In certain embodiments, method 600 is used to train models to generate predictions associated with kidney health. Predications associated with kidney disease may include (1) predictions of one or more symptoms associated with kidney disease, (2) predictions as to the kidney health of a patient, including the presence and/or severity of kidney disease (e.g., a user illustrated in FIG. 1), and/or (3) predictions as to optimal treatment for a patient.

Method 600 begins, at block 602, by a training server system, such as training server system 140 illustrated in FIG. 1, retrieving data from a historical records database, such as historical records database 112 illustrated in FIG. 1. As mentioned herein, historical records database 112 may provide a repository of up-to-date information and historical information for users of a continuous analyte monitoring system and connected mobile health application, such as users of continuous analyte monitoring system 104 and application 106 illustrated in FIG. 1, as well as data for one or more patients who are not, or were not previously, users of continuous analyte monitoring system 104 and/or application 106. In certain embodiments, historical records database 112 may include one or more data sets of historical patients with no kidney disease or varying stages of kidney disease.

Retrieval of data from historical records database 112 by training server system 140, at block 602, may include the retrieval of all, or any subset of, information maintained by historical records database 112. For example, where historical records database 112 stores information for 100,000 patients (e.g., non-users and users of continuous analyte monitoring system 104 and application 106), data retrieved by training server system 140 to train one or more machine learning models may include information for all 100,000 patients or only a subset of the data for those patients, e.g., data associated with only 50,000 patients or only data from the last ten years.

As an illustrative example, integrating with on premises or cloud based medical record databases through Fast Healthcare Interoperability Resources (FHIR), web application programming interfaces (APIs), Health Level 7 (HL7), and or other computer interface language may enable aggregation of healthcare historical records for baseline assessment in addition to the aggregation of de-identifiable patient data from a cloud based repository.

As an illustrative example, at block 602, training server system 140 may retrieve information for 100,000 patients with varying stages of kidney disease stored in historical records database 112 to train a model to predict the presence and/or severity of kidney disease in a user. Each of the 100,000 patients may have a corresponding data record (e.g., based on their corresponding user profile)), stored in historical records database 112. Each user profile 118 may include information, such as information discussed with respect to FIG. 3.

The training server system 140 then uses information in each of the records to train an artificial intelligence or ML model (for simplicity referred to as “ML model” herein). Examples of types of information included in a patient's user profile were provided above. The information in each of these records may be featurized (e.g., manually or by training server system 140), resulting in features that can be used as input features for training the ML model. For example, a patient record may include or be used to generate features related to an age of a patient, a gender of the patient, an occupation of the patient, analyte levels for the patient over time, analyte level rates of change and/or trends for the patient over time, physiological parameters associated with different kidney disease stages for the patient over time, etc. Features used to train the machine learning model(s) may vary in different embodiments.

In certain embodiments, each historical patient record retrieved from historical records database 112 is further associated with a label indicating whether the patient was healthy or experienced some variation of kidney disease, a previously determined kidney disease diagnosis and/or stage of kidney disease for the patient, a kidney disease risk assessment, treatment(s), and/or similar metrics. What the record is labeled with would depend on what the model is being trained to predict.

At block 604, method 600 continues by training server system 140 training one or more machine learning models based on the selected historical patient record. In some embodiments, the training server does so by providing the features (e.g., extracted at block 606) as input into a model. This model may be a new model initialized with random weights and parameters, or may be partially or fully pre-trained (e.g., based on prior training rounds). Based on the input features, the model-in-training generates some output. In certain embodiments, the output may indicate whether the patient was healthy or experienced some variation of kidney disease, a kidney disease diagnosis and/or kidney disease stage for the patient, a risk assessment, previously recommended treatments, or similar metrics. In certain embodiments, the output may indicate whether the patient experienced one or more symptoms commonly associated with kidney disease or the level of risk the patient was at for experiencing one or more symptoms commonly associated with kidney disease.

In certain embodiments, training server system 140 compares this generated output with the actual label associated with the historical patient record to compute a loss based on the difference between the actual result and the generated result. This loss is then used to refine one or more internal weights and parameters of the model (e.g., via backpropagation) such that the model learns to predict the kidney health of the user, including the presence and/or severity of kidney disease (or its recommended treatments) more accurately.

One of a variety of machine learning algorithms may be used for training the model(s) described above. For example, one of a supervised learning algorithm, a neural network algorithm, a deep neural network algorithm, etc. may be used.

At block 606, training server system 140 deploys the trained model(s) to make predictions associated with kidney health during runtime. In some embodiments, this includes transmitting some indication of the trained model(s) (e.g., a weights vector) that can be used to instantiate the model(s) on another device. For example, training server system 140 may transmit the weights of the trained model(s) to decision support engine 114. The model(s) can then be used to assess, in real-time, the presence and/or severity of kidney disease of a user using application 106, provide treatment recommendations, and/or make other types of predictions discussed above. In certain embodiments, the training server system 140 may continue to train the model(s) in an “online” manner by using input features and labels associated with new patient records.

Further, similar methods for training illustrated in FIG. 6 using historical patient records may also be used to train models using patient-specific records to create more personalized models for making predictions associated with kidney health. For example, a model trained using historical patient records that is deployed for a particular user, may be further re-trained after deployment. For example, the model may be re-trained after the model is deployed for a specific patient to create a more personalized model for the patient. The more personalized model may be able to more accurately predict symptoms for the patient based on the patient's own data (as opposed to only historical patient record data), including the patient's own potassium thresholds.

### Example Methods and Systems for Providing Decision Support Around Kidney Health and/or Diabetes

As mentioned, in certain embodiments, the decision support system described herein is designed to provide decision support in the form of risk assessment and treatment for kidney disease and/or glucose homeostasis. For example, a method may be used to provide risk stratification and treatment recommendations for the treatment and/or prevention of kidney disease, hyperkalemia, hypokalemia, hyperglycemia, and/or hypoglycemia.

In certain embodiments, the method is a decision tree, which is a tree-like collection of (1) attribute nodes linked to two or more sub-trees and (2) decision nodes (also commonly referred to as leaf nodes). An attribute node computes some outcome based on attribute value(s) of an instance (e.g., in this case, a user) being evaluated, where each possible outcome is associated with one of the sub-trees. A decision node is a node which does not split into any more nodes and provides a decision based on previously evaluated attribute(s) for the instance. An instance may be classified by starting at a root node of the method and working down/through the method until a decision node is encountered.

For example, the method described herein may be used to classify a user, and more specifically, classify a user as (1) a patient at risk of hyperkalemia, (2) a patient at risk of hypokalemia, or (3) a patient neither at risk of hyperkalemia, nor hypokalemia, and further provide preventative and/or treatment recommendations based on the initial classification. Accordingly, attribute nodes of the method may include the evaluation of continuously measured potassium and/or glucose levels (e.g., attribute values) of a user against threshold potassium and/or glucose thresholds, respectively. Further, in some cases, attribute nodes of the method include the evaluation of user medication and/or user activity related to insulin dosing, dialysis treatment, potassium consumption, glucose consumption, etc. Decision nodes of the method may include recommended actions for correcting and/or maintaining potassium homeostasis, and additionally in some cases, recommended actions for correcting and/or maintaining glucose homeostasis. Example recommended actions may include recommendations for potassium ingestion, glucose ingestion, water ingestion, insulin dosing, dialysis, potassium binder ingestion, and/or the like.

In certain embodiments, decision support risk stratification and treatment recommendations may be based on a comparison of a user's potassium levels to different potassium thresholds. For example, decision support risk stratification and treatment recommendations may be based on a comparison of a user's potassium levels to one or more different potassium thresholds. Different potassium thresholds utilized in the method include (1) an intensely elevated potassium threshold, (2) a modestly elevated potassium threshold, (3) an elevated potassium threshold, (4) a depressed potassium threshold, (5) an intensely depressed potassium threshold.

In certain embodiments, decision support risk stratification and treatment recommendations may be based on a comparison of a user's glucose levels to one or more different glucose thresholds. For example, different glucose thresholds utilized in the method include (1) an elevated glucose threshold and (2) a depressed glucose threshold.

In certain embodiments, one or more of these potassium and/or glucose thresholds are predefined for all users (e.g., based on historical data). In certain embodiments, one or more of these potassium and/or glucose thresholds are predefined for different populations of similarly situated users (e.g., based on historical data for similarly situated users). In certain embodiments, one or more of these thresholds are individualized for different users through a user or a healthcare professional (HCP) manually setting the threshold(s).

In certain embodiments, one or more of these thresholds may be individualized for different users based on a user's potassium baseline and/or glucose baseline, respectively. For example, as mentioned herein, each user may have a different potassium baseline. In certain embodiments, a user's potassium baseline may be determined by calculating an average of potassium levels of the user over a specified amount of time where significant fluctuations (e.g., rapidly increasing or rapidly decreasing potassium levels) are not expected. For example, the baseline potassium for a user may be determined over a period of time when the user is sleeping, sitting in a chair, or other periods of time where the user is sedentary and not consuming food and/or medication (e.g., insulin administration, medication known to affect potassium levels, etc.) which would reduce or increase potassium levels (e.g., where no external conditions exist that would affect the potassium baseline exist). However, specific, personalized potassium thresholds may be constantly changing for a user. Thus, in certain embodiments, DAM 116 may continuously calculate a potassium baseline, time-stamp the calculated potassium baseline, and store the corresponding information in the user's profile 118. Further, in certain embodiments, potassium thresholds that are (1) for a population of users or (2) individualized for a particular user, may be altered based, at least in part, on a number of analytes being measured for input to reflect the knowledge attained from each of the other analytes being measured. Similar methods may be used for calculating, determining, and/or storing glucose baselines of a user.

Because potassium baselines and/or glucose baselines may be different for each user, potassium thresholds and/or glucose thresholds set relative to user's potassium baseline and/or glucose baseline, respectively, may also be different for each user. In certain embodiments, an individual threshold may be set based on a set difference above or below a baseline. For example, a first user with significant kidney impairment may have a higher potassium baseline, and potassium threshold(s) specific to the first user may be set based on a 0.5 mmol/L difference compared to the first user's baseline. Thus, where the first user's potassium baseline is 4.5 mmol/L, an elevated potassium threshold specific to the first user may be set to 5.0 mmol/L, and, in certain examples, a depressed potassium threshold specific to the first user may also be set to 4.0 mmol/L. On the other hand, a second user with healthy kidney function may have a lower potassium baseline than the first user, for example, a potassium baseline of 3.8 mmol/L. Accordingly, an elevated potassium threshold specific to the second user may be set to 4.3 mmol/L, and/or a depressed potassium threshold specific to the second user may be set to 3.3 mmol/L (e.g., where a 0.5 mmol/L different is used).

In certain examples, user-specific elevated and depressed analyte thresholds (e.g., potassium and/or glucose) may be determined separately, and only the elevated analyte threshold or the depressed analyte threshold is set based on a difference to a user's analyte baseline. In certain examples, a user-specific elevated analyte threshold may be adjusted by an absolute value (e.g., an absolute set difference) that is different (e.g., greater or lesser) from an absolute value of an adjustment of a user-specific depressed analyte threshold, based on the difference to the user's analyte baseline. In further embodiments, analyte thresholds such as potassium thresholds and/or glucose thresholds may instead be set based on population-based guidelines, and may not be modified or set based on a user's corresponding analyte baseline.

In certain examples of analyte thresholds, the determination of elevated and depressed analyte levels may be made based on a potassium range for a healthy user. For example, a potassium range of 3.0 mmol/L to 5.5 mmol/L may represent a potassium range with a healthy user, based on population data. In this example, a user with a potassium baseline of 4 mmol/L would only be notified of an elevated potassium threshold if the user's baseline potassium exceeds 5.5. mmol/L. Conversely, a user would be notified of a depressed potassium level if the user's baseline potassium falls below 3.0 mmol/L.

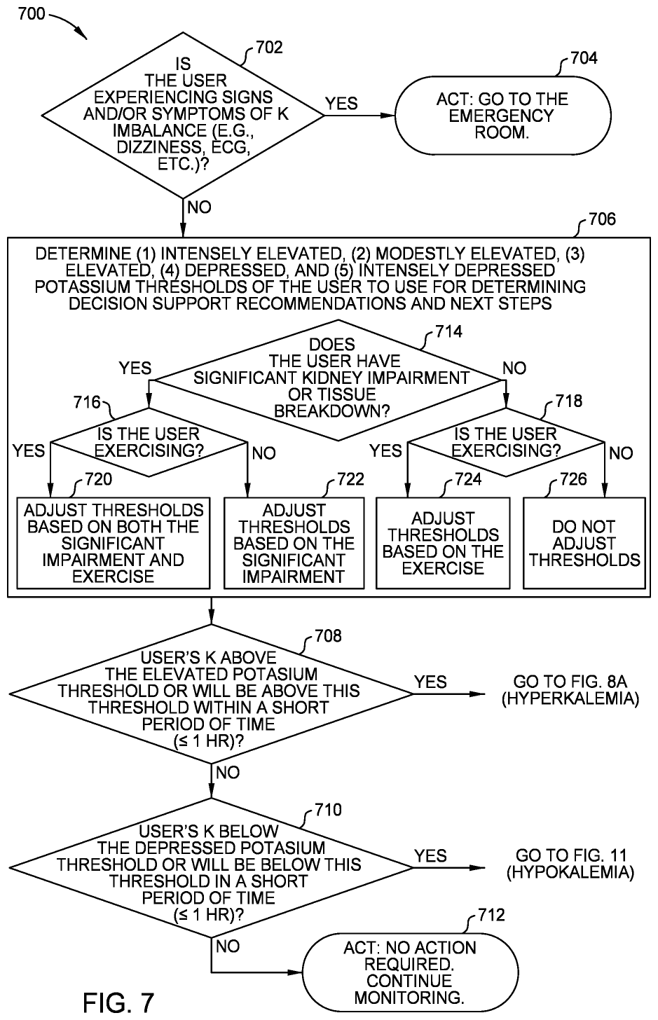

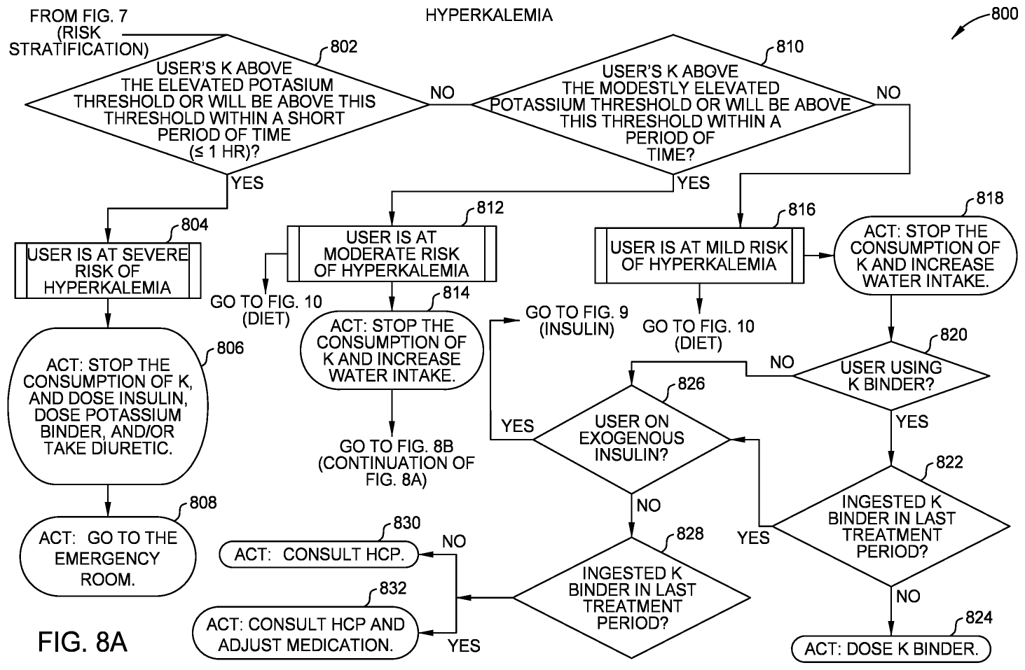

As described below with respect to FIGS. 7-13, the method described herein may be divided into five groupings: (1) risk stratification, (2) hyperkalemia decision support, (3) hypokalemia decision support, (4) glucose/exogenous insulin decision support, and (5) diet decision support. For example, the method begins (e.g., at a root node) by first stratifying users into different risk categories based on their risk of hyperkalemia or hypokalemia. Thus, the method may be split into two sections, including (1) a first section for users determined to be at risk of hyperkalemia and (2) a second section for users determined to be at risk of hypokalemia. A user determined to be at risk of hyperkalemia may proceed down the first section of the method, which provides treatment recommendations for users at risk of hyperkalemia. In other words, the recommendations may include diet recommendations, medication recommendation, and/or recommendations to take one or more actions that take into consideration the user's risk of hyperkalemia. Alternatively, a user determined to be at risk of hypokalemia may proceed down the second section of the method, which provides treatment recommendations for users at risk of hypokalemia. In other words, the recommendations may include diet recommendations, medication recommendation, and/or recommendations to take one or more actions that take into consideration the user's risk of hypokalemia.

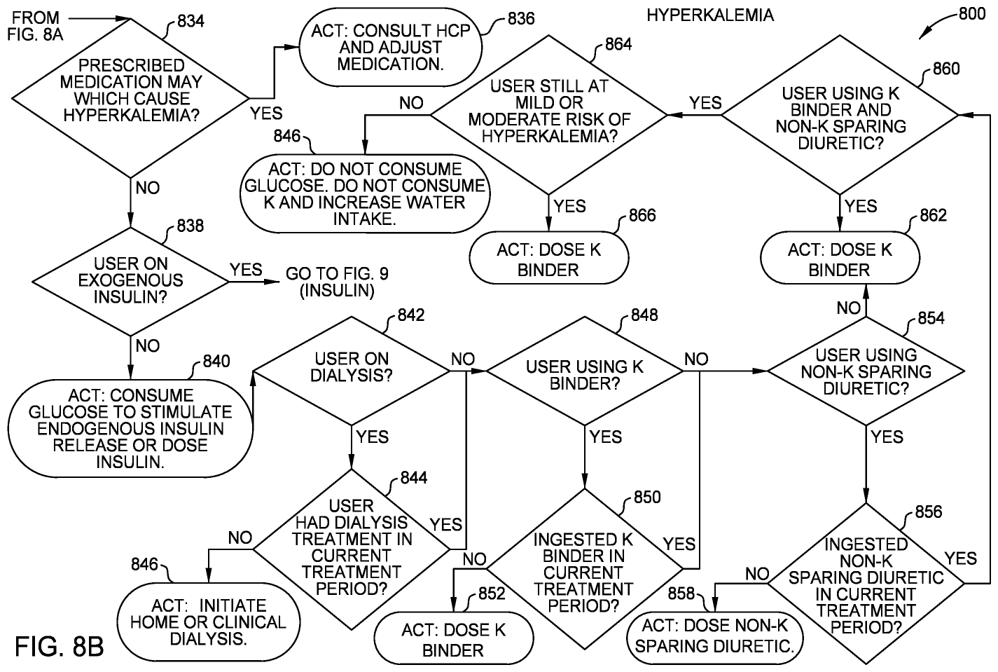

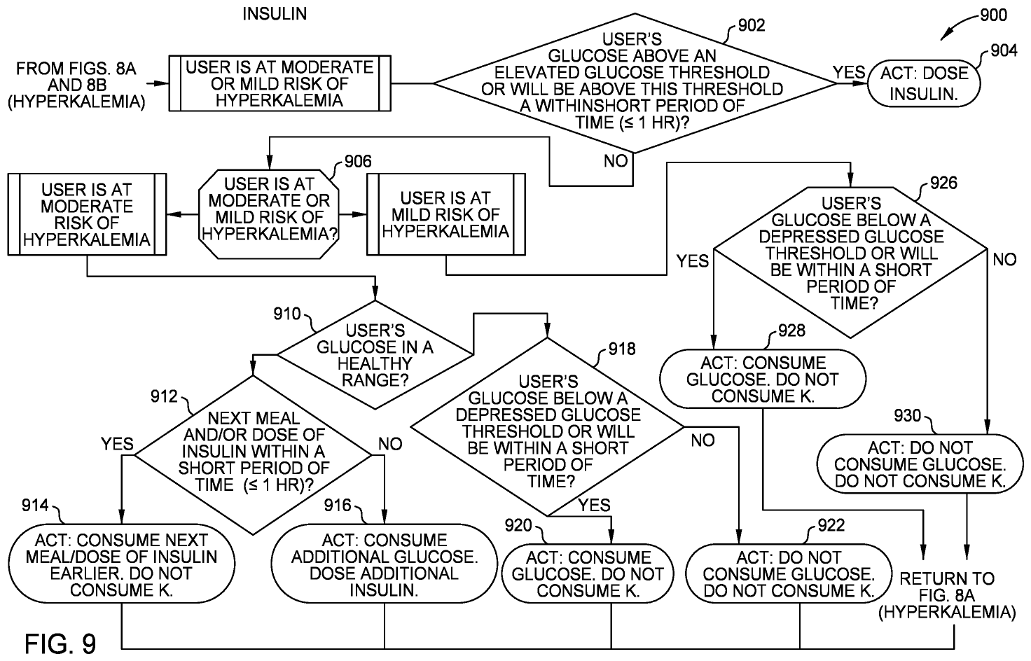

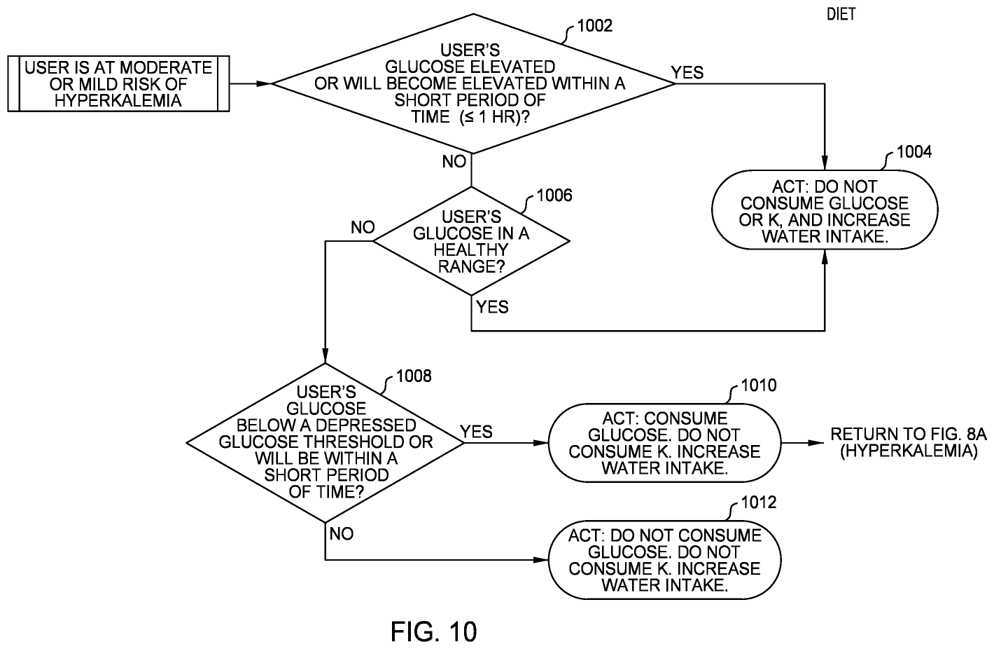

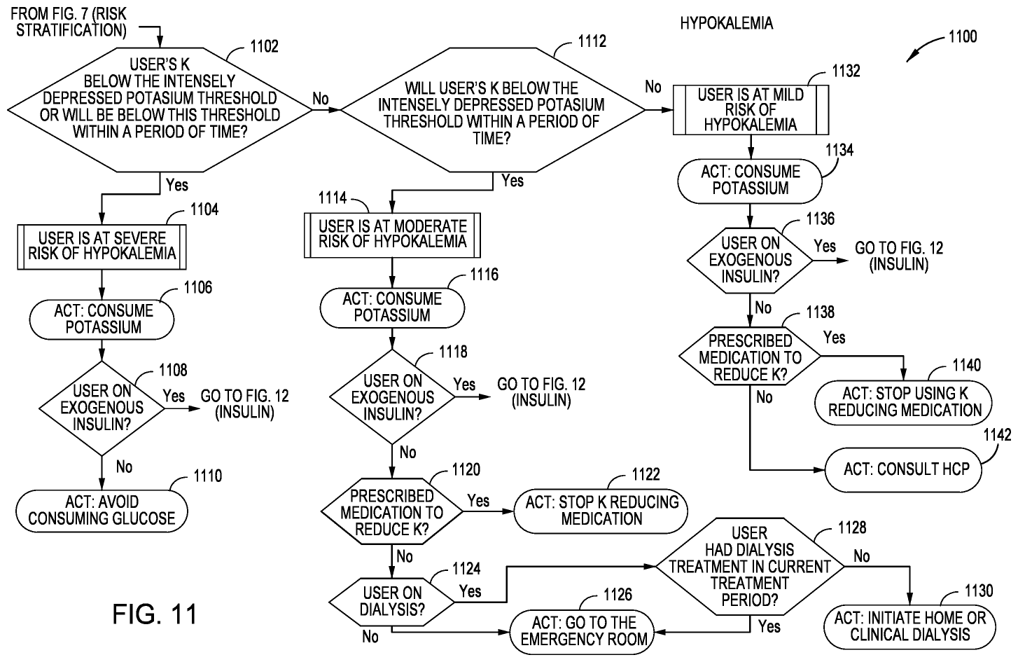

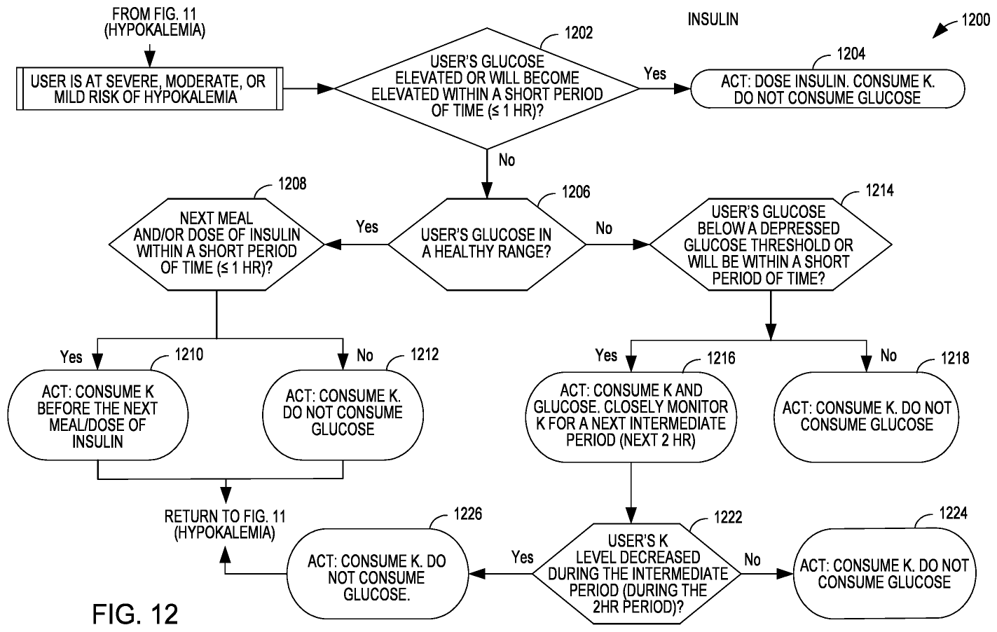

FIGS. 7-13 illustrate, as a whole, an example method used for providing such risk stratification and treatment (or preventative) recommendations to a user. More specifically, FIG. 7 is an example workflow stratifying users into different risk categories based on their risk of hyperkalemia or hypokalemia, FIGS. 8A, 8B, 9, and 10 make up the first section of the method (e.g., for users determined to be at risk of hyperkalemia), while FIGS. 11, 12, and 13 make up the second section of the method (e.g., for users determined to be at risk of hypokalemia). In certain embodiments, decision support system 100 illustrated in FIG. 1 may be configured to collect data, such as inputs 128 and metrics 130, including for example, analyte data (e.g., from a continuous potassium monitor (CPM) and/or a continuous glucose monitor (CGM)) and patient information mentioned above, and use this information as input for the method illustrated in FIGS. 7-13 to (1) classify a user into different risk categories based on their risk of hyperkalemia or hypokalemia and (2) provide one or more recommendations for treatment (or prevention) for the user based the classification. More specifically, decision support engine 114 of decision support system 100 may be configured to perform such (1) classification and (2) provide the one or more recommendations. In some cases, decision support engine 114, after making the one or more recommendations, may provide the output recommendation(s) to the user (e.g., through application 106 illustrated in FIG. 1), to a user's caretaker (e.g., a parent, a relative, a guardian, a teacher, a nurse, etc.), to a user's physician, or any other individual that has an interest in the wellbeing of the user for purposes of improving the user's health, such as, in some cases by effectuating the recommended treatment and/or preventative actions. FIGS. 7-13 are described in more detail below.

FIG. 7 is an example workflow 700 for stratifying a user's risk of disease(s) commonly associated with kidney disease, including hyperkalemia and hypokalemia, according to certain embodiments of the present disclosure. Workflow 700 is described below with reference to FIGS. 1 and 2 and their components. For example, workflow 700 may be performed by components of decision support system 100 illustrated in FIG. 1 to continuously monitor one or more analytes of a user, such as user 102 illustrated in FIG. 1, during a plurality of time periods and stratify users into different risk categories based on the continuously monitored analyte(s). In certain embodiments, decision support system 100 is configured to continuously monitor potassium levels of the user and use workflow 700 to stratify a user's risk of hyperkalemia and/or hypokalemia based on the potassium levels of the patient.

Workflow 700 begins at block 702 by decision support system 100, and more specifically decision support engine 114 of decision support system 100, determining whether the user is experiencing signs and/or symptoms of potassium imbalance. As described previously, a sign is an objective, observable phenomenon that can be identified by another person or device. On the other hand, a symptom is a subjective experience of a patient.

Signs and symptoms of potassium imbalance may be determined through several means, including the use of one or more analyte sensors (e.g., a CPM) and one or more non-analyte sensors (e.g., heart rate monitor, blood pressure monitor, respiration monitor, ECG, accelerometer, temperature sensor, etc.). For example, in certain embodiments, decision support engine 114 determines whether the user is experiencing (or will experience) one or more symptoms of potassium imbalance in accordance with workflow 400A of FIG. 4A and/or workflow 400B of FIG. 4B, based on analyte data from one or more analyte sensors and/or non-analyte data from one or more non-analyte sensors.

In certain embodiments, decision support engine 114 determines whether the user is experiencing (or will experience) one or more or symptoms, and/or is showing one or more signs, of potassium imbalance based on user inputted symptoms and/or signs. For example, a user may manually input into decision support system 100 that the user is experiencing one or more signs or symptoms.

In certain embodiments, decision support engine 114 determines whether the user is experiencing (or will experience) one or more or symptoms, and/or is showing one or more signs, of potassium imbalance based on user input confirming such symptoms and/or signs. For example, decision support system 100 may confirm one or more signs and/or symptoms (e.g., in some cases predicted in accordance with workflows 400A and 400B of FIGS. 4A and 4B, respectively). In some cases, decision support system 100 prompts the user based on analyte data (e.g., elevated or increasing potassium levels, depressed or decreasing potassium levels, etc.) and/or non-analyte data (e.g., increased respiration, ECG changes, blood pressure changes). In other cases, decision support system 100 may confirm one or more signs and/or symptoms based on contextual data (e.g., cameras) other input data (e.g. sensor data), or observations and/or inputs from a person other than the patient (e.g., a healthcare provider).

In some cases, decision support engine 114 may notify the user of potassium imbalance based on abnormal analyte data (e.g., potassium data) collected from one or more analyte sensors. In some cases, decision support system 114 may determine signs and/or symptoms of potassium imbalance. Signs and/or symptoms of potassium imbalance may include muscle weakness or paralysis, nausea, tingling, or cardiac conduction abnormalities, and/or cardiac arrhythmias, including sinus bradycardia, sinus arrest, slow idioventricular rhythms, ventricular tachycardia, ventricular fibrillation, and/or asystole. Additionally, electrocardiogram (ECG) abnormalities may be a sign of hyperkalemia. For example, a tall peaked T wave with a shortened QT interval, followed by a progressive lengthening of the PR interval (e.g., the PR interval is the time from the beginning of the P wave (atrial depolarization) to the beginning of the QRS complex (ventricular depolarization)) and QRS duration may be a sign of hyperkalemia. In addition, the P wave may disappear, and QRS may widen to a sine wave. One or more of these signs and/or symptoms (and/or other signs and symptoms not listed) of hyperkalemia and/or hypokalemia may indicate a serious condition requiring immediate medical attention.

As such, where at block 702, decision support engine 114 determines that the user is experiencing signs and/or symptoms of potassium imbalance, at block 704, decision support engine 114 provides a recommendation for the user to go to the emergency room. In other words, urgent medical attention beyond available at-home treatments is recommended by decision support engine 114.

Any of the signs or symptoms described above may indicate a severe risk of hyperkalemia and/or hypokalemia, or other critical medical condition which may not be resolved with at-home treatments readily available to users. Thus, due to the significant risk associated with signs and/or symptoms of potassium imbalance, the recommendation for a user having one or more signs and/or symptoms is to go to the emergency room. Workflow 700 may terminate after providing this recommendation given urgent medical attention beyond available at-home treatments is recommended.

Although potassium levels could be indicative of signs and/or symptoms of potassium imbalance, in certain cases, a user may experience the signs and/or symptoms of potassium imbalance described above, even in cases where the user's potassium levels do not indicate a severe risk. Therefore, as shown in FIG. 7, decision support engine 114 may make a recommendation for a user to seek immediate medical attention irrespective of the user's potassium levels (and their comparison to one or more potassium thresholds, in some cases, personalized for the user).

Alternatively, where at block 702, decision support engine 114 determines the user is not experiencing signs and/or symptoms of potassium imbalance, at block 706, decision support engine 114 determines different potassium thresholds to use for evaluating continuously monitored potassium levels of the user (e.g., for risk stratification). The different potassium thresholds determined by decision support engine 114 may include (1) an intensely elevated potassium threshold, (2) a modestly elevated potassium threshold, (3) an elevated potassium threshold, (4) a depressed potassium threshold, and/or (5) an intensely depressed potassium threshold.

In certain embodiments, the different potassium thresholds represent a potassium level value of a patient (e.g., 3.0 mmol/L, 5.5 mmol/L, etc.). In certain embodiments, the different potassium thresholds represent potassium level rates of change. As described herein, a potassium level rate of change refers to a rate that indicates how one or more time-stamped potassium measurements or values change in relation to one or more other time-stamped potassium measurements or values. Potassium level rates of change may be determined over one or more seconds, minutes, hours, days, etc.

Per FIG. 7, decision support engine 114 determines one or more of these thresholds based, at least in part, on (1) whether the user has significant kidney impairment or tissue breakdown and/or (2) whether the user is currently exercising (e.g., currently engaging in physical exertion) or just finished exercising (e.g., immediately prior in time).

In particular, several conditions may result in elevated potassium levels, including both chronically elevated and acutely elevated serum potassium levels. Some conditions resulting in acutely elevated serum potassium may lead to rapidly increasing potassium levels and severe hyperkalemia. For example, ongoing tissue breakdown, ongoing potassium absorption, and/or significant non-anion gap metabolic acidosis or respiratory acidosis may result in large and/or rapid releases of potassium into the serum. Further, some conditions that result in chronically elevated serum potassium levels may also mask the changes in a user's potassium level. For example, CKD, especially later stages of CKD, may result in chronically elevated serum potassium.

In certain embodiments, to account for situations where these conditions elevate potassium levels of a user, the thresholds upon which decision support engine 114 relies are adjusted (e.g., increased). For example, thresholds for decision support for a user with CKD may be increased to account for the effect of CKD on potassium levels. As such, in certain embodiments, (1) an intensely elevated potassium threshold, (2) a modestly elevated potassium threshold, (3) an elevated potassium threshold, (4) a depressed potassium threshold, and/or (5) an intensely depressed potassium threshold of the user with CKD may be higher than (1) an intensely elevated potassium threshold, (2) a modestly elevated potassium threshold, (3) an elevated potassium threshold, (4) a depressed potassium threshold, and/or (5) an intensely depressed potassium threshold for a user without CKD.

In certain embodiments, adjustments to the potassium thresholds include an adjustment of the potassium threshold to a particular threshold. In certain embodiments, predefined thresholds are associated with different conditions. For example, an elevated potassium threshold associated with CKD may be 5.8 mmol/L. Thus, a user with CKD may have an adjusted potassium threshold of 5.8 mmol/L.

In certain embodiments, adjustments to the potassium thresholds include adjusting such thresholds relative to a user's potassium baseline. In certain embodiments, predefined adjustments (or changes) are associated with different conditions. For example, an adjustment of +0.5 mmol/L may be associated with CKD. Thus, a user with CKD may have an adjusted potassium threshold equal to their potassium baseline plus 0.5 mmol/L.

In certain embodiments, adjustments to the potassium thresholds include adjusting such thresholds relative to a user's potassium rate of change. For example, such thresholds may include different rates of potassium change. These different rates of potassium change may be adjusted by a common/single absolute value (e.g., a rate of change value) or different absolute values. In certain embodiments, predefined adjustment, rate of change values are associated with different conditions.

Further, in certain embodiments, the thresholds, upon which decision support engine 114 relies, are adjusted (e.g., increased) based on information about exercise performed by the user. In particular, serum potassium levels of a user may be affected by exercise. Specifically, potassium is released from inside cells (i.e., intracellular) into the serum. Potassium levels may increase up to 8 mmol/L during exercise and may normalize within minutes after stopping exercise. Because potassium levels may be significantly elevated and/or rapidly elevated during exercise while significantly dropping following cessation of the exercise, thresholds upon which decision support engine 114 relies may need to be adjusted for periods of exercise and/or for periods following cessation of the exercise. For example, thresholds during exercise may be increased to account for the effect of exercise on potassium levels. In another example, thresholds for a decreasing rate of change may be reduced following exercise to account for the effect of exercise on potassium levels following cessation of the exercise.

In certain embodiments, potassium thresholds for a user that is exercising (e.g., referred to herein as “exercise potassium thresholds”) are standardized based on historical data or data from similarly situated users. Historical data may include potassium levels, rates of change, and/or trends from previous exercise sessions. Exercise potassium thresholds may be individualized for each patient through a user and/or HCP inputted thresholds. Exercise potassium thresholds may further be individualized based on each user's potassium baseline. For example, exercise potassium thresholds may be set relative to a patient's baseline potassium levels, set as a ratio relative to the patient's baseline potassium levels, etc. Exercise potassium thresholds may be adjusted based on the user's potassium levels, rates of change, and/or trends before, during and following exercise.

In certain embodiments, decision support engine 114 uses exercise regimen metrics of metrics 130 stored in user profile 118, as illustrated in FIG. 1, to determine whether and/or when a user is exercising. As described herein, exercise regimen metrics may indicate one or more of what type of activities the user engages in and when, the corresponding intensity of such activities, a frequency with which the user engages in such activities, etc. Exercise regimen metrics may be calculated based on one or more of analyte sensor data input (e.g., from a lactate monitor, a glucose monitor, etc.), non-analyte sensor data input (e.g., non-analyte sensor data input from an accelerometer, a heart rate monitor, a respiration rate sensor, etc.), calendar input, user input, etc.

In certain embodiments, user input may include user inputted exercise information and/or confirmation of exercise. For example, in some cases, a user may provide input into the decision support system that the user is exercising. In some other cases, decision support system 100 may prompt a user and ask for confirmation of exercise. Decision support system 100 may prompt a user based on analyte data (e.g., elevated or increasing potassium levels), other analytes (e.g., lactate data, glucose data, etc.), and/or non-analyte data (e.g., increased respiration, increased HR, accelerometer).

As such, potassium thresholds, for the user, used in the method may be based, at least in part, on (1) whether the user has significant kidney impairment or tissue breakdown and/or (2) whether the user is currently exercising (e.g., currently engaging in physical exertion) or just finished exercising (e.g., immediately prior in time). For example, decision support for a user with significant impairment is based on thresholds adjusted based on conditions which result in elevated potassium levels in a user (e.g., referred to herein as “Adjusted Significant Impairment Elevated Thresholds”). In another example, decision support for a user while exercising is based on thresholds adjusted based the user currently, or immediately prior, engaging in some level of exercise (e.g., referred to herein as “Exercise Elevated Thresholds”). Accordingly, (1) an intensely elevated potassium threshold, (2) a modestly elevated potassium threshold, and/or (3) an elevated potassium threshold for the patient may be adjusted based on changes in the user's profile, some of which may include changes in: potassium baseline, kidney function/health, diabetes/glucose homeostasis, medication, treatment, other comorbidities (e.g., hypertension, liver disease, cardiac disease, metabolic disease, etc.), diet, exercise, adverse event(s) (e.g., hyperkalemia/hypokalemia, hyperglycemia/hypoglycemia, cardiac event, etc.), signs of hyperkalemia/hypokalemia, and/or symptoms of hyperkalemia/hypokalemia.

For example, as shown in FIG. 7, at block 714, decision support engine 114 determines whether the user has significant kidney impairment and/or tissue breakdown. In cases where decision support engine 114 determines, at block 714, that the user does have significant kidney impairment and/or tissue breakdown, at block 716, decision support engine 114 further determines whether the user is exercising (or was exercising immediately prior).

Exercise-induced changes (e.g., elevations) in potassium levels can be drastic in rate and magnitude. Typically, for users with healthy kidney function, exercise-induced changes in potassium levels are transient in nature. However, for users with impaired kidney function, such exercise-induced potassium levels may linger, which can lead to further complications, such as, hyperkalemia and arrhythmia. Thus, identification of exercise may be crucial in determining a user's risk of hyperkalemia. In certain embodiments, determination of whether the user is exercising (or was exercising immediately prior) is based on a rate of change in the user's potassium levels, a duration of elevated potassium levels, and/or variability (or stability) of potassium levels. In certain embodiments, at block 716, decision support engine 114 may further determine whether the user has good control of exercise-induced potassium levels, and thus, is at low risk of lingering exercise-induced potassium levels, or if whether the user has reduced control of exercise-induced potassium levels, and thus, is at high risk of lingering exercise-induced potassium levels. Such determination may be based on the user's own historical data comprising duration of exercise-induced elevated potassium levels, and/or exercise-induced variability of potassium levels. The determination at block 716 may then be utilized in, e.g., FIG. 8A for determining the risk of hyperkalemia for the user.

In cases where decision support engine 114 determines, at block 716, that the user is exercising (or was exercising immediately prior), at block 720, decision support engine 114 adjusts potassium thresholds of the user based on both the significant impairment and the exercise. In other cases where decision support engine 114 determines, at block 716, that the user is not exercising (or was not exercising immediately prior), at block 722, decision support engine 114 adjusts potassium thresholds of the user based on the significant impairment (and not based on the exercise) (e.g., uses Adjusted Significant Impairment Elevated Thresholds for the remainder of the method).

In cases where decision support engine 114 determines, at block 714, that the user does not have significant kidney impairment and/or tissue breakdown, at block 718, decision support engine 114 further determines whether the user is exercising (or was exercising immediately prior). In certain embodiments, determination of whether the user is exercising (or was exercising immediately prior) is based on a rate of change in the user's potassium levels, a duration of elevated potassium levels, and/or variability (or stability) of potassium levels.

In cases where decision support engine 114 determines, at block 718, that the user is exercising (or was exercising immediately prior), at block 724, decision support engine 114 adjusts potassium thresholds of the user based on the exercise (and not based on the significant impairment) (e.g., uses Exercise Elevated Thresholds for the remainder of the method). In other cases where decision support engine 114 determines, at block 718, that the user is not exercising (or was not exercising immediately prior), at block 726, decision support engine 114 does not adjust potassium thresholds of the user based on either the significant impairment and/or exercise.

As an illustrative example, subsequent to block 706, decision support engine 114 may rely on (1) an intensely elevated potassium threshold equal to 6.5 mmol/L, (2) a modestly elevated potassium threshold equal to 5.5 mmol/L, (3) an elevated potassium threshold equal to 5.0 mmol/L, (4) a depressed potassium threshold equal to 3.5 mmol/L, and (6) an intensely depressed potassium threshold equal to 2.5 mmol/L to provide decision support to the user, and more specifically, classify a user as (1) a user at risk of hyperkalemia, (2) a patient at risk of hypokalemia, or (3) a patient neither at risk of hyperkalemia, nor hypokalemia, and further provide preventative and/or treatment recommendations based on the initial classification. In other words, decision support engine 114 may use these thresholds throughout the remainder of the method to classify the user and provide one or more recommendations.

Further in FIG. 7, at block 708, decision support engine 114 determines whether the user's potassium level is above the elevated potassium threshold (e.g., determined at block 706) (e.g., >5.0 mmol/L) or the user's potassium levels will be above the elevated potassium threshold within a short period of time (e.g., ≤1 hour). The short period of time may be predefined. In certain embodiments, elevated potassium levels may be determined by the user's potassium level reaching and/or crossing the elevated potassium threshold. In certain embodiments, elevated potassium levels may be determined by the user's potassium rate of change and/or trend indicating the user is likely to reach and/or cross the elevated potassium threshold within the defined short time period.

In some cases where decision support engine 114 determines, at block 708, that the user's potassium level is above the elevated potassium threshold (e.g., user's potassium level >5.0 mmol/L) or the user's potassium level will be above the elevated potassium threshold within the short period of time, decision support engine 114 determines the user is at risk of hyperkalemia. Thus, as shown in FIG. 7, the method proceeds to FIGS. 8A and 8B (e.g., beginning with FIG. 8A). FIGS. 8A and 8B illustrate an example method 800 used for providing decision support around kidney disease for a user determined to be at risk of hyperkalemia.

Although elevated potassium levels may not require immediate medical attention, a user's potassium levels may need to be promptly reduced to healthy levels. Furthermore, where the user's potassium level is not currently elevated, but may soon become elevated (e.g., elevated within the defined short time period), decision support system 100 may provide recommendations for the user to initiate potassium-lowering medication to prevent elevated potassium levels, or reduce the rate at which potassium levels have been elevating. Lowering potassium levels or a rapid rate of change thereof may reduce instances of hyperkalemia, reduce instances of significant/urgent intervention, help with kidney disease management, and/or increase effectiveness and availability of interventions. Example method 800 illustrated in FIGS. 8A and 8B may help decision support system 100, and more specifically decision support engine 114, in determining whether potassium levels of the user require immediate medical attention, available at-home treatments, and/or other actions to recommend to the user for purposes of reducing the user's potassium level.

In some other cases where decision support engine 114 determines, at block 708, that the user's potassium level is not above the elevated potassium threshold (e.g., user's potassium levels<5.0 mmol/L) and the user's potassium level will not be above the elevated potassium threshold within the short period of time, decision support engine 114 determines the user is not at risk of hyperkalemia. Accordingly, at block 710, decision support engine 114 determines whether the user's potassium level is below the depressed potassium threshold (e.g., determined at block 706) (e.g., <3.5 mmol/L) and/or whether the user's potassium level will be below the depressed potassium threshold within the short period of time (e.g., ≤1 hour). In certain embodiments, depressed potassium levels may be determined by the user's potassium level reaching and/or crossing the depressed potassium threshold. In certain embodiments, depressed potassium levels may be determined by the user's potassium rate of change and/or trend indicating the user is likely to reach and/or cross the depressed potassium threshold.

In some cases where decision support engine 114 determines, at block 710, that the user's potassium level is below the elevated potassium threshold (e.g., user's potassium level<3.5 mmol/L) and/or the user's potassium levels will be below the depressed potassium threshold within the short period of time, decision support engine 114 determines the user is at risk of hypokalemia. Thus, as shown in FIG. 7, the method proceeds to FIG. 11. FIG. 11 illustrates an example method 1100 used for providing decision support around kidney disease for a user determined to be at risk of hypokalemia.

Although depressed potassium levels may not require immediate medical attention (e.g., similar to elevate potassium levels), a user's potassium level may need to be promptly increased to healthy levels. Furthermore, where the user's potassium level is not currently depressed, but may soon become depressed (e.g., depressed within the defined short time period), decision support system 100 may provide recommendations for the user to perform one or more potassium-increasing actions (e.g., consuming potassium). Such recommendations may help reduce instances of hypokalemia, reduce instances of significant/urgent intervention, help with kidney disease management, and/or increase effectiveness and availability of interventions. Example method 1100 illustrated in FIG. 11 may help decision support system 100, and more specifically decision support engine 114, in determining whether potassium levels of the user require immediate medical attention, available at-home treatments, and/or other actions to recommend to the user for purposes of increasing the user's potassium level.

In some other cases where decision support engine 114 determines, at block 710, that the user's potassium level is not below the depressed potassium threshold (e.g., user's potassium level >3.5 mmol/L) and the user's potassium levels will not be below the depressed potassium threshold within the short period of time, decision support engine 114 determines that the user's potassium level is in a healthy range and is projected to remain in the healthy range. As such, no action may be required given the user's potassium level is in the healthy range. However, decision support system 100 may continue to monitor the user's potassium level for any changes.

As described above, decision support engine 114 may proceed to FIGS. 8A and 8B (e.g., beginning with FIG. 8A) to determine one or more recommendations to provide to a user classified as being at risk of hyperkalemia (e.g., in FIG. 7). FIGS. 8A and 8B illustrate an example method 800 used for providing decision support around kidney disease for a user determined to be at risk of hyperkalemia.

Method 800 is described below with reference to FIGS. 1 and 2 and their components. For example, components of decision support system 100 illustrated in FIG. 1 may use method 800 to determine treatment (or preventative) recommendations to recommend a user for purposes of reducing the user's elevated potassium level and/or preventing the user's elevated potassium level from increasing further.

Method 800 begins at block 802 by decision support system 100, and more specifically decision support engine 114 of decision support system 100, determining whether the user's potassium level is above the intensely elevated potassium threshold (e.g., determined at block 706 in FIG. 7) (e.g., >6.5 mmol/L) and/or whether the user's potassium level will be above the intensely elevated potassium threshold within a period of time, e.g., a short period of time (e.g., ≤1 hour), an intermediate period of time (e.g., ≤2 hours), or an extended period of time (e.g., 2 hours or more). The period of time may be predefined.

In certain embodiments where decision support engine 114 determines, at block 802, that the user's potassium level is above the intensely elevated potassium threshold (e.g., user's potassium level >6.5 mmol/L) and/or the user's potassium level will be above the intensely elevated potassium threshold within the period of time, decision support engine 114 determines, at block 804, that the user is at severe risk of hyperkalemia. Accordingly, as shown in FIG. 8A, at block 806, decision support engine 114 recommends the user stop any consumption (e.g., current or future consumption) of potassium. In certain embodiments where the user is at severe risk of hyperkalemia, at block 806, decision support engine 114 may further recommend to the user to dose insulin and/or dose a potassium binder and/or take a diuretic to urgently clear extracellular potassium levels. Thereafter, at block 808, decision support engine 114 recommends that the user visit the emergency room for treatment and/or analysis by a healthcare professional. In certain embodiments, decision support engine 114 may simultaneously recommend to the user to stop consumption of potassium and visit the emergency room for treatment and/or analysis by a healthcare professional.

In particular, severely (or intensely) elevated potassium levels (e.g., potassium levels >6.5 mmol/L) may be associated with a severe risk of hyperkalemia. Further, severely elevated potassium levels may be at increased risk of signs and/or symptoms of hyperkalemia and should be immediately reduced. As such, users with severely elevated potassium levels may be recommended to (1) seek immediate medical care at an emergency room and/or (2) immediately cease consumption of potassium.

Even where the user's potassium level is not yet at a severely elevated potassium level, where a user's potassium rate of change indicates that the user's potassium level will (or is likely to) reach the intensely elevated threshold (e.g., >6.5 mmol/L), in the time period, then the user's potassium level may still be associated with a severe risk of hyperkalemia. Users with severely elevated potassium levels should (1) seek immediate medical care at an emergency room and/or (2) immediately cease any consumption of potassium.

In certain other embodiments where decision support engine 114 determines, at block 802, that the user's potassium level is not above the intensely elevated potassium threshold (e.g., user's potassium level ≤6.5 mmol/L) and/or the user's potassium level will not be above the intensely elevated potassium threshold within the period of time, decision support engine 114 determines, at block 810, whether the user's potassium level is above the modestly elevated potassium threshold (e.g., determined at block 706 in FIG. 7) (e.g., >5.5 mmol/L) and/or whether the user's potassium level will be above the modestly elevated potassium threshold within the defined period of time, e.g., within an intermediate period of time (e.g., ≤2 hours).

In certain embodiments where decision support engine 114 determines, at block 810, that the user's potassium level is above the modestly elevated potassium threshold (e.g., user's potassium level >5.5 mmol/L) and/or the user's potassium level will be above the modestly elevated potassium threshold within the defined period of time, decision support engine 114 determines, at block 812, that the user is at moderate risk of hyperkalemia.

In particular, a user's elevated potassium level and/or a potassium rate of change, indicating that the user's potassium level will reach a modestly elevated threshold (e.g., >5.5 mmol/L) in the defined time period, e.g., in the defined intermediate time period (e.g., in the next 2 hours), may be associated with a moderate risk of hyperkalemia. A moderate risk of hyperkalemia may not be considered a hyperkalemic emergency requiring rapid acting therapies; however, a user with a moderate risk of hyperkalemia should seek to promptly (e.g., within 6 to 12 hours) reduce their potassium level. In certain embodiments, the user may use at home treatments and/or remedies to promptly reduce their potassium level. In certain embodiments, decision support system 100 recommends available therapies to the user for purposes of reducing the user's potassium levels to a healthy range. In certain embodiments, decision support system 100 recommends available at-home remedies to the user for purposes of reducing the user's potassium levels to a healthy range. In certain embodiments, decision support system 100 recommends that the user immediately cease consumption of potassium. In certain embodiments, decision support system 100 may determine that there is no suitable available therapy, and in such cases, a patient may be recommended to seek immediate medical care.

In certain embodiments where decision support engine 114 determines, at block 812, that the user is at moderate risk of hyperkalemia, decision support engine 114 may proceed to FIG. 10 to determine one or more diet recommendations for the treatment of kidney disease and/or for glucose homeostasis to a user classified as being at risk of hyperkalemia (e.g., in FIG. 7).

In certain embodiments, as shown in FIG. 8A, at block 814, decision support engine 114 recommends that the user stop any consumption (e.g., current or future consumption) of potassium and increase water intake. Further, at block 834 (e.g., illustrated in FIG. 8B), decision support engine 114 determines whether the user is prescribed medication which may cause hyperkalemia. In particular, some medications may increase potassium levels and/or otherwise induce hyperkalemia (e.g. drugs that inhibit the renin-angiotensin-aldosterone system (RAAS), nonsteroidal anti-inflammatory drugs, hypovolemia, etc.). In such embodiments, the user's potassium levels may be monitored for an extended period of time, e.g., 2 hours or more, or 7 days or more, to account for users who have been prescribed or put on a new medication, such as a non-potassium-sparing diuretic. In some examples, newly prescribed medications may necessitate the monitoring of a patient's analyte levels for an extended period of time for a meaningful determination of whether the prescribed medication may actually cause hyperkalemia.

In certain embodiments where decision support engine 114 determines, at block 834, that the user is prescribed medication which may cause hyperkalemia, at block 836, decision support engine 114 recommends that the user consult a healthcare professional for purposes of adjusting the medication currently prescribed for the user.

In certain other embodiments (e.g., not illustrated) where decision support engine 114 determines, at block 834, that the user is prescribed medication which may cause hyperkalemia, decision support engine 114 recommends an adjustment to the administration of such medication. Decision support engine 114's adjustment recommendation may be based, at least in part, on the medication itself, the medication parameters (e.g., type, dosage, timing, frequency etc.), the user's potassium level, and/or user's potassium rate of change.

Medication adjustment(s) recommended by a healthcare professional and/or decision support system 100 may include (1) skipping a dose, (2) taking a lower dose, (3) taking a dose with a meal, (4) taking a dose at a later time, and/or (5) taking a dose with another potassium-reducing mechanism (e.g., other medication). Further, the medication adjustment(s) recommended by the healthcare professional and/or decision support system 100 may include (1) recommended temporary adjustments and/or (2) recommended permanent adjustments (e.g., adjustments to the administration of a medication beyond the next dose).

In certain other embodiments where decision support engine 114 determines, at block 834, that the user is not prescribed medication which may cause hyperkalemia, at block 838, decision support engine 114 determines whether the user is on exogenous insulin (e.g., injects and/or infuses insulin via an insulin pump).

In certain embodiments where decision support engine 114 determines, at block 838, that the user is on exogenous insulin, decision support engine 114 may proceed to FIG. 9 to determine one or more recommendations to provide for the treatment of kidney disease and/or for glucose homeostasis to a user classified as being at risk of hyperkalemia (e.g., in FIG. 7). In particular, insulin stimulates potassium and/or glucose uptake by the cells, while reducing serum (e.g., extracellular) potassium and/or glucose levels. Thus, a user on exogenous insulin may be recommended to administer additional insulin, depending upon the user's glucose level. FIG. 9 illustrates an example method 900 that evaluates a user's glucose level prior to recommending a user administer exogenous insulin and/or consume glucose to stimulate the endogenous release of insulin.

In certain embodiments where decision support engine 114 determines, at block 838, that the user is not on exogenous insulin, at block 840, decision support engine 114 recommends the user consume glucose to stimulate the endogenous release of insulin. For example, even where a user is not on exogenous insulin, the user may increase their insulin level by stimulating insulin release. Insulin is released when glucose levels rise. Therefore, decision support engine 114 may recommend, to a user, to consume additional glucose to increase serum glucose levels. Increasing serum glucose levels may trigger insulin release in the user. The released insulin may help to reduce glucose levels and/or potassium levels of the user, thereby helping to reduce the user's risk of hyperkalemia.

In certain embodiments, where decision support engine 114 determines, at block 838, that the user is not on exogenous insulin, at block 840, decision support engine 114 recommends the user dose insulin to reduce potassium and/or glucose levels of the user. For example, for a user who is experiencing a moderate risk of potassium (e.g., elevated potassium), administering insulin may help to reduce a user's potassium level to be within a healthy potassium range (e.g., 3.0-5.0 mmol/L). Further, decision support engine 114 may recommend for insulin to be administered to reduce a glucose level of the user to be within a healthy glucose range (e.g., 70-200 mg/dL). Accordingly, insulin may help to reduce both glucose and potassium levels of the user, while also reducing the user's risk of hyperkalemia and/or hyperglycemia.

In certain embodiments, at block 840, decision support engine 114 recommends the user dose a fast-acting or rapid-acting insulin. For example, the recommendation may be to dose ten units' fast insulin to reduce a glucose level of the user to be within the healthy glucose range.

At block 842, decision support engine 114 determines whether the user is on dialysis.

Dialysis is a treatment for kidney failure that rids the body of unwanted toxins, waste products, and excess fluids by filtering a user's blood. Users with end stage renal disease (ESRD) (e.g., CKD stage 5) may be prescribed dialysis treatment to supplement and/or replace filtering generally performed by the kidney, given dialysis helps to keep the potassium, phosphorus, and sodium levels in a patient's body balanced. Dialysis treatment may be hemodialysis or peritoneal dialysis (e.g., In hemodialysis, blood is pumped out of a user's body to an artificial kidney machine, and returned to the body by tubes that connect the user to the machine. In peritoneal dialysis, the inside lining of the user's stomach acts as a natural filter.). Dialysis treatments may be given to a user on a set schedule (e.g., 3 to 4 times a week for a few hours). Dialysis treatment may be at a dialysis clinical (e.g., hemodialysis) or through at home treatment (e.g., peritoneal dialysis). Dialysis treatment may help to reduce a user's potassium level to a healthy level. Therefore, if dialysis treatment is available, dialysis treatment may be used to help effectively reduce potassium levels and/or prevent hyperkalemia in a user.

In certain embodiments where decision support engine 114 determines, at block 842, that the user is on dialysis, at block 844, decision support engine 114 further determines whether the user had a dialysis treatment in the current treatment period. Decision support engine 114 may make this determination because, in some cases, dialysis treatments may not be given together too close in time. For example, a user may be instructed to carry out a dialysis treatment for three days each week, and more specifically, on Monday, Thursday, and Saturday. Thus, a first treatment period may include Monday-Wednesday, a second treatment period may include Thursday-Friday, and a third treatment period may include Saturday-Sunday. Accordingly, where a user performed a dialysis treatment on Monday and today is only Tuesday, the user may be said to be “within the current treatment period” and another dialysis treatment may not be given because a dialysis treatment was already performed for the current treatment period (e.g., the first treatment period). However, in a situation where today is Thursday, another dialysis treatment may be given because a dialysis treatment has not yet been performed for the current treatment period (e.g., the second treatment period).

In certain embodiments where decision support engine 114 determines, at block 844, that the user has not yet had a dialysis treatment in the current treatment period, decision support engine 114 recommends, at block 846, that the user seek dialysis treatment and/or determine if dialysis treatment is available to the user (e.g., based on the user's access to dialysis treatment). For example, a user may not always have access to dialysis treatment, given some users may be required to go to dialysis clinic, as opposed to perform an at-home dialysis treatment. Dialysis treatment access may be may be limited because at the time treatment is required clinics might be closed, a consultation with a HCP may be required, etc.

Alternatively, in certain embodiments where decision support engine 114 determines, at block 844, that the user has had a dialysis treatment in the current treatment period, decision support engine 114 may not recommend the user seek another dialysis treatment. Instead, at block 848, decision support engine 114 determines whether the user is on (e.g., is using, is prescribed, etc.) a potassium binder. Potassium binders are medications which bind to potassium and allow for the removal of potassium from a user's body. Thus, potassium binders may help to reduce potassium levels.

Further, in certain embodiments, decision support engine 114 determines, at block 848, whether the user is on (e.g., is using, is prescribed, etc.) a potassium binder after determining, at block 842 that the user is not on dialysis.

In certain embodiments where decision support engine 114 determines, at block 848, that the user is on (e.g., is using, is prescribed, etc.) a potassium binder, at block 850, decision support engine 114 determines whether the user has ingested the potassium binder in a current treatment period. For example, potassium binders may take approximately four to six hours to reduce potassium levels. Therefore, a user may not be recommended to take a second dose of a potassium binder within a treatment period where the potassium binder has already (previously in the treatment period) been ingested to allow the initial dose of the potassium binder to act. As such, decision support engine 114 recommends to a user to administer a potassium binder (if available), unless a potassium binder has been administered in the current treatment period. Additionally, in certain embodiments, decision support engine 114 may note when a potassium binder has been recommended and/or administered (e.g., by the user) and not recommend another dose until the current treatment period has ended (e.g., four to six hours from the previous potassium binder dose).

As such, in certain embodiments where decision support engine 114 determines, at block 850, that the user has not yet dosed a potassium binder in the current treatment period, decision support engine 114 recommends, at block 852, that the user dose the potassium binder. Alternatively, in certain embodiments where decision support engine 114 determines, at block 850, that the user has dosed a potassium binder in the current treatment period, decision support engine 114 may not recommend the user dose a potassium binder. Instead, at block 854, decision support engine 114 determines whether the user is on (e.g., is using, is prescribed, etc.) a non-potassium sparing diuretic. A non-potassium sparing diuretic is a diuretic used to treat hypertension and/or edema, which may cause potassium loss in the urine. Because the diuretic dose is capable of causing potassium loss, the diuretic may be used to reduce serum potassium levels of a patient.

Further, in certain embodiments, decision support engine 114 determines, at block 854, whether the user is on (e.g., is using, is prescribed, etc.) a non-potassium sparing diuretic after determining, at block 848, that the user is not on (e.g., is not using, is not prescribed, etc.) a potassium binder.

In certain embodiments where decision support engine 114 determines, at block 854, that the user is on (e.g., is using, is prescribed, etc.) a non-sparing diuretic, at block 856, decision support engine 114 determines whether the user has ingested the non-potassium sparing diuretic in a current treatment period. For example, non-potassium sparing diuretics may take up to twenty-four hours for full effectiveness, and a repeat dose may not be recommended within this time period (e.g., within a current treatment period). Therefore, a user may not be recommended to take a second dose of a non-potassium sparing diuretic within a treatment period where the non-potassium sparing diuretic has already (previously in the current treatment period) been ingested to allow the initial dose of the non-potassium sparing diuretic to act. As such, decision support engine 114 recommends to a user to administer a non-potassium sparing diuretic (if available), unless the non-potassium sparing diuretic has been administered in the current treatment period. Additionally, in certain embodiments, decision support engine 114 may note when a non-potassium sparing diuretic has been recommended and/or administered (e.g., by the user) and not recommend another dose until the current treatment period has ended (e.g., twenty-four from the previous ingestion of the non-potassium sparing diuretic).

As such, in certain embodiments where decision support engine 114 determines, at block 856, that the user has not yet ingested a non-potassium sparing diuretic in the current treatment period, decision support engine 114 recommends, at block 858, that the user ingest the non-potassium sparing diuretic. Alternatively, in certain embodiments where decision support engine 114 determines, at block 856, that the user has ingested a non-potassium sparing diuretic in the current treatment period, decision support engine 114 may not recommend the user ingest the non-potassium sparing diuretic. Instead, at block 860, decision support engine 114 determines whether the user is on (e.g., is using, is prescribed, etc.) both a potassium binder and the non-potassium sparing diuretic.

In certain embodiments where the decision support engine 114 determines, at block 860, that the user is not on (e.g., is using, is prescribed, etc.) both a potassium binder and the non-potassium sparing diuretic, the decision support engine 114 (1) determines that the user does not have an adequate at-home treatment available to reduce the user's potassium levels back to a healthy range and (2) recommends, at block 862, the user seek immediate medical attention (e.g., go the emergency room) to reduce potassium levels and prevent hyperkalemia, or for patients with moderately elevated potassium levels and no symptoms, to consult a health care professional.

Further, in certain embodiments, decision support engine 114 (1) determines that the user does not have an adequate at-home treatment available to reduce the user's potassium levels back to a healthy range and (2) recommends, at block 862, the user seek immediate medical attention (e.g., go the emergency room) to reduce potassium levels and prevent hyperkalemia after determining, at block 854, that the user is not on (e.g., is not using, is not prescribed, etc.) a non-potassium sparing diuretic.

In certain embodiments where the decision support engine 114 determines, at block 860, that the user is on (e.g., is using, is prescribed, etc.) both a potassium binder and the non-potassium sparing diuretic, the decision support engine 114 determines, at block 864, whether the user is still at mild or moderate risk of hyperkalemia after a predetermined period of time has ended (e.g., four to twenty-four hours from the previous ingestion of the non-potassium sparing diuretic).

As such, in certain embodiments where decision support engine 114 determines, at block 864, that the user is still at mild or moderate risk of hyperkalemia after the predetermined period of time, at block 866, decision support engine 114 recommends, at block 852, that the user dose the potassium binder. Alternatively, in certain embodiments, where decision support engine 114 (1) determines, at block 864, that the user is no longer at mild or moderate risk of hyperkalemia after the predetermined period of time, at block 868, decision support engine 114 may recommend that the user consider other methods of reducing their potassium level, without consuming glucose and/or potassium and while increasing water intake.

Referring back to block 810 (e.g., illustrated in FIG. 8A), in certain embodiments where decision support engine 114 determines, at block 810, that the user's potassium level is not above the modestly elevated potassium threshold (e.g., user's potassium level ≤5.5 mmol/L) and/or the user's potassium level will not be above the modestly elevated potassium threshold within the defined period of time, decision support engine 114 determines, at block 816, that the user is at mild risk of hyperkalemia.

In particular, a user's elevated potassium level and/or a potassium rate of change, indicating that the user's potassium level will reach the elevated threshold but not reach the modestly elevated threshold (e.g., ≤5.5 mmol/L) in the defined time period, e.g., in the defined intermediate time period (e.g., in the next 2 hours), may be associated with a mild risk of hyperkalemia. A user with a mild risk of hyperkalemia has an elevated potassium level which may need to be lowered to a healthy level, but may not need to be rapidly and/or promptly lowered. Users with CKD may have chronically elevated potassium and may need to lower their potassium level long term (e.g., chronically). These users may use at home treatments to lower their potassium level and maintain their potassium level in a healthy range. Users without a known CKD, and/or other factors which may cause chronically elevated potassium levels (e.g., medications that inhibit the RAAS), may be recommended to speak with a healthcare professional regarding their elevated potassium.

In certain embodiments where decision support engine 114 determines, at block 816, that the user is at mild risk of hyperkalemia, decision support engine 114 may proceed to FIG. 10 to determine one or more diet recommendations for the treatment of kidney disease and/or for glucose homeostasis to a user classified as being at risk of hyperkalemia (e.g., in FIG. 7).

In certain embodiments, as shown in FIG. 8A, at block 818, decision support engine 114 recommends the user stop any consumption (e.g., current or future consumption) of potassium and increase water intake. Further, at block 820, decision support engine 114 determines whether the user is on (e.g., is using, is prescribed, etc.) a potassium binder. As mentioned, potassium binders are medications which bind to potassium and allow for the removal of potassium from a user's body; thus, potassium binders may help to reduce a potassium level of the user.

In certain embodiments where decision support engine 114 determines, at block 820, that the user is on (e.g., is using, is prescribed, etc.) a potassium binder, at block 822, decision support engine 114 determines whether the user has ingested the potassium binder in a current treatment period.

In certain embodiments where decision support engine 114 determines, at block 822, that the user has not yet dosed a potassium binder in the current treatment period, decision support engine 114 recommends, at block 824, that the user dose the potassium binder. Alternatively, in certain embodiments where decision support engine 114 determines, at block 822, that the user has dosed a potassium binder in the current treatment period, decision support engine 114 may not recommend the user dose a potassium binder. Instead, at block 826, decision support engine 114 determines whether the user is on exogenous insulin.

In certain embodiments where decision support engine 114 determines, at block 826, that the user is on exogenous insulin, decision support engine 114 may proceed to FIG. 9 to determine one or more recommendations to provide for the treatment of kidney disease and/or for glucose homeostasis to a user classified as being at risk of hyperkalemia (e.g., in FIG. 7). As mentioned, insulin stimulates potassium and/or glucose uptake by the cells, while reducing serum (e.g., extracellular) potassium and/or glucose levels. Thus, a user on exogenous insulin may be recommended to administer additional insulin, depending upon the user's glucose level. FIG. 9 illustrates an example method 900 that evaluates a user's glucose level prior to recommending a user administer exogenous insulin and/or consume glucose to stimulate the endogenous release of insulin.

In certain embodiments where decision support engine 114 determines, at block 826, that the user is not on exogenous insulin, at block 828, decision support engine 114 determines whether the user is prescribed medication that may cause hyperkalemia.

Further, in certain embodiments, decision support engine 114 determines, at block 828, whether the user is prescribed medication which may cause hyperkalemia after determining, at block 822, that the user is not on (e.g., is not using, is not prescribed, etc.) a potassium binder. As mentioned, some medications may increase potassium levels and/or otherwise induce hyperkalemia (e.g. drugs that inhibit the RAAS, nonsteroidal anti-inflammatory drugs, hypovolemia, etc.).

In certain embodiments where decision support engine 114 determines, at block 828, that the user is prescribed medication that may cause hyperkalemia, at block 832, decision support engine 114 recommends that the user consult a healthcare professional for purposes of adjusting the medication currently prescribed for the user. In certain other embodiments (e.g., not illustrated) where decision support engine 114 determines, at block 828, that the user is prescribed medication which may cause hyperkalemia, decision support engine 114 recommends an adjustment to the administration of such medication. Decision support engine 114's adjustment recommendation may be based, at least in part, on the medication itself, the medication parameters (e.g., type, dosage, timing, frequency etc.), the user's potassium level, and/or user's potassium rate of change.

Alternatively, in certain other embodiments where decision support engine 114 determines, at block 828, that the user is not prescribed medication that may cause hyperkalemia, at block 830, decision support engine 114 recommends that the user consult a healthcare professional. For example, a user with elevated potassium levels, which are not caused by a medication, likely has CKD. Accordingly, the user (e.g., having elevated potassium levels and without an at-home treatment to reduce potassium levels, (e.g., insulin, potassium binder, etc.)) may need to consult with a healthcare professional to be prescribed an at-home treatment to reduce risk of hyperkalemia.

For a user with an elevated potassium level, which does not have CKD, and is not prescribed (and/or is not using) a medication causing a potassium level of the user to elevate, one or more other causes may be causing the elevated potassium level of the user. For example, there may be another cause of the user's elevate potassium level, including an unknown kidney disease, an acute kidney injury (AKI), and/or other cause. As such, a consultation with a healthcare professional may be recommended.

As shown in FIGS. 8A and 8B, in certain embodiments where decision support engine 114 determines that the user is on exogenous insulin (e.g., at block 826 in FIG. 8A and block 838 in FIG. 8B), decision support engine 114 may proceed to FIG. 9 to determine one or more recommendations to provide for the treatment of kidney disease and/or for glucose homeostasis to a user classified as being at risk of hyperkalemia (e.g., in FIG. 7). More specifically, decision support engine 114 may proceed to FIG. 9 to determine one or more recommendations for users determined to be at moderate or mild risk of hyperkalemia, per FIGS. 8A and 8B (e.g., blocks 812 and 816).

FIG. 9 illustrates an example method 900 used for providing recommendations of treatment for kidney disease and/or glucose homeostasis for a user determined to be at risk of hyperkalemia, according to certain embodiments of the present disclosure. Method 900 is described below with reference to FIGS. 1 and 2 and their components. For example, components of decision support system 100 illustrated in FIG. 1 may use method 900 to determine treatment (or preventative) recommendations to recommend to a user for purposes of reducing the user's elevated potassium level and/or preventing the user's elevated potassium level from increasing further.

Method 900 begins at block 902 by decision support system 100, and more specifically decision support engine 114 of decision support system 100, determining whether the user's glucose level is above an elevated glucose threshold (e.g., >200 milligrams per deciliter (mg/dL)) and/or whether the user's glucose level will be above the elevated glucose threshold within a short period of time (e.g., ≤1 hour). The short period of time may be predefined.

In certain embodiments where decision support engine 114 determines, at block 902, that the user's glucose level is above the elevated glucose threshold (e.g., user's glucose level >200 mg/dL or >150 mg/dL) and/or the user's glucose level will be above the elevated glucose threshold within the short period of time, decision support engine 114 recommends, at block 904, that the user dose insulin to reduce potassium and/or glucose levels of the user. For example, decision support engine 114 may recommend for insulin to be administered to reduce a glucose level of the user to be within a healthy glucose range (e.g., 70-200 mg/dL). Further, for a user who is experiencing a mild or moderate risk of potassium (e.g., elevated potassium), administering insulin may help to reduce a user's potassium level to be within a healthy potassium range (e.g., 3.0-5.0 mmol/L). Accordingly, insulin may help to reduce both glucose and potassium levels of the user, while also reducing the user's risk of hyperkalemia and/or hyperglycemia.

In certain embodiments, at block 904, decision support engine 114 recommends the user dose a fast-acting or rapid-acting insulin. For example, the recommendation may be to dose ten units' fast insulin to reduce a glucose level of the user to be within the healthy glucose range.

Alternatively, in certain embodiments where decision support engine 114 determines, at block 902, that the user's glucose level is not above the elevated glucose threshold (e.g., user's glucose level ≤200 mg/dL) and/or the user's glucose level will not be above the elevated glucose threshold within the short period of time, decision support engine 114 further determines, at block 906, whether the user is at moderate or mild risk of hyperkalemia. As described with respect to FIGS. 8A and 8B, decision support engine 114 made this determination at block 810. Decision support engine 114 may use this prior determination to determine which path of method 900 to continue following for purposes of providing decision support to the user.

Where decision support engine 114 determines, at block 906, that the user is at moderate risk of hyperkalemia, at block 910, decision support engine 114 determines whether the user's glucose level is within a healthy glucose range. In certain embodiments where decision support engine 114 determines, at block 910, that the user's glucose level is within a healthy glucose range, at block 912, decision support engine 114 determines whether the user's next meal and/or dose of insulin is within a short period of time (e.g., ≤1 hour).

In particular, a user having a glucose level within in a healthy glucose range may nevertheless dose insulin where the user also is at a moderate risk of hyperkalemia. Because potassium needs to be promptly reduced (as described above for a user at moderate risk of hyperkalemia) and insulin may reduce potassium levels, decision support engine 114 may recommend that the user dose insulin. However, decision support engine 114, in making this recommendation, may also need to consider what the user's glucose level is at. For example, a glucose level within a healthy glucose range may also be reduced with a dose of insulin; therefore, decision support engine 114 may consider when the user's next scheduled meal and/or dose of insulin will be prior to providing a recommendation. Decision support engine 114 may determine a user's next meal and/or dose of insulin using various methods including prompting the user, monitoring the user's calendar, monitor's the user's adherence to a meal schedule, examining historical data of the user, using one or more metrics 130 stored in user profile 118 as shown in FIG. 3, etc.

In certain embodiments where decision support engine 114 determines, at block 912, that the user's next meal and/or dose of insulin is within a short period of time (e.g., ≤1 hour), at block 914, decision support engine 114 recommends the user consume the user's next meal and/or dose insulin earlier (or as soon as possible) to promptly reduce potassium. This recommendation may allow the dose of insulin to reduce a potassium level of the user and help to reduce the user's moderate risk of hyperkalemia, while also avoiding hypoglycemia.

Alternatively, in certain embodiments where decision support engine 114 determines, at block 912, that the user's next meal and/or dose of insulin is not within the short period of time, at block 916, decision support engine 114 recommends the user act to (1) consume additional glucose (e.g., an additional meal and/or snack) and (2) dose additional insulin. This recommendation may allow the dose of insulin to reduce a potassium level of the user and help to reduce the user's moderate risk of hyperkalemia, while also avoiding hypoglycemia.

In certain embodiments, the recommendation given by decision support engine 114 at block 916 includes (1) an amount of glucose and/or carbohydrate the user is to consume, (2) a specific food recommendation, (3) a recommendation to avoid consuming potassium, (4) an insulin dosage, and/or (5) an insulin type. For example, a recommendation given by decision support engine 114 at block 916 may recommend that the user consume 15-25 grams of glucose (or equivalent carbohydrate) and dose 10 units of fast-acting insulin.

Subsequent to the recommendations given at blocks 914 and 916, decision support engine 114 may return to FIG. 8A to continue to treat an elevated potassium level of the user, should the user's potassium continue to be elevated and require further attention.

Returning back to block 910, in certain embodiments, decision support engine 114 determines that the user's glucose level is not within a healthy glucose range. Accordingly, at block 918, decision support engine 114 determines whether the user's glucose level is below a depressed glucose threshold (e.g., <70 mg/dL) and/or whether the user's glucose level will be below the depressed glucose threshold within a short period of time (e.g., ≤1 hour). Decision support engine 114 is expected to determine, at block 918, that the user's glucose level is below the depressed glucose threshold and/or will be within the short period of time given decision support engine 114 (1) previously determined, at block 902, that the user's glucose level was not above the elevated glucose threshold (e.g., user's glucose level ≤200 mg/dL) and/or would not be within the short period of time and (2) previously determined, at block 910, that the user's glucose level was not within a healthy glucose range (e.g., not within 70-200 mg/dL). As such, the user's glucose level is expected to be below the depressed glucose threshold (e.g., <70 mg/dL).

Thus, where decision support engine 114 determines, at block 910, that the user's glucose level is below the depressed glucose threshold and/or will be below the depressed glucose threshold within the short period of time, at block 920, decision support engine 114 recommends the user (1) consume glucose and (2) avoid consuming potassium. In particular, depressed and/or falling glucose levels (e.g., <70 mg/dL within the next hour) may indicate that a user needs to consume glucose to prevent hypoglycemia. However, if the user also has an elevated potassium level, then the user may also need avoid consuming additional potassium. Therefore, decision support engine 114 recommends to the user to consume glucose/carbohydrate, but not potassium. The recommendation may include (1) a recommended amount of glucose/carbohydrate to consume, (2) specific food recommendations, and/or (3) recommendation of food to avoid consuming, such as potassium.

Subsequent to the recommendations given at block 920, decision support engine 114 may return to FIG. 8A to continue to treat an elevated potassium level of the user, should the user's potassium continue to be elevated and require further attention.

In certain embodiments where decision support engine 114 determines, at block 910, that the user's glucose level is not below the depressed glucose threshold and/or at block 918, that the user's glucose level will not be below the depressed glucose threshold within the short period of time, at block 922, decision support engine 114 may recommend that the user consider other methods of reducing their potassium levels, without consuming glucose and/or potassium. In certain embodiments, decision support engine 114 may further recheck glucose levels of the user. Specially, at block 902, decision support engine 114 would have determined that the user's glucose level is not elevated (e.g., not >200 mg/dL) and further would have determined, at block 910, that the user's glucose level is not within a healthy range (e.g., not between 70-200 mg/dL); thus, it is expected that decision support engine 114 determines, at block 918, that the user's glucose level is, in fact, depressed.

Returning back to block 906, in certain embodiments, decision support engine 114 determines that the user is at mild risk of hyperkalemia. As described with respect to FIGS. 8A and 8B, decision support engine 114 may have previously made this determination at block 810. Decision support engine 114 may use this prior determination at block 906. Thus, after classifying the user as being at a mild risk of hyperkalemia, a block 926, decision support engine 114 determines whether the user's glucose level is below a depressed glucose threshold (e.g., <70 mg/dL) and/or whether the user's glucose level will be below the depressed glucose threshold within a short period of time (e.g., ≤1 hour).

In certain embodiments where decision support engine 114 determines, at block 926, that the user's glucose level is below the depressed glucose threshold and/or will be below the depressed glucose threshold within the short period of time, at block 928, decision support engine 114 recommends the user (1) consume glucose and (2) avoid consuming potassium. The recommendation may include (1) a recommended amount of glucose/carbohydrate to consume, (2) specific food recommendations, and/or (3) recommendation of food to avoid consuming, such as potassium.

Alternatively, in certain embodiments where decision support engine 114 determines, at block 926, that the user's glucose level is not below the depressed glucose threshold and/or will not be below the depressed glucose threshold within the short period of time, decision support engine determines the user's glucose level is within a healthy range (e.g., within 70-200 mg/dL). Decision support engine 114 makes this determination based on (1) previously determining, at block 902, that the user's glucose level was not above the elevated potassium threshold (e.g., user's glucose level ≤200 mg/dL) and/or would not be within the short period of time and (2) determining, at block 926, that the user's glucose level is not below the depressed glucose threshold (e.g., user's glucose level >70 mg/dL) and/or will not be within the short period of time. Accordingly, at block 930, decision support engine 114 may recommend that the user consider other methods of reducing their potassium level, without consuming glucose and/or potassium. For example, in certain embodiments, the user may take a small dose of insulin.

In particular, where a user's glucose level is within a healthy range, then the user may not be recommended to consume either glucose or potassium. Therefore, decision support engine 114 may recommend that the user avoid consuming food at the current moment.

Subsequent to the recommendations given at blocks 928 and 930, decision support engine 114 may return to FIG. 8A, e.g., block 802, to continue to treat an elevated potassium level of the user, should the user's potassium continue to be elevated and require further attention.

In certain embodiments, decision support engine 114 may further proceed to FIG. 10 to determine one or more diet recommendations for users determined to be at moderate or mild risk of hyperkalemia, per FIGS. 8A and 8B. In particular, users with CKD and/or at risk of potassium imbalance (e.g., prescribed medications which affect the user's potassium level), may need to closely monitor their diet to improve potassium imbalance and/or reduce their risk of hyperkalemia. As described in more detail below, diet recommendations for users at milder or mild risk of hyperkalemia may include a low-potassium diet and/or a low-carbohydrate (e.g., glucose) diet. Dietary recommendations may be given based on a user's potassium level, glucose level, and/or dietary requirements.

A user at mild or moderate risk of hyperkalemia may be recommended to avoid the consumption of additional potassium. However, depending upon the user's glucose level, a user may need to consume glucose. Therefore, in certain embodiments, diet recommendations may be based on both potassium and glucose.

FIG. 10 illustrates an example method 1000 used for providing diet recommendations for a user determined to be at risk of hyperkalemia, according to certain embodiments of the present disclosure. Method 1000 is described below with reference to FIGS. 1 and 2 and their components. For example, components of decision support system 100 illustrated in FIG. 1 may use method 1000 to determine treatment (or preventative) recommendations to recommend to a user for purposes of reducing the user's elevated potassium level and/or preventing the user's elevated potassium level from increasing further. In certain embodiments, decision support engine 114 may proceed to FIG. 10 to determine one or more recommendations for users determined to be at moderate or mild risk of hyperkalemia per FIGS. 8A and 8B (e.g., blocks 812 and 816).

Method 1000 begins at block 1002 by decision support system 100, and more specifically decision support engine 114 of decision support system 100, determining whether the user's glucose level is above an elevated glucose threshold (e.g., >200 milligrams per deciliter (mg/dL)) and/or whether the user's glucose level will be above the elevated glucose threshold within a short period of time (e.g., ≤1 hour). The short period of time may be predefined.

In certain embodiments where decision support engine 114 determines, at block 1002, that the user's glucose level is above the elevated glucose threshold (e.g., user's glucose level >200 mg/dL) and/or the user's glucose level will be above the elevated glucose threshold within the short period of time, decision support engine 114 recommends, at block 1004, that the user (1) avoid consuming glucose and (2) avoid consuming potassium. Although high glucose levels may stimulate insulin release in the user, and insulin may then reduce both glucose and potassium levels of the user, at elevated glucose levels, additional glucose consumption may not be recommended. As such, decision support engine 114 may recommend that the user not consume glucose, nor potassium. Such recommendations given by decision support engine 114 may be general (e.g., a message of “do not eat” provided to the user), or specific (e.g., a message of “do not consume glucose or potassium” provided to the user).

In certain embodiments where decision support engine 114 determines, at block 1002, that the user's glucose level is above the elevated glucose threshold and/or the user's glucose level will be above the elevated glucose threshold within the short period of time, the recommendation given by decision support engine 114 at block 1004 includes (1) a specific food recommendation, or (2) a recommendation of specific foods to avoid consuming. For example, a recommendation given by decision support engine 114 at block 1004 may recommend to the user one or more types of meals, snacks, and/or beverages for consumption (e.g., foods that are low in glucose and potassium). In further examples, a recommendation given by decision support engine 114 at block 1004 may recommend to the user one or more types of meals, snacks, and/or beverages to avoid (e.g., foods that are high in glucose and potassium). In certain embodiments, at block 1004, decision support engine 114 may recommend that the user increases their water intake. In certain embodiments, at block 1004, a user may input a meal, snack, and/or beverage into, e.g., application 106, and decision support engine 114 may determine whether the meal, snack, and/or beverages may be consumed or should be avoided by the user. For example, the user may input food and drink information by providing a photograph through an application that is configured to recognize food types and quantities, and/or by scanning a bar code or menu.

In certain embodiments where decision support engine 114 determines, at block 1002, that the user's glucose level is not above the elevated glucose threshold (e.g., user's glucose level <200 mg/dL) and/or the user's glucose level will not be above the elevated glucose threshold within the short period of time, decision support engine 114 determines, at block 1006, whether the user's glucose level is within a healthy glucose range (e.g., between 70-200 mg/dL). In certain embodiments where the user's glucose level is within a healthy glucose range, decision support engine 114 also recommends, at block 1004, that the user (1) avoid consuming glucose and (2) avoid consuming potassium. Where the user's glucose level is within a healthy range and the user's potassium level is elevated, neither the consumption of potassium, nor glucose, may be recommended. Although increasing glucose levels may stimulate insulin release, where the user's glucose level is within a healthy range, additional glucose may not be recommended. In addition, when the user's potassium level is elevated, additional potassium may not be recommended.

In certain embodiments where the user's glucose level is within a healthy glucose range, the recommendation given by decision support engine 114 at block 1004 includes (1) a specific food recommendation, or (2) a recommendation of specific foods to avoid consuming. For example, a recommendation given by decision support engine 114 at block 1004 may recommend to the user one or more types of meals, snacks, and/or beverages for consumption (e.g., foods that are low in glucose and potassium). In further examples, a recommendation given by decision support engine 114 at block 1004 may recommend to the user one or more types of meals, snacks, and/or beverages to avoid (e.g., foods that are high in glucose and potassium).

In certain embodiments where the user's glucose level is not within a healthy range, decision support engine 114 determines, at block 1008, whether the user's glucose level is below a depressed glucose threshold (e.g., <70 mg/dL) and/or whether the user's glucose level will be below the depressed glucose threshold within a short period of time (e.g., ≤1 hour). Decision support engine 114 is expected to determine, at block 1008, that the user's glucose level is below the depressed glucose threshold and/or will be within the short period of time given decision support engine 114 (1) previously determined, at block 1002, that the user's glucose level was not above the elevated glucose threshold (e.g., user's glucose level ≤200 mg/dL) and/or would not be within the short period of time and (2) previously determined, at block 1006, that the user's glucose level was not within a healthy glucose range (e.g., not within 70-200 mg/dL). As such, the user's glucose level is expected to be below the depressed glucose threshold (e.g., <70 mg/dL).

Thus, where decision support engine 114 determines, at block 1008, that the user's glucose level is below the depressed glucose threshold and/or will be below the depressed glucose threshold within the short period of time, at block 1010, decision support engine 114 recommends the user (1) consume glucose, (2) avoid consuming potassium and (3) increase water intake. In particular, depressed and/or falling glucose levels (e.g., <70 mg/dL within the next hour) may indicate that a user needs to consume glucose to prevent hypoglycemia. However, if the user also has an elevated potassium level, then the user may also need avoid consuming additional potassium. Therefore, decision support engine 114 recommends to the user to consume glucose/carbohydrate, but not potassium, and increase water intake. The recommendation may include (1) a recommended amount of glucose/carbohydrate to consume, (2) specific food recommendations, (3) a recommended increase in water intake, and/or (4) recommendation of food to avoid consuming, such as potassium. In still further embodiments, at block 1010, decision support engine 114 may recommend the user administer a dose of glucagon, and may provide instructions on how to administer such dose of glucagon.

In certain embodiments, at block 1010, a user may input a meal, snack, and/or beverage into, e.g., application 106, and decision support engine 114 may determine whether the meal, snack, and/or beverages may be consumed or should be avoided by the user. For example, the user may input food and drink information by providing a photograph through an application that is configured to recognize food types and quantities, and/or by scanning a bar code or menu.

Subsequent to the recommendations given at block 1010, decision support engine 114 may return to FIG. 8A to continue to treat an elevated potassium level of the user, should the user's potassium continue to be elevated and require further attention.

In certain embodiments where decision support engine 114 determines, at block 1008, that the user's glucose level is not below the depressed glucose threshold and/or will not be below the depressed glucose threshold within the short period of time, at block 1012, decision support engine 114 may recommend that the user consider other methods of reducing their potassium levels, without consuming glucose and/or potassium, such as increasing water intake. In certain embodiments, decision support engine 114 may further recheck glucose levels of the user. Specially, at block 1002, decision support engine 114 would have determined that the user's glucose level is not elevated (e.g., not >200 mg/dL) and further would have determined, at block 1006, that the user's glucose level is not within a healthy range (e.g., not between 70-200 mg/dL); thus, it is expected that decision support engine 114 determines, at block 1008, that the user's glucose level is, in fact, depressed.

Returning to FIG. 7, in some cases, a user may be classified as being at risk of hypokalemia, instead of hyperkalemia. More specifically, decision support engine 114 may determine, at block 710 in FIG. 7, that a user is at risk of hypokalemia where the user's potassium level is below an elevated potassium threshold (e.g., user's potassium level <3.5 mmol/L) and/or the user's potassium levels will be below the depressed potassium threshold within the short period of time. In such cases, as described above, instead of proceeding to FIGS. 8A and 8B, decision support engine 114 may proceed to FIG. 11 to determine one or more recommendations to provide to a user classified as being at risk of hypokalemia (e.g., in FIG. 7). FIG. 11 illustrates an example method 1100 used for providing decision support around kidney disease for a user determined to be at risk of hypokalemia.

Method 1100 is described below with reference to FIGS. 1 and 2 and their components. For example, components of decision support system 100 illustrated in FIG. 1 may use method 1100 to determine treatment (or preventative) recommendations to provide to a user for purposes of increasing the user's depressed potassium level and/or preventing the user's depressed potassium level from being further reduced.

Method 1100 begins at block 1102 by decision support system 100, and more specifically decision support engine 114 of decision support system 100, determining whether the user's potassium level is below the intensely depressed potassium threshold (e.g., determined at block 706 in FIG. 7) (e.g., <2.5 mmol/L) and/or whether the user's potassium level will be below the intensely depressed potassium threshold within a period of time, e.g., a short period of time (e.g., ≤1 hour), an intermediate period of time (e.g., ≤2 hours), or an extended period of time (e.g., 2 hours or more). The period of time may be predefined.

In certain embodiments where decision support engine 114 determines, at block 1102, that the user's potassium level is below the intensely depressed potassium threshold (e.g., user's potassium level <2.5 mmol/L) and/or the user's potassium level will be below the intensely depressed potassium threshold within the period of time, decision support engine 114 determines, at block 1104 that the user is at severe risk of hypokalemia. Accordingly, as shown in FIG. 11, at block 1106, decision support engine 114 recommends the user consume potassium.

A user with depressed potassium levels and/or a potassium rate of change which may put a user at below an intensely depressed threshold (e.g., <2.5 mmol/L) within, e.g., a short time period (e.g., ≤1 hour), is associated with a severe risk of hypokalemia and may have signs and/or symptoms of hypokalemia which may, in some cases, result in a medical emergency. As such, potassium levels may need to be increased and/or prevented from further reduction. Consuming potassium is one such method of increasing the user's potassium.

Further, at block 1108, decision support engine 114 determines whether the user is on exogenous insulin.

In certain embodiments where decision support engine 114 determines, at block 1108, that the user is on exogenous insulin, decision support engine 114 may proceed to FIG. 12 to determine one or more recommendations to provide for the treatment of kidney disease and/or for glucose homeostasis to a user classified as being at risk of hypokalemia (e.g., in FIG. 7). As mentioned, insulin stimulates potassium and/or glucose uptake by the cells, while reducing serum (e.g., extracellular) potassium and/or glucose levels. Thus, a user on exogenous insulin may be recommended to administer insulin to control glucose level, even though the insulin may further reduce the user's potassium level. At the same time, the user may need to consume additional potassium, without glucose, to increase the user's potassium level without increasing the user's glucose level. FIG. 12 illustrates an example method 1200 that evaluates a user's glucose level prior to recommending a user administer exogenous insulin and/or consume potassium to increase the user's potassium level.

In certain embodiments where decision support engine 114 determines, at block 1108, that the user is not on exogenous insulin, at block 1110, decision support engine 114 recommends the user avoid consuming glucose. In particular, in cases where a user is not on exogenous insulin, the user may increase their insulin level by stimulating insulin release. Insulin is released when glucose levels rise. Therefore, decision support engine 114 may recommend to a user to avoid the consumption of glucose, when consuming potassium, to avoid stimulating endogenous insulin release which may lower the user's already depressed potassium level. In some cases, not illustrated, decision support engine 114 may further recommend the user go to the emergency room.

In certain other embodiments where decision support engine 114 determines, at block 1102, that the user's potassium level is not below the intensely depressed potassium threshold (e.g., user's potassium level >2.5 mmol/L) and/or the user's potassium level will not be below the intensely depressed potassium threshold within the period of time, decision support engine 114 determines, at block 1112, whether the user's potassium level will be below the intensely depressed potassium threshold within the time period, e.g., within an intermediate period of time (e.g., ≤2 hours). The period of time may be predefined.

In certain embodiments where decision support engine 114 determines, at block 1112, that the user's potassium level will be below the intensely depressed potassium threshold within the period of time, decision support engine 114 determines, at block 1114, that the user is at moderate risk of hypokalemia.

A moderate risk of hypokalemia may not require emergency medical attention if the user as long as the user is able to safely increase potassium levels to healthy range. However, if a user does not have adequate available at-home treatments, decision support engine 114 may recommend a user to seek medical attention, as described below.

For a user determined to be at moderate risk of hypokalemia, at block 1116, decision support engine 114, recommends that the user consume potassium. Further, at block 1118, decision support engine 114 determines whether the user is on exogenous insulin. In certain embodiments where decision support engine 114 determines, at block 1118, that the user is on exogenous insulin, decision support engine 114 may proceed to FIG. 12 to determine one or more recommendations to provide for the treatment of kidney disease and/or for glucose homeostasis to a user classified as being at risk of hypokalemia (e.g., in FIG. 7).

In certain other embodiments where decision support engine 114 determines, at block 1118, that the user is not on exogenous insulin, decision support engine 114 determines, at block 1120, whether the user is prescribed medication that may reduce the user's potassium level. Some medications may reduce a user's potassium level, including, for example, a non-potassium sparing diuretic. As described herein, a non-potassium sparing diuretic is a diuretic used to treat hypertension and/or edema which may cause potassium loss in the urine. Because the diuretic dose is capable of causing potassium loss, the diuretic may be used to reduce serum potassium levels of a patient. In such embodiments, the user's potassium levels may be monitored for an extended period of time, e.g., 2 hours or more, or 7 days or more, to account for users who have been prescribed or put on a new medication, such as a non-potassium-sparing diuretic. In some examples, newly prescribed medications may necessitate the monitoring of a patient's analyte levels for an extended period of time for a meaningful determination of whether the prescribed medication may actually cause reduced potassium levels.

In certain embodiments where decision support engine 114 determines, at block 1120, that the user is prescribed medication that may reduce the user's potassium level, at block 1122, decision support engine 114 recommends the user stop using the prescribed medication, skip one or more doses of the medication, adjust a dosage of the medication, and/or adjust the medication itself to another type of medication to avoid further reducing the user's potassium level. In certain embodiments, a user with a chronic and/or repeated low potassium level may be recommended to consult with a healthcare professional regarding changing to a potassium-sparing diuretic to avoid having a depressed potassium level and, accordingly, reduce the user's risk of hypokalemia.

In certain other embodiments where decision support engine 114 determines, at block 1120, that the user is not prescribed medication that may reduce the user's potassium level, at block 1124, decision support engine 114 determines whether the user is on dialysis. As described herein, dialysis is a treatment for kidney failure that rids the body of unwanted toxins, waste products, and excess fluids by filtering a user's blood.

In certain embodiments where decision support engine 114 determines, at block 1124, that the user is not on dialysis, at block 1126, decision support engine 114 recommends that the user seek medical attention (e.g., consult with a healthcare professional).

In certain other embodiments where decision support engine 114 determines, at block 1124, that the user is on dialysis, at block 1128, decision support engine 114 further determines whether the user had a dialysis treatment in the current treatment period. As described herein, decision support engine 114 may make this determination because, in some cases, dialysis treatments may not be given together too close in time.

In certain embodiments where decision support engine 114 determines, at block 1128, that the user has not yet had a dialysis treatment in the current treatment period, decision support engine 114 recommends, at block 1130, that the user seek dialysis treatment (e.g., clinical or at-home treatment) and/or determine if dialysis treatment is available to the user (e.g., based on the user's access to dialysis treatment).

Alternatively, in certain embodiments where decision support engine 114 determines, at block 1128, that the user has had a dialysis treatment in the current treatment period, decision support engine 114 may not recommend the user seek another dialysis treatment. Instead, at block 1126, decision support engine 114 recommends that the user seek medical attention (e.g., consult with a healthcare professional).

Returning to block 1112, in certain other embodiments, decision support engine 114 determines that the user's potassium level will not be below the intensely depressed potassium threshold within the period of time. Accordingly, decision support engine 114 determines, at block 1132, that the user is at mild risk of hypokalemia.

A mild risk of hypokalemia is associated with lower than healthy potassium levels, including chronically low potassium levels. Such low potassium levels may be attributed to, in some cases, medications which lower potassium (e.g., insulin, non-potassium sparing diuretics). A potassium level of a user with a mild risk of hypokalemia may not need to be immediately raised; however, decision support engine 114 may recommend actions to increase and/or prevent further decrease of potassium levels, as described below. Additionally, a chronically (e.g., long-term and/or repeated) depressed potassium level of a user may indicate a need to change medications and/or increase potassium in diet (e.g., consume potassium (e.g., a potassium drink)). In other words, a user experiencing a depressed potassium level (e.g., below the depressed potassium threshold) may be recommended to increase their potassium level to a healthy range to reduce risk of hypokalemia and/or a medical emergency occurring.

For a user determined to be at mild risk of hypokalemia, at block 1134, decision support engine 114, recommends that the user consume potassium. Further, at block 1136, decision support engine 114 determines whether the user is on exogenous insulin. In certain embodiments where decision support engine 114 determines, at block 1136, that the user is on exogenous insulin, decision support engine 114 may proceed to FIG. 12 to determine one or more recommendations to provide for the treatment of kidney disease and/or for glucose homeostasis to a user classified as being at risk of hypokalemia (e.g., in FIG. 7).

In certain other embodiments where decision support engine 114 determines, at block 1136, that the user is not on exogenous insulin, decision support engine 114 determines, at block 1138, whether the user is prescribed medication that may reduce the user's potassium level.

In certain embodiments where decision support engine 114 determines, at block 1138, that the user is prescribed medication that may reduce the user's potassium level, at block 1140, decision support engine 114 recommends the user stop using the prescribed medication, skip one or more doses of the medication, adjust a dosage of the medication, and/or adjust the medication itself to another type of medication to avoid further reducing the user's potassium level. In certain embodiments, a user with a chronic and/or repeated low potassium level may be recommended to consult with a healthcare professional regarding changing to a potassium-sparing diuretic to avoid having a depressed potassium level and, accordingly, reduce the user's risk of hypokalemia.

In certain other embodiments where decision support engine 114 determines, at block 1138, that the user is not prescribed medication that may reduce the user's potassium level, decision support engine 114 determines that the user does not have treatment to increase the user's potassium level. Thus, block 1142, decision support engine 114 may recommend the user to consult with a healthcare professional to determine the cause and/or potential mechanisms to increase the user's potassium level.

As shown in FIG. 11, in certain embodiments where decision support engine 114 determines that the user is on exogenous insulin (e.g., at block 1108, block 1118, and block 1136 in FIG. 11), decision support engine 114 may proceed to FIG. 12 to determine one or more recommendations to provide for the treatment of kidney disease and/or for glucose homeostasis to a user classified as being at risk of hypokalemia (e.g., in FIG. 7). More specifically, decision support engine 114 may proceed to FIG. 12 to determine one or more recommendations for users determined to be at severe, moderate, or mild risk of hypokalemia, per FIG. 11.

FIG. 12 illustrates an example method 1200 used for providing recommendations of treatment for kidney disease and/or glucose homeostasis for a user determined to be at risk of hypokalemia, according to certain embodiments of the present disclosure. Method 1200 is described below with reference to FIGS. 1 and 2 and their components. For example, components of decision support system 100 illustrated in FIG. 1 may use method 1200 to determine treatment (or preventative) recommendations to recommend to a user for purposes of increasing the user's depressed potassium level and/or preventing the user's depressed potassium level from decreasing further.

Method 1200 begins at block 1202 by decision support system 100, and more specifically decision support engine 114 of decision support system 100, determining whether the user's glucose level is above an elevated glucose threshold (e.g., >200 milligrams per deciliter (mg/dL)) and/or whether the user's glucose level will be above the elevated glucose threshold within a short period of time (e.g., ≤1 hour). The short period of time may be predefined.

In certain embodiments where decision support engine 114 determines, at block 1202, that the user's glucose level is above the elevated glucose threshold (e.g., user's glucose level >200 mg/dL) and/or the user's glucose level will be above the elevated glucose threshold within the short period of time, decision support engine 114 recommends, at block 1204, that the user (1) dose insulin to reduce glucose and (2) consume potassium to avoid increasing the user's risk of hypokalemia. Decision support engine 114 may further recommend that the user (3) avoid the consumption of glucose.

Because insulin will reduce both potassium and glucose serum levels of the user, where a user has elevated an glucose level and a depressed potassium level, exogenous insulin may be recommended for lowering glucose, but may not be recommended to address depressed potassium levels. Therefore, for a user with elevated glucose levels (e.g., >200 mg/dL) or soon to be elevated glucose levels within a defined short time period (e.g., <1 hour), insulin administration may be recommended to reduce a user's glucose level to a healthy range. However, insulin administration may further reduce the already depressed potassium level of the user. Thus, decision support engine 114 may also recommend to a user to consume potassium only.

In certain embodiments, at block 1204, decision support engine 114 recommends the user dose a fast-acting or rapid-acting insulin. For example, the recommendation may be to dose ten units' fast insulin to reduce a glucose level of the user to be within the healthy glucose range.

Alternatively, in certain embodiments where decision support engine 114 determines, at block 1202, that the user's glucose level is not above the elevated glucose threshold (e.g., user's glucose level ≤200 mg/dL) and/or the user's glucose level will not be above the elevated glucose threshold within the short period of time, decision support engine 114 further determines, at block 1206, whether the user's glucose level is within a healthy glucose range. In certain embodiments where decision support engine 114 determines, at block 1206, that the user's glucose level is within a healthy glucose range, at block 1208, decision support engine 114 determines whether the user's next meal and/or dose of insulin is within a short period of time (e.g., ≤1 hour).

In particular, a user having a glucose level in a healthy glucose range may be consuming a meal and/or dosing insulin soon (e.g., within the short period of time). Thus, even if insulin is not currently recommended based on the user's glucose levels, insulin may soon be required because of the user's impending behavior (e.g., consuming a meal).

In certain embodiments where decision support engine 114 determines, at block 1208, that the user's next meal and/or dose of insulin is within a short period of time (e.g., ≤1 hour), at block 1210, decision support engine 114 recommends the user consume potassium before the user's next meal and/or administer a dose of insulin to prevent further reduction of the user's potassium level. For example, because insulin will further reduce the user's already depressed potassium level, additional potassium may be recommended to raise the user's potassium level. As such, decision support engine 114 may recommend that the user consume foods containing both glucose and potassium.

Alternatively, in certain embodiments where decision support engine 114 determines, at block 1208, that the user's next meal and/or dose of insulin is not within the short period of time, at block 1212, decision support engine 114 recommends the user act to (1) consume potassium and (2) avoid consuming glucose. In certain embodiments, the recommendation given by decision support engine 114 at block 1208 includes (1) an amount of potassium the user is to consume, (2) a specific food recommendation, and/or (3) a recommendation to avoid consuming glucose.

Subsequent to the recommendations given at blocks 1210 and 1212, decision support engine 114 may return to FIG. 11 to continue to treat the depressed potassium level of the user, should the user's potassium continue to be depressed and require further attention.

Returning back to block 1206, in certain embodiments, decision support engine 114 determines that the user's glucose level is not within a healthy glucose range. Accordingly, at block 1214, decision support engine 114 determines whether the user's glucose level is below a depressed glucose threshold (e.g., <70 mg/dL) and/or whether the user's glucose level will be below the depressed glucose threshold within a short period of time (e.g., ≤1 hour). Decision support engine 114 is expected to determine, at block 1206, that the user's glucose level is below the depressed glucose threshold and/or will be within the short period of time given decision support engine 114 (1) previously determined, at block 1202, that the user's glucose level was not above the elevated glucose threshold (e.g., user's glucose level ≤200 mg/dL) and/or would not be within the short period of time and (2) previously determined, at block 1206, that the user's glucose level was not within a healthy glucose range (e.g., not within 70-200 mg/dL). As such, the user's glucose level is expected to be below the depressed glucose threshold (e.g., <70 mg/dL).

Thus, in certain embodiments where decision support engine 114 determines, at block 1214, that the user's glucose level is not below the depressed glucose threshold and/or will not be below the depressed glucose threshold within the short period of time, at block 1218, decision support engine 114 may recommend that the user (1) consume potassium and (2) avoid consuming glucose. In certain embodiments, decision support engine 114 may further recheck glucose levels of the user.

Alternatively, in certain embodiments where decision support engine 114 determines, at block 1214, that the user's glucose level is below the depressed glucose threshold and/or will be below the depressed glucose threshold within the short period of time, at block 1216, decision support engine 114 recommends the user consume potassium and glucose.

Further, in certain embodiments, decision support engine closely monitors the user's potassium level for a next intermediate period (e.g., 1-24 hours). In particular, even users on exogenous insulin may have endogenous insulin production. Therefore, the consumption of glucose (e.g., at block 1216) may stimulate endogenous insulin release by the user, which may reduce the user's serum potassium level. Therefore, if the user's potassium level decreases following consumption of glucose and potassium, then additional potassium, but not glucose, may need to be consumed to reduce the user's risk of hypokalemia.

Accordingly, at block 1222, decision support engine 114 determines whether the user's potassium level decreased during the intermediate period (e.g., during a 2 hour window).

In certain embodiments where decision support engine 114 determines, at block 1222, that the user's potassium level did decrease during the intermediate period, at block 1226, decision support engine 114 recommends that the user (1) consume additional potassium and (2) avoid consuming glucose. Subsequent to the recommendation given at block 1226, decision support engine 114 may return to FIG. 11 to continue to treat the depressed potassium level of the user, should the user's potassium continue to be depressed and require further attention.

In certain embodiments where decision support engine 114 determines, at block 1222, that the user's potassium level did not decrease during the intermediate period, at block 1224, decision support engine 114 may recommend that the user (1) consume potassium and (2) avoid consuming glucose. In certain embodiments, decision support engine 114 further recommends that the user continue to monitor the user's glucose level and the user's potassium level. In certain embodiments, where such determination has previously occurred and may be related to medication, decision support engine 114 further recommends that the user contact a healthcare provider.

In certain embodiments, decision support engine 114 may further proceed to FIG. 13 to determine one or more diet recommendations for users determined to be at severe, moderate, or mild risk of hyperkalemia, per FIG. 11. In particular, users at risk of potassium imbalance (e.g., prescribed medications which affect the user's potassium level), may need to closely monitor their diet to improve potassium imbalance and/or reduce their risk of hypokalemia. As described in more detail below, diet recommendations for users at severe, moderate, or mild risk of hypokalemia may include a high-potassium diet and/or a low-carbohydrate (e.g., glucose) diet. Dietary recommendations may be given based on a user's potassium level, glucose level, and/or dietary requirements.

FIG. 13 illustrates an example method 1300 used for providing diet recommendations for a user determined to be at risk of hypokalemia, according to certain embodiments of the present disclosure. Method 1300 is described below with reference to FIGS. 1 and 2 and their components. For example, components of decision support system 100 illustrated in FIG. 1 may use method 1300 to determine treatment (or preventative) recommendations to recommend to a user for purposes of increasing the user's depressed potassium level and/or preventing the user's depressed potassium level from decreasing further.

Method 1300 begins at block 1302 by decision support system 100, and more specifically decision support engine 114 of decision support system 100, determining whether the user's glucose level is above an elevated glucose threshold (e.g., >200 milligrams per deciliter (mg/dL)) and/or whether the user's glucose level will be above the elevated glucose threshold within a short period of time (e.g., ≤1 hour). The short period of time may be predefined.

In certain embodiments where decision support engine 114 determines, at block 1302, that the user's glucose level is above the elevated glucose threshold (e.g., user's glucose level >200 mg/dL) and/or the user's glucose level will be above the elevated glucose threshold within the short period of time, decision support engine 114 recommends, at block 1304, that the user (1) consume potassium and (2) avoid consuming glucose.

In certain embodiments where decision support engine 114 determines, at block 1302, that the user's glucose level is above the elevated glucose threshold and/or the user's glucose level will be above the elevated glucose threshold within the short period of time, the recommendation given by decision support engine 114 at block 1304 includes (1) a specific food recommendation, or (2) a recommendation of specific foods to avoid consuming. For example, a recommendation given by decision support engine 114 at block 1304 may recommend to the user one or more types of meals, snacks, and/or beverages for consumption (e.g., foods that are high in potassium and/or low in glucose). In further examples, a recommendation given by decision support engine 114 at block 1004 may recommend to the user one or more types of meals, snacks, and/or beverages to avoid (e.g., foods that are high in glucose and/or low in potassium). In certain embodiments, at block 1004, a user may input a meal, snack, and/or beverage into, e.g., application 106, and decision support engine 114 may determine whether the meal, snack, and/or beverages may be consumed or should be avoided by the user. For example, the user may input food and drink information by providing a photograph through an application that is configured to recognize food types and quantities, and/or by scanning a bar code or menu.

Alternatively, in certain embodiments where decision support engine 114 determines, at block 1302, that the user's glucose level is not above the elevated glucose threshold (e.g., user's glucose level <200 mg/dL) and/or the user's glucose level will not be above the elevated glucose threshold within the short period of time, decision support engine 114 determines, at block 1306, whether the user's glucose level is within a healthy glucose range (e.g., between 70-200 mg/dL). In certain embodiments where the user's glucose level is within a healthy glucose range, decision support engine 114 recommends, at block 1308, that the user (1) consume potassium and (2) consume low, or no, glucose. Specifically, where a user's glucose level is in a healthy glucose range, the consumption of glucose may not be recommended. However, a small amount of glucose may be consumed. Where a user's potassium level is depressed, the consumption of potassium may also be recommended. Therefore, decision support engine 114 may recommend the user consume potassium and, in some cases, may recommend foods containing a small amount of glucose (i.e., low glucose content to avoid raising the user's glucose level).

In certain embodiments where decision support engine 114 determines, at block 1302, that the user's glucose level is not above the elevated glucose threshold and/or the user's glucose level will not be above the elevated glucose threshold within the short period of time, the recommendation given by decision support engine 114 at block 1304 includes (1) a specific food recommendation, or (2) a recommendation of specific foods to avoid consuming. For example, a recommendation given by decision support engine 114 at block 1304 may recommend to the user one or more types of meals, snacks, and/or beverages for consumption (e.g., e.g., foods that are high in potassium and/or low in glucose). In further examples, a recommendation given by decision support engine 114 at block 1004 may recommend to the user one or more types of meals, snacks, and/or beverages to avoid (e.g., foods that are high in glucose and/or low in potassium).

In certain embodiments where the user's glucose level is not within a healthy range, decision support engine 114 determines, at block 1310, whether the user's glucose level is below a depressed glucose threshold (e.g., <70 mg/dL) and/or whether the user's glucose level will be below the depressed glucose threshold within a short period of time (e.g., ≤1 hour). Decision support engine 114 is expected to determine, at block 1310, that the user's glucose level is below the depressed glucose threshold and/or will be within the short period of time given decision support engine 114 (1) previously determined, at block 1302, that the user's glucose level was not above the elevated glucose threshold (e.g., user's glucose level ≤200 mg/dL) and/or would not be within the short period of time and (2) previously determined, at block 1306, that the user's glucose level was not within a healthy glucose range (e.g., not within 70-200 mg/dL). As such, the user's glucose level is expected to be below the depressed glucose threshold (e.g., <70 mg/dL).

Thus, where decision support engine 114 determines, at block 1310, that the user's glucose level is below the depressed glucose threshold and/or will be below the depressed glucose threshold within the short period of time, at block 1312, decision support engine 114 recommends the user consume potassium and glucose. In particular, depressed and/or falling glucose levels (e.g., <70 mg/dL within the next hour) may indicate that a user needs to consume glucose to prevent hypoglycemia. Where a user's potassium level is depressed, the consumption of potassium may also be recommended. Therefore, decision support engine 114 may recommend the user consume potassium glucose. The recommendation may include (1) a recommended amount of glucose/carbohydrate to consume, (2) an amount of potassium to consume, and/or (3) specific food recommendations.

In certain embodiments, where decision support engine 114 determines, at block 1310, that the user's glucose level is below the depressed glucose threshold and/or will be below the depressed glucose threshold within the short period of time, the recommendation given by decision support engine 114 at block 1312 includes a specific food recommendation. For example, a recommendation given by decision support engine 114 at block 1312 may recommend to the user one or more types of meals, snacks, and/or beverages for consumption (e.g., e.g., foods that are high in potassium and/or high in glucose).

Subsequent to the recommendations given at block 1312, decision support engine 114 may return to FIG. 11 to continue to treat a depressed potassium level of the user, should the user's potassium continue to be depressed and require further attention.

In certain embodiments where decision support engine 114 determines, at block 1310, that the user's glucose level is not below the depressed glucose threshold and/or will not be below the depressed glucose threshold within the short period of time, at block 1314, decision support engine 114 may recommend that the user (1) consume potassium, (2) avoid consuming glucose, and/or (3) recommend that the user consider other methods of increasing their potassium levels, without consuming glucose and/or potassium. In certain embodiments, decision support engine 114 may further recheck glucose levels of the user.

In certain embodiments where decision support engine 114 determines, at block 1310, that the user's glucose level is not below the depressed glucose threshold and/or will not be below the depressed glucose threshold within the short period of time, the recommendation given by decision support engine 114 at block 1314 includes (1) a specific food recommendation, or (2) a recommendation of specific foods to avoid consuming. For example, a recommendation given by decision support engine 114 at block 1314 may recommend to the user one or more types of meals, snacks, and/or beverages for consumption (e.g., e.g., foods that are high in potassium and/or low in glucose). In further examples, a recommendation given by decision support engine 114 at block 1004 may recommend to the user one or more types of meals, snacks, and/or beverages to avoid (e.g., foods that are high in glucose and/or low in potassium).

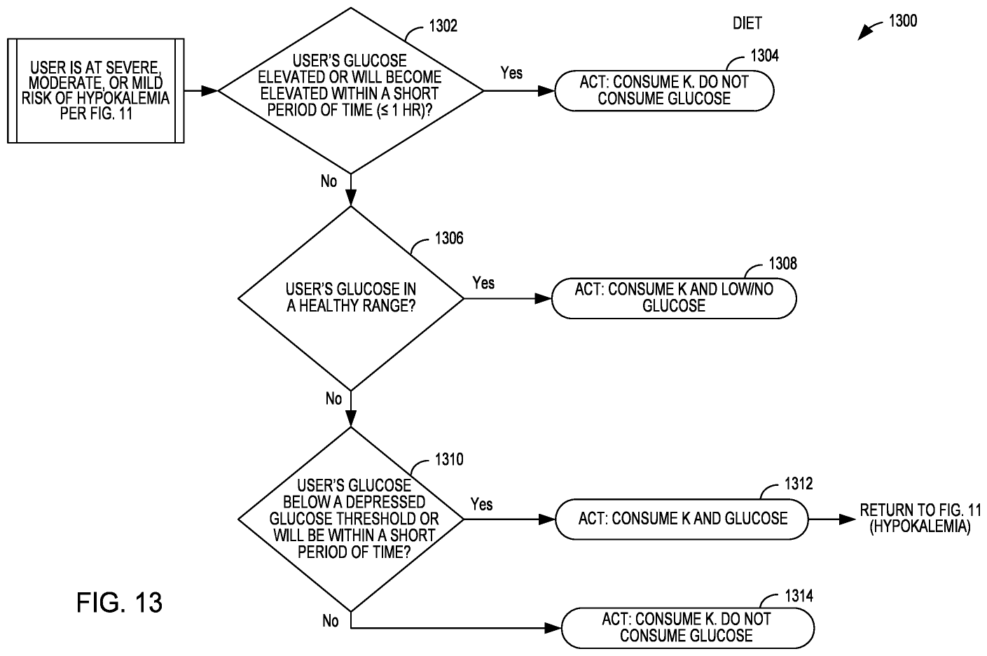

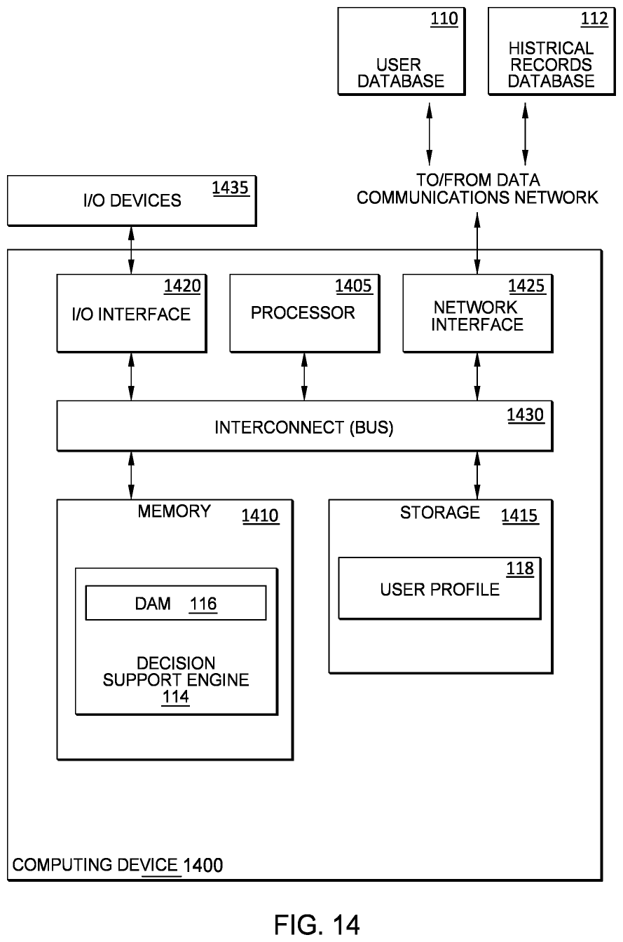

FIG. 14 is a block diagram depicting a computing device 1400 configured for (1) predicting one or more symptoms associated with kidney disease and/or (2) diagnosing, staging, treating, and assessing risks of kidney disease, according to certain embodiments disclosed herein. Although depicted as a single physical device, in embodiments, computing device 1400 may be implemented using virtual device(s), and/or across a number of devices, such as in a cloud environment. As illustrated, computing device 1400 includes a processor 1405, memory 1410, storage 1415, a network interface 1425, and one or more I/O interfaces 1420. In the illustrated embodiment, processor 1405 retrieves and executes programming instructions stored in memory 1410, as well as stores and retrieves application data residing in storage 1415. Processor 1405 is generally representative of a single CPU and/or GPU, multiple CPUs and/or GPUs, a single CPU and/or GPU having multiple processing cores, and the like. Memory 1410 is generally included to be representative of a random access memory (RAM). Storage 1415 may be any combination of disk drives, flash-based storage devices, and the like, and may include fixed and/or removable storage devices, such as fixed disk drives, removable memory cards, caches, optical storage, network attached storage (NAS), or storage area networks (SAN).

In some embodiments, I/O devices 1435 (such as keyboards, monitors, etc.) can be connected via the I/O interface(s) 1420. Further, via network interface 1425, computing device 1400 can be communicatively coupled with one or more other devices and components, such as user database 110 and/or historical records database 112. In certain embodiments, computing device 1400 is communicatively coupled with other devices via a network, which may include the Internet, local network(s), and the like. The network may include wired connections, wireless connections, or a combination of wired and wireless connections. As illustrated, processor 1405, memory 1410, storage 1415, network interface(s) 1425, and I/O interface(s) 1420 are communicatively coupled by one or more interconnects 1430. In certain embodiments, computing device 1400 is representative of display device 107 associated with the user. In certain embodiments, as discussed above, display device 107 can include the user's laptop, computer, smartphone, and the like. In another embodiment, computing device 1400 is a server executing in a cloud environment.

In the illustrated embodiment, storage 1415 includes user profile 118. Memory 1410 includes decision support engine 114, which itself includes DAM 116. Decision support engine 114 is executed by computing device 1400 to perform operations in workflow 400 of FIG. 4, operations of method 500 in FIG. 5, operations of method 600 in FIG. 6, and/or use the method illustrated in FIGS. 7-13 for providing decision support in the form of risk assessment and treatment for kidney disease and/or glucose homeostasis.

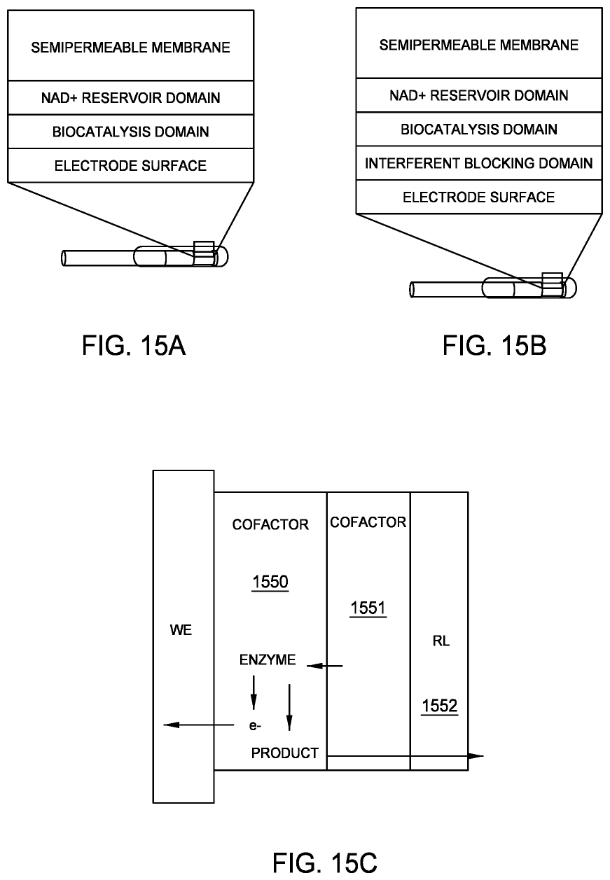

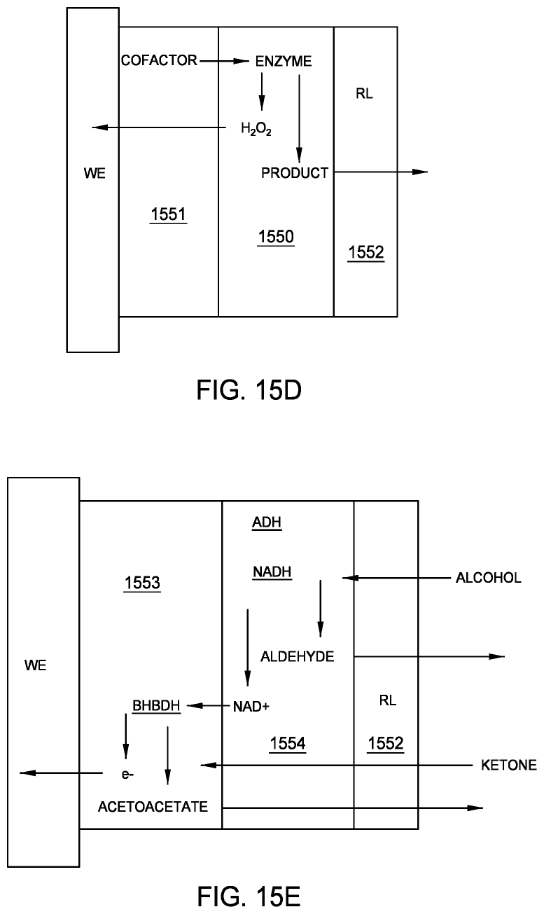

As described above, continuous analyte monitoring system 104, described in relation to FIG. 1, may be a multi-analyte sensor system including a multi-analyte sensor. FIG. 15-19 describe example multi-analyte sensors used to measure multiple analytes.

The phrases “analyte-measuring device,” “analyte-monitoring device,” “analyte-sensing device,” and/or “multi-analyte sensor device” as used herein are broad phrases, and are to be given their ordinary and customary meaning to a person of ordinary skill in the art (and are not to be limited to a special or customized meaning), and refer without limitation to an apparatus and/or system responsible for the detection of, or transduction of a signal associated with, a particular analyte or combination of analytes. For example, these phrases may refer without limitation to an instrument responsible for detection of a particular analyte or combination of analytes. In one example, the instrument includes a sensor coupled to circuitry disposed within a housing, and configure to process signals associated with analyte concentrations into information. In one example, such apparatuses and/or systems are capable of providing specific quantitative, semi-quantitative, qualitative, and/or semi qualitative analytical information using a biological recognition element combined with a transducing (detecting) element.

The terms “biosensor” and/or “sensor” as used herein are broad terms and are to be given their ordinary and customary meaning to a person of ordinary skill in the art (and are not to be limited to a special or customized meaning), and refer without limitation to a part of an analyte measuring device, analyte-monitoring device, analyte sensing device, and/or multi-analyte sensor device responsible for the detection of, or transduction of a signal associated with, a particular analyte or combination of analytes. In one example, the biosensor or sensor generally comprises a body, a working electrode, a reference electrode, and/or a counter electrode coupled to body and forming surfaces configured to provide signals during electrochemically reactions. One or more membranes can be affixed to the body and cover electrochemically reactive surfaces. In one example, such biosensors and/or sensors are capable of providing specific quantitative, semi-quantitative, qualitative, semi qualitative analytical signals using a biological recognition element combined with a transducing (detecting) element.

The phrases “sensing portion,” “sensing membrane,” and/or “sensing mechanism” as used herein are broad phrases, and are to be given their ordinary and customary meaning to a person of ordinary skill in the art (and are not to be limited to a special or customized meaning), and refer without limitation to the part of a biosensor and/or a sensor responsible for the detection of, or transduction of a signal associated with, a particular analyte or combination of analytes. In one example, the sensing portion, sensing membrane, and/or sensing mechanism generally comprise an electrode configured to provide signals during electrochemically reactions with one or more membranes covering electrochemically reactive surface. In one example, such sensing portions, sensing membranes, and/or sensing mechanisms can provide specific quantitative, semi-quantitative, qualitative, semi qualitative analytical signals using a biological recognition element combined with a transducing (detecting) element.

The phrases “biointerface membrane” and “biointerface layer” as used interchangeably herein are broad phrases, and are to be given their ordinary and customary meaning to a person of ordinary skill in the art (and are not to be limited to a special or customized meaning), and refer without limitation to a permeable membrane (which can include multiple domains) or layer that functions as a bioprotective interface between host tissue and an implantable device. The terms “biointerface” and “bioprotective” are used interchangeably herein.

The term “cofactor” as used herein is a broad term, and is to be given its ordinary and customary meaning to a person of ordinary skill in the art (and is not to be limited to a special or customized meaning), and refers without limitation to one or more substances whose presence contributes to or is required for analyte-related activity of an enzyme. Analyte-related activity can include, but is not limited to, any one of or a combination of binding, electron transfer, and chemical transformation. Cofactors are inclusive of coenzymes, non-protein chemical compounds, metal ions and/or metal organic complexes. Coenzymes are inclusive of prosthetic groups and co-substrates.

The term “continuous” as used herein is a broad term, and is to be given its ordinary and customary meaning to a person of ordinary skill in the art (and is not to be limited to a special or customized meaning), and refers without limitation to an uninterrupted or unbroken portion, domain, coating, or layer.

The phrases “continuous analyte sensing” and “continuous multi-analyte sensing” as used herein are broad phrases, and are to be given their ordinary and customary meaning to a person of ordinary skill in the art (and is not to be limited to a special or customized meaning), and refers without limitation to the period in which monitoring of analyte concentration is continuously, continually, and/or intermittently (but regularly) performed, for example, from about every second or less to about one week or more. In further examples, monitoring of analyte concentration is performed from about every 2, 3, 5, 7, 10, 15, 20, 25, 30, 35, 40, 45, 50, 55, or 60 seconds to about every 1.25, 1.50, 1.75, 2.00, 2.25, 2.50, 2.75, 3.00, 3.25, 3.50, 3.75, 4.00, 4.25, 4.50, 4.75, 5.00, 5.25, 5.50, 5.75, 6.00, 6.25, 6.50, 6.75, 7.00, 7.25, 7.50, 7.75, 8.00, 8.25, 8.50, 8.75, 9.00, 9.25, 9.50 or 9.75 minutes. In further examples, monitoring of analyte concentration is performed from about 10, 20, 30, 40 or 50 minutes to about every 1, 2, 3, 4, 5, 6, 7 or 8 hours. In further examples, monitoring of analyte concentration is performed from about every 8 hours to about every 12, 16, 20, or 24 hours. In further examples, monitoring of analyte concentration is performed from about every day to about every 1.5, 2, 3, 4, 5, 6, or 7 days. In further examples, monitoring of analyte concentration is performed from about every week to about every 1.5, 2, 3 or more weeks.

The term “coaxial” as used herein is to be construed broadly to include sensor architectures having elements aligned along a shared axis around a core that can be configured to have a circular, elliptical, triangular, polygonal, or other cross-section such elements can include electrodes, insulating layers, or other elements that can be positioned circumferentially around the core layer, such as a core electrode or core polymer wire.

The term “coupled” as used herein is a broad term, and is to be given its ordinary and customary meaning to a person of ordinary skill in the art (and is not to be limited to a special or customized meaning), and refers without limitation to two or more system elements or components that are configured to be at least one of electrically, mechanically, thermally, operably, chemically or otherwise attached. For example, an element is “coupled” if the element is covalently, communicatively, electrostatically, thermally connected, mechanically connected, magnetically connected, or ionically associated with, or physically entrapped, adsorbed to or absorbed by another element. Similarly, the phrases “operably connected”, “operably linked”, and “operably coupled” as used herein may refer to one or more components linked to another component(s) in a manner that facilitates transmission of at least one signal between the components. In some examples, components are part of the same structure and/or integral with one another as in covalently, electrostatically, mechanically, thermally, magnetically, ionically associated with, or physically entrapped, or absorbed (i.e. “directly coupled” as in no intervening element(s)). In other examples, components are connected via remote means. For example, one or more electrodes can be used to detect an analyte in a sample and convert that information into a signal; the signal can then be transmitted to an electronic circuit. In this example, the electrode is “operably linked” to the electronic circuit. The phrase “removably coupled” as used herein may refer to two or more system elements or components that are configured to be or have been electrically, mechanically, thermally, operably, chemically, or otherwise attached and detached without damaging any of the coupled elements or components. The phrase “permanently coupled” as used herein may refer to two or more system elements or components that are configured to be or have been electrically, mechanically, thermally, operably, chemically, or otherwise attached but cannot be uncoupled without damaging at least one of the coupled elements or components. covalently, electrostatically, ionically associated with, or physically entrapped, or absorbed

The term “discontinuous” as used herein is a broad term, and is to be given its ordinary and customary meaning to a person of ordinary skill in the art (and is not to be limited to a special or customized meaning), and refers without limitation to disconnected, interrupted, or separated portions, layers, coatings, or domains.

The term “distal” as used herein is a broad term, and is to be given its ordinary and customary meaning to a person of ordinary skill in the art (and is not to be limited to a special or customized meaning), and refers without limitation to a region spaced relatively far from a point of reference, such as an origin or a point of attachment.

The term “domain” as used herein is a broad term, and is to be given its ordinary and customary meaning to a person of ordinary skill in the art (and is not to be limited to a special or customized meaning), and refers without limitation to a region of a membrane system that can be a layer, a uniform or non-uniform gradient (for example, an anisotropic region of a membrane), or a portion of a membrane that is capable of sensing one, two, or more analytes. The domains discussed herein can be formed as a single layer, as two or more layers, as pairs of bi-layers, or as combinations thereof.

The term “electrochemically reactive surface” as used herein is a broad term, and is to be given its ordinary and customary meaning to a person of ordinary skill in the art (and is not to be limited to a special or customized meaning), and refers without limitation to the surface of an electrode where an electrochemical reaction takes place. In one example this reaction is faradaic and results in charge transfer between the surface and its environment. In one example, hydrogen peroxide produced by an enzyme-catalyzed reaction of an analyte being oxidized on the surface results in a measurable electronic current. For example, in the detection of glucose, glucose oxidase produces hydrogen peroxide (H₂O₂) as a byproduct. The H₂O₂ reacts with the surface of the working electrode to produce two protons (2H⁺) two electrons (2e⁻) and one molecule of oxygen (O₂), which produces the electronic current being detected. In a counter electrode, a reducible species, for example, O₂ is reduced at the electrode surface so as to balance the current generated by the working electrode.

The term “electrolysis” as used herein is a broad term, and is to be given its ordinary and customary meaning to a person of ordinary skill in the art (and is not to be limited to a special or customized meeting), and refers without limitation to electrooxidation or electroreduction (collectively, “redox”) of a compound, either directly or indirectly, by one or more enzymes, cofactors, or mediators.

The terms “indwelling,” “in dwelling,” “implanted,” or “implantable” as used herein are broad terms, and are to be given their ordinary and customary meaning to a person of ordinary skill in the art (and are not to be limited to a special or customized meaning), and refer without limitation to objects including sensors that are inserted, or configured to be inserted, subcutaneously (i.e. in the layer of fat between the skin and the muscle), intracutaneously (i.e. penetrating the stratum corneum and positioning within the epidermal or dermal strata of the skin), or transcutaneously (i.e. penetrating, entering, or passing through intact skin), which may result in a sensor that has an in vivo portion and an ex vivo portion. The term “indwelling” also encompasses an object which is configured to be inserted subcutaneously, intracutaneously, or transcutaneously, whether or not it has been inserted as such.

The terms “interferants” and “interfering species” as used herein are broad terms, and are to be given their ordinary and customary meaning to a person of ordinary skill in the art (and are not to be limited to a special or customized meaning), and refer without limitation to effects and/or species that interfere with the measurement of an analyte of interest in a sensor to produce a signal that does not accurately represent the analyte measurement. In one example of an electrochemical sensor, interfering species are compounds which produce a signal that is not analyte-specific due to a reaction on an electrochemically active surface.

The term “in vivo” as used herein is a broad term, and is to be given its ordinary and customary meaning to a person of ordinary skill in the art (and is not to be limited to a special or customized meaning), and without limitation is inclusive of the portion of a device (for example, a sensor) adapted for insertion into and/or existence within a living body of a host.

The term “ex vivo” as used herein is a broad term, and is to be given its ordinary and customary meaning to a person of ordinary skill in the art (and is not to be limited to a special or customized meaning), and without limitation is inclusive of a portion of a device (for example, a sensor) adapted to remain and/or exist outside of a living body of a host.

The term and phrase “mediator” and “redox mediator” as used herein are broad terms and phrases, and are to be given their ordinary and customary meaning to a person of ordinary skill in the art (and is not to be limited to a special or customized meaning), and refers without limitation to any chemical compound or collection of compounds capable of electron transfer, either directly, or indirectly, between an analyte, analyte precursor, analyte surrogate, analyte-reduced or analyte-oxidized enzyme, or cofactor, and an electrode surface held at a potential. In one example the mediator accepts electrons from, or transfer electrons to, one or more enzymes or cofactors, and/or exchanges electrons with the sensor system electrodes. In one example, mediators are transition-metal coordinated organic molecules which are capable of reversible oxidation and reduction reactions. In other examples, mediators may be organic molecules or metals which are capable of reversible oxidation and reduction reactions.

The term “membrane” as used herein is a broad term, and is to be given its ordinary and customary meaning to a person of ordinary skill in the art (and is not to be limited to a special or customized meaning), and refers without limitation to a structure configured to perform functions including, but not limited to, protection of the exposed electrode surface from the biological environment, diffusion resistance (limitation) of the analyte, service as a matrix for a catalyst (e.g., one or more enzymes) for enabling an enzymatic reaction, limitation or blocking of interfering species, provision of hydrophilicity at the electrochemically reactive surfaces of the sensor interface, service as an interface between host tissue and the implantable device, modulation of host tissue response via drug (or other substance) release, and combinations thereof. When used herein, the terms “membrane” and “matrix” are meant to be interchangeable.

The phrase “membrane system” as used herein is a broad phrase, and is to be given its ordinary and customary meaning to a person of ordinary skill in the art (and is not to be limited to a special or customized meaning), and refers without limitation to a permeable or semi-permeable membrane that can be comprised of two or more domains, layers, or layers within a domain, and is typically constructed of materials of a few microns thickness or more, which is permeable to oxygen and is optionally permeable to, e.g., glucose or another analyte. In one example, the membrane system comprises an enzyme, which enables an analyte reaction to occur whereby a concentration of the analyte can be measured.

The term “planar” as used herein is to be interpreted broadly to describe sensor architecture having a substrate including at least a first surface and an opposing second surface, and for example, comprising a plurality of elements arranged on one or more surfaces or edges of the substrate. The plurality of elements can include conductive or insulating layers or elements configured to operate as a circuit. The plurality of elements may or may not be electrically or otherwise coupled. In one example, planar includes one or more edges separating the opposed surfaces.

The term “proximal” as used herein is a broad term, and is to be given its ordinary and customary meaning to a person of ordinary skill in the art (and is not to be limited to a special or customized meaning), and refers without limitation to the spatial relationship between various elements in comparison to a particular point of reference. For example, some examples of a device include a membrane system having a biointerface layer and an enzyme domain or layer. If the sensor is deemed to be the point of reference and the enzyme domain is positioned nearer to the sensor than the biointerface layer, then the enzyme domain is more proximal to the sensor than the biointerface layer.

The phrases “sensing portion,” “sensing membrane,” and/or “sensing mechanism” as used herein are broad phrases, and are to be given their ordinary and customary meaning to a person of ordinary skill in the art (and are not to be limited to a special or customized meaning), and refer without limitation to the part of a biosensor and/or a sensor responsible for the detection of, or transduction of a signal associated with, a particular analyte or combination of analytes. In one example, the sensing portion, sensing membrane, and/or sensing mechanism generally comprise an electrode configured to provide signals during electrochemically reactions with one or more membranes covering electrochemically reactive surface. In one example, such sensing portions, sensing membranes, and/or sensing mechanisms are capable of providing specific quantitative, semi-quantitative, qualitative, semi qualitative analytical signals using a biological recognition element combined with a transducing and/or detecting element.

During general operation of the analyte measuring device, biosensor, sensor, sensing region, sensing portion, or sensing mechanism, a biological sample, for example, blood or interstitial fluid, or a component thereof contacts, either directly, or after passage through one or more membranes, an enzyme, for example, glucose oxidase, DNA, RNA, or a protein or aptamer, for example, one or more periplasmic binding protein (PBP) or mutant or fusion protein thereof having one or more analyte binding regions, each region capable of specifically or reversibly binding to and/or reacting with at least one analyte. The interaction of the biological sample or component thereof with the analyte measuring device, biosensor, sensor, sensing region, sensing portion, or sensing mechanism results in transduction of a signal that permits a qualitative, semi-qualitative, quantitative, or semi-qualitative determination of the analyte level, for example, glucose, ketone, lactate, potassium, etc., in the biological sample.

In one example, the sensing region or sensing portion can comprise at least a portion of a conductive substrate or at least a portion of a conductive surface, for example, a wire (coaxial) or conductive trace or a substantially planar substrate including substantially planar trace(s), and a membrane. In one example, the sensing region or sensing portion can comprise a non-conductive body, a working electrode, a reference electrode, and a counter electrode (optional), forming an electrochemically reactive surface at one location on the body and an electronic connection at another location on the body, and a sensing membrane affixed to the body and covering the electrochemically reactive surface. In some examples, the sensing membrane further comprises an enzyme domain, for example, an enzyme domain, and an electrolyte phase, for example, a free-flowing liquid phase comprising an electrolyte-containing fluid described further below. The terms are broad enough to include the entire device, or only the sensing portion thereof (or something in between).

In another example, the sensing region can comprise one or more periplasmic binding protein (PBP) including mutant or fusion protein thereof, or aptamers having one or more analyte binding regions, each region capable of specifically and reversibly binding to at least one analyte. Alterations of the aptamer or mutations of the PBP can contribute to or alter one or more of the binding constants, long-term stability of the protein, including thermal stability, to bind the protein to a special encapsulation matrix, membrane or polymer, or to attach a detectable reporter group or “label” to indicate a change in the binding region or transduce a signal corresponding to the one or more analytes present in the biological fluid. Specific examples of changes in the binding region include, but are not limited to, hydrophobic/hydrophilic environmental changes, three-dimensional conformational changes, changes in the orientation of amino/nucleic acid side chains in the binding region of proteins, and redox states of the binding region. Such changes to the binding region provide for transduction of a detectable signal corresponding to the one or more analytes present in the biological fluid.

In one example, the sensing region determines the selectivity among one or more analytes, so that only the analyte which has to be measured leads to (transduces) a detectable signal. The selection may be based on any chemical or physical recognition of the analyte by the sensing region, where the chemical composition of the analyte is unchanged, or in which the sensing region causes or catalyzes a reaction of the analyte that changes the chemical composition of the analyte.

The term “sensitivity” as used herein is a broad term, and is to be given its ordinary and customary meaning to a person of ordinary skill in the art (and is not to be limited to a special or customized meaning), and refers without limitation to an amount of signal (e.g., in the form of electrical current and/or voltage) produced by a predetermined amount (unit) of the measured analyte. For example, in one example, a sensor has a sensitivity (or slope) of from about 1 to about 100 picoAmps of current for every 1 mg/dL of analyte.

The phrases “signal medium” or “transmission medium” shall be taken to include any form of modulated data signal, carrier wave, and so forth. The phrase “modulated data signal” means a signal that has one or more of its characteristics set or changed in such a matter as to encode information in the signal.

The terms “transducing” or “transduction” and their grammatical equivalents as are used herein are broad terms, and are to be given their ordinary and customary meaning to a person of ordinary skill in the art (and is not to be limited to a special or customized meaning), and refer without limitation to optical, electrical, electrochemical, acoustical/mechanical, or colorimetrical technologies and methods. Electrochemical properties include current and/or voltage, inductance, capacitance, impedance, transconductance, and potential. Optical properties include absorbance, fluorescence/phosphorescence, fluorescence/phosphorescence decay rate, wavelength shift, dual wave phase modulation, bio/chemiluminescence, reflectance, light scattering, and refractive index. For example, the sensing region transduces the recognition of analytes into a semi-quantitative or quantitative signal.

As used herein, the phrase “transducing element” as used herein is a broad phrase, and are to be given their ordinary and customary meaning to a person of ordinary skill in the art (and is not to be limited to a special or customized meaning), and refers without limitation to analyte recognition moieties capable of facilitating, directly or indirectly, with detectable signal transduction corresponding to the presence and/or concentration of the recognized analyte. In one example, a transducing element is one or more enzymes, one or more aptamers, one or more ionophores, one or more capture antibodies, one or more proteins, one or more biological cells, one or more oligonucleotides, and/or one or more DNA or RNA moieties. Transcutaneous continuous multi-analyte sensors can be used in vivo over various lengths of time. The continuous multi-analyte sensor systems discussed herein can be transcutaneous devices, in that a portion of the device may be inserted through the host's skin and into the underlying soft tissue while a portion of the device remains on the surface of the host's skin. In one aspect, in order to overcome the problems associated with noise or other sensor function in the short-term, one example employs materials that promote formation of a fluid pocket around the sensor, for example architectures such as a porous biointerface membrane or matrices that create a space between the sensor and the surrounding tissue. In some examples, a sensor is provided with a spacer adapted to provide a fluid pocket between the sensor and the host's tissue. It is believed that this spacer, for example a biointerface material, matrix, structure, and the like as described in more detail elsewhere herein, provides for oxygen and/or glucose transport to the sensor.

### Membrane Systems

Membrane systems disclosed herein are suitable for use with implantable devices in contact with a biological fluid. For example, the membrane systems can be utilized with implantable devices, such as devices for monitoring and determining analyte levels in a biological fluid, for example, devices for monitoring glucose levels for individuals having diabetes. In some examples, the analyte-measuring device is a continuous device. The analyte-measuring device can employ any suitable sensing element to provide the raw signal, including but not limited to those involving enzymatic, chemical, physical, electrochemical, spectrophotometric, amperometric, potentiometric, polarimetric, calorimetric, radiometric, immunochemical, or like elements.

Suitable membrane systems for the aforementioned multi-analyte systems and devices can include, for example, membrane systems disclosed in U.S. Pat. Nos. 6,015,572, 5,964,745, and 6,083,523, which are incorporated herein by reference in their entireties for their teachings of membrane systems.

In general, the membrane system includes a plurality of domains, for example, an electrode domain, an interference domain, an enzyme domain, a resistance domain, and a biointerface domain. The membrane system can be deposited on the exposed electroactive surfaces using known thin film techniques (for example, vapor deposition, spraying, electrodepositing, dipping, brush coating, film coating, drop-let coating, and the like). Additional steps may be applied following the membrane material deposition, for example, drying, annealing, and curing (for example, UV curing, thermal curing, moisture curing, radiation curing, and the like) to enhance certain properties such as mechanical properties, signal stability, and selectivity. In a typical process, upon deposition of the resistance domain membrane, a biointerface/drug releasing layer having a “dry film” thickness of from about 0.05 micron (μm), or less, to about 1, 2, 3, 4, 5, 6, 7, 8, 9, 10, 11, 12, 13, 14, 15, or 16 μm is formed. “Dry film” thickness refers to the thickness of a cured film cast from a coating formulation by standard coating techniques.

In certain examples, the biointerface/drug releasing layer is formed of a biointerface polymer, wherein the biointerface polymer comprises one or more membrane domains comprising polyurethane and/or polyurea segments and one or more zwitterionic repeating units. In some examples, the biointerface/drug releasing layer coatings are formed of a polyurethane urea having carboxyl betaine groups incorporated in the polymer and non-ionic hydrophilic polyethylene oxide segments, wherein the polyurethane urea polymer is dissolved in organic or non-organic solvent system according to a pre-determined coating formulation, and is crosslinked with an isocyanate crosslinker and cured at a moderate temperature of about 50° C. The solvent system can be a single solvent or a mixture of solvents to aid the dissolution or dispersion of the polymer. The solvents can be the ones selected as the polymerization media or added after polymerization is completed. The solvents are selected from the ones having lower boiling points to facilitate drying and to be lower in toxicity for implant applications. Examples of these solvents include aliphatic ketone, ester, ether, alcohol, hydrocarbons, and the like. Depending on the final thickness of the biointerface/drug releasing layer and solution viscosity (as related to the percent of polymer solid), the coating can be applied in a single step or multiple repeated steps of the chosen process such as dipping to build the desired thickness. Yet in other examples, the bioprotective polymers are formed of a polyurethane urea having carboxylic acid groups and carboxyl betaine groups incorporated in the polymer and non-ionic hydrophilic polyethylene oxide segments, wherein the polyurethane urea polymer is dissolved in an organic or non-organic solvent system in a coating formulation, and is crosslinked with an a carbodiimide (e.g., 1-ethyl-3-(3-dimethylaminopropyl)carbodiimide (EDC)) and cured at a moderate temperature of about 50° C.

In other examples, the biointerface/drug releasing layer coatings are formed of a polyurethane urea having sulfobetaine groups incorporated in the polymer and non-ionic hydrophilic polyethylene oxide segments, wherein the polyurethane urea polymer is dissolved in an organic or non-organic solvent system according to a pre-determined coating formulation, and is crosslinked with an isocyanate crosslinker and cured at a moderate temperature of about 50° C. The solvent system can be a single solvent or a mixture of solvents to aid the dissolution or dispersion of the polymer. The solvents can be the ones selected as the polymerization media or added after polymerization is completed. The solvents are selected from the ones having lower boiling points to facilitate drying and to be lower in toxicity for implant applications. Examples of these solvents include aliphatic ketone, ester, ether, alcohol, hydrocarbons, and the like. Depending on the final thickness of the biointerface/drug releasing layer and solution viscosity (as related to the percent of polymer solid), the coating can be applied in a single step or multiple repeated steps of the chosen process such as dipping to build the desired thickness. Yet in other examples, the biointerface polymers are formed of a polyurethane urea having unsaturated hydrocarbon groups and sulfobetaine groups incorporated in the polymer and non-ionic hydrophilic polyethylene oxide segments, wherein the polyurethane urea polymer is dissolved in an organic or non-organic solvent system in a coating formulation, and is crosslinked in the presence of initiators with heat or irradiation including UV, LED light, electron beam, and the like, and cured at a moderate temperature of about 50° C. Examples of unsaturated hydrocarbon includes allyl groups, vinyl groups, acrylate, methacrylate, alkenes, alkynes, and the like.

In some examples, tethers are used. A tether is a polymer or chemical moiety which does not participate in the (electro)chemical reactions involved in sensing, but forms chemical bonds with the (electro)chemically active components of the membrane. In some examples these bonds are covalent. In one example, a tether may be formed in solution prior to one or more interlayers of a membrane being formed, where the tether bonds two (electro)chemically active components directly to one another or alternately, the tether(s) bond (electro)chemically active component(s) to polymeric backbone structures. In another example, (electro)chemically active components are comixed along with crosslinker(s) with tunable lengths (and optionally polymers) and the tethering reaction occurs as in situ crosslinking. Tethering may be employed to maintain a predetermined number of degrees of freedom of NAD(P)H for effective enzyme catalysis, where “effective” enzyme catalysis causes the analyte sensor to continuously monitor one or more analytes for a period of from about 5 days to about 15 days or more.

### Membrane Fabrication

Polymers can be processed by solution-based techniques such as spraying, dipping, casting, electrospinning, vapor deposition, spin coating, coating, and the like. Water-based polymer emulsions can be fabricated to form membranes by methods similar to those used for solvent-based materials. In both cases the evaporation of a volatile liquid (e.g., organic solvent or water) leaves behind a film of the polymer. Cross-linking of the deposited film or layer can be performed through the use of multi-functional reactive ingredients by a number of methods. The liquid system can cure by heat, moisture, high-energy radiation, ultraviolet light, or by completing the reaction, which produces the final polymer in a mold or on a substrate to be coated.

In some examples, the wetting property of the membrane (and by extension the extent of sensor drift exhibited by the sensor) can be adjusted and/or controlled by creating covalent cross-links between surface-active group-containing polymers, functional-group containing polymers, polymers with zwitterionic groups (or precursors or derivatives thereof), and combinations thereof. Cross-linking can have a substantial effect on film structure, which in turn can affect the film's surface wetting properties. Crosslinking can also affect the film's tensile strength, mechanical strength, water absorption rate and other properties.

Cross-linked polymers can have different cross-linking densities. In certain examples, cross-linkers are used to promote cross-linking between layers. In other examples, in replacement of (or in addition to) the cross-linking techniques described above, heat is used to form cross-linking. For example, in some examples, imide and amide bonds can be formed between two polymers as a result of high temperature. In some examples, photo cross-linking is performed to form covalent bonds between the polycationic layers(s) and polyanionic layer(s). One major advantage to photo-cross-linking is that it offers the possibility of patterning. In certain examples, patterning using photo-cross linking is performed to modify the film structure and thus to adjust the wetting property of the membranes and membrane systems, as discussed herein.

Polymers with domains or segments that are functionalized to permit cross-linking can be made by methods at least as discussed herein. For example, polyurethaneurea polymers with aromatic or aliphatic segments having electrophilic functional groups (e.g., carbonyl, aldehyde, anhydride, ester, amide, isocyano, epoxy, allyl, or halo groups) can be crosslinked with a crosslinking agent that has multiple nucleophilic groups (e.g., hydroxyl, amine, urea, urethane, or thiol groups). In further examples, polyurethaneurea polymers having aromatic or aliphatic segments having nucleophilic functional groups can be crosslinked with a crosslinking agent that has multiple electrophilic groups. Still further, polyurethaneurea polymers having hydrophilic segments having nucleophilic or electrophilic functional groups can be crosslinked with a crosslinking agent that has multiple electrophilic or nucleophilic groups. Unsaturated functional groups on the polyurethane urea can also be used for crosslinking by reacting with multivalent free radical agents. Non-limiting examples of suitable cross-linking agents include isocyanate, carbodiimide, glutaraldehyde, aziridine, silane, or other aldehydes, epoxy, acrylates, free-radical based agents, ethylene glycol diglycidyl ether (EGDE), poly(ethylene glycol) diglycidyl ether (PEGDE), or dicumyl peroxide (DCP). In one example, from about 0.1% to about 15% w/w of cross-linking agent is added relative to the total dry weights of cross-linking agent and polymers added when blending the ingredients. In another example, about 1% to about 10% w/w of cross-linking agent is added relative to the total dry weights of cross-linking agent and polymers added when blending the ingredients. In yet another example, about 5% to about 15% w/w of cross-linking agent is added relative to the total dry weights of cross-linking agent and polymers added when blending the ingredients. During the curing process, substantially all of the cross-linking agent is believed to react, leaving substantially no detectable unreacted cross-linking agent in the final film.

Polymers disclosed herein can be formulated into mixtures that can be drawn into a film or applied to a surface using methods such as spraying, self-assembling monolayers (SAMs), painting, dip coating, vapor depositing, molding, 3-D printing, lithographic techniques (e.g., photolithograph), micro- and nano-pipetting printing techniques, silk-screen printing, etc.). The mixture can then be cured under high temperature (e.g., from about 30° C. to about 150° C.). Other suitable curing methods can include ultraviolet, e-beam, or gamma radiation, for example.

In some circumstances, using continuous multianalyte monitoring systems including sensor(s) configured with bioprotective and/or drug releasing membranes, it is believed that that foreign body response is the dominant event surrounding extended implantation of an implanted device and can be managed or manipulated to support rather than hinder or block analyte transport. In another aspect, in order to extend the lifetime of the sensor, one example employs materials that promote vascularized tissue ingrowth, for example within a porous biointerface membrane. For example, tissue in-growth into a porous biointerface material surrounding a sensor may promote sensor function over extended periods of time (e.g., weeks, months, or years). It has been observed that in-growth and formation of a tissue bed can take up to 3 weeks. Tissue ingrowth and tissue bed formation is believed to be part of the foreign body response. As will be discussed herein, the foreign body response can be manipulated by the use of porous bioprotective materials that surround the sensor and promote ingrowth of tissue and microvasculature over time.

Accordingly, a sensor as discussed in examples herein may include a biointerface layer. The biointerface layer, like the drug releasing layer, may include, but is not limited to, for example, porous biointerface materials including a solid portion and interconnected cavities, all of which are described in more detail elsewhere herein. The biointerface layer can be employed to improve sensor function in the long term (e.g., after tissue ingrowth).

Accordingly, a sensor as discussed in examples herein may include a drug releasing membrane at least partially functioning as or in combination with a biointerface membrane. The drug releasing membrane may include, for example, materials including a hard-soft segment polymer with hydrophilic and optionally hydrophobic domains, all of which are described in more detail elsewhere herein, can be employed to improve sensor function in the long term (e.g., after tissue ingrowth). In one example, the materials including a hard-soft segment polymer with hydrophilic and optionally hydrophobic domains are configured to release a combination of a derivative form of dexamethasone or dexamethasone acetate with dexamethasone such that one or more different rates of release of the anti-inflammatory is achieved and the useful life of the sensor is extended. Other suitable drug releasing membranes of the present disclosure can be selected from silicone polymers, polytetrafluoroethylene, expanded polytetrafluoroethylene, polyethylene-co-tetrafluoroethylene, polyolefin, polyester, polycarbonate, biostable polytetrafluoroethylene, homopolymers, copolymers, terpolymers of polyurethanes, polypropylene (PP), polyvinylchloride (PVC), polyvinylidene fluoride (PVDF), polyvinyl alcohol (PVA), poly vinyl acetate, ethylene vinyl acetate (EVA), polybutylene terephthalate (PBT), polymethylmethacrylate (PMMA), polyether ether ketone (PEEK), polyamides, polyurethanes and copolymers and blends thereof, polyurethane urea polymers and copolymers and blends thereof, cellulosic polymers and copolymers and blends thereof, poly(ethylene oxide) and copolymers and blends thereof, poly(propylene oxide) and copolymers and blends thereof, polysulfones and block copolymers thereof including, for example, di-block, tri-block, alternating, random and graft copolymers cellulose, hydrogel polymers, poly(2-hydroxyethyl methacrylate, pHEMA) and copolymers and blends thereof, hydroxyethyl methacrylate, (HEMA) and copolymers and blends thereof, polyacrylonitrile-polyvinyl chloride (PAN-PVC) and copolymers and blends thereof, acrylic copolymers and copolymers and blends thereof, nylon and copolymers and blends thereof, polyvinyl difluoride, polyanhydrides, poly(l-lysine), poly(L-lactic acid), hydroxyethylmetharcrylate and copolymers and blends thereof, and hydroxyapeptite and copolymers and blends thereof.

### Exemplary Multi Analyte Sensor Membrane Configurations

Continuous multi-analyte sensors with various membrane configurations suitable for facilitating signal transduction corresponding to analyte concentrations, either simultaneously, intermittently, and/or sequentially are provided. In one example, such sensors can be configured using a signal transducer, comprising one or more transducing elements (“TL”). Such continuous multi-analyte sensor can employ various transducing means, for example, amperometry, voltametric, potentiometry, and impedimetric methods, among other techniques.

In one example, the transducing element comprises one or more membranes that can comprise one or more layers and or domains, each of the one or more layers or domains can independently comprise one or more signal transducers, e.g., enzymes, RNA, DNA, aptamers, binding proteins, etc. As used herein, transducing elements includes enzymes, ionophores, RNA, DNA, aptamers, binding proteins and are used interchangeably.

In one example, the transducing element is present in one or more membranes, layers, or domains formed over a sensing region. In one example, such sensors can be configured using one or more enzyme domains, e.g., membrane domains including enzyme domains, also referred to as EZ layers (“EZLs”), each enzyme domain may comprise one or more enzymes. Reference hereinafter to an “enzyme layer” is intended to include all or part of an enzyme domain, either of which can be all or part of a membrane system as discussed herein, for example, as a single layer, as two or more layers, as pairs of bi-layers, or as combinations thereof.

In one example, the continuous multi-analyte sensor uses one or more of the following analyte-substrate/enzyme pairs: for example, sarcosine oxidase in combination with creatinine amidohydrolase, creatine amidohydrolase being employed for the sensing of creatinine. Other examples of analytes/oxidase enzyme combinations that can be used in the sensing region include, for example, alcohol/alcohol oxidase, cholesterol/cholesterol oxidase, glactose:galactose/galactose oxidase, choline/choline oxidase, glutamate/glutamate oxidase, glycerol/glycerol-3phosphate oxidase (or glycerol oxidase), bilirubin/bilirubin oxidase, ascorbic/ascorbic acid oxidase, uric acid/uric acid oxidase, pyruvate/pyruvate oxidase, hypoxanthine:xanthine/xanthine oxidase, glucose/glucose oxidase, lactate/lactate oxidase, L-amino acid oxidase, and glycine/sarcosine oxidase. Other analyte-substrate/enzyme pairs can be used, including such analyte-substrate/enzyme pairs that comprise genetically altered enzymes, immobilized enzymes, mediator-wired enzymes, dimerized and/or fusion enzymes.

### NAD Based Multi Analyte Sensor Platform

Nicotinamide adenine dinucleotide (NAD(P)⁺/NAD(P)H) is a coenzyme, e.g., a dinucleotide that consists of two nucleotides joined through their phosphate groups. One nucleotide contains an adenine nucleobase and the other nicotinamide. NAD exists in two forms, e.g., an oxidized form (NAD(P)+) and reduced form (NAD(P)H) (H=hydrogen). The reaction of NAD+ and NADH is reversible, thus, the coenzyme can continuously cycle between the NAD(P)⁺/and NAD(P)H forms essentially without being consumed.

In one example, one or more enzyme domains of the sensing region of the presently disclosed continuous multi-analyte sensor device comprise an amount of NAD+ or NADH for providing transduction of a detectable signal corresponding to the presence or concentration of one or more analytes. In one example, one or more enzyme domains of the sensing region of the presently disclosed continuous multi-analyte sensor device comprise an excess amount of NAD+ or NADH for providing extended transduction of a detectable signal corresponding to the presence or concentration of one or more analytes.

In one example, NAD, NADH, NAD+, NAD(P)+, ATP, flavin adenine dinucleotide (FAD), magnesium (Mg++), pyrroloquinoline quinone (PQQ), and functionalized derivatives thereof can be used in combination with one or more enzymes in the continuous multi-analyte sensor device. In one example, NAD, NADH, NAD+, NAD(P)+, ATP, flavin adenine dinucleotide (FAD), magnesium (Mg++), pyrroloquinoline quinone (PQQ), and functionalized derivatives are incorporated in the sensing region. In one example, NAD, NADH, NAD+, NAD(P)+, ATP, flavin adenine dinucleotide (FAD), magnesium (Mg++), pyrroloquinoline quinone (PQQ), and functionalized derivatives are dispersed or distributed in one or more membranes or domains of the sensing region.

In one aspect of the present disclosure, continuous sensing of one or more or two or more analytes using NAD+ dependent enzymes is provided in one or more membranes or domains of the sensing region. In one example, the membrane or domain provides retention and stable recycling of NAD+ as well as mechanisms for transducing NADH oxidation or NAD+ reduction into measurable current with amperometry. In one example, described below, continuous, sensing of multi-analytes, either reversibly bound or at least one of which are oxidized or reduced by NAD+ dependent enzymes, for example, ketones (beta-hydroxybutyrate dehydrogenase), glycerol (glycerol dehydrogenase), cortisol (11β-hydroxysteroid dehydrogenase), glucose (glucose dehydrogenase), alcohol (alcohol dehydrogenase), aldehydes (aldehyde dehydrogenase), and lactate (lactate dehydrogenase) is provided. In other examples, described below, membranes are provided that enable the continuous, on-body sensing of multiple analytes which utilize FAD-dependent dehydrogenases, such as fatty acids (Acyl-CoA dehydrogenase).

Exemplary configurations of one or more membranes or portions thereof are an arrangement for providing retention and recycling of NAD+ are provided. Thus, an electrode surface of a conductive wire (coaxial) or a planar conductive surface is coated with at least one layer comprising at least one enzyme as depicted in FIG. 15A. With reference to FIG. 15B, one or more optional layers may be positioned between the electrode surface and the one or more enzyme domains. For example, one or more interference domains (also referred to as “interferent blocking layer”) can be used to reduce or eliminate signal contribution from undesirable species present, or one or more electrodes (not shown) can used to assist with wetting, system equilibrium, and/or start up. As shown in FIGS. 15A-15B, one or more of the membranes provides a NAD+ reservoir domain providing a reservoir for NAD+. In one example, one or more interferent blocking membranes is used, and potentiostat is utilized to measure H₂O₂ production or O2 consumption of an enzyme such as or similar to NADH oxidase, the NAD+ reservoir and enzyme domain positions can be switched, to facilitate better consumption and slower unnecessary outward diffusion of excess NAD+. Exemplary sensor configurations can be found in U.S. Provisional Patent Application No. 63/321,340, “CONTINUOUS ANALYTE MONITORING SENSOR SYSTEMS AND METHODS OF USING THE SAME,” filed Mar. 18, 2022, and incorporated by reference in its entirety herein.

In one example, one or more mediators that are optimal for NADH oxidation are incorporated in the one or more electrode domains or enzyme domains. In one example, organic mediators, such as phenanthroline dione, or nitrosoanilines are used. In another example, metallo-organic mediators, such as ruthenium-phenanthroline-dione or osmium(bpy)₂Cl, polymers containing covalently coupled organic mediators or organometallic coordinated mediators polymers for example polyvinylimidizole-Os(bpy)₂Cl, or polyvinylpyridine-organometallic coordinated mediators (including ruthenium-phenanthroline dione) are used. Other mediators can be used as discussed further below.

In humans, serum levels of beta-hydroxybutyrate (BHB) are usually in the low micromolar range but can rise up to about 6-8 mM. Serum levels of BHB can reach 1-2 mM after intense exercise or consistent levels above 2 mM are reached with a ketogenic diet that is almost devoid of carbohydrates. Other ketones are present in serum, such as acetoacetate and acetone, however, most of the dynamic range in ketone levels is in the form of BHB. Thus, monitoring of BHB, e.g., continuous monitoring is useful for providing health information to a user or health care provider.

Another example of a continuous ketone analyte detection configuration employing electrode-associated mediator-coupled diaphorase/NAD+/dehydrogenase is depicted below:

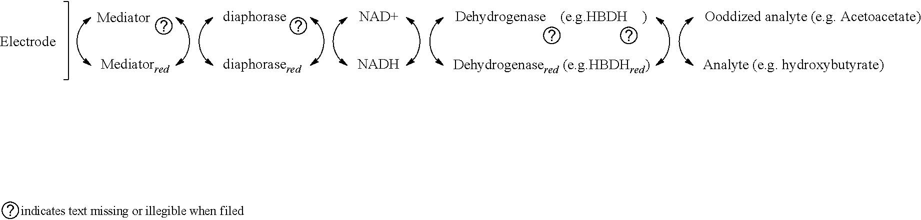

In one example, the diaphorase is electrically coupled to the electrode with organometallic coordinated mediator polymer. In another example, the diaphorase is covalently coupled to the electrode with an organometallic coordinated mediator polymer. Alternatively, multiple enzyme domains can be used in an enzyme layer, for example, separating the electrode-associated diaphorase (closest to the electrode surface) from the more distal adjacent NAD+ or the dehydrogenase enzyme, to essentially decouple NADH oxidation from analyte (ketone) oxidation. Alternatively, NAD+ can be more proximal to the electrode surface than an adjacent enzyme domain comprising the dehydrogenase enzyme. In one example, the NAD+ and/or HBDH are present in the same or different enzyme domain, and either can be immobilized, for example, using amine reactive crosslinker (e.g., glutaraldehyde, epoxides, NHS esters, imidoesters). In one example, the NAD+ is coupled to a polymer and is present in the same or different enzyme domain as HBDH. In one example, the molecular weight of NAD+ is increased to prevent or eliminate migration from the sensing region, for example the NAD+ is dimerized using its C6 terminal amine with any amine-reactive crosslinker. In one example, NAD+ may be covalently coupled to an aspect of the enzyme domain having a higher molecular weight than the NAD+ which may improve a stability profile of the NAD+, improving the ability to retain and/or immobilize the NAD+ in the enzyme domain. For example, dextran-NAD.

In one example, the sensing region comprises one or more NADH:acceptor oxidoreductases and one or more NAD-dependent dehydrogenases. In one example, sensing region comprises one or more NADH:acceptor oxidoreductases and one or more NAD(P)-dependent dehydrogenases with NAD(P)+ or NAD(P)H as cofactors present in sensing region. In one example, the sensing region comprises an amount of diaphorase.

In one example, a ketone sensing configuration suitable for combination with another analyte sensing configuration is provided. Thus, an EZL layer of about 1-20 μm thick is prepared by presenting a EZL solution composition in 10 mM HEPES in water having about 20 μL 500 mg/mL HBDH, about 20 μL [500 mg/mL NAD(P)H, 200 mg/mL polyethylene glycol-diglycol ether (PEG-DGE) of about 400 MW], about 20 μL 500 mg/mL diaphorase, about 40 μL 250 mg/mL poly vinyl imidazole-osmium bis(2,2′-bipyridine)chloride (PVI-Os(bpy)2Cl) to a substrate such as a working electrode, so as to provide, after drying, about 15-40% by weight HBDH, about 5-30% diaphorase about 5-30% NAD(P)H, about 10-50% PVI-Os(bpy)₂Cl and about 1-12% PEG-DGE(400 MW). The substrates discussed herein that may include working electrodes may be formed from gold, platinum, palladium, rhodium, iridium, titanium, tantalum, chromium, and/or alloys or combinations thereof, or carbon (e.g., graphite, glassy carbon, carbon nanotubes, graphene, or doped diamond, as well combinations thereof.

To the above enzyme domain was contacted a resistance domain, also referred to as a resistance layer (“RL”). In one example, the RL comprises about 55-100% PVP, and about 0.1-45% PEG-DGE. In another example, the RL comprises about 75-100% PVP, and about 0.3-25% PEG-DGE. In yet another example, the RL comprises about 85-100% PVP, and about 0.5-15% PEG-DGE. In yet another example, the RL comprises essentially 100% PVP.

The exemplary continuous ketone sensor as depicted in FIGS. 15A-15B comprising NAD(P)H reservoir domain is configured so that NAD(P)H is not rate-limiting in any of the enzyme domains of the sensing region. In one example, the loading of NAD(P)H in the NAD(P)H reservoir domain is greater than about 20%, 30%, 40% or 50% w/w. The one or more of the membranes or portions of one or more membrane domains (hereinafter also referred to as “membranes”) may also contain a polymer or protein binder, such as zwitterionic polyurethane, and/or albumin. Alternatively, in addition to NAD(P)H, the membrane may contain one or more analyte specific enzymes (e.g. HBDH, glycerol dehydrogenase, etc.), so that optionally, the NAD(P)H reservoir membrane also provides a catalytic function. In one example, the NAD(P)H is dispersed or distributed in or with a polymer(or protein), and may be crosslinked to an extent that still allows adequate enzyme/cofactor functionality and/or reduced NAD(P)H flux within the domain.

In one example, NADH oxidase enzyme alone or in combination with superoxide dismutase (SOD) is used in the one or more membranes of the sensing region. In one example, an amount of superoxide dismutase (SOD) is used that is capable of scavenging some or most of one or more free radicals generated by NADH oxidase. In one example, NADH oxidase enzyme alone or in combination with superoxide dismutase (SOD) is used in combination with NAD(P)H and/or a functionalized polymer with NAD(P)H immobilized onto the polymer from a C6 terminal amine in the one or more membranes of the sensing region.

In one example, the NAD(P)H is immobilized to an extent that maintains NAD(P)H catalytic functionality. In one example, dimerized NAD(P)H is used to entrap NAD(P)H within one or more membranes by crosslinking their respective C6 terminal amine together with appropriate amine-reactive crosslinker such as glutaraldehyde or PEG-DGE.

The aforementioned continuous ketone sensor configurations can be adapted to other analytes or used in combination with other sensor configurations. For example, analyte(s)-dehydrogenase enzyme combinations can be used in any of the membranes of the sensing region include; glycerol (glycerol dehydrogenase); cortisol (11β-hydroxysteroid dehydrogenase); glucose (glucose dehydrogenase); alcohol (alcohol dehydrogenase); aldehydes (aldehyde dehydrogenase); and lactate (lactate dehydrogenase).

In one example, a semipermeable membrane is used in the sensing region or adjacent thereto or adjacent to one or more membranes of the sensing region so as to attenuate the flux of at least one analyte or chemical species. In one example, the semipermeable membrane attenuates the flux of at least one analyte or chemical species so as to provide a linear response from a transduced signal. In another example, the semipermeable membrane prevents or eliminates the flux of NAD(P)H out of the sensing region or any membrane or domain. In one example, the semipermeable membrane can be an ion selective membrane selective for an ion analyte of interest, such as ammonium ion.

In another example, a continuous multi-analyte sensor configuration comprising one or more enzymes and/or at least one cofactor was prepared. FIG. 15C depicts this exemplary configuration, of an enzyme domain 1550 comprising an enzyme (Enzyme) with an amount of cofactor (Cofactor) that is positioned proximal to at least a portion of a working electrode (“WE”) surface, where the WE comprises an electrochemically reactive surface. In one example, a second membrane 1551 comprising an amount of cofactor is positioned adjacent the first enzyme domain. The amount of cofactor in the second membrane can provide an excess for the enzyme, e.g., to extend sensor life. One or more resistance domains 1552 (“RL”) are positioned adjacent the second membrane (or can be between the membranes). The RL can be configured to block diffusion of cofactor from the second membrane. Electron transfer from the cofactor to the WE transduces a signal that corresponds directly or indirectly to an analyte concentration.

FIG. 15D depicts an alternative enzyme domain configuration comprising a first membrane 1551 with an amount of cofactor that is positioned more proximal to at least a portion of a WE surface. Enzyme domain 1550 comprising an amount of enzyme is positioned adjacent the first membrane.

In the membrane configurations depicted in FIGS. 15C-15D, production of an electrochemically active species in the enzyme domain diffuses to the WE surface and transduces a signal that corresponds directly or indirectly to an analyte concentration. In some examples, the electrochemically active species comprises hydrogen peroxide. For sensor configurations that include a cofactor, the cofactor from the first layer can diffuse to the enzyme domain to extend sensor life, for example, by regenerating the cofactor. For other sensor configurations, the cofactor can be optionally included to improve performance attributes, such as stability. For example, a continuous ketone sensor can comprise NAD(P)H and a divalent metal cation, such as Mg⁺². One or more resistance domains RL can be positioned adjacent the second membrane (or can be between the layers). The RL can be configured to block diffusion of cofactor from the second membrane and/or interferents from reaching the WE surface. Other configurations can be used in the aforementioned configuration, such as electrode, resistance, bio-interfacing, and drug releasing membranes, layers or domains. In other examples, continuous analyte sensors including one or more cofactors that contribute to sensor performance.

FIG. 15E depicts another continuous multi-analyte membrane configuration, where {beta}-hydroxybutyrate dehydrogenase BHBDH in a first enzyme domain 1553 is positioned proximate to a working electrode WE and second enzyme domain 1554, for example, comprising alcohol dehydrogenase (ADH) and NADH is positioned adjacent the first enzyme domain. One or more resistance domains RL 1552 may be deployed adjacent to the second enzyme domain 1554. In this configuration, the presence of the combination of alcohol and ketone in serum works collectively to provide a transduced signal corresponding to at least one of the analyte concentrations, for example, ketone. Thus, as the NADH present in the more distal second enzyme domain consumes alcohol present in the serum environment, NADH is oxidized to NAD(P)H that diffuses into the first membrane layer to provide electron transfer of the BHBDH catalysis of acetoacetate ketone and transduction of a detectable signal corresponding to the concentration of the ketone. In one example, an enzyme can be configured for reverse catalysis and can create a substrate used for catalysis of another enzyme present, either in the same or different layer or domain. Other configurations can be used in the aforementioned configuration, such as electrode, resistance, bio-interfacing, and drug releasing membranes, layers, or domains. Thus, a first enzyme domain that is more distal from the WE than a second enzyme domain may be configured to generate a cofactor or other element to act as a reactant (and/or a reactant substrate) for the second enzyme domain to detect the one or more target analytes.

### Alcohol Sensor Configurations

In one example, a continuous alcohol (e.g., ethanol) sensor device configuration is provided. In one example, one or more enzyme domains comprising alcohol oxidase (AOX) is provided and the presence and/or amount of alcohol is transduced by creation of hydrogen peroxide, alone or in combination with oxygen consumption or with another substrate-oxidase enzyme system, e.g., glucose-glucose oxidase, in which hydrogen peroxide and or oxygen and/or glucose can be detected and/or measured qualitatively or quantitatively, using amperometry.

In one example, the sensing region for the aforementioned enzyme substrate-oxidase enzyme configurations has one or more enzyme domains comprises one or more electrodes. In one example, the sensing region for the aforementioned enzyme substrate-oxidase enzyme configurations has one or more enzyme domains, with or without the one or more electrodes, further comprises one or interference blocking membranes (e.g. permselective membranes, charge exclusion membranes) to attenuate one or more interferents from diffusing through the membrane to the working electrode. In one example, the sensing region for the aforementioned substrate-oxidase enzyme configurations has one or more enzyme domains, with or without the one or more electrodes, and further comprises one or resistance domains with or without the one or more interference blocking membranes to attenuate one or more analytes or enzyme substrates. In one example, the sensing region for the aforementioned substrate-oxidase enzyme configurations has one or more enzyme domains, with or without the one or more electrodes, one or more resistance domains with or without the one or more interference blocking membranes further comprises one or biointerface membranes and/or drug releasing membranes, independently, to attenuate one or more analytes or enzyme substrates and attenuate the immune response of the host after insertion.

In one example, the one or more interference blocking membranes are deposited adjacent the working electrode and/or the electrode surface. In one example, the one or interference blocking membranes are directly deposited adjacent the working electrode and/or the electrode surface. In one example, the one or interference blocking membranes are deposited between another layer or membrane or domain that is adjacent the working electrode or the electrode surface to attenuate one or all analytes diffusing thru the sensing region but for oxygen. Such membranes can be used to attenuate alcohol itself as well as attenuate other electrochemically actives species or other analytes that can otherwise interfere by producing a signal if they diffuse to the working electrode.

In one example, the working electrode used comprised platinum and the potential applied was about 0.5 volts.

In one example, sensing oxygen level changes electrochemically, for example in a Clark type electrode setup, or in a different configuration can be carried out, for example by coating the electrode with one or more membranes of one or more polymers, such as NAFION™. Based on changes of potential, oxygen concentration changes can be recorded, which correlate directly or indirectly with the concentrations of alcohol. When appropriately designed to obey stoichiometric behavior, the presence of a specific concentration of alcohol should cause a commensurate reduction in local oxygen in a direct (linear) relation with the concentration of alcohol. Accordingly, a multi-analyte sensor for both alcohol and oxygen can therefore be provided.

In another example, the above mentioned alcohol sensing configuration can include one or more secondary enzymes that react with a reaction product of the alcohol/alcohol oxidase catalysis, e.g., hydrogen peroxide, and provide for a oxidized form of the secondary enzyme that transduces an alcohol-dependent signal to the WE/RE at a lower potential than without the secondary enzyme. Thus, in one example, the alcohol/alcohol oxidase is used with a reduced form of a peroxidase, for example horse radish peroxidase. The alcohol/alcohol oxidase can be in same or different layer as the peroxidase, or they may be spatially separated distally from the electrode surface, for example, the alcohol/alcohol oxidase being more distal from the electrode surface and the peroxidase being more proximal to the electrode surface, or alternatively, the alcohol/alcohol oxidase being more proximal from the electrode surface and the peroxidase being more distal to the electrode surface. In one example, the alcohol/alcohol oxidase, being more distal from the electrode surface and the peroxidase, further includes any combination of electrode, interference, resistance, and biointerface membranes to optimize signal, durability, reduce drift, or extend end of use duration.

In another example, the above mentioned alcohol sensing configuration can include one or more mediators. In one example, the one or more mediators are present in, on, or about one or more electrodes or electrode surfaces and/or are deposited or otherwise associated with the surface of the working electrode (WE) or reference electrode (RE). In one example, the one or more mediators eliminate or reduce direct oxidation of interfering species that may reach the WE or RE. In one example, the one or more mediators provide a lowering of the operating potential of the WE/RE, for example, from about 0.6V to about 0.3V or less on a platinum electrode, which can reduce or eliminates oxidation of endogenous interfering species. Examples of one or mediators are provided below. Other electrodes, e.g., counter electrodes, can be employed.

In one example, other enzymes or additional components may be added to the polymer mixture(s) that constitute any part of the sensing region to increase the stability of the aforementioned sensor and/or reduce or eliminate the biproducts of the alcohol/alcohol oxidase reaction. Increasing stability includes storage or shelf life and/or operational stability (e.g., retention of enzyme activity during use). For example, byproducts of enzyme reactions may be undesirable for increased shelf life and/or operational stability, and may thus be desirable to reduce or remove. In one example, xanthine oxidase can be used to remove bi-products of one or more enzyme reactions.

In another example, a dehydrogenase enzyme is used with a oxidase for the detection of alcohol alone or in combination with oxygen. Thus, in one example, alcohol dehydrogenase is used to oxidize alcohol to aldehyde in the presence of reduced nicotinamide adenine dinucleotide (NAD(P)H) or reduced nicotinamide adenine dinucleotide phosphate (NAD(P)+). So as to provide a continuous source of NAD(P)H or NAD(P)+, NADH oxidase or NADPH oxidases is used to oxidize the NAD(P)H or NAD(P)+, with the consumption of oxygen. In another example, Diaphorase can be used instead of or in combination with NADH oxidase or NADPH oxidases. Alternatively, an excess amount of NAD(P)H can be incorporated into the one or more enzyme domains and/or the one or more electrodes in an amount so as to accommodate the intended duration of planned life of the sensor.

In the aforementioned dual enzyme configuration, a signal can be sensed either by: (1) an electrically coupled (e.g., “wired”) alcohol dehydrogenase (ADH), for example, using an electro-active hydrogel polymer comprising one or more mediators; or (2) oxygen electrochemical sensing to measure the oxygen consumption of the NADH oxidase. In an alternative example, the co-factor NAD(P)H or NAD(P)+ may be coupled to a polymer, such as dextran, the polymer immobilized in the enzyme domain along with ADH. This provides for retention of the co-factor and availability thereof for the active site of ADH. In the above example, any combination of electrode, interference, resistance, and biointerface membranes can be used to optimize signal, durability, reduce drift, or extend end of use duration. In one example, electrical coupling, for example, directly or indirectly, via a covalent or ionic bond, to at least a portion of a transducing element, such as an aptamer, an enzyme or cofactor and at least a portion of the electrode surface is provided. A chemical moiety capable of assisting with electron transfer from the enzyme or cofactor to the electrode surface can be used and includes one or more mediators as described below.

In one example, any one of the aforementioned continuous alcohol sensor configurations are combined with any one of the aforementioned continuous ketone monitoring configurations to provide a continuous multi-analyte sensor device as further described below. In one example a continuous glucose monitoring configuration combined with any one of the aforementioned continuous alcohol sensor configurations and any one of the aforementioned continuous ketone monitoring configurations to provide a continuous multi-analyte sensor device as further described below.

### Uric Acid Sensor Configurations

In another example, a continuous uric acid sensor device configuration is provided. Thus, in one example, uric acid oxidase (UOX) can be included in one or more enzyme domains and positioned adjacent the working electrode surface. The catalysis of the uric acid using UOX, produces hydrogen peroxide which can be detected using, among other techniques, amperometry, voltametric and impedimetric methods. In one example, to reduce or eliminate the interference from direct oxidation of uric acid on the electrode surface, one or more electrode, interference, and/or resistance domains can be deposited on at least a portion of the working electrode surface. Such membranes can be used to attenuate diffusion of uric acid as well as other analytes to the working electrode that can interfere with signal transduction.

In one alternative example, a uric acid continuous sensing device configuration comprises sensing oxygen level changes about the WE surface, e.g., for example, as in a Clark type electrode setup, or the one or more electrodes can comprise, independently, one or more different polymers such as NAFION™, polyzwitterion polymers, or polymeric mediator adjacent at least a portion of the electrode surface. In one example, the electrode surface with the one or more electrode domains provide for operation at a different or lower voltage to measure oxygen. Oxygen level and its changes in can be sensed, recorded, and correlated to the concentration of uric acid based using, for example, using conventional calibration methods.

In one example, alone or in combination with any of the aforementioned configurations, uric acid sensor configurations, so as to lower the potential at the WE for signal transduction of uric acid, one or more coatings can be deposited on the WE surface. The one or more coatings may be deposited or otherwise formed on the WE surface and/or on other coatings formed thereon using various techniques including, but not limited to, dipping, electrodepositing, vapor deposition, spray coating, etc. In one example, the coated WE surface can provide for redox reactions, e.g., of hydrogen peroxide, at lower potentials (as compared to 0.6 V on platinum electrode surface without such a coating. Example of materials that can be coated or annealed onto the WE surface includes, but are not limited to Prussian Blue, Medola Blue, methylene blue, methylene green, methyl viologen, ferrocyanide, ferrocene, cobalt ion, and cobalt phthalocyanine, and the like.

In one example, one or more secondary enzymes, cofactors and/or mediators (electrically coupled or polymeric mediators) can be added to the enzyme domain with UOX to facilitate direct or indirect electron transfer to the WE. In such configurations, for example, regeneration of the initial oxidized form of secondary enzyme is reduced by the WE for signal transduction. In one example, the secondary enzyme is horse radish peroxidase (HRP).

### Choline Sensor Configurations

In one example continuous choline sensor device can be provided, for example, using choline oxidase enzyme that generates hydrogen peroxide with the oxidation of choline. Thus, in one example, at least one enzyme domain comprises choline oxidase (COX) adjacent at least one WE surface, optionally with one or more electrodes and/or interference membranes positioned in between the WE surface and the at least one enzyme domain. The catalysis of the choline using COX results in creation of hydrogen peroxide which can be detectable using, among other techniques, amperometry, voltametric and impedimetric methods.

In one example, the aforementioned continuous choline sensor configuration is combined with any one of the aforementioned continuous alcohol sensor configurations, and continuous uric acid sensor configurations to provide a continuous multi-analyte sensor device as further described below. This continuous multi-analyte sensor device can further include continuous glucose monitoring capability. Other membranes can be used in the aforementioned continuous choline sensor configuration, such as electrode, resistance, bio-interfacing, and drug releasing membranes.

### Cholesterol Sensor Configurations

In one example, continuous cholesterol sensor configurations can be made using cholesterol oxidase (CHOX), in a manner similar to previously described sensors. Thus, one or more enzyme domains comprising CHOX can be positioned adjacent at least one WE surface. The catalysis of free cholesterol using CHOX results in creation of hydrogen peroxide which can be detectable using, among other techniques, amperometry, voltametric and impedimetric methods.

An exemplary cholesterol sensor configuration using a platinum WE, where at least one interference membrane is positioned adjacent at least one WE surface, over which there is at least one enzyme domain comprising CHOX, over which is positioned at least one resistance domain to control diffusional characteristics was prepared.

The method described above and the cholesterol sensors described can measure free cholesterol, however, with modification, the configuration can measure more types of cholesterol as well as total cholesterol concentration. Measuring different types of cholesterol and total cholesterol is important, since due to low solubility of cholesterol in water significant amount of cholesterol is in unmodified and esterified forms. Thus, in one example, a total cholesterol sample is provided where a secondary enzyme is introduced into the at least one enzyme domain, for example, to provide the combination of cholesterol esterase with CHOX Cholesteryl ester, which essentially represents total cholesterols can be measured indirectly from signals transduced from cholesterol present and formed by the esterase.

In one example, the aforementioned continuous (total) cholesterol sensor configuration is combined with any one of the aforementioned continuous alcohol sensor configurations and/or continuous uric acid sensor configurations to provide a continuous multi-analyte sensor system as further described below. This continuous multi-analyte sensor device can further include continuous glucose monitoring capability. Other membrane configurations can be used in the aforementioned continuous cholesterol sensor configuration, such as one or more electrode domains, resistance domains, bio-interfacing domains, and drug releasing membranes.

### Bilirubin Sensor and Ascorbic Acid Sensor Configurations

In one example, continuous bilirubin and ascorbic acid sensors are provided. These sensors can employ bilirubin oxidase and ascorbate oxidase, respectively. However, unlike some oxidoreductase enzymes, the final product of the catalysis of analytes of bilirubin oxidase and ascorbate oxidase is water instead of hydrogen peroxide. Therefore, redox detection of hydrogen peroxide to correlate with bilirubin or ascorbic acid is not possible. However, these oxidase enzymes still consume oxygen for the catalysis, and the levels of oxygen consumption correlates with the levels of the target analyte present. Thus, bilirubin and ascorbic acid levels can be measured indirectly by electrochemically sensing oxygen level changes, as in a Clark type electrode setup, for example.

Alternatively, a different configuration for sensing bilirubin and ascorbic acid can be employed. For example, an electrode domain including one or more electrode domains comprising electron transfer agents, such as NAFION™, polyzwitterion polymers, or polymeric mediator can be coated on the electrode. Measured oxygen levels transduced from such enzyme domain configurations can be correlated with the concentrations of bilirubin and ascorbic acid levels. In one example, an electrode domain comprising one or more mediators electrically coupled to a working electrode can be employed and correlated to the levels of bilirubin and ascorbic acid levels.

In one example, the aforementioned continuous bilirubin and ascorbic acid sensor configurations can be combined with any one of the aforementioned continuous alcohol sensor configurations, continuous uric acid sensor configurations, continuous cholesterol sensor configurations to provide a continuous multi-analyte sensor device as further described below. This continuous multi-analyte sensor device can further include continuous glucose monitoring capability. Other membranes can be used in the aforementioned continuous bilirubin and ascorbic acid sensor configuration, such as electrode, resistance, bio-interfacing, and drug releasing membranes.

### One-Working-Electrode Configurations for Dual Analyte Detection

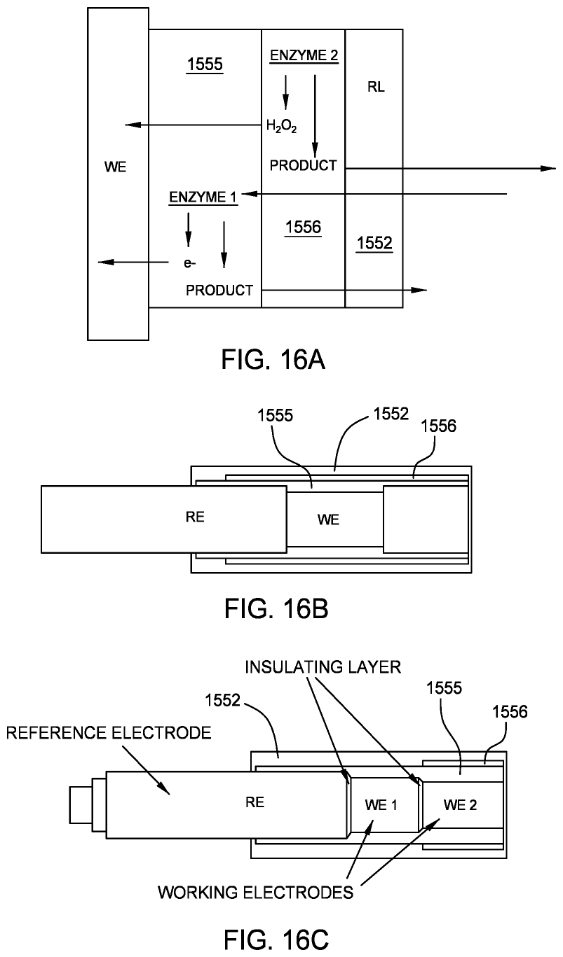

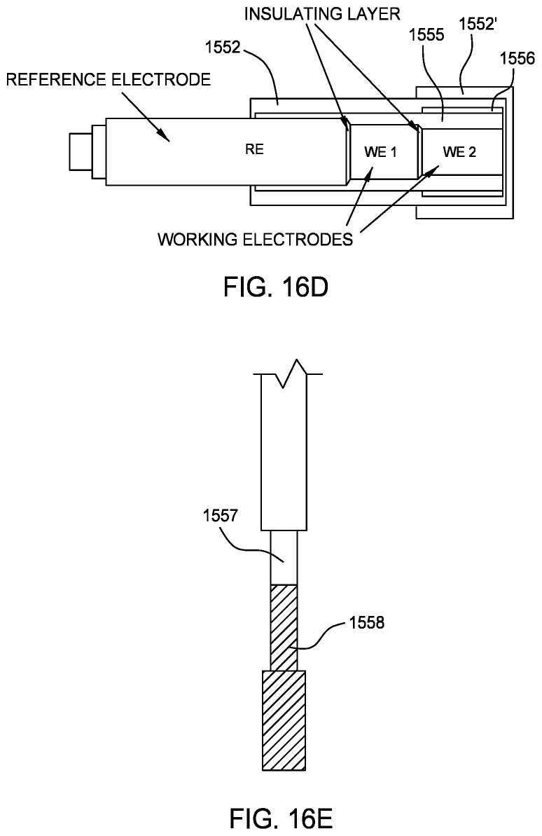

In one example, at least a dual enzyme domain configuration in which each layer contains one or more specific enzymes and optionally one or more cofactors is provided. In a broad sense, one example of a continuous multi-analyte sensor configuration is depicted in FIG. 16A where a first membrane 1555 (EZL1) comprising at least one enzyme (Enzyme 1) of the at least two enzyme domain configuration is proximal to at least one surface of a WE. One or more analyte-substrate enzyme pairs with Enzyme 1 transduces at least one detectable signal to the WE surface by direct electron transfer or by mediated electron transfer that corresponds directly or indirectly to an analyte concentration. Second membrane 1556 (EZL2) with at least one second enzyme (Enzyme 2) is positioned adjacent 1555 ELZ1, and is generally more distal from WE than EZL1. One or more resistance domains (RL) 1552 can be provided adjacent EZL2 1556, and/or between EZL1 1555 and EZL2 1556. The different enzymes catalyze the transformation of the same analyte, but at least one enzyme in EZL2 1556 provides hydrogen peroxide and the other at least one enzyme in EZL1 1555 does not provide hydrogen peroxide. Accordingly, each measurable species (e.g., hydrogen peroxide and the other measurable species that is not hydrogen peroxide) generates a signal associated with its concentration.

For example, in the configuration shown in FIG. 16A, a first analyte diffuses through RL 1552 and into EZL2 1556 resulting in peroxide via interaction with Enzyme 2. Peroxide diffuses at least through EZL1 1555 to WE and transduces a signal that corresponds directly or indirectly to the first analyte concentration. A second analyte, which is different from the first analyte, diffuses through RL 1552 and EZL2 1556 and interacts with Enzyme 1, which results in electron transfer to WE and transduces a signal that corresponds directly or indirectly to the second analyte concentration.

As shown in FIG. 16B, the above configuration is adapted to a conductive wire electrode construct, where at least two different enzyme-containing layers are constructed on the same WE with a single active surface. In one example, the single WE is a wire, with the active surface positioned about the longitudinal axis of the wire. In another example, the single WE is a conductive trace on a substrate, with the active surface positioned about the longitudinal axis of the trace. In one example, the active surface is substantially continuous about a longitudinal axis or a radius.

In the configuration described above, at least two different enzymes can be used and catalyze the transformation of different analytes, with at least one enzyme in EZL2 1556 providing hydrogen peroxide and the at least other enzyme in EZL1 1555 not providing hydrogen peroxide, e.g., providing electron transfer to the WE surface corresponding directly or indirectly to a concentration of the analyte.

In one example, an inner layer of the at least two enzyme domains EZL1, EZL2 1555, 1556 comprises at least one immobilized enzyme in combination with at least one mediator that can facilitate lower bias voltage operation of the WE than without the mediator. In one example, for such direct electron transductions, a potential P1 is used. In one example, at least a portion of the inner layer EZL1 1555 is more proximal to the WE surface and may have one or more intervening electrode domains and/or overlaying interference and/or bio-interfacing and/or drug releasing membranes, provided that the at least one mediator can facilitate low bias voltage operation with the WE surface. In another example, at least a portion of the inner layer EZL1 1555 is directly adjacent the WE.

The second layer of at least dual enzyme domain (the outer layer EZL2 1556) of FIG. 16B contains at least one enzyme that result in one or more catalysis reactions that eventually generate an amount of hydrogen peroxide that can electrochemically transduce a signal corresponding to the concentration of the analyte(s). In one example, the generated hydrogen peroxide diffuses through layer EZL2 1556 and through the inner layer EZL1 1555 to reach the WE surface and undergoes redox at a potential of P2, where P2≠P1. In this way electron transfer and electrolysis (redox) can be selectively controlled by controlling the potentials P1, P2 applied at the same WE surface. Any applied potential durations can be used for P1, P2, for example, equal/periodic durations, staggered durations, random durations, as well as various potentiometric sequences, cyclic voltammetry etc. In some examples, impedimetric sensing may be used. In one example, a phase shift (e.g., a time lag) may result from detecting two signals from two different working electrodes, each signal being generated by a different EZL (EZL1, EZL2, 1555, 1556) associated with each electrode. The two (or more) signals can be broken down into components to detect the individual signal and signal artifacts generated by each of EZL1 1555 and EZL2 1556 in response to the detection of two analytes. In some examples, each EZL detects a different analyte. In other examples, both EZLs detect the same analyte.

In another alternative exemplary configuration, as shown in FIGS. 16C-16D a multienzyme domain configuration as described above is provided for a continuous multi-analyte sensor device using a single WE with two or more active surfaces is provided. In one example, the multienzyme domain configurations discussed herein are formed on a planar substrate. In another example, the single WE is coaxial, e.g., configured as a wire, having two or more active surfaces positioned about the longitudinal axis of the wire. Additional wires can be used, for example, as a reference and/or counter electrode. In another example, the single WE is a conductive trace on a substrate, with two or more active surfaces positioned about the longitudinal axis of the trace. At least a portion of the two or more active surfaces are discontinuous, providing for at least two physically separated WE surfaces on the same WE wire or trace. (e.g., WE1, WE2). In one example, the first analyte detected by WE1 is glucose, and the second analyte detected by WE2 is lactate. In another example, the first analyte detected by WE1 is glucose, and the second analyte detected by WE2 is ketones.

Thus, FIGS. 16C-16D depict exemplary configurations of a continuous multi-analyte sensor construct in which EZL1 1555, EZL2 1556 and RL 1552 (resistance domain) as described above, arranged, for example, by sequential dip coating techniques, over a single coaxial wire comprising spatially separated electrode surfaces WE1, WE2. One or more parameters, independently, of the enzyme domains, resistance domains, etc., can be controlled along the longitudinal axis of the WE, for example, thickness, length along the axis from the distal end of the wire, etc. In one example, at least a portion of the spatially separated electrode surfaces are of the same composition. In another example, at least a portion of the spatially separated electrode surfaces are of different composition. In FIGS. 16C-16D, WE1 represents a first working electrode surface configured to operate at P1, for example, and is electrically insulated from second working electrode surface WE2 that is configured to operate at P2, and RE represents a reference electrode RE electrically isolated from both WE1, WE2. One resistance domain is provided in the configuration of FIG. 16C that covers the reference electrode and WE1, WE2. An addition resistance domain is provided in the configuration of FIG. 16D that covers extends over essentially WE2 only. Additional electrodes, such as a counter electrode can be used. Such configurations (whether single wire or dual wire configurations) can also be used to measure the same analyte using two different techniques. Using different signal generating sequences as well as different RLs, the data collected from two different mode of measurements provides increase fidelity, improved performance and device longevity. A non-limiting example is a glucose oxidase (H₂O₂ producing) and glucose dehydrogenase (electrically coupled) configuration. Measurement of Glucose at two potentials and from two different electrodes provides more data points and accuracy. Such approaches may not be needed for glucose sensing, but the can be applied across the biomarker sensing spectrum of other analytes, alone or in combination with glucoses sensing, such as ketone sensing, ketone/lactate sensing, and ketone/glucose sensing.

In an alternative configuration of that depicted in FIGS. 16C-16D, two or more wire electrodes, which can be colinear, wrapped, or otherwise juxtaposed, are presented, where WE1 is separated from WE2, for example, from other elongated shaped electrode. Insulating layer electrically isolates WE1 from WE2. In this configuration, independent electrode potential can be applied to the corresponding electrode surfaces, where the independent electrode potential can be provided simultaneously, sequentially, or randomly to WE1, WE2. In one example, electrode potentials presented to the corresponding electrode surfaces WES1, WES2, are different. One or more additional electrodes can be present such as a reference electrode and/or a counter electrode. In one example, WES2 is positioned longitudinally distal from WES1 in an elongated arrangement. Using, for example, dip coating methods, WES1 and WES2 are coated with enzyme domain EZL1, while WES2 is coated with different enzyme domain EZL2. Based on the dipping parameters, or different thickness of enzyme domains, multi-layered enzyme domains, each layer independently comprising different loads and/or compositions of enzyme and/or cofactors, mediators can be employed. Likewise, one or more resistance domains (RL) can be applied, each can be of a different thickness along the longitudinal axis of the electrode, and over different electrodes and enzyme domains by controlling dip length and other parameters, for example. With reference to FIG. 16D, such an arrangement of RL's is depicted, where an additional RL 1552′ is adjacent WES2 but substantially absent from WES1.

In one example of measuring two different analytes, the above configuration comprising enzyme domain EZL1 1555 comprising one or more enzyme(s) and one or more mediators for at least one enzyme of EZL1 to provide for direct electron transfer to the WES1 and determining a concentration of at least a first analyte. In addition, enzyme domain EZL2 1556 can comprise at least one enzyme that provides peroxide (e.g., hydrogen peroxide) or consumes oxygen during catalysis with its substrate. The peroxide or the oxygen produced in EZL2 1556 migrates to WES2 and provides a detectable signal that corresponds directly or indirectly to a second analyte. For example, WES2 can be carbon, wired to glucose dehydrogenase to measure glucose, while WES1 can be platinum, that measures peroxided produced from lactate oxidase/lactate in EZL2 1556. The combinations of electrode material and enzyme(s) as disclosed herein are examples and non-limiting.

In one example, the potentials of P1 and P2 can be separated by an amount of potential so that both signals (from direct electron transfer from EZL1 1555 and from hydrogen peroxide redox at WE) can be separately activated and measured. In one example, the electronic module of the sensor can switch between two sensing potentials continuously in a continuous or semi-continuous periodic manner, for example a period (t1) at potential P1, and period (t2) at potential P2 with optionally a rest time with no applied potential. Signal extracted can then be analyzed to measure the concentration of the two different analytes. In another example, the electronic module of the sensor can undergo cyclic voltammetry, providing changes in current when swiping over potentials of P1 and P2 can be correlated to transduced signal coming from either direct electron transfer or electrolysis of hydrogen peroxide, respectably. In one example, the modality of sensing is non-limiting and can include different amperometry techniques, e.g., cyclic voltammetry. In one example, an alternative configuration is provided but hydrogen peroxide production in EZL2 is replaced by another suitable electrolysis compound that maintains the P2≠P1 relationship, such as oxygen, and at least one enzyme-substrate combination that provide the other electrolysis compound.

For example, a continuous multi-analyte sensor configuration, for choline and glucose, in which enzyme domains EZ1 1555, EZ2 1556 were associated with different WEs, e.g., platinum WE2, and gold WE1 was prepared. In this exemplary case, EZL1 1555 contained glucose oxidase and a mediator coupled to WE1 to facilitate electron direct transfer upon catalysis of glucose, and EZL2 1556 contained choline oxidase that will catalyze choline and generate hydrogen peroxide for electrolysis at WE2. The EZL's were coated with resistance domains; upon cure and readiness they underwent cyclic voltammetry in the presence of glucose and choline. A wired glucose oxidase enzyme to a gold electrode is capable of transducing signal at 0.2 volts, therefore, by analyzing the current changes at 0.2 volts, the concentration of glucose can be determined. The data also demonstrates that choline concentration is also inferentially detectable at the WE2 platinum electrode if the CV trace is analyzed at the voltage P2.

In one example, either electrode WE1 or WE2 can be, for example, a composite material, for example a gold electrode with platinum ink deposited on top, a carbon/platinum mix, and or traces of carbon on top of platinum, or porous carbon coating on a platinum surface. In one example, with the electrode surfaces containing two distinct materials, for example, carbon used for the wired enzyme and electron transfer, while platinum can be used for hydrogen peroxide redox and detection. As shown in FIG. 16E, an example of such composite electrode surfaces is shown, in which an extended platinum covered wire 1557 is half coated with carbon 1558, to facilitate multi sensing on two different surfaces of the same electrode. In one example WE2 can be grown on or extend from a portion of the surface or distal end of WE1, for example, by vapor deposition, sputtering, or electrolytic deposition and the like.

Additional examples include a composite electrode material that may be used to form one or both of WE1 and WE2. In one example, a platinum-carbon electrode WE1, comprising EZL1 with glucose dehydrogenase is wired to the carbon surface, and outer EZL2 comprising lactate oxidase generating hydrogen peroxide that is detectable by the platinum surface of the same WE1 electrode. Other examples of this configuration can include ketone sensing (beta-hydroxybutyrate dehydrogenase electrically coupled enzyme in EZL1 1555) and glucose sensing (glucose oxidase in EZL2 1556). Other membranes can be used in the aforementioned configuration, such as electrode, resistance, bio-interfacing, and drug releasing membranes. In other examples, one or both of the working electrodes (WE1, WE2) may be gold-carbon (Au—C), palladium-carbon (Pd—C), iridium-carbon (Ir—C), rhodium-carbon (Rh—C), or ruthenium-carbon (Ru—C). In some examples, the carbon in the working electrodes discussed herein may instead or additionally include graphene, graphene oxide, or other materials suitable for forming the working electrodes, such as commercially available carbon ink.

### Glycerol Sensor Configurations

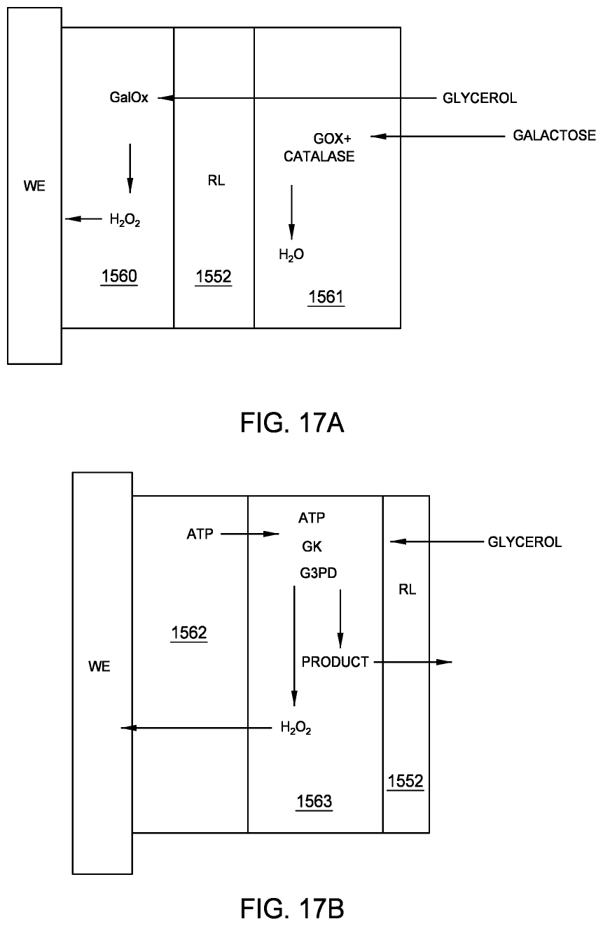

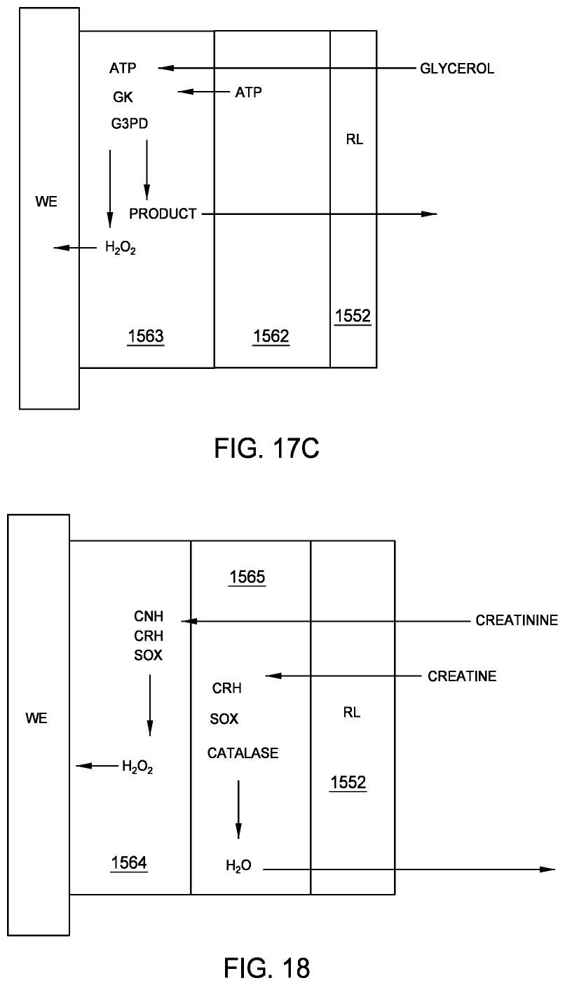

As shown in FIG. 17A, an exemplary continuous glycerol sensor configuration is depicted where a first enzyme domain EZL1 1560 comprising galactose oxidase is positioned proximal to at least a portion of a WE surface. A second enzyme domain EZL2 1561 comprising glucose oxidase and catalase is positioned more distal from the WE. As shown in FIG. 17A, one or more resistance domains (RL) 1552 are positioned between EZL1 1560 and EZL2 1561. Additional RLs can be employed, for example, adjacent to EZL2 1561. Modification of the one or more RL membranes to attenuate the flux of either analyte and increase glycerol to galactose sensitivity ratio is envisaged. The above glycerol sensing configuration provides for a glycerol sensor that can be combined with one or more additional sensor configurations as disclosed herein.

Glycerol can be catalyzed by the enzyme galactose oxidase (GalOx), however, GalOx has an activity ratio of 1%-5% towards glycerol. In one example, the activity of GalOx towards this secondary analyte glycerol can be utilized. The relative concentrations of glycerol in vivo are much higher that galactose (˜2 umol/l for galactose, and ˜100 umol/l for glycerol), which compliments the aforementioned configurations.

If the GalOx present in EZL1 1560 membrane is not otherwise functionally limited, then the GalOx will catalyze most if not all of the glycerol that passes through the one or more RLs. The signal contribution from the glycerol present will be higher as compared to the signal contribution from galactose. In one example, the one or more RL's are chemically configured to provide a higher influx of glycerol or a lower influx of galactose.

In another example, a glycol sensor configuration is provided using multiple working electrodes WEs that provides for utilizing signal transduced from both WEs. Utilizing signal transduced from both WEs can provide increasing selectivity. In one example EZL1 1560 and EZL2 1561 comprise the same oxidase enzyme (e.g., galactose oxidase) with different ratios of enzyme loading, and/or a different immobilizing polymer and/or different number and layers of RL's over the WEs. Such configurations provide for measurement of the same target analyte with different sensitivities, resulting in a dual measurement. Using a mathematical algorithm to correct for noise and interference from a first signal, and inputting the first signal from one sensing electrode with a first analyte sensitivity ratio into the mathematical algorithm, allows for the decoupling of the second signal corresponding to the desired analyte contributions. Modification of the sensitivity ratio of the one or more EZL's to distinguish signals from the interfering species and the analyte(s) of interest can be provided by adjusting one or more of enzyme source, enzyme load in EZL's, chemical nature/diffusional characteristics of EZL's, chemical/diffusional characteristics of the at least one RL's, and combinations thereof.

As discussed herein, a secondary enzyme domain can be utilized to catalyze the non-target analyte(s), reducing their concentration and limiting diffusion towards the sensing electrode through adjacent membranes that contains the primary enzyme and necessary additives. In this example, the most distal enzyme domain, EZL2, 1561 is configured to catalyze a non-target analyte that would otherwise react with EZL1, thus providing a potentially less accurate reading of the target analyte (glycerol) concentration. This secondary enzyme domain can act as a “selective diffusion exclusion membrane” by itself, or in some other configurations can be placed above or under a resistant layer (RL) 1552. In this example, the target analyte is glycerol and GalOX is used to catalyze glycerol to form a measurable species (e.g., hydrogen peroxide).

In one example, a continuous glycerol sensor configuration is provided using at least glycerol oxidase, which provides hydrogen peroxide upon reaction and catalysis of glycerol. Thus, in one example, enzyme domain comprising glycerol oxidase can be positioned adjacent at least a portion of a WE surface and hydrogen peroxide is detected using amperometry. In another example, enzyme domain comprising glycerol oxidase is used for sensing oxygen level changes, for example, in a Clark type electrode setup. Alternatively, at least a portion of the WE surface can be coated with one more layers of electrically coupled polymers, such as a mediator system discussed below, to provide a coated WE capable of electron transfer from the enzyme at a lower potential. The coated WE can then operate at a different and lower voltage to measure oxygen and its correlation to glycerol concentration.

In another example, a glycerol sensor configuration is provided using glycerol-3-phosphate oxidase in the enzyme domain. In one example, ATP is used as the cofactor. Thus, as shown in FIGS. 17B and 17C, exemplary sensor configurations are depicted where in one example (FIG. 17B), one or more cofactors (e.g. ATP) 1562 is proximal to at least a portion of an WE surface. One or more enzyme domains 1563 comprising glycerol-3-phospohate oxidase (G3PD), lipase, and/or glycerol kinase (GK) and one or more regenerating enzymes capable of continuously regenerating the cofactor are contained in an enzyme domain are adjacent the cofactor, or more distal from the WE surface than the cofactor layer 1562. Examples of regenerating enzymes that can be used to provide ATP regeneration include, but are not limited to, ATP synthase, pyruvate kinase, acetate kinase, and creatine kinase. The one or more regenerating enzymes can be included in one or more enzyme domains, or in a separate layer.

An alternative configuration is shown in FIG. 17C, where one or more enzyme domains 1563 comprising G3PD, at least one cofactor and at least one regenerating enzyme, are positioned proximal to at least a portion of WE surface, with one or more cofactor reservoirs 1562 adjacent to the enzyme domains comprising G3PD and more distal from the WE surface, and one or more RL's 1552 are positioned adjacent the cofactor reservoir. In either of these configurations, an additional enzyme domain comprising lipase can be included to indirectly measure triglyceride, as the lipase will produce glycerol for detection by the aforementioned glycerol sensor configurations.

In another example, a glycerol sensor configuration is provided using dehydrogenase enzymes with cofactors and regenerating enzymes. In one example, cofactors that can be incorporated in the one or more enzyme domains include one or more of NAD(P)H, NADP+, and ATP. In one example, e.g., for use of NAD(P)H a regenerating enzyme can be NADH oxidase or diaphorase to convert NADH, the product of the dehydrogenase catalysis back to NAD(P)H. Similar methodologies can be used for creating other glycerol sensors, for example, glycerol dehydrogenase, combined with NADH oxidase or diaphorase can be configured to measure glycerol or oxygen.

In one example, mathematical modeling can be used to identify and remove interference signals, measuring very low analyte concentrations, signal error and noise reduction so as to improve and increase of multi-analyte sensor end of life. For example, with a two WE electrode configuration where WE1 is coated with a first EZL while WE2 is coated with two or more different EZL, optionally with one or more resistance domains (RL) a mathematical correction such interference can be corrected for, providing for increasing accuracy of the measurements.

Changes of enzyme load, immobilizing polymer and resistance domain characteristics over each analyte sensing region can result in different sensitive ratios between two or more target analyte and interfering species. If the signal are collected and analyzed using mathematical modeling, a more precise concentration of the target analytes can be calculated.

One example in which use of mathematical modeling can be helpful is with glycerol sensing, where galactose oxidase is sensitive towards both galactose and glycerol. The sensitivity ratio of galactose oxidase to glycerol is about is 1%-5% of its sensitivity to galactose. In such case, modification of the sensitivity ratio to the two analytes is possible by adjusting the one or more parameters, such as enzyme source, enzyme load, enzyme domain (EZL) diffusional characteristics, RL diffusional characteristics, and combinations thereof. If two WEs are operating in the sensor system, signal correction and analysis from both WEs using mathematical modeling provides high degree of fidelity and target analyte concentration measurement.

In the above configurations, the proximity to the WE of one or more of these enzyme immobilizing layers discussed herein can be different or reversed, for example if the most proximal to the WE enzyme domain provides hydrogen peroxide, this configuration can be used.

In some examples, the target analyte can be measured using one or multiple of enzyme working in concert. In one example, ATP can be immobilized in one or more EZL membranes, or can be added to an adjacent layer alone or in combination with a secondary cofactor, or can get regenerated/recycled for use in the same EZL or an adjacent third EZL. This configuration can further include a cofactor regenerator enzyme, e.g., alcohol dehydrogenase or NADH oxidase to regenerate NAD(P)H. Other examples of cofactor regenerator enzymes that can be used for ATP regeneration are ATP synthase, pyruvate kinase, acetate kinase, creatine kinase, and the like.

In one example, the aforementioned continuous glycerol sensor configurations can be combined with any one of the aforementioned continuous alcohol sensor configurations, continuous uric acid sensor configurations, continuous cholesterol sensor configurations, continuous bilirubin/ascorbic acid sensor configurations, ketone sensor configurations, choline sensor configurations to provide a continuous multi-analyte sensor device as further described below. This continuous multi-analyte sensor device can further include continuous glucose monitoring capability. Other configurations can be used in the aforementioned continuous glycerol sensor configuration, such as electrode, resistance, bio-interfacing, and drug releasing membranes.

### Creatinine Sensor Configurations

In one example, continuous creatinine sensor configurations are provided, such configurations containing one or more enzymes and/or cofactors. Creatinine sensor configurations are examples of continuous analyte sensing systems that generate intermediate, interfering products, where these intermediates/interferents are also present in the biological fluids sampled. The present disclosure provides solutions to address these technical problems and provide for accurate, stable, and continuous creatinine monitoring alone or in combination with other continuous multi-analyte sensor configurations.

Creatinine sensors, when in use, are subject to changes of a number of physiologically present intermediate/interfering products, for example sarcosine and creatine, that can affect the correlation of the transduced signal with the creatinine concentration. The physiological concentration range of sarcosine, for example, is an order of magnitude lower that creatinine or creatine, so signal contribution from circulating sarcosine is typically minimal. However, changes in local physiological creatine concentration can affect the creatinine sensor signal. In one example, eliminating or reducing such signal contribution is provided.

Thus, in one example, eliminating or reducing creatine signal contribution of a creatinine sensor comprises using at least one enzyme that will consume the non-targeted interfering analyte, in this case, creatine. For example, two enzyme domains are used, positioned adjacent to each other. At least a portion of a first enzyme domain is positioned proximal to at least a portion of a WE surface, the first enzyme domain comprising one or more enzymes selected from creatinine amidohydrolase (CNH), creatine amidohydrolase (CRH), and sarcosine oxidase (SOX). A second enzyme domain, adjacent the first enzyme domain and more distal from the WE surface, comprises one or more enzymes using creatine as their substrate so as to eliminate or reduce creatine diffusion towards the WE. In one example, combinations of enzymes include CRH, SOX, creatine kinase, and catalase, where the enzyme ratios are tuned to provide ample number of units such that circulating creatine will at least partially be consumed by CRH providing sarcosine and urea, whereas the sarcosine produced will at least partially be consumed by SOX, providing an oxidized form of glycine (e.g. glycine aldehyde) which will at least be partially consumed by catalase. In an alternative configuration of the above, the urea produced by the CRH catalysis can at least partially be consumed by urease to provide ammonia, with the aqueous form (NH4+) being detected via an ion-selective electrode (e.g., nonactin ionophore). Such an alternative potentiometric sensing configuration may provide an alternative to amperometric peroxide detection (e.g., improved sensitivity, limits of detection, and lack of depletion of the reference electrode, alternate pathways/mechanisms). This dual-analyte-sensing example may include a creatinine-potassium sensor having potentiometric sensing at two different working electrodes. In this example, interference signals can be identified and corrected. In one alternative example, the aforementioned configuration can include multi-modal sensing architectures using a combination of amperometry and potentiometry to detect concentrations of peroxide and ammonium ion, measured using amperometry and potentiometry, respectively, and correlated to measure the concentration of the creatinine. In one example, the aforementioned configurations can further comprise one or more configurations (e.g., without enzyme) separating the two enzyme domains to provide complementary or assisting diffusional separations and barriers.

In yet another example, a method to isolate the signal and measure essentially only creatinine is to use a second WE that measures the interfering species (e.g., creatine) and then correct for the signal using mathematical modeling. Thus, for example, signal from the WE interacting with creatine is used as a reference signal. Signal from another WE interacting with creatinine is from corrected for signal from the WE interacting with creatine to selectively determine creatinine concentration.

In yet another example, sensing creatinine is provided by measuring oxygen level changes electrochemically, for example in a Clark type electrode setup, or using one or more electrodes coated with layers of different polymers such as NAFION™ and correlating changes of potential based on oxygen changes, which will indirectly correlate with the concentrations of creatinine.

In yet another example, sensing creatinine is provided by using sarcosine oxidase wired to at least one WE using one or more electrically coupled mediators. In this approach, concentration of creatinine will indirectly correlate with the electron transfer generated signal collected from the WE.

For the aforementioned creatinine sensor configurations based on hydrogen peroxide and/or oxygen measurements the one or more enzymes can be in a single enzyme domain, or the one or more enzymes, independently, can be in one or more enzyme domains, or any other combination thereof, in which in each layer at least one enzyme is present. For the aforementioned creatinine sensor configurations based on use of an electrically coupled sarcosine oxidase containing layer, the layer positioned adjacent to the electrode and is electrically coupled to at least a portion of the electrode surface using mediators.

In another example, the aforementioned creatinine sensor configurations can be sensed using potentiometry by using urease enzyme (UR) that creates ammonium from urea, the urea created by CRH from creatine, the creatine being formed from the interaction of creatinine with CNH. Thus, ammonium can be measured by the above configuration and correlated with the creatinine concentration. Alternatively, creatine amidohydrolase (CI) or creatinine deiminase can be used to create ammonia gas, which under physiological conditions of a transcutaneous sensor, would provide ammonium ion for signal transduction.

In yet another example, sensing creatinine is provided by using one or more enzymes and one or more cofactors. Some non-limiting examples of such configurations include creatinine deaminase (CD) providing ammonium from creatinine, glutamate dehydrogenase (GLDH) providing peroxide from the ammonium, where hydrogen peroxide correlates with levels of present creatinine. The above configuration can further include a third enzyme glutamate oxidase (GLOD) to further break down glutamate formed from the GDLH and create additional hydrogen peroxide. Such combinations of enzymes, independently, can be in one or more enzyme domains, or any other combination thereof, in which in each domain or layer, at least one enzyme is present.

In yet another example, sensing creatinine is provided by the combination of creatinine amidohydrolase (CNH), creatine kinase (CK) and pyruvate kinase (PK), where pyruvate, created by PK can be detected by one or more of either lactate dehydrogenase (LDH) or pyruvate oxidase (POX) enzymes configured independently, where one or more of the aforementioned enzyme are present in one layer, or, in which in each of a plurality of layers comprises at least one enzyme, any other combination thereof.

In such sensor configurations where one or more cofactors and/or regenerating enzymes for the cofactors are used, providing excess amounts of one or more of NADH, NAD(P)H and ATP in any of the one or more configurations can be employed, and one or more diffusion resistance domains can be introduced to limit or prevent flux of the cofactors from their respective membrane(s). Other configurations can be used in the aforementioned configurations, such as electrode, resistance, bio-interfacing, and drug releasing membranes.

In yet another example, creatinine detection is provided by using creatinine deiminase in one or more enzyme domains and providing ammonium to the enzyme domain(s) via catalysis of creatinine. Ammonium ion can then be detected potentiometrically or by using composite electrodes that undergo redox when exposed to ammonium ion, for example NAFION™/polyaniline composite electrodes, in which polyaniline undergoes redox in the presence of ammonium at the electrode under potential. Ammonium concentration can then be correlated to creatinine concentration.

FIG. 18 depicts an exemplary continuous sensor configuration for creatinine. In the example of FIG. 18, the sensor includes a first enzyme domain 1564 comprising CNH, CRH, and SOX are adjacent a working electrode WE, e.g., platinum. A second enzyme domain 1565 is positioned adjacent the first enzyme domain and is more distal from the WE. One or more resistance domains (RL) 1552 can be positioned adjacent the second enzyme domain or between the first and second layers. Creatinine is diffusible through the RL and the second enzyme domain to the first enzyme domain where it is converted to peroxide and transduces a signal corresponding to its concentration. Creatine is diffusible through the RL and is converted in the second enzyme domain to sarcosine and urea, the sarcosine being consumed by the sarcosine oxidase and the peroxide generated is consumed by the catalase, thus preventing transduction of the creatine signal.

For example, variations of the above configuration are possible for continuous monitoring of creatinine alone or in combination with one or more other analytes. Thus, one alternative approach to sensing creatinine could be sensing oxygen level changes electrochemically, for example in a Clark-type electrode setup. In one example, the WE can be coated with layers of different polymers, such as NAFION™ and based on changes of potential oxygen changes, the concentrations of creatinine can be correlated. In yet another example, one or more enzyme most proximal to the WE, i.e., sarcosine oxidase, can be “wired” to the electrode using one or more mediators. Each of the different enzymes in the above configurations can be distributed inside a polymer matrix or domain to provide one enzyme domain. In another example, one or more of the different enzymes discussed herein can be formed as the enzyme domain and can be formed layer by layer, in which each layer has at least one enzyme present. In an example of a “wired” enzyme configuration with a multilayered membrane, the wired enzyme domain would be most proximal to the electrode. One or more interferent layers can be deposited among the multilayer enzyme configuration so as to block of non-targeted analytes from reaching electrodes.

In one example, the aforementioned continuous creatinine sensor configurations can be combined with any one of the aforementioned continuous alcohol sensor configurations, continuous uric acid sensor configurations, continuous cholesterol sensor configurations, continuous bilirubin/ascorbic acid sensor configurations, ketone sensor configurations, choline sensor configurations, glycerol sensor configurations to provide a continuous multi-analyte sensor device as further described below. This continuous multi-analyte sensor device can further include continuous glucose monitoring capability.

### Lactose Sensor Configurations

In one example, a continuous lactose sensor configuration, alone or in combination with another analyte sensing configuration comprising one or more enzymes and/or cofactors is provided. In a general sense, a lactose sensing configuration using at least one enzyme domain comprising lactase enzyme is used for producing glucose and galactose from the lactose. The produced glucose or galactose is then enzymatically converted to a peroxide for signal transduction at an electrode. Thus, in one example, at least one enzyme domain EZL1 comprising lactase is positioned proximal to at least a portion of a WE surface capable of electrolysis of hydrogen peroxide. In one example, glucose oxidase enzyme (GOX) is included in EZL1, with one or more cofactors or electrically coupled mediators. In another example, galactose oxidase enzyme (GalOx) is included in EZL1, optionally with one or more cofactors or mediators. In one example, glucose oxidase enzyme and galactose oxidase are both included in EZL1. In one example, glucose oxidase enzyme and galactose oxidase are both included in EZL1, optionally with one or more cofactors or electrically coupled mediators.

One or more additional EZL's (e.g. EZL2) can be positioned adjacent the EZL1, where at least a portion of EZL2 is more distal from at least a portion of WE than EZL1. In one example, one or more layers can be positioned in between EZL1 and EZL2, such layers can comprise enzyme, cofactor or mediator or be essentially devoid of one or more of enzymes, cofactors or mediators. In one example, the one or more layers positioned in between EZL1 and EZL2 is essentially devoid of enzyme, e.g., no purposefully added enzyme. In one example one or layers can be positioned adjacent EZL2, being more distal from at least a portion of EZL1 than EZL2, and comprise one or more of the enzymes present in either EZL1 or EZL2.

In one example of the aforementioned lactose sensor configurations, the peroxide generating enzyme can be electrically coupled to the electrode using coupling mediators. The transduced peroxide signals from the aforementioned lactose sensor configurations can be correlated with the level of lactose present.

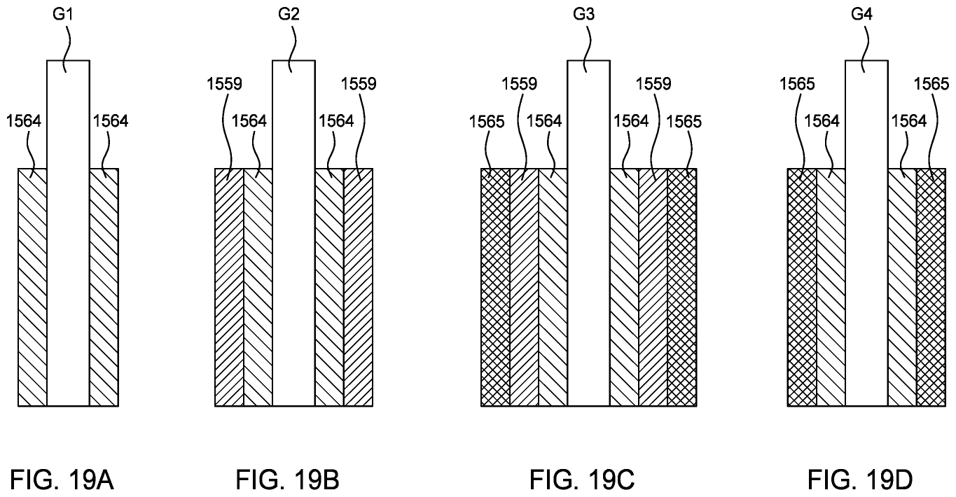

FIG. 19A-19D depict alternative continuous lactose sensor configurations. Thus, in an enzyme domain EZL1 1564 most proximal to WE (G1), comprising GalOx and lactase, provides a lactose sensor that is sensitive to galactose and lactose concentration changes and is essentially non-transducing of glucose concentration. As shown in FIGS. 19B-19D, additional layers, including non-enzyme containing layers 1559, and an enzyme domain1565 (e.g., a lactase enzyme containing layer), and optionally, electrode, resistance, bio-interfacing, and drug releasing membranes. (not shown) are used. Since changes in physiological galactose concentration are minimal, the transduced signal would essentially be from physiological lactose fluctuations.

In one example, the aforementioned continuous lactose sensor configurations can be combined with any one of the aforementioned continuous alcohol sensor configurations, continuous uric acid sensor configurations, continuous cholesterol sensor configurations, continuous bilirubin/ascorbic acid sensor configurations, ketone sensor configurations, choline sensor configurations, glycerol sensor configurations, creatinine sensor configurations to provide a continuous multi-analyte sensor device as further described below. This continuous multi-analyte sensor device can further include continuous glucose monitoring capability. Other membranes can be used in the aforementioned sensor configuration, such as electrode, resistance, bio-interfacing, and drug releasing membranes.

### Urea Sensor Configurations

Similar approach as described above can also be used to create a continuous urea sensor. For example urease (UR), which can break down the urea and to provide ammonium can be used in an enzyme domain configuration. Ammonium can be detected with potentiometry or by using a composite electrodes, e.g., electrodes that undergo redox when exposed to ammonium. Example electrodes for ammonium signal transduction include, but are not limited to, NAFION™/polyaniline composite electrodes, in which polyaniline undergoes redox in the presence of ammonium at an applied potential, with essentially direct correlation of signal to the level of ammonium present in the surrounding. This method can also be used to measure other analytes such as glutamate using the enzyme glutaminase (GLUS).

In one example, the aforementioned continuous uric acid sensor configurations can be combined with any one of the aforementioned continuous alcohol sensor configurations and/or continuous uric acid sensor configurations and/or continuous cholesterol sensor configurations and/or continuous bilirubin/ascorbic acid sensor configurations and/or continuous ketone sensor configurations and/or continuous choline sensor configurations and/or continuous glycerol sensor configurations and/or continuous creatinine sensor configurations and/or continuous lactose sensor configurations to provide a continuous multi-analyte sensor device as further described below. This continuous multi-analyte sensor device can further include continuous glucose monitoring capability. Other membranes can be used in the aforementioned uric acid sensor configuration, such as electrode, resistance, bio-interfacing, and drug releasing membranes.

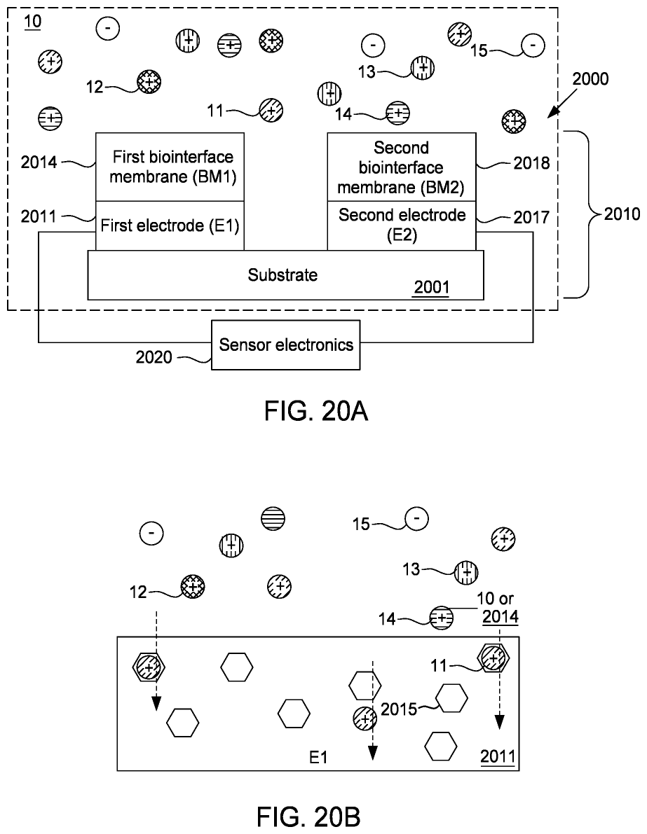

In certain embodiments, continuous analyte monitoring system 104 may be a potassium sensor, as discussed in reference to FIG. 1. FIGS. 20-23 describe an example sensor device used to measure an electrophysiological signal and/or concentration of a target analyte (e.g., potassium), according to certain embodiments of the present disclosure.

The term “ion” as used herein is a broad term, and is to be given its ordinary and customary meaning to a person of ordinary skill in the art (and is not to be limited to a special or customized meaning), and refers without limitation to an atom or molecule with a net electric charge due to the loss or gain of one or more electrons. Ions in a biological fluid may be referred to as “electrolytes.” Nonlimiting examples of ions in biological fluids include sodium (Na⁺), potassium (K⁺), magnesium (Mg²⁺), calcium (Ca²⁺), hydrogen (H⁺), lithium (Li⁺), chloride (Cl⁻), sulfide (S²⁻), sulfite (SO₃²⁻), sulfate (SO₄²⁻), phosphate (PO₄³⁻), and ammonium (NH₄⁺). An ion is an example of an analyte.

FIG. 20A schematically illustrates an example configuration and component of a device 2000 for measuring an electrophysiological signal and/or concentration of a target analyte such as a target ion 11 in a biological fluid 10 in vivo. Turning first to FIG. 20, device 2000 includes indwelling sensor 2010 and sensor electronics 2020. Sensor 2010 includes substrate 2001, first electrode (E1) 2011 disposed on the substrate, and a second electrode (E2) 2017 disposed on the substrate. First electrode 2011 may be referred to as a working electrode (WE), while second electrode 2017 may be referred to as a reference electrode (RE). The sensor electronics 2020 may be configured to generate a signal corresponding to an electromotive force (EMF). In some examples, the EMF is at least partially based on a potential difference that is generated between the first electrode 2011 and the second electrode 2017 responsive to biological fluid 10 conducting the electrophysiological signal to first electrode 2011. Sensor electronics 2020 may be configured to use the signal to generate an output corresponding to a measurement of the signal. In various examples, the EMF is at least partially based on a potential difference between (i) either the first electrode 2011 or the second electrode 2017 and (ii) another electrode which is spaced apart from the first electrode or second electrode.

Additionally, or alternatively, in some examples, device 2000 may include an ionophore, such as ionophore 2015 as shown in FIG. 20B, disposed on the substrate 2001 and configured to selectively transport the target ion 11 to or within the first electrode 2011. The EMF may be at least partially based on a potential difference may be generated between the first electrode 2011 and the second electrode 2017 responsive to the ionophore transporting the target ion to or through the first electrode 2011. The sensor electronics 2020 (and/or an external device that receives the signal via a suitable wired or wireless connection) may be configured to use the signal to generate an output corresponding to a measurement of the concentration of the target ion in the biological fluid. Further details regarding the configuration and use of sensor electronics 2020 are provided further below.

Optionally, the first electrode 2011 may be used to measure an electrophysiological signal in addition to ion concentration. In other examples, such as when device 2000 is configured to detect an electrophysiological signal but not an ion concentration, first electrode 2011 need not include an ionophore, such as ionophore 2015 as shown in FIG. 20B. In other examples, the first electrode 2011 may include an ionophore that is inactive such that it does not interfere with the measurement of the electrophysiological signal.

In a manner such as illustrated in FIG. 20A, biological fluid 10 may include a plurality of ions 11, 12, 13, 14, and 15. Device 2000 may be configured to measure the concentration of ion 11, and accordingly such ion may be referred to as a “target” ion. Target ion 11 may be any suitable ion, and in nonlimiting examples is selected from the group consisting of sodium (Nat), potassium (K⁺), magnesium (Mg²⁺), calcium (Ca²⁺, hydrogen (H⁺), lithium (Li⁺), chloride (Cl⁻), sulfite (SO₃²⁻), sulfate (SO₄²⁻), phosphate (PO₄³⁻), and ammonium (NH₄⁺). Ions 12, 13, 14, and 15 may be others of the group consisting of sodium (Na⁺), potassium (K⁺), magnesium (Mg²⁺), calcium (Ca²⁺), hydrogen (H⁺), lithium (Li⁺), chloride (Cl⁻), sulfide (S²⁻), sulfite (SO₃²⁻), sulfate (SO₄²⁻), phosphate (PO₄³⁻), and ammonium (NH₄⁺). Ions 12, 13, 14, and 15 may be considered interferants to the measurement of target ion 11 because they have the potential interfere with the measurement of target ion 11 by sensor to produce a signal that does not accurately represent the concentration of target ion 11. Ionophore, such as ionophore 2015 as shown in FIG. 20B, may be selected so as to selectively transport target ion 11 to or within first electrode 2011 and to inhibit, fully, partially and/or substantially, the transport of one or more of ions 12, 13, 14, or 15 to or within first electrode 2011. For example, as illustrated in FIG. 20B, ionophore 115 may selectively transport, or selectively bind, target ions 11 from biological fluid 10 or from biointerface membrane 2014 (if provided, e.g., as described below) to and within first electrode 2011, while ions 12, 13, 14, and 15 may substantially remain within biological fluid 10 or biointerface membrane 2014. Accordingly, contributions to the potential difference between first electrode 2011 and second electrode 2017 responsive to the transport of ions to or within first electrode 2011 substantially may be primarily caused by target ion 11 instead of by one or more of ions 12, 13, 14, or 15.

A wide variety of ionophores 2015 may be used to selectively transport corresponding ions in a manner such as described with reference to FIGS. 20A-20B. For example, where the target ion 11 is hydrogen (via peroxide), the ionophore 2015 may be tridodecylamine, 4-nonadecylpyridine, N,N-dioctadecylmethylamine, octadecyl isonicotinate, calix[4]-aza-crown. Or, for example, where the target ion 11 is lithium, the ionophore 115 may be ETH 149, N,N,N,N′,N″,N″-hexacyclohexyl-4,4′,4″-propylidynetris(3-oxabutyramide), or 6,6-Dibenzyl-1,4,8-11-tetraoxacyclotetradecane. Or, for example, where the target ion 11 is sulfite, the ionophore 2015 may be octadecyl 4-formylbenzoate. Or, for example, where the target ion 11 is sulfate, the ionophore 2015 may be 1,3-[bis(3-phenylthioureidomethyl)]benzene or zinc phthalocyanine. Or, for example, where the target ion 11 is phosphate, the ionophore 2015 may be 9-decyl-1,4,7-triazacyclodecane-8,10-dione. Or, for example, where the target ion 11 is sodium, the ionophore 2015 may be 4-tert-butylcalix[4]arene-tetraacetic acid tetraethyl ester (sodium ionophore X) or calix[4]arene-25,26,27,28-tetrol (calix[4]arene). Or, for example, where the target ion 11 is potassium, the ionophore 2015 may be potassium ionophore II (BB15C5) or valinomycin. Or, for example, where the target ion is magnesium, the ionophore 2015 may be 4,5-bis(benzoylthio)-1,3-dithiole-2-thione (Bz2dmit) or 1,3,5-Tri s[10-(1-adamantyl)-7,9-dioxo-6,10-diazaundecyl]benzene (magnesium ionophore VI). Or, for example, where the target ion is calcium, the ionophore 2015 may be calcium ionophore I (ETH 1001) or calcium ionophore II (ETH129). Or, for example, where the target ion is chloride, the ionophore 2015 may be tridodecylmethylammonium chloride (TDMAC). Or, for example, where the target ion is ammonium, the ionophore 2015 may be nonactin.

In the nonlimiting example illustrated in FIG. 20A, ionophore 2015 may be provided within first electrode 2011, and in such example the first electrode may be referred to as an ion-selective electrode (ISE), since the ionophore 2015 selectively transports the target ion 11. In some examples, first electrode 2011 may include a conductive polymer optionally having ionophore 2015 therein. Illustratively, the conductive polymer may be present in an amount of about 90 to about 99.5 weight percent in the first electrode 2011. The ionophore 2015 may be present in an amount of about 0.5 to about 10 weight percent in the first electrode. In some examples, the conductive polymer may be selected from the group consisting of: poly(3,4-ethylenedioxythiophene) (PEDOT), poly(3,4-ethylenedioxythiophene) polystyrene sulfonate (PEDOT:PSS), polyaniline (PANT), poly(pyrrole) (PPy), or poly(3-octylthiophene) (POT).

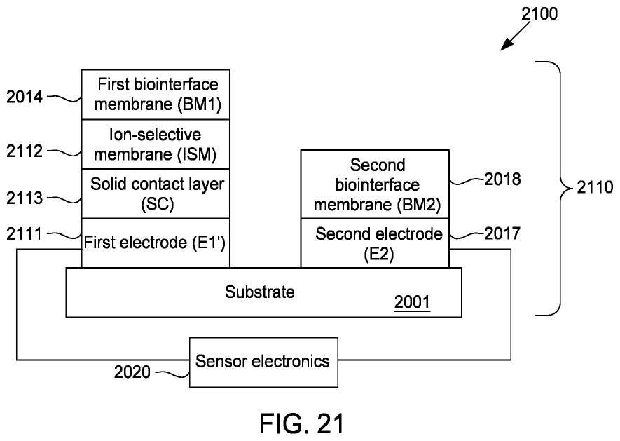

While conductive polymers (such as listed above) suitably may be used in a first electrode 111 that excludes ionophore 2015, other materials alternatively may be used, some nonlimiting examples of which are described below with reference to FIG. 21. Optionally, ionophore 2015 may be provided in a membrane which is disposed on a first electrode 2011 (which electrode may exclude ionophore 2015), e.g., such as will be described below with reference to FIG. 21.

First electrode 2011 may be configured in such a manner as to enhance its biocompatibility. For example, first electrode 2011 may substantially exclude any plasticizer, which otherwise may leach into the biological fluid 10, potentially causing toxicity and/or a degradation in device performance. As used herein, the “substantial” exclusion of materials such as plasticizers is intended to mean that the first electrode 2011 or other aspects discussed herein do not contain detectable quantities of the “substantially” excluded material. In some examples, the first electrode 2011 may consist essentially of the conductive polymer, optionally in addition to the ionophore 2015. In some examples, the first electrode 2011 may consist essentially of the conductive polymer, the ionophore 2015, and an additive with ion exchanger capability. Such an additive may act as an ion exchanger. In one example, the additive contributes to the ion selectivity. In another example, the additive may not provide ion selectivity. For example, the additive may help to provide a substantially even concentration of the ion in the membrane. Additionally, or alternatively, the additive may help any change in ion concentration in the biofluid to cause an ion exchange within the membrane that may induce a non-selective potential difference. Additionally, or alternatively, the ionophore and the ion exchanger may form a complex which improves the ionophore's selectivity towards the target ion as compared to the selectivity of the ionophore alone.

Optionally, the additive may include a lipophilic salt. In nonlimiting examples, the lipophilic salt is selected from the group consisting of sodium tetrakis[3,5-bis(trifluoromethyl)phenyl]borate (NaTPFB), sodium tetraphenylborate (NaTPB), potassium tetrakis [3,5-bis(trifluoromethyl)phenyl]borate (KTFPB), and potassium tetrakis(4-chlorophenyl)borate (KTClPB). The additive may be present in an amount of about 0.01 to about 1 weight percent in the first electrode, or other suitable amount.

Other materials within sensor 2010 may be selected. For example, substrate 2001 may include a material selected from the group consisting of: metal, glass, transparent conductive oxide, semiconductor, dielectric, ceramic, and polymer (such as biopolymer or synthetic polymer). In some examples, second electrode 2017 may include a metal, a metal alloy, a transition metal oxide, a transparent conductive oxide, a carbon material, a doped semiconductor, a binary semiconductor, a ternary semiconductor, or a conductive polymer. The binary semiconductor may include any two elements suitable for use in a semiconductor. The ternary semiconductor may include two or more binary semiconductors. In examples where a metal or a metal alloy is used, the metal or metals used can be selected from the group consisting of: gold, platinum, silver, iridium, rhodium, ruthenium, nickel, chromium, and titanium. The metal optionally may be oxidized or optionally may be in the form of a metal salt. A nonlimiting example of an oxidized metal which may be used in second electrode 2017 is iridium oxide. The carbon material may be selected from the group consisting of: carbon paste, graphene oxide, carbon nanotubes, C60, porous carbon nanomaterial, mesoporous carbon, glassy carbon, hybrid carbon nanomaterial, graphite, and doped diamond. The doped semiconductor may be selected from the group consisting of: silicon, germanium, silicon-germanium, zinc oxide, gallium arsenide, indium phosphide, gallium nitride, cadmium telluride, indium gallium arsenide, and aluminum arsenide. The transition metal oxide may be selected from the group of: titanium dioxide (TiO₂), iridium dioxide (IrO₂), platinum dioxide (PtO₂), zinc oxide (ZnO), copper oxide (CuO), cerium dioxide (CeO₂), ruthenium(IV) oxide (RuO₂), tantalum pentoxide (Ta₂O₅), titanium dioxide (TiO₂), molybdenum dioxide (MoO₂), and manganese dioxide (MnO₂). The metal alloy may be selected from the group consisting of: platinum-iridium (Pt—Ir), platinum-silver (Pt—Ag), platinum-gold (Pt—Au), gold-iridium (Au—Ir), gold-copper (Au—Cu), gold-silver (Au—Ag), and cobalt-iron (Co—Fe).

The conductive polymer that may be used for the sensor 2010 may be selected from the group consisting of: poly(3,4-ethylenedioxythiophene) (PEDOT), poly(3,4-ethylenedioxythiophene) polystyrene sulfonate (PEDOT:PSS), polyaniline (PANT), poly(pyrrole) (PPy), or poly(3-octylthiophene) (POT). That is, first electrode 2011 and second electrode 2017 optionally may be formed of the same material as one another, or may be formed using different materials than one another. In the nonlimiting example illustrated in FIG. 20A, first electrode 2011 and second electrode 2017 may be disposed directly on substrate 2001, or alternatively may be disposed on substrate 2001 via one or more intervening layers (not illustrated).

The biocompatibility of sensor 2010 optionally may be further enhanced by providing a biointerface membrane over one or more component(s) of sensor 2010. For example, in the nonlimiting configuration illustrated in FIG. 20A, a first biointerface membrane (BM1) 2014 may be disposed on the ionophore 2015 and the first electrode 2011. In another example, the first biointerface membrane (BM1) 2014 may be disposed on the ionophore 2015 and the first electrode 2011, and a second biointerface membrane (BM2) 2018 may be disposed on the second electrode 2017. Although FIG. 20A may suggest that the biointerface membrane(s) have a rectangular shape for simplicity of illustration, it should be apparent that the membrane(s) may conform to the shape of any underlying layers. In some examples, the biointerface membrane(s) may be configured to inhibit biofouling of the ionophore 2015, the first electrode 2011, and/or the second electrode 2017. Nonlimiting examples of materials which may be included in the biointerface membrane(s) include hard segments and/or soft segments. Examples of hard and soft segments used for the biointerface membrane 2014/2014′/2018 or other biointerface membranes as discussed herein include aromatic polyurethane hard segments with Si groups, aliphatic hard segments, polycarbonate soft segments or any combination thereof. In other examples of biointerface membrane(s) such as 2014/2014′/2018 or other biointerface membranes discussed herein, PVP may not be included. In this example where no PVP is included, the biointerface membrane (2018, 2014, 2014′, or other biointerface membranes as discussed herein) may include polyurethane and PDMS. In some examples, which may be combined with other examples herein, the biointerface membranes discussed herein may include one or more zwitterionic compounds.

Whereas ionophore 2015 is included within first electrode 2011 in the example described with reference to FIG. 20A, in the example illustrated in FIG. 21 first electrode 2111 does not include ionophore 2015 (and thus may be referred to as E1′ rather than E1). Instead, ionophore 2015 may be within an ion-selective membrane (ISM) 2112 disposed on the first electrode 2111. Ionophores 2015 may selectively transport target ion 11 to first electrode 2111 in a manner similar to that described with reference to FIGS. 20A-20B, and such transport may cause a potential difference between the first electrode 2111 and second electrode 2017 based upon which sensor electronics 2020 may generate an output corresponding to a measurement of the concentration of target ion 11 in biological fluid 10. It will be appreciated that in examples in which device 2000 is used to measure an electrophysiological signal and is not used to measure an ion concentration, ISM 2112 may be omitted.

In a manner similar to that described with reference to first electrode 2011, ion-selective membrane 2112 substantially may exclude any plasticizer. In some examples, ion-selective membrane 2112 may consist essentially of a biocompatible polymer and ionophore 2015 configured to selectively bind the target ion. Alternatively, in some examples, the ion-selective membrane 2112 may consist essentially of a biocompatible polymer, an ionophore 2015 configured to selectively bind the target ion 11, and an additive with ion exchanger capability, such as a lipophilic salt. Nonlimiting examples of lipophilic salts, and nonlimiting amounts of additives, biocompatible polymers, and ionophores are provided above with reference to FIGS. 20A-20B. Whereas first electrode 2011 includes a conductive polymer so as to be able to provide ionophore 2015 therein while retaining the electrical conductivity of an electrode, additional types of materials may be used in ion-selective membrane 2112 because the ion-selective membrane 2112 need not be used as an electrode. For example, the biocompatible polymer of the ion-selective membrane 2112 may include a hydrophobic polymer. Illustratively, the hydrophobic polymer may be selected from the group consisting of silicone, fluorosilicone (FS), polyurethane, polyurethaneurea, polyurea. In one example, the biocompatible polymer of the ISM 2112 (or other ion-selective membranes or other membranes discussed here) may include one or more block copolymers, which may be segmented block copolymers. In one example, the hydrophobic polymer may be a segmented block copolymer comprising polyurethane and/or polyurea segments, and/or polyester segments, and one or more of polycarbonate, polydimethylsiloxane (PDMS), methylene diphenyl diisocyanate (MDI), polysulfone (PSF), methyl methacrylate (MMA), poly(ε-caprolactone) (PCL), and 1,4-butanediol (BD). In other examples, the hydrophobic polymer may alternately or additionally include poly(vinyl chloride) (PVC), fluoropolymer, polyacrylate, and/or polymethacrylate.

In one example, the biocompatible polymer may include a hydrophilic block copolymer instead of or in addition to one or more hydrophobic copolymers. Illustratively, the hydrophilic block copolymer may include one or more hydrophilic blocks selected from the group consisting of polyethylene glycol (PEG) and cellulosic polymers. Additionally, or alternatively, the block copolymer may include one or more hydrophobic blocks selected from the group consisting of polydimethylsiloxane (PDMS) polytetrafluoroethylene, polyethylene-co-tetrafluoroethylene, polyolefin, polyester, polycarbonate, biostable polytetrafluoroethylene, homopolymers, copolymers, terpolymers of polyurethanes, polypropylene (PP), polyvinylchloride (PVC), polyvinylidene fluoride (PVDF), polybutylene terephthalate (PBT), polymethylmethacrylate (PMMA), polyether ether ketone (PEEK), polyurethanes, poly(propylene oxide) and copolymers and blends thereof. In one example, the ion-selective membrane 2112 does not contain PVP, or other plasticizers.

In one example, the biocompatible polymer of the ion-selective membrane 2112 includes from about 0.1 wt. % silicone to about 80 wt. % silicone. In one example, the ion-selective membrane 2112, or other ion-selective membranes discussed herein, includes from about 5 wt. % silicone to about 25 wt. % silicone. In yet another example, the ion-selective membrane 2112, or other ion-selective membranes discussed herein, includes from about 35 wt. % silicone to about 65 wt. % silicone. In yet another example, the ion-selective membrane 2112, or other ion-selective membranes discussed herein, includes from about 30 wt. % silicone to about 50 wt. % silicone.

In certain examples, the ISM 2112 or other ISMs discussed herein may include one or more block copolymers or segmented block copolymers. The segmented block copolymer may include hard segments and soft segments. In this example, the hard segments may include aromatic or aliphatic diisocyanates are used to prepare hard segments of segmented block copolymer. In one example, the aliphatic or aromatic diisocyanate used to provide hard segment of polymer includes one or more of norbornane diisocyanate (NBDI), isophorone diisocyanate (IPDI), tolylene diisocyanate (TDI), 1,3-phenylene diisocyanate (MPDI), trans-1,3-bis(isocyanatomethyl) cyclohexane (1,3-H6XDI), bicyclohexylmethane-4,4′-diisocyanate (HMDI), 4,4′-diphenylmethane diisocyanate (MDI), trans-1,4-bis(isocyanatomethyl) cyclohexane (1,4-H6XDI), 1,4-cyclohexyl diisocyanate (CHDI), 1,4-phenylene diisocyanate (PPDI), 3,3′-Dimethyl-4,4′-biphenyldiisocyanate (TODI), 1,6-hexamethylene diisocyanate (HDI), or combinations thereof. In one example, the hard segments may be from about 5 wt. % to about 90 wt. % of the segmented block copolymer of the ISM 2112. In another example, the hard segments may be from about 15 wt. % to about 75 wt. %. In yet another example, the hard segments may be from about 25 wt. % to about 55 wt. %. It will be appreciated that ion-selective membrane 2112 and first electrode 2011 may be prepared in any suitable manner. Illustratively, the polymer, ionophore 2015, and any additive may be dispersed in appropriate amounts in a suitable organic solvent (e.g., tetrahydrofuran, isopropyl alcohol, acetone, or methyl ethyl ketone). The mixture may be coated onto substrate 2001 (or onto a layer thereon) using any suitable technique, such as dipping and drying, spray-coating, inkjet printing, aerosol jet dispensing, slot-coating, electrodeposition, electrospraying, electrospinning, chemical vapor deposition, plasma polymerization, physical vapor deposition, spin-coating, or the like. The organic solvent may be removed so as to form a solid material corresponding to ion-selective membrane 2112 or first electrode 2011. Other layers in device 2000 or device 2100, such as electrodes, solid contact layers, and/or biological membranes, may be formed using techniques described elsewhere herein or otherwise known in the art.

Whereas first electrode 2011 includes a conductive polymer so as to be able to provide ionophore 2015 therein while retaining the electrical conductivity of an electrode, additional types of materials may be used in first electrode 2111 because an ionophore need not be provided therein. Nonlimiting example materials for use in first electrode 2111 of device 2100 are provided above with reference to second electrode 2017, e.g., a metal, a metal alloy, a transition metal oxide, a transparent conductive oxide, a carbon material, a doped semiconductor, a binary semiconductor, a ternary semiconductor, or a conductive polymer such as described above with reference to FIG. 20A.

In some examples, the ion-selective membrane is in direct contact with the first electrode. In other examples, such as illustrated in FIG. 21, sensor 2110 further may include a solid contact layer 2113 disposed between the first electrode 2111 and the ion-selective membrane 2112. Solid contact layer 2113 may perform the function of enhancing the reproducibility and stability of the EMF by converting the signal into a measurable electrical potential signal. Additionally, or alternatively, solid contact layer 2113 may inhibit transport of water from the biological fluid 10 to the first electrode 2111 and/or accumulation of water at the first electrode 2111. Solid contact layer 2113 may include any suitable material or combination of materials. Nonlimiting example materials for use in solid contact layer 2113 are provided above with reference to second electrode 2017, e.g., a metal, a carbon material, a doped semiconductor, or a conductive polymer such as described above with reference to FIG. 20A. Alternatively, solid contact layer 2113 may include a redox couple which has a well-controlled concentration ratio of oxidized/reduced species that may be used to stabilize the interfacial electrical potential. The redox couple may include metallic centers with different oxidation states. Illustratively, the metallic centers may be selected from the group consisting of Co(II) and Co(III); Ir(II) and Ir(III); and Os(II) and Os(III). In alternative examples, the solid contact layer 213 may include a mixed conductor, or mixed ion-electron conductor, such as strontium titanate (SrTiO₃), titanium dioxide (TiO₂), (La,Ba,Sr)(Mn,Fe,Co)O_{3-d},La₂CuO_{4+d}, cerium(IV) oxide (CeO₂), lithium iron phosphate (LiFePO₄), and LiMnPO₄.

It will further be appreciated that sensor 2110 may have any suitable configuration. In the nonlimiting example illustrated in FIG. 21, substrate 2001 may be planar or substantially planar.

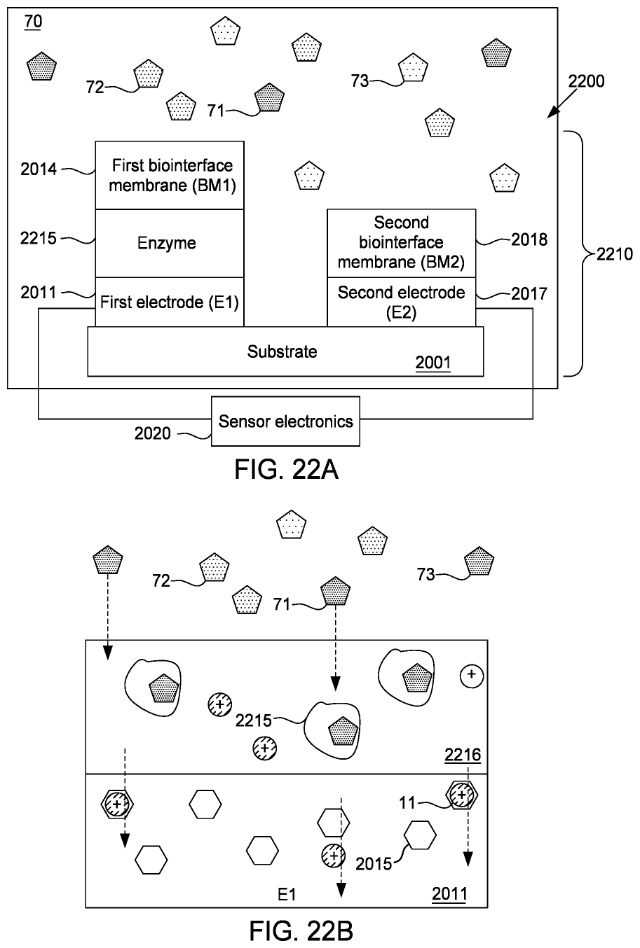

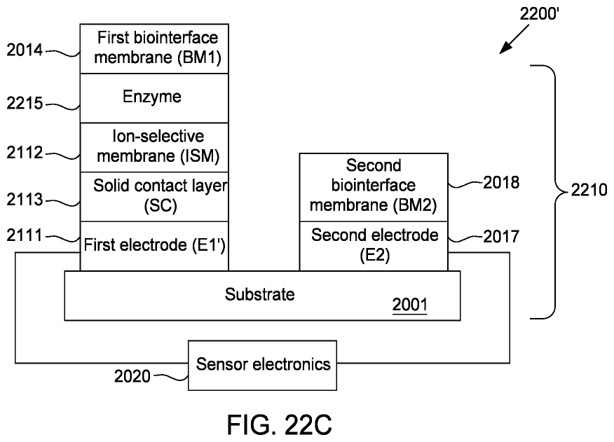

In the nonlimiting example illustrated in FIG. 22A, the ionophore may be located within first electrode (E1) 2011 disposed on the substrate and may be configured similarly as described with reference to FIG. 20A. Alternatively, in the nonlimiting example illustrated in FIG. 22C, the ionophore may be located within ion-selective membrane 2112 which may be configured in a manner such as described with reference to FIG. 21, and the first electrode 2111 may be configured in a manner such as described with reference to FIG. 21. First electrode 2011 or 2111 may be referred to as a working electrode (WE), while second electrode 2017 may be referred to as a reference electrode (RE).

The sensor electronics 2020 may be configured to generate a signal corresponding to an electromotive force (EMF). In some examples, the EMF is at least partially based on a potential difference that is generated between the first electrode and the second electrode responsive to the ionophore transporting the target ion to the first electrode. The sensor electronics 2020 may be configured to use the signal to generate an output corresponding to a measurement of the concentration of the target ion in the biological fluid, and/or may be configured to transmit the signal to an external device configured to use the signal to generate an output corresponding to a measurement of the concentration of the target ion in the biological fluid. Optionally, in some examples, the EMF is at least partially based on a potential difference that is generated between the first electrode and the second electrode responsive to biological fluid 10 conducting the electrophysiological signal to first electrode 111, and sensor electronics 2020 may be configured to use the signal to generate an output corresponding to a measurement of the electrophysiological signal.

In a manner such as illustrated in FIG. 22A, biological fluid 10 may include a plurality of analytes 71, 72, and 73. Device 2200 may be configured to measure the concentration of analyte 71, and accordingly such analyte may be referred to as a “target” analyte. As illustrated in FIG. 22B, enzyme 2215 may be located within enzyme layer 2216, and may selectively act upon target analyte 71 from biological fluid 10 or from biointerface membrane 2014 (if provided, e.g., as illustrated in FIG. 22A and configured similarly as described with reference to FIGS. 20A and 21). The action of enzyme 2215 upon the target analyte 71 generates the target ion 11. Ionophore 2015 within first electrode 2011 or within ion-selective membrane 2112 may selectively transport, or selectively bind, target ions 11 from enzyme 2215 to and within first electrode 2011 or first electrode 2111.

It will be appreciated that target analyte 71 may be any suitable analyte, enzyme 2215 may be any suitable enzyme that generates a suitable ion responsive to action upon that analyte, and ionophore 2015 may be any suitable ionophore that selectively transports and/or binds that ion generated by enzyme 2215 so as to generate an EMF based upon which the concentration of analyte 71 may be determined (whether using sensor electronics 2020 or an external device to which the sensor electronics 2020 transmits the electrophysiological signal and/or signal corresponding to ion concentration). Nonlimiting examples of analytes, enzymes, and ionophores that may be used together are listed below in Table 1.

TABLE 1

| Analyte | Enzyme | Ion generated | Ionophore |
|---|---|---|---|
| Urea | Urease | Ammonium | Nonactin |
| Glucose | Glucose | H+ (via | Tridodecylamine, 4- |
| | oxidase | peroxide) | Nonadecylpyridine, N,N- |
| | | | Dioctadecylmethylamine, |
| | | | Octadecyl isonicotinate, |
| | | | Calix[4]-aza-crown |
| Creatinine | Creatinine | Ammonium | Nonactin |
| | deaminase | | |
| Lactate | Lactate | H+ (via | Tridodecylamine, 4- |
| | oxidase | peroxide) | Nonadecylpyridine, N,N- |
| | | | Dioctadecylmethylamine, |
| | | | Octadecyl isonicotinate, |
| | | | Calix[4]-aza-crown |
| Cholesterol | Cholesterol | H+ (via | Tridodecylamine, 4- |
| | oxidase | peroxide) | Nonadecylpyridine, N,N- |
| | | | Dioctadecylmethylamine, |
| | | | Octadecyl isonicotinate, |
| | | | Calix[4]-aza-crown |
| Glutamate | Glutamate | Ammonium | Nonactin |
| | oxidase/ | | |
| | Glutamate | | |
| | dehydrogenase | | |
| Galactose | Galactose/ | H+ (via | Tridodecylamine, 4- |
| | oxidase | peroxide) | Nonadecylpyridine, N,N- |
| | | | Dioctadecylmethylamine, |
| | | | Octadecyl isonicotinate, |
| | | | Calix[4]-aza-crown |

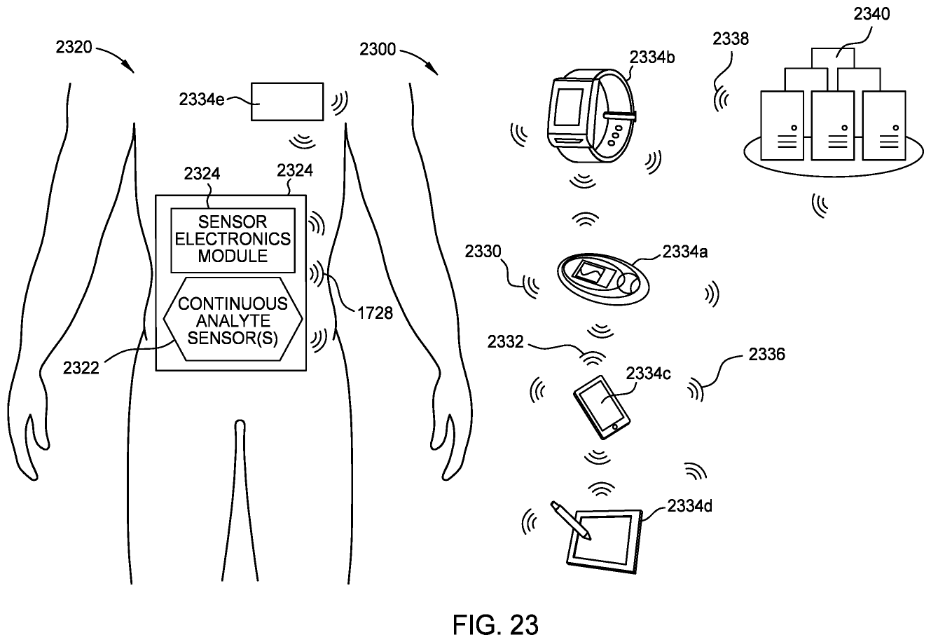

FIG. 23 is a diagram depicting an example continuous analyte monitoring system 2300 configured to measure one or more target ions and/or other analytes as discussed herein. The monitoring system 2300 includes an analyte sensor system 2324 operatively connected to a host 2320 and a plurality of display devices 2334 a-e according to certain aspects of the present disclosure. It should be noted that the display device 2334e alternatively or in addition to being a display device, may be a medicament delivery device that can act cooperatively with the analyte sensor system 2324 to deliver medicaments to host 2320. The analyte sensor system 2324 may include a sensor electronics module 2326 and a continuous analyte sensor 2322 associated with the sensor electronics module 2326. The sensor electronics module 2326 may be in direct wireless communication with one or more of the plurality of the display devices 2334a-e via wireless communications signals.

As will be discussed in greater detail below, display devices 2334a-e may also communicate amongst each other and/or through each other to analyte sensor system 2324. For ease of reference, wireless communications signals from analyte sensor system 2324 to display devices 2334a-e can be referred to as “uplink” signals 2328. Wireless communications signals from, e.g., display devices 2334a-e to analyte sensor system 2324 can be referred to as “downlink” signals 2330. Wireless communication signals between two or more of display devices 2334a-e may be referred to as “crosslink” signals 2332. Additionally, wireless communication signals can include data transmitted by one or more of display devices 2334a-d via “long-range” uplink signals 2336 (e.g., cellular signals) to one or more remote servers 2340 or network entities, such as cloud-based servers or databases, and receive long-range downlink signals 2338 transmitted by remote servers 2340.

The sensor electronics module 2326 includes sensor electronics that are configured to process sensor information and generate transformed sensor information. In certain embodiments, the sensor electronics module 2326 includes electronic circuitry associated with measuring and processing data from continuous analyte sensor 2322, including prospective algorithms associated with processing and calibration of the continuous analyte sensor data. The sensor electronics module 2326 can be integral with (non-releasably attached to) or releasably attachable to the continuous analyte sensor 2322 achieving a physical connection therebetween. The sensor electronics module 2326 may include hardware, firmware, and/or software that enables analyte level measurement. For example, the sensor electronics module 2326 can include a potentiostat, a power source for providing power to continuous analyte sensor 2322, other components useful for signal processing and data storage, and a telemetry module for transmitting data from itself to one or more display devices 2334a-e. Electronics can be affixed to a printed circuit board (PCB), or the like, and can take a variety of forms. For example, the electronics can take the form of an integrated circuit (IC), such as an Application-Specific Integrated Circuit (ASIC), a microcontroller, and/or a processor. Examples of systems and methods for processing sensor analyte data are described in more detail herein and in U.S. Pat. Nos. 7,310,544 and 6,931,327 and U.S. Patent Publication Nos. 2005/0043598, 2007/0032706, 2007/0016381, 2008/0033254, 2005/0203360, 2005/0154271, 2005/0192557, 2006/0222566, 2007/0203966 and 2007/0208245, each of which are incorporated herein by reference in their entirety for all purposes.

Display devices 2334a-e are configured for displaying, alarming, and/or basing medicament delivery on the sensor information that has been transmitted by the sensor electronics module 2326 (e.g., in a customized data package that is transmitted to one or more of display devices 2334a-e based on their respective preferences). Each of the display devices 2334a-e can include a display such as a touchscreen display for displaying sensor information to a user (most often host 2320 or a caretaker/medical professional) and/or receiving inputs from the user. In some embodiments, the display devices 2334a-e may include other types of user interfaces such as a voice user interface instead of or in addition to a touchscreen display for communicating sensor information to the user of the display device 2334a-e and/or receiving user inputs. In some embodiments, one, some or all of the display devices 2334a-e are configured to display or otherwise communicate the sensor information as it is communicated from the sensor electronics module 2326 (e.g., in a data package that is transmitted to respective display devices 2334a-e), without any additional prospective processing required for calibration and real-time display of the sensor information.

In the embodiment of FIG. 23, one of the plurality of display devices 2334a-e may be a custom display device 2334a specially designed for displaying certain types of displayable sensor information associated with analyte values received from the sensor electronics module 2326 (e.g., a numerical value and an arrow, in some embodiments). In some embodiments, one of the plurality of display devices 2334a-e may be a handheld device 2334c, such as a mobile phone based on the Android, iOS operating system or other operating system, a palm-top computer and the like, where handheld device 2334c may have a relatively larger display and be configured to display a graphical representation of the continuous sensor data (e.g., including current and historic data). Other display devices can include other hand-held devices, such as a tablet 2334d, a smart watch 2334b, a medicament delivery device 2334e, a blood glucose meter, and/or a desktop or laptop computer.

As discussed above, because the different display devices 2334a-e provide different user interfaces, content of the data packages (e.g., amount, format, and/or type of data to be displayed, alarms, and the like) can be customized (e.g., programmed differently by the manufacture and/or by an end user) for each particular display device and/or display device type. Accordingly, in the embodiment of FIG. 20A, one or more of display devices 2334a-e can be in direct or indirect wireless communication with the sensor electronics module 2326 to enable a plurality of different types and/or levels of display and/or functionality associated with the sensor information, which is described in more detail elsewhere herein.

### Example Embodiments

Embodiment 1: A method for determining a likelihood of one or more symptoms commonly associated with potassium imbalance occurring in real-time or within a specified period of time for the patient, the method comprising: monitoring one or more analytes of the patient during a plurality of time periods to obtain analyte data, the one or more analytes including at least potassium and the analyte data containing potassium data; processing the analyte data to determine at least one rate of change of potassium of the patient; and determining a likelihood that the patient is experiencing a symptom using the at least one rate of change of potassium of the patient and a trained model or one or more rules.

Embodiment 2: The method of Embodiment 1, further comprising: transmitting an indication to the patient prompting the patient to confirm or deny whether the patient is experiencing the symptom; and receiving a response from the patient confirming or denying experiencing the symptom.

Embodiment 3: The method of Embodiment 2, wherein the indication is transmitted when the likelihood that the patient is experiencing the symptom is above a predefined threshold.

Embodiment 4: The method of Embodiment 2, further comprising: when the patient confirms the patient is experiencing the symptom, associating physiological parameters of the patient to the patient experiencing the symptom; and using the association to update the trained model or the one or more rules.

Embodiment 5: The method of Embodiment 2, further comprising: when the patient denies the patient is experiencing the symptom, initiating one or more diagnostic tests, wherein the one or more diagnostic tests confirm or deny the patient is experiencing the symptom.

Embodiment 6: The method of Embodiment 5, wherein the one or more diagnostic tests comprise at least one of: using haptic feedback to communicate with the patient, wherein the patient is confirmed or denied to be experiencing the symptom based on a response of the patient to the haptic feedback; using a respiration sensor to determine a respiration rate of the patient, wherein the patient is confirmed or denied to be experiencing the symptom based on the determined respiration rate; using an electromyography (EMG) sensor to determine a muscle response of the patient, wherein the patient is confirmed or denied to be experiencing the symptom based on the determined muscle response; triggering the patient to squeeze a widget to determine a strength of the patient, wherein the patient is confirmed or denied to be experiencing the symptom based on a determined strength; or using a heart rate monitor to determine a resting heart rate of the patient, wherein the patient is confirmed or denied to be experiencing the symptom based on the resting heart rate.

Embodiment 7: The method of Embodiment 6, further comprising: when the one or more diagnostic tests confirm the patient is experiencing the symptom, associating physiological parameters of the patient to the patient experiencing the symptom; and using the association to update the trained model or the one or more rules.

Embodiment 8: The method of Embodiment 5, further comprising: when the one or more diagnostic tests deny the patient is experiencing the symptom, associating physiological parameters of the patient to the patient not experiencing the symptom; and using the association to update the trained model or the one or more rules.

Embodiment 9: The method of Embodiment 2, wherein the symptom comprises: potassium fluctuations, numbness, tingling, shortness of breath, muscle weakness, arrhythmia, death, or a symptom selected by the patient.

Embodiment 10: The method of Embodiment 2, wherein the one or more analytes further include at least one of: glucose, creatinine, blood urea nitrogen (BUN), ammonia, C-peptide, and cystatin C.

Embodiment 11: The method of Embodiment 1, further comprising: monitoring non-analyte sensor data of the patient during the plurality of time periods using one or more other non-analyte sensors.

Embodiment 12: The method of Embodiment 11, wherein the one or more other non-analyte sensors comprise at least one of an insulin pump, an accelerometer, a temperature sensor, an electrocardiogram (ECG) sensor, a heart rate monitor, a blood pressure sensor, an impedance, a peritoneal dialysis machine, a hemodialysis machine, a continuous positive airway pressure machine, a body sounds sensor, or a respiratory sensor.

Embodiment 13: The method of Embodiment 2, wherein the likelihood that the patient is experiencing the symptom is determined using a model trained using training data, wherein the training data comprises records of at least one of: historical patients with varying stages of kidney disease; or the patient over time.

Embodiment 14: The method of Embodiment 13, wherein the model is trained using training data prior to deployment of the model, when the model is deployed, or any combination thereof.

Embodiment 15: The method of Embodiment 2, further comprising: obtaining at least one of demographic information, food consumption information, activity level information, medication information, health and sickness information, or disease stage information related to the patient; and wherein the likelihood that the patient is experiencing the symptom is determined further using at least one of the food consumption information, the activity level information, the medication information, the health and sickness information, or the disease stage information related to the patient.

### Additional Considerations

The methods disclosed herein comprise one or more steps or actions for achieving the methods. The method steps and/or actions may be interchanged with one another without departing from the scope of the claims. In other words, unless a specific order of steps or actions is specified, the order and/or use of specific steps and/or actions may be modified without departing from the scope of the claims.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

The previous description is provided to enable any person skilled in the art to practice the various aspects described herein. Various modifications to these aspects will be readily apparent to those skilled in the art, and the generic principles defined herein may be applied to other aspects. Thus, the claims are not intended to be limited to the aspects shown herein, but is to be accorded the full scope consistent with the language of the claims, wherein reference to an element in the singular is not intended to mean “one and only one” unless specifically so stated, but rather “one or more.” Unless specifically stated otherwise, the term “some” refers to one or more. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112(f) unless the element is expressly recited using the phrase “means for” or, in the case of a method claim, the element is recited using the phrase “step for.”

While various examples of the invention have been described above, it should be understood that they have been presented by way of example only, and not by way of limitation. Likewise, the various diagrams may depict an example architectural or other configuration for the disclosure, which is done to aid in understanding the features and functionality that can be included in the disclosure. The disclosure is not restricted to the illustrated example architectures or configurations, but can be implemented using a variety of alternative architectures and configurations. Additionally, although the disclosure is described above in terms of various example examples and aspects, it should be understood that the various features and functionality described in one or more of the individual examples are not limited in their applicability to the particular example with which they are described. They instead can be applied, alone or in some combination, to one or more of the other examples of the disclosure, whether or not such examples are described, and whether or not such features are presented as being a part of a described example. Thus the breadth and scope of the present disclosure should not be limited by any of the above-described example examples.

All references cited herein are incorporated herein by reference in their entirety. To the extent publications and patents or patent applications incorporated by reference contradict the disclosure contained in the specification, the specification is intended to supersede and/or take precedence over any such contradictory material.

Unless otherwise defined, all terms (including technical and scientific terms) are to be given their ordinary and customary meaning to a person of ordinary skill in the art, and are not to be limited to a special or customized meaning unless expressly so defined herein.

Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide example instances of the item in discussion, not an exhaustive or limiting list thereof; adjectives such as ‘known’, ‘normal’, ‘standard’, and terms of similar meaning should not be construed as limiting the item described to a given time period or to an item available as of a given time, but instead should be read to encompass known, normal, or standard technologies that may be available or known now or at any time in the future; and use of terms like ‘preferably,’ ‘preferred,’ ‘desired,’ or ‘desirable,’ and words of similar meaning should not be understood as implying that certain features are critical, essential, or even important to the structure or function of the invention, but instead as merely intended to highlight alternative or additional features that may or may not be utilized in a particular example of the invention. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

The term “comprising as used herein is synonymous with “including,” “containing,” or “characterized by” and is inclusive or open-ended and does not exclude additional, unrecited elements or method steps.

All numbers expressing quantities of ingredients, reaction conditions, and so forth used in the specification are to be understood as being modified in all instances by the term ‘about.’ Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

Furthermore, although the foregoing has been described in some detail by way of illustrations and examples for purposes of clarity and understanding, it is apparent to those skilled in the art that certain changes and modifications may be practiced. Therefore, the description and examples should not be construed as limiting the scope of the invention to the specific examples and examples described herein, but rather to also cover all modification and alternatives coming with the true scope and spirit of the invention.

1. A monitoring system, comprising:
a continuous analyte sensor configured generate analyte measurements associated with analyte levels of a patient; and
a sensor electronics module coupled to the continuous analyte sensor and configured to receive and process the analyte measurements.

2. The monitoring system of claim 1, wherein the continuous analyte sensor comprises:
a substrate,
a working electrode disposed on the substrate,
a reference electrode disposed on the substrate, wherein the analyte measurements generated by the continuous analyte sensor correspond to an electromotive force at least in part based on a potential difference generated between the working electrode and the reference electrode.

3. The monitoring system of claim 1, wherein:
the continuous analyte sensor is a continuous potassium sensor, and
the analyte measurements include potassium measurements.

4. The monitoring system of claim 3, further comprising:
a memory comprising executable instructions;
one or more processors in data communication with the sensor electronics module and configured by the executable instructions to:
receive analyte data associated with the analyte measurements from the sensor electronics module, the analyte measurements associated with one or more analytes and generated by the continuous analyte sensor over a plurality of time periods, the analyte data comprising potassium data;
process the analyte data from the plurality of time periods to determine at least one rate of change of potassium for the patient based on the potassium data; and
generate a disease prediction using the analyte data for the one or more analytes, including the potassium data and the at least one rate of change of potassium for the patient.

5. The monitoring system of claim 4, wherein the one or more analytes of the patient are monitored continuously, semi-continuously, or periodically during the plurality of time periods.

6. The monitoring system of claim 4, wherein the disease prediction comprises at least one of:
an indication of a presence of one or more diseases in the patient;
an indication of a severity of the one or more diseases in the patient;
an indication of a level of risk of the patient being diagnosed with the one or more diseases; and
an indication of a level of improvement or deterioration of the one or more diseases in the patient.

7. The monitoring system of claim 6, wherein the indication of the level of improvement or the deterioration of the one or more diseases in the patient is based, at least in part, on at least one of:
a procedure previously performed on the patient;
a drug previously ingested by the patient;
one or more actions taken by the patient; or
one or more other clinical actions.

8. The monitoring system of claim 4, wherein the disease prediction comprises at least one of:
an indication of a level of risk of the patient being diagnosed with at least one of hyperkalemia or hypokalemia within a first threshold amount of time;
an indication of a level of risk of the patient experiencing a cardiac event within a second threshold amount of time; or
an indication of a level of risk of the patient experiencing a severe medical consequence due to potassium imbalance of the patient within a third threshold amount of time.

9. The monitoring system of claim 4, wherein the processor is further configured to generate one or more recommendations for treatment based, at least in part, on the disease prediction.

10. The monitoring system of claim 9, wherein the one or more recommendations for treatment are generated further based on at least one of:
a predicted effect of the one or more recommendations on one or more organs of the patient;
a predicted effect of the one or more recommendations on one or more conditions of the patient;
insulin levels of the patient; or
one or more other medications previously prescribed for the patient.

11. The monitoring system of claim 9, wherein the one or more recommendations for treatment comprise at least one of:
drug prescription recommendations;
medical supplement recommendations;
insulin dosage recommendations;
invasive or non-invasive procedure recommendations;
medical device recommendations for use by the patient;
lifestyle modification recommendations;
exercise recommendations; or
diet modification recommendations.

12. The monitoring system of claim 11, wherein the insulin dosage recommendation comprises a recommended combination dosage of insulin and glucose.

13. The monitoring system of claim 4, wherein the one or more analytes further include at least one of: glucose, creatinine, blood urea nitrogen (BUN), ammonia, C-peptide, and cystatin C.

14. The monitoring system of claim 4, wherein the processor is further configured to:
monitor other sensor data of the patient during the plurality of time periods using one or more other non-analyte sensors, wherein the disease prediction is further generated using the other sensor data.

15. The monitoring system of claim 14, wherein the one or more other non-analyte sensors comprise at least one of an insulin pump, an accelerometer, a temperature sensor, an electrocardiogram (ECG) sensor, a heart rate monitor, a blood pressure sensor, an impedance, a peritoneal dialysis machine, a hemodialysis machine, a continuous positive airway pressure machine, a body sound sensor, or a respiratory sensor.

16. The monitoring system of claim 4, wherein the disease prediction is generated using a model trained using training data, wherein the training data comprises records of historical patients with varying stages of kidney disease.

17. The monitoring system of claim 4, wherein the processor is further configured to:
obtain at least one of demographic information, food consumption information, activity level information, medication information, health and sickness information, or disease stage information related to the patient; and
wherein the disease prediction is generated further using at least one of the food consumption information, the activity level information, the medication information, the health and sickness information, or the disease stage information related to the patient.
